SEQUENCE LISTING (110 sequences)

SEQ ID NO: 1 - DNA, 1826 bp, Sus scrofa
aggtgctgggattaatgggaaaagttttggcaggggccggaggaccctgcggagcctgccggacggtggcggtggcgcggggggcagccaagtcagcgtcgtgtggtgttttgggtatgcacgtggtactcacacagtggctgctgttcaccgacagatgaggacagatgcaccaaagaggttgacaggaactgccctgtagaggtccatctgcactcagacccagatgatgccagagctatgaccgggcctgcaggcgtggcgccgaggggaagtcagccatggagcagccgccggaggaagcccctgaggtccgggaagaggagaagaaaaaggaagtggcagaggccgaaggaggcccagagctcaatgggggaccagagcactcgcttccctccagcagctgtacagatctctcccagagctgctctccacccgcgctgctggaccagctgcagatgggctgcgacggggcctcgtgcggtggcctcagcatggagtgccgggtgtgcggggacaaggcatcaggcttccactacggagtccacgcttgcgaggggtgcaagggcttcttccgccggacaatccgcatgaagctggagtacgagaagtgtgagcggatctgcaagatccagaagaagaaccgcaacaagtgccagtactgccgcttccagaaatgcctggcgctgggcatgtctcacaacgccattcgctttggccggatgcccgaggcagagaaaaggaagctggtggctgggctgacggcaaacgaggggagtcagcacaacccgcaggtggctgacctgaaggccttctccaagcacctctacagcgcctacctgaaaaacttcaacatgaccaaaaagaaggcccgcgccatcctcaccggcaaggccagccacacagcgccctttgtgatccacgacatcgagacgttgtggcaggccgagaagggcctggtgtggaagcagctggtgaatggcctgccgccctacaaggagatcagcgtgcacgtcttctaccgctgccagtgcaccacggtggagacggtgcgcgagctgaccgagttcgccaagagcatccccagcttcgaccacttcttcctcaacgaccaggtgacccttctcaagtacggcgtgcacgaggccatcttcgccatgctggcctccatcgtcaataaggatgggctgctggtggccaacggcactggttttgtcacccgcgagttcctgcgcagcatccgaaagcccttcagtgacatcattgagcccaagtttgagttcgctgtcaagttcaatgccctggaactcgatgatagtgacctggctctcttcatcgcagccatcattctgtgtggagaccggccaggcctcatgaacgtgtcacaggtggaggccatccaggacaccatcctgcgtgccctcgagttccacctgcaggccaaccaccccgacgcccagtacctcttccccaagctgctgcagaagatggcagacctgcggcagctggtcaccgagcacgcccagatgatgcagcggatcaagaagaccgagaccgagacctcgctgcaccccctgctccaggagatctacaaggacatgtactgaggggtgcgccttgggcctcccaacaggcctcccggagcaggtggacggcgcggggacagacactgcctgcgggacgtttccgttgaccagcccgagccctcagccgagcagcaggtcacaggctcagccagacgcacggcctcccactccttatagccctgcctcctctccctcctcagctcccctctctctcatctctttgctctttcttttccacct

SEQ ID NO: 2 - protein, 441 aa, Sus scrofa
MEQPPEEAPEVREEEKKKEVAEAEGGPELNGGPEHSLPSSSCTDLSQSCSPPALLDQLQMGCDGASCGGLSMECRVCGDKASGFHYGVHACEGCKGFFRRTIRMKLEYEKCERICKIQKKNRNKCQYCRFQKCLALGMSHNAIRFGRMPEAEKRKLVAGLTANEGSQHNPQVADLKAFSKHLYSAYLKNFNMTKKKARAILTGKASHTAPFVIHDIETLWQAEKGLVWKQLVNGLPPYKEISVHVFYRCQCTTVETVRELTEFAKSIPSFDHFFLNDQVTLLKYGVHEAIFAMLASIVNKDGLLVANGTGFVTREFLRSIRKPFSDIIEPKFEFAVKFNALELDDSDLALFIAAIILCGDRPGLMNVSQVEAIQDTILRALEFHLQANHPDAQYLFPKLLQKMADLRQLVTEHAQMMQRIKKTETETSLHPLLQEIYKDMY

SEQ ID NO: 3 - DNA, 3734 bp, Homo sapiens
gcggagcgtgtgacgctgcggccgccgcggacctggggattaatgggaaaagttttggcaggagcgggagaattctgcggagcctgcgggacggcggcggtggcgccgtaggcagccgggacagtgttgtacagtgttttgggcatgcacgtgatactcacacagtggcttctgctcaccaacagatgaagacagatgcaccaacgaggctgatgggaaccaccctgtagaggtccatctgcgttcagacccagacgatgccagagctatgactgggcctgcaggtgtggcgccgaggggagatcagccatggagcagccacaggaggaagcccctgaggtccgggaagaggaggagaaagaggaagtggcagaggcagaaggagccccagagctcaatgggggaccacagcatgcacttccttccagcagctacacagacctctcccggagctcctcgccaccctcactgctggaccaactgcagatgggctgtgacggggcctcatgcggcagcctcaacatggagtgccgggtgtgcggggacaaggcatcgggcttccactacggtgttcatgcatgtgaggggtgcaagggcttcttccgtcgtacgatccgcatgaagctggagtacgagaagtgtgagcgcagctgcaagattcagaagaagaaccgcaacaagtgccagtactgccgcttccagaagtgcctggcactgggcatgtcacacaacgctatccgttttggtcggatgccggaggctgagaagaggaagctggtggcagggctgactgcaaacgaggggagccagtacaacccacaggtggccgacctgaaggccttctccaagcacatctacaatgcctacctgaaaaacttcaacatgaccaaaaagaaggcccgcagcatcctcaccggcaaagccagccacacggcgccctttgtgatccacgacatcgagacattgtggcaggcagagaaggggctggtgtggaagcagttggtgaatggcctgcctccctacaaggagatcagcgtgcacgtcttctaccgctgccagtgcaccacagtggagaccgtgcgggagctcactgagttcgccaagagcatccccagcttcagcagcctcttcctcaacgaccaggttacccttctcaagtatggcgtgcacgaggccatcttcgccatgctggcctctatcgtcaacaaggacgggctgctggtagccaacggcagtggctttgtcacccgtgagttcctgcgcagcctccgcaaacccttcagtgatatcattgagcctaagtttgaatttgctgtcaagttcaacgccctggaacttgatgacagtgacctggccctattcattgcggccatcattctgtgtggagaccggccaggcctcatgaacgttccacgggtggaggctatccaggacaccatcctgcgtgccctcgaattccacctgcaggccaaccaccctgatgcccagtacctcttccccaagctgctgcagaagatggctgacctgcggcaactggtcaccgagcacgcccagatgatgcagcggatcaagaagaccgaaaccgagacctcgctgcaccctctgctccaggagatctacaaggacatgtactaacggcggcacccaggcctccctgcagactccaatggggccagcactggaggggcccacccacatgacttttccattgaccagcccttgagcacccggcctggagcagcagagtcccacgatcgccctcagacacatgacacccacggcctctggctccctgtgccctctctcccgcttcctccagccagctctcttcctgtctttgttgtctccctctttctcagttcctctttcttttctaattcctgttgctctgtttcttcctttctgtaggtttctctcttcccttctcccttgccctccctttctctctccaccccccacgtctgtcctcctttcttattctgtgagatgttttgtattatttcaccagcagcatagaacaggacctctgcttttgcacaccttttccccaggagcagaagagagtggggcctgccctctgccccatcattgcacctgcaggcttaggtcctcacttctgtctcctgtcttcagagcaaaagacttgagccatccaaagaaacactaagctctctgggcctgggttccagggaaggctaagcatggcctggactgactgcagccccctatagtcatggggtccctgctgcaaaggacagtgggcaggaggccccaggctgagagccagatgcctccccaagactgtcattgcccctccgatgctgaggccacccactgacccaactgatcctgctccagcagcacacctcagccccactgacacccagtgtccttccatcttcacactggtttgccaggccaatgttgctgatggccccctgcactggccgctggacggcactctcccagcttggaagtaggcagggttccctccaggtgggcccccacctcactgaagaggagcaagtctcaagagaaggaggggggattggtggttggaggaagcagcacacccaattctgcccctaggactcggggtctgagtcctggggtcaggccagggagagctcggggcaggccttccgccagcactcccactgcccccctgcccagtagcagccgcccacattgtgtcagcatccagggccagggcctggcctcacatccccctgctcctttctctagctggctccacgggagttcaggccccactccccctgaagctgcccctccagcacacacacataagcactgaaatcactttacctgcaggctccatgcacctcccttccctccctgaggcaggtgagaacccagagagaggggcctgcaggtgagcaggcagggctgggccaggtctccggggaggcaggggtcctgcaggtcctggtgggtcagcccagcacctgctcccagtgggagcttcccgggataaactgagcctgttcattctgatgtccatttgtcccaatagctctactgccctccccttcccctttactcagcccagctggccacctagaagtctccctgcacagcctctagtgtccggggaccttgtgggaccagtcccacaccgctggtccctgccctcccctgctcccaggttgaggtgcgctcacctcagagcagggccaaagcacagctgggcatgccatgtctgagcggcgcagagccctccaggcctgcaggggcaaggggctggctggagtctcagagcacagaggtaggagaactggggttcaagcccaggcttcctgggtcctgcctggtcctccctcccaaggagccattctgtgtgtgactctgggtggaagtgcccagcccctgcccctacgggcgctgcagcctcccttccatgccccaggatcactctctgctggcaggattcttcccgctccccacctacccagctgatgggggttggggtgcttcctttcaggccaaggctatgaagggacagctgctgggacccacctccccctccccggccacatgccgcgtccctgccccgacccgggtctggtgctgaggatacagctcttctcagtgtctgaacaatctccaaaattgaaatgtatatttttgctaggagccccagcttcctgtgtttttaatataaatagtgtacacagactgacgaaactttaaataaatgggaattaaatatttaa

SEQ ID NO: 4 - protein, 441 aa, Homo sapiens
MEQPQEEAPEVREEEEKEEVAEAEGAPELNGGPQHALPSSSYTDLSRSSSPPSLLDQLQMGCDGASCGSLNMECRVCGDKASGFHYGVHACEGCKGFFRRTIRMKLEYEKCERSCKIQKKNRNKCQYCRFQKCLALGMSHNAIRFGRMPEAEKRKLVAGLTANEGSQYNPQVADLKAFSKHIYNAYLKNFNMTKKKARSILTGKASHTAPFVIHDIETLWQAEKGLVWKQLVNGLPPYKEISVHVFYRCQCTTVETVRELTEFAKSIPSFSSLFLNDQVTLLKYGVHEAIFAMLASIVNKDGLLVANGSGFVTREFLRSLRKPFSDIIEPKFEFAVKFNALELDDSDLALFIAAIILCGDRPGLMNVPRVEAIQDTILRALEFHLQANHPDAQYLFPKLLQKMADLRQLVTEHAQMMQRIKKTETETSLHPLLQEIYKDMY

SEQ ID NO: 5 - DNA, 945 bp, Sus scrofa
atgttccagcccgcagagcccggccaggagtgggccatggaggggccccgggacgcgctcaagaaggagcggctactggatgaccgccacgacagcggcctggactccatgaaggacgaggagtacgagcagatggtgaaggagctgcgcgagatccgcctcgagccgcaggaggcgccccgcggcgccgagccctggaagcagcagctcaccgaggacggagactcgttcctgcacttggccatcatccatgaagagaaggcactgaccatggaagtggtccgccaagtgaagggagatctggcttttcttaacttccagaacaacctgcagcagactccactccacttggcggtgatcaccaaccagccagaaatcgctgaggcacttctggaagctggctgtgatcctgagctccgagactttcgaggaaatacccctctacaccttgcctgtgagcagggctgcctggccagtgtgggagtcctgactcagccccgcgggacccagcacctccactccattctgcaggccaccaactacaatggccacacatgtctgcacttagcctcgatccatggctacctgggcattgtggagctgttggtgtctttgggtgctgatgtcaacgctcaggagccctgcaatggccgaaccgccctgcatcttgcggtggacctgcagaatcccgacctggtgtcgctcttgttgaagtgtggggctgatgtcaacagagtcacctaccagggctactccccgtaccagctcacctggggccgcccaagcactcggatacagcagcagctgggccagctgaccctagaaaacctccagatgcttccagagagcgaggatgaggagagctatgacacggagtcagagttcacagaggatgagctgccctatgacgactgcgtgcttggaggccagcgcctgacgttatga

SEQ ID NO: 6 - protein, 314 aa, Sus scrofa
MFQPAEPGQEWAMEGPRDALKKERLLDDRHDSGLDSMKDEEYEQMVKELREIRLEPQEAPRGAEPWKQQLTEDGDSFLHLAIIHEEKALTMEVVRQVKGDLAFLNFQNNLQQTPLHLAVITNQPEIAEALLEAGCDPELRDFRGNTPLHLACEQGCLASVGVLTQPRGTQHLHSILQATNYNGHTCLHLASIHGYLGIVELLVSLGADVNAQEPCNGRTALHLAVDLQNPDLVSLLLKCGADVNRVTYQGYSPYQLTWGRPSTRIQQQLGQLTLENLQMLPESEDEESYDTESEFTEDELPYDDCVLGGQRLTL

SEQ ID NO: 7 - DNA, 1550 bp, Homo sapiens
tgccgccgtcccgcccgccagcgccccagcgaggaagcagcgcgcagcccgcggcccagcgcacccgcagcagcgcccgcagctcgtccgcgccatgttccaggcggccgagcgcccccaggagtgggccatggagggcccccgcgacgggctgaagaaggagcggctactggacgaccgccacgacagcggcctggactccatgaaagacgaggagtacgagcagatggtcaaggagctgcaggagatccgcctcgagccgcaggaggtgccgcgcggctcggagccctggaagcagcagctcaccgaggacggggactcgttcctgcacttggccatcatccatgaagaaaaggcactgaccatggaagtgatccgccaggtgaagggagacctggctttcctcaacttccagaacaacctgcagcagactccactccacttggctgtgatcaccaaccagccagaaattgctgaggcacttctgggagctggctgtgatcctgagctccgagactttcgaggaaatacccccctacaccttgcctgtgagcagggctgcctggccagcgtgggagtcctgactcagtcctgcaccaccccgcacctccactccatcctgaaggctaccaactacaatggccacacgtgtctacacttagcctctatccatggctacctgggcatcgtggagcttttggtgtccttgggtgctgatgtcaatgctcaggagccctgtaatggccggactgcccttcacctcgcagtggacctgcaaaatcctgacctggtgtcactcctgttgaagtgtggggctgatgtcaacagagttacctaccagggctattctccctaccagctcacctggggccgcccaagcacccggatacagcagcagctgggccagctgacactagaaaaccttcagatgctgccagagagtgaggatgaggagagctatgacacagagtcagagttcacggagttcacagaggacgagctgccctatgatgactgtgtgtttggaggccagcgtctgacgttatgagtgcaaaggggctgaaagaacatggacttgtatatttgtacaaaaaaaaagttttatttttctaaaaaaagaaaaaagaagaaaaaatttaaagggtgtacttatatccacactgcacactgcctagcccaaaacgtcttattgtggtaggatcagccctcattttgttgcttttgtgaactttttgtaggggacgagaaagatcattgaaattctgagaaaacttcttttaaacctcacctttgtggggtttttggagaaggttatcaaaaatttcatggaaggaccacattttatatttattgtgcttcgagtgactgaccccagtggtatcctgtgacatgtaacagccaggagtgttaagcgttcagtgatgtggggtgaaaagttactacctgtcaaggtttgtgttaccctcctgtaaatggtgtacataatgtattgttggtaattattttggtacttttatgatgtatatttattaaagagatttttacaaatg

SEQ ID NO: 8 - protein, 317 aa, Homo sapiens
MFQAAERPQEWAMEGPRDGLKKERLLDDRHDSGLDSMKDEEYEQMVKELQEIRLEPQEVPRGSEPWKQQLTEDGDSFLHLAIIHEEKALTMEVIRQVKGDLAFLNFQNNLQQTPLHLAVITNQPEIAEALLGAGCDPELRDFRGNTPLHLACEQGCLASVGVLTQSCTTPHLHSILKATNYNGHTCLHLASIHGYLGIVELLVSLGADVNAQEPCNGRTALHLAVDLQNPDLVSLLLKCGADVNRVTYQGYSPYQLTWGRPSTRIQQQLGQLTLENLQMLPESEDEESYDTESEFTEFTEDELPYDDCVFGGQRLTL

SEQ ID NO: 9 - DNA, 6307 bp, Artificial Sequence
  plasmid vector
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtaccctacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagaagcttctgatagactaattgacatcatttgagtcaattggaggtgtacctgtggatgtatttcaagggaattctgtggaatgtgtgtcagttagggtgtggaaagtccccaggctccccaggcaggcagaagtatgcaaagcatcgaggatgtacgggccagatatacgcgataacttcgtataatgtatgctatacgaagttatcgcgtgaggttttcaccgtcatcaccgaaacgcgcgaggcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggctaccatggagaagttactattccgaagttcctattctctagaaagtataggaacttcaagcttggcactggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcggccaattgggccaccggtgctagccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatgaccgagtacaagcccacggtgcgcctcgccacccgcgacgacgtcccccgggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgcccacaagacccgcagcgcccgaccgaaaggagcgcacgaccccatgcatcgaatcgatatcgcggccgcgactctagatcataatcagcccgggggtgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaaccagctggggctcgacattctagttgtggtttgtccaaactcatcaatgtatcttatcatgtctggatcccatcacaaagctctgacctcaatcctatagaaaggaggaatgagccaaaattcacccaacttattgtgggaagcttgtggaaggctactcgaaatgtttgacccaagttaaacaatttaaaggcaatgctaccaaatactaattgagtgtatgttaacttctgacccactgggaatgtgatgaaagaaataaaagctgaaatgaatcattctctctactattattctgatatttcacattcttaaaataaagtggtgatcctaactgaccttaagacagggaatctttactcggattaaatgtcaggaattgtgaaaaagtgagtttaaatgtatttggctaaggtgtatgtaaacttccgacttcaactgtagggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 10 - DNA, 6221 bp, Artificial Sequence
  Plasmid vector
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtaccctacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagaagcttctgatagactaattgacatcatttgagtcaattggaggtgtacctgtggatgtatttcaagggaattctgtggaatgtgtgtcagttagggtgtggaaagtccccaggctccccaggcaggcagaagtatgcaaagcatcgaggatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatacacttgtagtcttgcaacatggtaacgatgagttagcaacatgccttacaaggagagaaaaagcaccgtgcatgccgattggtggaagtaaggtggtacgatcgtgccttattaggaaggcaacagacaggtctgacatggattggacgaaccactgaattccgcattgcagagataattgtatttaagtgcctagctcgatacaataaacgccatttgaccattcaccacattggtgtgcacctccaaagcttgatatctaccatggagaagttactattccgaagttcctattctctagaaagtataggaacttcaagcttggcactggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcatagcggccgtaaattccgcccctctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataagcttgccacaaccatgaccgagtacaagcccacggtgcgcctcgccacccgcgacgacgtcccccgggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaagatcccccgggggatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaaccagctggggctcgacattctagttgtggtttgtccaaactcatcaatgtatcttatcatgtctggatcccatcacaaagctctgacctcaatcctatagaaaggaggaatgagccaaaattcacccaacttattgtgggaagcttgtggaaggctactcgaaatgtttgacccaagttaaacaatttaaaggcaatgctaccaaatactaattgagtgtatgttaacttctgacccactgggaatgtgatgaaagaaataaaagctgaaatgaatcattctctctactattattctgatatttcacattcttaaaataaagtggtgatcctaactgaccttaagacagggaatctttactcggattaaatgtcaggaattgtgaaaaagtgagtttaaatgtatttggctaaggtgtatgtaaacttccgacttcaactgtagggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 11 - DNA, 6269 bp, Artificial Sequence
  Plasmid vector
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtaccctacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagaagcttctgatagactaattgacatcatttgagtcaattggaggtgtacctgtggatgtatttcaagggaattctgtggaatgtgtgtcagttagggtgtggaaagtccccaggctccccaggcaggcagaagtatgcaaagcatcgaggatgtacgggccagatatacgcgtgaggttttcaccgtcatcaccgaaacgcgcgaggcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggctaccatggagaagttactattccgaagttcctattctctagaaagtataggaacttcaagcttggcactggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcggccaattgggccaccggtgctagccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatgaccgagtacaagcccacggtgcgcctcgccacccgcgacgacgtcccccgggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgcccacaagacccgcagcgcccgaccgaaaggagcgcacgaccccatgcatcgaatcgatatcgcggccgcgactctagatcataatcagcccgggggtgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaaccagctggggctcgacattctagttgtggtttgtccaaactcatcaatgtatcttatcatgtctggatcccatcacaaagctctgacctcaatcctatagaaaggaggaatgagccaaaattcacccaacttattgtgggaagcttgtggaaggctactcgaaatgtttgacccaagttaaacaatttaaaggcaatgctaccaaatactaattgagtgtatgttaacttctgacccactgggaatgtgatgaaagaaataaaagctgaaatgaatcattctctctactattattctgatatttcacattcttaaaataaagtggtgatcctaactgaccttaagacagggaatctttactcggattaaatgtcaggaattgtgaaaaagtgagtttaaatgtatttggctaaggtgtatgtaaacttccgacttcaactgtagggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 12 - DNA, 6346 bp, Artificial Sequence
  Plasmid vector
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtaccctacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagaagcttctgatagactaattgacatcatttgagtcaattggaggtgtacctgtggatgtatttcaagggaattctgtggaatgtgtgtcagttagggtgtggaaagtccccaggctccccaggcaggcagaagtatgcaaagcatcgaggatgtacgggccagatatacgcgataacttcgtataatgtatgctatacgaagttatcgcgtgaggttttcaccgtcatcaccgaaacgcgcgaggcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggctaccatggagaagttactattccgaagttcctattctctagaaagtataggaacttcaagcttggcactggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcggccaattgggccaccggtgctagccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatgaccgagtacaagcccacggtgcgcctcgccacccgcgacgacgtcccccgggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgcccacaagacccgcagcgcccgaccgaaaggagcgcacgaccccatgcatcgaatcgatatcgcggccgcgactctagatcataatcagcccgggggtgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaaccagctggggcgcgattaacttcgtataaagtctcctatacgaagttatcgcgccattctagttgtggtttgtccaaactcatcaatgtatcttatcatgtctggatcccatcacaaagctctgacctcaatcctatagaaaggaggaatgagccaaaattcacccaacttattgtgggaagcttgtggaaggctactcgaaatgtttgacccaagttaaacaatttaaaggcaatgctaccaaatactaattgagtgtatgttaacttctgacccactgggaatgtgatgaaagaaataaaagctgaaatgaatcattctctctactattattctgatatttcacattcttaaaataaagtggtgatcctaactgaccttaagacagggaatctttactcggattaaatgtcaggaattgtgaaaaagtgagtttaaatgtatttggctaaggtgtatgtaaacttccgacttcaactgtagggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 13 - DNA, 2473 bp, Homo sapiens
aagcttctccatgtgtcatgggatatagctcatccttattatgttgggtgggggttggacagttacccagacttgtcatgtggacctggagcttatgaggtcattcacataggcagtgaaagaacctctcccatatacgtgaatgcctgtctcccaaatggggcaacctgtgggcagaataagggacttctcagccctagaatgttgaggtttccccaacccctcccttgcatacacacacacacaaacactccctcagctgtatccactgccctctttcccacaccctagctttgcccagcagtcaaaggctcacacataccatcttctccttaaggctcttattatgccgtgagtcagagggcgggaggcagatctggcagatactgagcccctgctaacccataagaccggtgtgacttccttgatctgagtctgctgccccagactgactgtcacgggctgggaagaggcagattccccccagatgaagtcagcagcagagcacaagggcatcagcgccaaagtaaggatgcttgattagttcttcagggcagagtgggctgtgcttcctctgccccagaaaatggcacagtccctgttctatgggaaaaagaatgtgaggtccctgggtgggctcagggaacagagaggtcatgaggaggggatagcactgcagaaaccaagggtgccttgtgagtcctccctctgtctttttaggcatgatccaggaacatgacaaaattagtgctttaaatagatttacttgggctaagagaaatgtgcctgtcaggaaaactatggggaatcaggacacttctcaaaattagccccactgagtattgtctttataattccttctttttggattagattgtaaaaaagagagtgtaaatgaatgatgtccatataataagttattagccaaccattaaaaagaaagggaagaaataaatcagtttggtttttacacacacatacagacacacacatataaacattgatcaacactgaaatgtttaatagtcattattttcgggtcgtaaaattcactgttcttcaatgaatacttgtagagcacatattatatgcagtagttttgataggttctaggggtatagtggaaaacataccaggtatacgctgctcttagcttattttccagtgggaaagatagacaataagcaagtgaacaaatgcaaataaattactctagattgttataagtgaaattaagtaccaatcctttagatatggtacacagagaaggatctctgacagaccccaacattgacactgaagctgaaaggcataaaagaaccagagacctggggaggggccggtgggcagaaggagagcaggtgccaagcccccaggtggagagctctgggctcatctcaggaaccgaaggccctcagtgaggtaagaatatacctctcagggagagattgacatgaattggggccccagaagaaggcagaagccaggtacccagggtcttttaaaccacggcagtgagtttgaatgttatttcaagtgtgctggtgcactgttggcacgggggagagatgtgctcaaatccccactctgaaagatttcttaagctatttctagagtatgatttacaacaggaaatggatgatttgattctgatctttatgccttcatgcatttaaaaaagtacttaagaaagtagtttggtttgtcattataaaaagcaatacttatttttatattgtgtagattcaatcttgtttccttgcctagagtgggccgtgctttggagttcttatgagcatggcattcctgagaacttctctaactgcagcctcgggcatagaggctgggcagcaagtggcagcagcagaggactcctagaagccttctacttgactctacttggcctaaagtcaaactccctccaccaaagacagagtttatttccacataggatggagttaaaaaatatattctgagagaggaagggcttgtggcccaagagaacaccccagaaataccaccccttcatgggaagtgactctatcttcaaacatataacccagcctggacatccccgaaagacacataactttccatttcatgcccttgaaagtgaatcttttggcctaataatgagaacaaactcattttgaaagtggaaaaattgagattcagagcagaagtttgactaaggtcacaaaacagtaggatgcctcactcagctccctgtgcctaggtcagaaaagcatcacaggaatagttgagctaccagaatcctctggccaggcaggagctgtgtgtccctgggaaatggggccctaaagggtttgctgcttaagatgcctgtggtgagtcaggaaggggttagaggaagttgaccaactagagtggtgaaacctgtccatcaccttcaacctggagggaggccaggctgcagaatgatataaagagtgccctgactcctgctcagctc

SEQ ID NO: 14 - DNA, 5361 bp, Homo sapiens
ctgcaaacccagcgcaactacggtcccccggtcagacccaggatggggccagaacggacaggggccgcgccgctgccgctgctgctggtgttagcgctcagtcaaggcattttaaattgttgtttggcctacaatgttggtctcccagaagcaaaaatattttccggtccttcaagtgaacagtttgggtatgcagtgcagcagtttataaatccaaaaggcaactggttactggttggttcaccctggagtggctttcctgagaaccgaatgggagatgtgtataaatgtcctgttgacctatccactgccacatgtgaaaaactaaatttgcaaacttcaacaagcattccaaatgttactgagatgaaaaccaacatgagcctcggcttgatcctcaccaggaacatgggaactggaggttttctcacatgtggtcctctgtgggcacagcaatgtgggaatcagtattacacaacgggtgtgtgttctgacatcagtcctgattttcagctctcagccagcttctcacctgcaactcagccctgcccttccctcatagatgttgtggttgtgtgtgatgaatcaaatagtatttatccttgggatgcagtaaagaattttttggaaaaatttgtacaaggccttgatataggccccacaaagacacaggtggggttaattcagtatgccaataatccaagagttgtgtttaacttgaacacatataaaaccaaagaagaaatgattgtagcaacatcccagacatcccaatatggtggggacctcacaaacacattcggagcaattcaatatgcaagaaaatatgcctattcagcagcttctggtgggcgacgaagtgctacgaaagtaatggtagttgtaactgacggtgaatcacatgatggttcaatgttgaaagctgtgattgatcaatgcaaccatgacaatatactgaggtttggcatagcagttcttgggtacttaaacagaaacgcccttgatactaaaaatttaataaaagaaataaaagcgatcgctagtattccaacagaaagatactttttcaatgtgtctgatgaagcagctctactagaaaaggctgggacattaggagaacaaattttcagcattgaaggtactgttcaaggaggagacaactttcagatggaaatgtcacaagtgggattcagtgcagattactcttctcaaaatgatattctgatgctgggtgcagtgggagcttttggctggagtgggaccattgtccagaagacatctcatggccatttgatctttcctaaacaagcctttgaccaaattctgcaggacagaaatcacagttcatatttaggttactctgtggctgcaatttctactggagaaagcactcactttgttgctggtgctcctcgggcaaattataccggccagatagtgctatatagtgtgaatgagaatggcaatatcacggttattcaggctcaccgaggtgaccagattggctcctattttggtagtgtgctgtgttcagttgatgtggataaagacaccattacagacgtgctcttggtaggtgcaccaatgtacatgagtgacctaaagaaagaggaaggaagagtctacctgtttactatcaaaaagggcattttgggtcagcaccaatttcttgaaggccccgagggcattgaaaacactcgatttggttcagcaattgcagctctttcagacatcaacatggatggctttaatgatgtgattgttggttcaccactagaaaatcagaattctggagctgtatacatttacaatggtcatcagggcactatccgcacaaagtattcccagaaaatcttgggatccgatggagcctttaggagccatctccagtactttgggaggtccttggatggctatggagatttaaatggggattccatcaccgatgtgtctattggtgcctttggacaagtggttcaactctggtcacaaagtattgctgatgtagctatagaagcttcattcacaccagaaaaaatcactttggtcaacaagaatgctcagataattctcaaactctgcttcagtgcaaagttcagacctactaagcaaaacaatcaagtggccattgtatataacatcacacttgatgcagatggattttcatccagagtaacctccagggggttatttaaagaaaacaatgaaaggtgcctgcagaagaatatggtagtaaatcaagcacagagttgccccgagcacatcatttatatacaggagccctctgatgttgtcaactctttggatttgcgtgtggacatcagtctggaaaaccctggcactagccctgcccttgaagcctattctgagactgccaaggtcttcagtattcctttccacaaagactgtggtgaggatggactttgcatttctgatctagtcctagatgtccgacaaataccagctgctcaagaacaaccctttattgtcagcaaccaaaacaaaaggttaacattttcagtaacactgaaaaataaaagggaaagtgcatacaacactggaattgttgttgatttttcagaaaacttgttttttgcatcattctccctaccggttgatgggacagaagtaacatgccaggtggctgcatctcagaagtctgttgcctgcgatgtaggctaccctgctttaaagagagaacaacaggtgacttttactattaactttgacttcaatcttcaaaaccttcagaatcaggcgtctctcagtttccaagccttaagtgaaagccaagaagaaaacaaggctgataatttggtcaacctcaaaattcctctcctgtatgatgctgaaattcacttaacaagatctaccaacataaatttttatgaaatctcttcggatgggaatgttccttcaatcgtgcacagttttgaagatgttggtccaaaattcatcttctccctgaaggtaacaacaggaagtgttccagtaagcatggcaactgtaatcatccacatccctcagtataccaaagaaaagaacccactgatgtacctaactggggtgcaaacagacaaggctggtgacatcagttgtaatgcagatatcaatccactgaaaataggacaaacatcttcttctgtatctttcaaaagtgaaaatttcaggcacaccaaagaattgaactgcagaactgcttcctgtagtaatgttacctgctggttgaaagacgttcacatgaaaggagaatactttgttaatgtgactaccagaatttggaacgggactttcgcatcatcaacgttccagacagtacagctaacggcagctgcagaaatcaacacctataaccctgagatatatgtgattgaagataacactgttacgattcccctgatgataatgaaacctgatgagaaagccgaagtaccaacaggagttataataggaagtataattgctggaatccttttgctgttagctctggttgcaattttatggaagctcggcttcttcaaaagaaaatatgaaaagatgaccaaaaatccagatgagattgatgagaccacagagctcagtagctgaaccagcagacctacctgcagtgggaaccggcagcatcccagccagggtttgctgtttgcgtgcatggatttctttttaaatcccatattttttttatcatgtcgtaggtaaactaacctggtattttaagagaaaactgcaggtcagtttggatgaagaaattgtggggggtgggggaggtgcggggggcaggtagggaaataatagggaaaatacctattttatatgatgggggaaaaaaagtaatctttaaactggctggcccagagtttacattctaatttgcattgtgtcagaaacatgaaatgcttccaagcatgacaacttttaaagaaaaatatgatactctcagattttaagggggaaaactgttctctttaaaatatttgtctttaaacagcaactacagaagtggaagtgcttgatatgtaagtacttccacttgtgtatattttaatgaatattgatgttaacaagaggggaaaacaaaacacaggttttttcaatttatgctgctcatccaaagttgccacagatgatacttccaagtgataattttatttataaactaggtaaaatttgttgttggttccttttataccacggctgccccttccacaccccatcttgctctaatgatcaaaacatgcttgaataactgagcttagagtatacctcctatatgtccatttaagttaggagagggggcgatatagagactaaggcacaaaattttgtttaaaactcagaatataacatttatgtaaaatcccatctgctagaagcccatcctgtgccagaggaaggaaaaggaggaaatttcctttctcttttaggaggcacaacagttctcttctaggatttgtttggctgactggcagtaacctagtgaatttttgaaagatgagtaatttctttggcaaccttcctcctcccttactgaaccactctcccacctcctggtggtaccattattatagaagccctctacagcctgactttctctccagcggtccaaagttatcccctcctttacccctcatccaaagttcccactccttcaggacagctgctgtgcattagatattaggggggaaagtcatctgtttaatttacacacttgcatgaattactgtatataaactccttaacttcagggagctattttcatttagtgctaaacaagtaagaaaaataagctagagtgaatttctaaatgttggaatgttatgggatgtaaacaatgtaaagtaaaacactctcaggatttcaccagaagttacagatgaggcactggaaaccaccaccaaattagcaggtgcaccttctgtggctgtcttgtttctgaagtactttttcttccacaagagtgaatttgacctaggcaagtttgttcaaaaggtagatcctgagatgatttggtcagattgggataaggcccagcaatctgcattttaacaagcaccccagtcactaggatgcagatggaccacactttgagaaacaccacccatttctactttttgcaccttattttctctgttcctgagcccccacattctctaggagaaacttagattaaaattcacagacactacatatctaaagctttgacaagtccttgacctctataaacttcagagtcctcattataaaatgggaagactgagctggagttcagcagtgatgctttttagttttaaaagtctatgatctgatctggacttcctataatacaaatacacaatcctccaagaatttgacttggaaaag

SEQ ID NO: 15 - DNA, 3546 bp, Homo sapiens
atggggccagaacggacaggggccgcgccgctgccgctgctgctggtgttagcgctcagtcaaggcattttaaattgttgtttggcctacaatgttggtctcccagaagcaaaaatattttccggtccttcaagtgaacagtttgggtatgcagtgcagcagtttataaatccaaaaggcaactggttactggttggttcaccctggagtggctttcctgagaaccgaatgggagatgtgtataaatgtcctgttgacctatccactgccacatgtgaaaaactaaatttgcaaacttcaacaagcattccaaatgttactgagatgaaaaccaacatgagcctcggcttgatcctcaccaggaacatgggaactggaggttttctcacatgtggtcctctgtgggcacagcaatgtgggaatcagtattacacaacgggtgtgtgttctgacatcagtcctgattttcagctctcagccagcttctcacctgcaactcagccctgcccttccctcatagatgttgtggttgtgtgtgatgaatcaaatagtatttatccttgggatgcagtaaagaattttttggaaaaatttgtacaaggccttgatataggccccacaaagacacaggtggggttaattcagtatgccaataatccaagagttgtgtttaacttgaacacatataaaaccaaagaagaaatgattgtagcaacatcccagacatcccaatatggtggggacctcacaaacacattcggagcaattcaatatgcaagaaaatatgcctattcagcagcttctggtgggcgacgaagtgctacgaaagtaatggtagttgtaactgacggtgaatcacatgatggttcaatgttgaaagctgtgattgatcaatgcaaccatgacaatatactgaggtttggcatagcagttcttgggtacttaaacagaaacgcccttgatactaaaaatttaataaaagaaataaaagcgatcgctagtattccaacagaaagatactttttcaatgtgtctgatgaagcagctctactagaaaaggctgggacattaggagaacaaattttcagcattgaaggtactgttcaaggaggagacaactttcagatggaaatgtcacaagtgggattcagtgcagattactcttctcaaaatgatattctgatgctgggtgcagtgggagcttttggctggagtgggaccattgtccagaagacatctcatggccatttgatctttcctaaacaagcctttgaccaaattctgcaggacagaaatcacagttcatatttaggttactctgtggctgcaatttctactggagaaagcactcactttgttgctggtgctcctcgggcaaattataccggccagatagtgctatatagtgtgaatgagaatggcaatatcacggttattcaggctcaccgaggtgaccagattggctcctattttggtagtgtgctgtgttcagttgatgtggataaagacaccattacagacgtgctcttggtaggtgcaccaatgtacatgagtgacctaaagaaagaggaaggaagagtctacctgtttactatcaaaaagggcattttgggtcagcaccaatttcttgaaggccccgagggcattgaaaacactcgatttggttcagcaattgcagctctttcagacatcaacatggatggctttaatgatgtgattgttggttcaccactagaaaatcagaattctggagctgtatacatttacaatggtcatcagggcactatccgcacaaagtattcccagaaaatcttgggatccgatggagcctttaggagccatctccagtactttgggaggtccttggatggctatggagatttaaatggggattccatcaccgatgtgtctattggtgcctttggacaagtggttcaactctggtcacaaagtattgctgatgtagctatagaagcttcattcacaccagaaaaaatcactttggtcaacaagaatgctcagataattctcaaactctgcttcagtgcaaagttcagacctactaagcaaaacaatcaagtggccattgtatataacatcacacttgatgcagatggattttcatccagagtaacctccagggggttatttaaagaaaacaatgaaaggtgcctgcagaagaatatggtagtaaatcaagcacagagttgccccgagcacatcatttatatacaggagccctctgatgttgtcaactctttggatttgcgtgtggacatcagtctggaaaaccctggcactagccctgcccttgaagcctattctgagactgccaaggtcttcagtattcctttccacaaagactgtggtgaggatggactttgcatttctgatctagtcctagatgtccgacaaataccagctgctcaagaacaaccctttattgtcagcaaccaaaacaaaaggttaacattttcagtaacactgaaaaataaaagggaaagtgcatacaacactggaattgttgttgatttttcagaaaacttgttttttgcatcattctccctaccggttgatgggacagaagtaacatgccaggtggctgcatctcagaagtctgttgcctgcgatgtaggctaccctgctttaaagagagaacaacaggtgacttttactattaactttgacttcaatcttcaaaaccttcagaatcaggcgtctctcagtttccaagccttaagtgaaagccaagaagaaaacaaggctgataatttggtcaacctcaaaattcctctcctgtatgatgctgaaattcacttaacaagatctaccaacataaatttttatgaaatctcttcggatgggaatgttccttcaatcgtgcacagttttgaagatgttggtccaaaattcatcttctccctgaaggtaacaacaggaagtgttccagtaagcatggcaactgtaatcatccacatccctcagtataccaaagaaaagaacccactgatgtacctaactggggtgcaaacagacaaggctggtgacatcagttgtaatgcagatatcaatccactgaaaataggacaaacatcttcttctgtatctttcaaaagtgaaaatttcaggcacaccaaagaattgaactgcagaactgcttcctgtagtaatgttacctgctggttgaaagacgttcacatgaaaggagaatactttgttaatgtgactaccagaatttggaacgggactttcgcatcatcaacgttccagacagtacagctaacggcagctgcagaaatcaacacctataaccctgagatatatgtgattgaagataacactgttacgattcccctgatgataatgaaacctgatgagaaagccgaagtaccaacaggagttataataggaagtataattgctggaatccttttgctgttagctctggttgcaattttatggaagctcggcttcttcaaaagaaaatatgaaaagatgaccaaaaatccagatgagattgatgagaccacagagctcagtagctga

SEQ ID NO: 16 - protein, 1181 aa, Homo sapiens
MGPERTGAAPLPLLLVLALSQGILNCCLAYNVGLPEAKIFSGPSSEQFGYAVQQFINPKGNWLLVGSPWSGFPENRMGDVYKCPVDLSTATCEKLNLQTSTSIPNVTEMKTNMSLGLILTRNMGTGGFLTCGPLWAQQCGNQYYTTGVCSDISPDFQLSASFSPATQPCPSLIDVVVVCDESNSIYPWDAVKNFLEKFVQGLDIGPTKTQVGLIQYANNPRVVFNLNTYKTKEEMIVATSQTSQYGGDLTNTFGAIQYARKYAYSAASGGRRSATKVMVVVTDGESHDGSMLKAVIDQCNHDNILRFGIAVLGYLNRNALDTKNLIKEIKAIASIPTERYFFNVSDEAALLEKAGTLGEQIFSIEGTVQGGDNFQMEMSQVGFSADYSSQNDILMLGAVGAFGWSGTIVQKTSHGHLIFPKQAFDQILQDRNHSSYLGYSVAAISTGESTHFVAGAPRANYTGQIVLYSVNENGNITVIQAHRGDQIGSYFGSVLCSVDVDKDTITDVLLVGAPMYMSDLKKEEGRVYLFTIKKGILGQHQFLEGPEGIENTRFGSAIAALSDINMDGFNDVIVGSPLENQNSGAVYIYNGHQGTIRTKYSQKILGSDGAFRSHLQYFGRSLDGYGDLNGDSITDVSIGAFGQVVQLWSQSIADVAIEASFTPEKITLVNKNAQIILKLCFSAKFRPTKQNNQVAIVYNITLDADGFSSRVTSRGLFKENNERCLQKNMVVNQAQSCPEHIIYIQEPSDVVNSLDLRVDISLENPGTSPALEAYSETAKVFSIPFHKDCGEDGLCISDLVLDVRQIPAAQEQPFIVSNQNKRLTFSVTLKNKRESAYNTGIVVDFSENLFFASFSLPVDGTEVTCQVAASQKSVACDVGYPALKREQQVTFTINFDFNLQNLQNQASLSFQALSESQEENKADNLVNLKIPLLYDAEIHLTRSTNINFYEISSDGNVPSIVHSFEDVGPKFIFSLKVTTGSVPVSMATVIIHIPQYTKEKNPLMYLTGVQTDKAGDISCNADINPLKIGQTSSSVSFKSENFRHTKELNCRTASCSNVTCWLKDVHMKGEYFVNVTTRIWNGTFASSTFQTVQLTAAAEINTYNPEIYVIEDNTVTIPLMIMKPDEKAEVPTGVIIGSIIAGILLLLALVAILWKLGFFKRKYEKMTKNPDEIDETTELSS

SEQ ID NO: 17 - DNA, 3700 bp, Homo sapiens
agccgccgccacccgccgcgcccgacacccgggaggccccgccagcccgcgggagaggcccagcgggagtcgcggaacagcaggcccgagcccaccgcgccgggccccggacgccgcgcggaaaagatgaatttacaaccaattttctggattggactgatcagttcagtttgctgtgtgtttgctcaaacagatgaaaatagatgtttaaaagcaaatgccaaatcatgtggagaatgtatacaagcagggccaaattgtgggtggtgcacaaattcaacatttttacaggaaggaatgcctacttctgcacgatgtgatgatttagaagccttaaaaaagaagggttgccctccagatgacatagaaaatcccagaggctccaaagatataaagaaaaataaaaatgtaaccaaccgtagcaaaggaacagcagagaagctcaagccagaggatattactcagatccaaccacagcagttggttttgcgattaagatcaggggagccacagacatttacattaaaattcaagagagctgaagactatcccattgacctctactaccttatggacctgtcttactcaatgaaagacgatttggagaatgtaaaaagtcttggaacagatctgatgaatgaaatgaggaggattacttcggacttcagaattggatttggctcatttgtggaaaagactgtgatgccttacattagcacaacaccagctaagctcaggaacccttgcacaagtgaacagaactgcaccagcccatttagctacaaaaatgtgctcagtcttactaataaaggagaagtatttaatgaacttgttggaaaacagcgcatatctggaaatttggattctccagaaggtggtttcgatgccatcatgcaagttgcagtttgtggatcactgattggctggaggaatgttacacggctgctggtgttttccacagatgccgggtttcactttgctggagatgggaaacttggtggcattgttttaccaaatgatggacaatgtcacctggaaaataatatgtacacaatgagccattattatgattatccttctattgctcaccttgtccagaaactgagtgaaaataatattcagacaatttttgcagttactgaagaatttcagcctgtttacaaggagctgaaaaacttgatccctaagtcagcagtaggaacattatctgcaaattctagcaatgtaattcagttgatcattgatgcatacaattccctttcctcagaagtcattttggaaaacggcaaattgtcagaaggagtaacaataagttacaaatcttactgcaagaacggggtgaatggaacaggggaaaatggaagaaaatgttccaatatttccattggagatgaggttcaatttgaaattagcataacttcaaataagtgtccaaaaaaggattctgacagctttaaaattaggcctctgggctttacggaggaagtagaggttattcttcagtacatctgtgaatgtgaatgccaaagcgaaggcatccctgaaagtcccaagtgtcatgaaggaaatgggacatttgagtgtggcgcgtgcaggtgcaatgaagggcgtgttggtagacattgtgaatgcagcacagatgaagttaacagtgaagacatggatgcttactgcaggaaagaaaacagttcagaaatctgcagtaacaatggagagtgcgtctgcggacagtgtgtttgtaggaagagggataatacaaatgaaatttattctggcaaattctgcgagtgtgataatttcaactgtgatagatccaatggcttaatttgtggaggaaatggtgtttgcaagtgtcgtgtgtgtgagtgcaaccccaactacactggcagtgcatgtgactgttctttggatactagtacttgtgaagccagcaacggacagatctgcaatggccggggcatctgcgagtgtggtgtctgtaagtgtacagatccgaagtttcaagggcaaacgtgtgagatgtgtcagacctgccttggtgtctgtgctgagcataaagaatgtgttcagtgcagagccttcaataaaggagaaaagaaagacacatgcacacaggaatgttcctattttaacattaccaaggtagaaagtcgggacaaattaccccagccggtccaacctgatcctgtgtcccattgtaaggagaaggatgttgacgactgttggttctattttacgtattcagtgaatgggaacaacgaggtcatggttcatgttgtggagaatccagagtgtcccactggtccagacatcattccaattgtagctggtgtggttgctggaattgttcttattggccttgcattactgctgatatggaagcttttaatgataattcatgacagaagggagtttgctaaatttgaaaaggagaaaatgaatgccaaatgggacacgggtgaaaatcctatttataagagtgccgtaacaactgtggtcaatccgaagtatgagggaaaatgagtactgcccgtgcaaatcccacaacactgaatgcaaagtagcaatttccatagtcacagttaggtagctttagggcaatattgccatggttttactcatgtgcaggttttgaaaatgtacaatatgtataatttttaaaatgttttattattttgaaaataatgttgtaattcatgccagggactgacaaaagacttgagacaggatggttattcttgtcagctaaggtcacattgtgcctttttgaccttttcttcctggactattgaaatcaagcttattggattaagtgatatttctatagcgattgaaagggcaatagttaaagtaatgagcatgatgagagtttctgttaatcatgtattaaaactgatttttagctttacaaatatgtcagtttgcagttatgcagaatccaaagtaaatgtcctgctagctagttaaggattgttttaaatctgttattttgctatttgcctgttagacatgactgatgacatatctgaaagacaagtatgttgagagttgctggtgtaaaatacgtttgaaatagttgatctacaaaggccatgggaaaaattcagagagttaggaaggaaaaaccaatagctttaaaacctgtgtgccattttaagagttacttaatgtttggtaacttttatgccttcactttacaaattcaagccttagataaaagaaccgagcaattttctgctaaaaagtccttgatttagcactatttacatacaggccatactttacaaagtatttgctgaatggggaccttttgagttgaatttattttattatttttattttgtttaatgtctggtgctttctatcacctcttctaatcttttaatgtatttgtttgcaattttggggtaagacttttttatgagtactttttctttgaagttttagcggtcaatttgcctttttaatgaacatgtgaagttatactgtggctatgcaacagctctcacctacgcgagtcttactttgagttagtgccataacagaccactgtatgtttacttctcaccatttgagttgcccatcttgtttcacactagtcacattcttgttttaagtgcctttagttttaacagttcactttttacagtgctatttactgaagttatttattaaatatgcctaaaatacttaaatcgga

SEQ ID NO: 18 - DNA, 3691 bp, Homo sapiens
gaattcggcacgagggcgggagaggcccagcgggagtcgcggaacagcaggcccgagcccaccgcgccgggccccggacgccgcgcggaaaagatgaatttacaaccaattttctggattggactgatcagttcagtttgctgtgtgtttgctcaaacagatgaaaatagatgtttaaaagcaaatgccaaatcatgtggagaatgtatacaagcagggccaaattgtgggtggtgcacaaattcaacatttttacaggaaggaatgcctacttctgcacgatgtgatgatttagaagccttaaaaaagaagggttgccctccagatgacatagaaaatcccagaggctccaaagatataaagaaaaataaaaatgtaaccaaccgtagcaaaggaacagcagagaagctcaagccagaggatattactcagatccaaccacagcagttggttttgcgattaagatcaggggagccacagacatttacattaaaattcaagagagctgaagactatcccattgacctctactaccttatggacctgtcttactcaatgaaagacgatttggagaatgtaaaaagtcttggaacagatctgatgaatgaaatgaggaggattacttcggacttcagaattggatttggctcatttgtggaaaagactgtgatgccttacattagcacaacaccagctaagctcaggaacccttgcacaagtgaacagaactgcaccagcccatttagctacaaaaatgtgctcagtcttactaataaaggagaagtatttaatgaacttgttggaaaacagcgcatatctggaaatttggattctccagaaggtggtttcgatgccatcatgcaagttgcagtttgtggatcactgattggctggaggaatgttacacggctgctggtgttttccacagatgccgggtttcactttgctggagatgggaaacttggtggcattgttttaccaaatgatggacaatgtcacctggaaaataatatgtacacaatgagccattattatgattatccttctattgctcaccttgtccagaaactgagtgaaaataatattcagacaatttttgcagttactgaagaatttcagcctgtttacaaggagctgaaaaacttgatccctaagtcagcagtaggaacattatctgcaaattctagcaatgtaattcagttgatcattgatgcatacaattccctttcctcagaagtcattttggaaaacggcaaattgtcagaaggagtaacaataagttacaaatcttactgcaagaacggggtgaatggaacaggggaaaatggaagaaaatgttccaatatttccattggagatgaggttcaatttgaaattagcataacttcaaataagtgtccaaaaaaggattctgacagctttaaaattaggcctctgggctttacggaggaagtagaggttattcttcagtacatctgtgaatgtgaatgccaaagcgaaggcatccctgaaagtcccaagtgtcatgaaggaaatgggacatttgagtgtggcgcgtgcaggtgcaatgaagggcgtgttggtagacattgtgaatgcagcacagatgaagttaacagtgaagacatggatgcttactgcaggaaagaaaacagttcagaaatctgcagtaacaatggagagtgcgtctgcggacagtgtgtttgtaggaagagggataatacaaatgaaatttattctggcaaattctgcgagtgtgataatttcaactgtgatagatccaatggcttaatttgtggaggaaatggtgtttgcaagtgtcgtgtgtgtgagtgcaaccccaactacactggcagtgcatgtgactgttctttggatactagtacttgtgaagccagcaacggacagatctgcaatggccggggcatctgtgagtgtggtgtctgtaagtgtacagatccgaagtttcaagggcaaacgtgtgagatgtgtcagacctgccttggtgtctgtgctgagcataaagaatgtgttcagtgcagagccttcaataaaggagaaaagaaagacacatgcacacaggaatgttcctattttaacattaccaaggtagaaagtcgggacaaattaccccagccggtccaacctgatcctgtgtcccattgtaaggagaaggatgttgacgactgttggttctattttacgtattcagtgaatgggaacaacgaggtcatggttcatgttgtggagaatccagagtgtcccactggtccagacatcattccaattgtagctggtgtggttgctggaattgttcttattggccttgcattactgctgatatggaagcttttaatgataattcatgacagaagggagtttgctaaatttgaaaaggagaaaatgaatgccaaatgggacacgggtgaaaatcctatttataagagtgccgtaacaactgtggtcaatccgaagtatgagggaaaatgagtactgcccgtgcaaatcccacaacactgaatgcgaagtagcaatttccatagtcacagttaggtagctttagggcaatattgccatggttttactcatgtgcaggttttgaaaatgtacaatatgtataatttttaaaatgttttattattttgaaaataatgttgtaattcatgccagggactgacaaaagacttgagacaggatggttattcttgtcagctaaggtcacattgtgcctttttgaccttttcttcctggactattgaaatcaagcttattggattaagtgatatttctatagcgattgaaagggcaatagttaaagtaatgagcatgatgagagtttctgttaatcatgtattaaaactgatttttagctttacaaatatgtcagtttgcagttatgcagaatccaaagtaaatgtcctgctagctagttaaggattgttttaaatctgttattttgctatttgcctgttagacatgactgatgacatatctgaaagacaagtatgttgagagttgctggtgtaaaatacgtttgaaatagttgatctacaaaggccatgggaaaaattcagagagttaggaaggaaaaaccaatagctttaaaacctgtgtgccattttaagagttacttaatgtttggtaacttttatgccttcactttacaaattcaagccttagataaaagaaccgagcaattttctgctaaaaagtccttgatttagcactatttacatacaggccatactttacaaagtatttgctgaatggggaccttttgagttgaatttattttattatttttattttgtttaatgtctggtgctttctatcacctcttctaatcttttaatgtatttgtttgcaattttggggtaagactttttttatgagtactttttctttgaagttttagcggtcaatttgcctttttaatgaacatgtgaagttatactgtggctatgcaacagctctcacctacgcgagtcttactttgagttagtgccataacagaccactgtatgtttacttctcaccatttgagttgcccatcttgtttcacactagtcacattcttgttttaagtgcctttagttttaacagttcactttttacagtgctatttactgaagttatttattaaatatgcctaaaatacttaaatcggaaaaaaaaaaaaaaaaaactcgac

SEQ ID NO: 19 - DNA, 2397 bp, Homo sapiens
atgaatttacaaccaattttctggattggactgatcagttcagtttgctgtgtgtttgctcaaacagatgaaaatagatgtttaaaagcaaatgccaaatcatgtggagaatgtatacaagcagggccaaattgtgggtggtgcacaaattcaacatttttacaggaaggaatgcctacttctgcacgatgtgatgatttagaagccttaaaaaagaagggttgccctccagatgacatagaaaatcccagaggctccaaagatataaagaaaaataaaaatgtaaccaaccgtagcaaaggaacagcagagaagctcaagccagaggatattactcagatccaaccacagcagttggttttgcgattaagatcaggggagccacagacatttacattaaaattcaagagagctgaagactatcccattgacctctactaccttatggacctgtcttactcaatgaaagacgatttggagaatgtaaaaagtcttggaacagatctgatgaatgaaatgaggaggattacttcggacttcagaattggatttggctcatttgtggaaaagactgtgatgccttacattagcacaacaccagctaagctcaggaacccttgcacaagtgaacagaactgcaccagcccatttagctacaaaaatgtgctcagtcttactaataaaggagaagtatttaatgaacttgttggaaaacagcgcatatctggaaatttggattctccagaaggtggtttcgatgccatcatgcaagttgcagtttgtggatcactgattggctggaggaatgttacacggctgctggtgttttccacagatgccgggtttcactttgctggagatgggaaacttggtggcattgttttaccaaatgatggacaatgtcacctggaaaataatatgtacacaatgagccattattatgattatccttctattgctcaccttgtccagaaactgagtgaaaataatattcagacaatttttgcagttactgaagaatttcagcctgtttacaaggagctgaaaaacttgatccctaagtcagcagtaggaacattatctgcaaattctagcaatgtaattcagttgatcattgatgcatacaattccctttcctcagaagtcattttggaaaacggcaaattgtcagaaggagtaacaataagttacaaatcttactgcaagaacggggtgaatggaacaggggaaaatggaagaaaatgttccaatatttccattggagatgaggttcaatttgaaattagcataacttcaaataagtgtccaaaaaaggattctgacagctttaaaattaggcctctgggctttacggaggaagtagaggttattcttcagtacatctgtgaatgtgaatgccaaagcgaaggcatccctgaaagtcccaagtgtcatgaaggaaatgggacatttgagtgtggcgcgtgcaggtgcaatgaagggcgtgttggtagacattgtgaatgcagcacagatgaagttaacagtgaagacatggatgcttactgcaggaaagaaaacagttcagaaatctgcagtaacaatggagagtgcgtctgcggacagtgtgtttgtaggaagagggataatacaaatgaaatttattctggcaaattctgcgagtgtgataatttcaactgtgatagatccaatggcttaatttgtggaggaaatggtgtttgcaagtgtcgtgtgtgtgagtgcaaccccaactacactggcagtgcatgtgactgttctttggatactagtacttgtgaagccagcaacggacagatctgcaatggccggggcatctgcgagtgtggtgtctgtaagtgtacagatccgaagtttcaagggcaaacgtgtgagatgtgtcagacctgccttggtgtctgtgctgagcataaagaatgtgttcagtgcagagccttcaataaaggagaaaagaaagacacatgcacacaggaatgttcctattttaacattaccaaggtagaaagtcgggacaaattaccccagccggtccaacctgatcctgtgtcccattgtaaggagaaggatgttgacgactgttggttctattttacgtattcagtgaatgggaacaacgaggtcatggttcatgttgtggagaatccagagtgtcccactggtccagacatcattccaattgtagctggtgtggttgctggaattgttcttattggccttgcattactgctgatatggaagcttttaatgataattcatgacagaagggagtttgctaaatttgaaaaggagaaaatgaatgccaaatgggacacgggtgaaaatcctatttataagagtgccgtaacaactgtggtcaatccgaagtatgagggaaaatga

SEQ ID NO: 20 - protein, 798 aa, Homo sapiens
MNLQPIFWIGLISSVCCVFAQTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGKFCECDNFNCDRSNGLICGGNGVCKCRVCECNPNYTGSACDCSLDTSTCEASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPDIIPIVAGVVAGIVLIGLALLLIWKLLMIIHDRREFAKFEKEKMNAKWDTGENPIYKSAVTTVVNPKYEGK

SEQ ID NO: 21 - DNA, 6765 bp, Homo sapiens
caaggcgattaagttgggtaacgccagggttttcccagtcacgacgttgtaaaacgacggccagtgagcgcgcgtaatacgactcactatagggcgaattggagctcggatccctatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagaagcttcccacaataagttgggtgaattttggctcattcctcctttctataggattgaggtcagagctttgtgatgggaattcgtttaaacgtcgacggcgcgcctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcggcgcgccgcggccgcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcggccgcttaattaagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggattaattaagaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttggatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctggatcccttgaaatacatccacaggtacacctccaattgactcaaatgatgtcaattagtctatcagaagcttgtggaaggctactcgaaatgtttgacccaagttaaacaatttaaaggcaatgctaccaaatactaattgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtatagggatcctctagctagagtcgacctcgagggggggcccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctg

SEQ ID NO: 22 - DNA, 4953 bp, Homo sapiens
ggtttccggagctgcggcggcgcagactgggagggggagccgggggttccgacgtcgcagccgagggaacaagccccaaccggatcctggacaggcaccccggcttggcgctgtctctccccctcggctcggagaggcccttcggcctgagggagcctcgccgcccgtccccggcacacgcgcagccccggcctctcggcctctgccggagaaacaggatggcccaatggaatcagctacagcagcttgacacacggtacctggagcagctccatcagctctacagtgacagcttcccaatggagctgcggcagtttctggccccttggattgagagtcaagattgggcatatgcggccagcaaagaatcacatgccactttggtgtttcataatctcctgggagagattgaccagcagtatagccgcttcctgcaagagtcgaatgttctctatcagcacaatctacgaagaatcaagcagtttcttcagagcaggtatcttgagaagccaatggagattgcccggattgtggcccggtgcctgtgggaagaatcacgccttctacagactgcagccactgcggcccagcaagggggccaggccaaccaccccacagcagccgtggtgacggagaagcagcagatgctggagcagcaccttcaggatgtccggaagagagtgcaggatctagaacagaaaatgaaagtggtagagaatctccaggatgactttgatttcaactataaaaccctcaagagtcaaggagacatgcaagatctgaatggaaacaaccagtcagtgaccaggcagaagatgcagcagctggaacagatgctcactgcgctggaccagatgcggagaagcatcgtgagtgagctggcggggcttttgtcagcgatggagtacgtgcagaaaactctcacggacgaggagctggctgactggaagaggcggcaacagattgcctgcattggaggcccgcccaacatctgcctagatcggctagaaaactggataacgtcattagcagaatctcaacttcagacccgtcaacaaattaagaaactggaggagttgcagcaaaaagtttcctacaaaggggaccccattgtacagcaccggccgatgctggaggagagaatcgtggagctgtttagaaacttaatgaaaagtgcctttgtggtggagcggcagccctgcatgcccatgcatcctgaccggcccctcgtcatcaagaccggcgtccagttcactactaaagtcaggttgctggtcaaattccctgagttgaattatcagcttaaaattaaagtgtgcattgacaaagactctggggacgttgcagctctcagaggatcccggaaatttaacattctgggcacaaacacaaaagtgatgaacatggaagaatccaacaacggcagcctctctgcagaattcaaacacttgaccctgagggagcagagatgtgggaatgggggccgagccaattgtgatgcttccctgattgtgactgaggagctgcacctgatcacctttgagaccgaggtgtatcaccaaggcctcaagattgacctagagacccactccttgccagttgtggtgatctccaacatctgtcagatgccaaatgcctgggcgtccatcctgtggtacaacatgctgaccaacaatcccaagaatgtaaacttttttaccaagcccccaattggaacctgggatcaagtggccgaggtcctgagctggcagttctcctccaccaccaagcgaggactgagcatcgagcagctgactacactggcagagaaactcttgggacctggtgtgaattattcagggtgtcagatcacatgggctaaattttgcaaagaaaacatggctggcaagggcttctccttctgggtctggctggacaatatcattgaccttgtgaaaaagtacatcctggccctttggaacgaagggtacatcatgggctttatcagtaaggagcgggagcgggccatcttgagcactaagcctccaggcaccttcctgctaagattcagtgaaagcagcaaagaaggaggcgtcactttcacttgggtggagaaggacatcagcggtaagacccagatccagtccgtggaaccatacacaaagcagcagctgaacaacatgtcatttgctgaaatcatcatgggctataagatcatggatgctaccaatatcctggtgtctccactggtctatctctatcctgacattcccaaggaggaggcattcggaaagtattgtcggccagagagccaggagcatcctgaagctgacccaggcgctgccccatacctgaagaccaagtttatctgtgtgacaccaacgacctgcagcaataccattgacctgccgatgtccccccgcactttagattcattgatgcagtttggaaataatggtgaaggtgctgaaccctcagcaggagggcagtttgagtccctcacctttgacatggagttgacctcggagtgcgctacctcccccatgtgaggagctgagaacggaagctgcagaaagatacgactgaggcgcctacctgcattctgccacccctcacacagccaaaccccagatcatctgaaactactaactttgtggttccagattttttttaatctcctacttctgctatctttgagcaatctgggcacttttaaaaatagagaaatgagtgaatgtgggtgatctgcttttatctaaatgcaaataaggatgtgttctctgagacccatgatcaggggatgtggcggggggtggctagagggagaaaaaggaaatgtcttgtgttgttttgttcccctgccctcctttctcagcagctttttgttattgttgttgttgttcttagacaagtgcctcctggtgcctgcggcatccttctgcctgtttctgtaagcaaatgccacaggccacctatagctacatactcctggcattgcactttttaaccttgctgacatccaaatagaagataggactatctaagccctaggtttctttttaaattaagaaataataacaattaaagggcaaaaaacactgtatcagcatagcctttctgtatttaagaaacttaagcagccgggcatggtggctcacgcctgtaatcccagcactttgggaggccgaggcggatcataaggtcaggagatcaagaccatcctggctaacacggtgaaaccccgtctctactaaaagtacaaaaaattagctgggtgtggtggtgggcgcctgtagtcccagctactcgggaggctgaggcaggagaatcgcttgaacctgagaggcggaggttgcagtgagccaaaattgcaccactgcacactgcactccatcctgggcgacagtctgagactctgtctcaaaaaaaaaaaaaaaaaaaagaaacttcagttaacagcctccttggtgctttaagcattcagcttccttcaggctggtaatttatataatccctgaaacgggcttcaggtcaaacccttaagacatctgaagctgcaacctggcctttggtgttgaaataggaaggtttaaggagaatctaagcattttagacttttttttataaatagacttattttcctttgtaatgtattggccttttagtgagtaaggctgggcagagggtgcttacaaccttgactccctttctccctggacttgatctgctgtttcagaggctaggttgtttctgtgggtgccttatcagggctgggatacttctgattctggcttccttcctgccccaccctcccgaccccagtccccctgatcctgctagaggcatgtctccttgcgtgtctaaaggtccctcatcctgtttgttttaggaatcctggtctcaggacctcatggaagaagagggggagagagttacaggttggacatgatgcacactatggggccccagcgacgtgtctggttgagctcagggaatatggttcttagccagtttcttggtgatatccagtggcacttgtaatggcgtcttcattcagttcatgcagggcaaaggcttactgataaacttgagtctgccctcgtatgagggtgtatacctggcctccctctgaggctggtgactcctccctgctggggccccacaggtgaggcagaacagctagagggcctccccgcctgcccgccttggctggctagctcgcctctcctgtgcgtatgggaacacctagcacgtgctggatgggctgcctctgactcagaggcatggccggatttggcaactcaaaaccaccttgcctcagctgatcagagtttctgtggaattctgtttgttaaatcaaattagctggtctctgaattaagggggagacgaccttctctaagatgaacagggttcgccccagtcctcctgcctggagacagttgatgtgtcatgcagagctcttacttctccagcaacactcttcagtacataataagcttaactgataaacagaatatttagaaaggtgagacttgggcttaccattgggtttaaatcatagggacctagggcgagggttcagggcttctctggagcagatattgtcaagttcatggccttaggtagcatgtatctggtcttaactctgattgtagcaaaagttctgagaggagctgagccctgttgtggcccattaaagaacagggtcctcaggccctgcccgcttcctgtccactgccccctccccatccccagcccagccgagggaatcccgtgggttgcttacctacctataaggtggtttataagctgctgtcctggccactgcattcaaattccaatgtgtacttcatagtgtaaaaatttatattattgtgaggttttttgtcttttttttttttttttttttttggtatattgctgtatctactttaacttccagaaataaacgttatataggaaccgtaaaaa

SEQ ID NO: 23 - DNA, 4978 bp, Homo sapiens
ggtttccggagctgcggcggcgcagactgggagggggagccgggggttccgacgtcgcagccgagggaacaagccccaaccggatcctggacaggcaccccggcttggcgctgtctctccccctcggctcggagaggcccttcggcctgagggagcctcgccgcccgtccccggcacacgcgcagccccggcctctcggcctctgccggagaaacagttgggacccctgattttagcaggatggcccaatggaatcagctacagcagcttgacacacggtacctggagcagctccatcagctctacagtgacagcttcccaatggagctgcggcagtttctggccccttggattgagagtcaagattgggcatatgcggccagcaaagaatcacatgccactttggtgtttcataatctcctgggagagattgaccagcagtatagccgcttcctgcaagagtcgaatgttctctatcagcacaatctacgaagaatcaagcagtttcttcagagcaggtatcttgagaagccaatggagattgcccggattgtggcccggtgcctgtgggaagaatcacgccttctacagactgcagccactgcggcccagcaagggggccaggccaaccaccccacagcagccgtggtgacggagaagcagcagatgctggagcagcaccttcaggatgtccggaagagagtgcaggatctagaacagaaaatgaaagtggtagagaatctccaggatgactttgatttcaactataaaaccctcaagagtcaaggagacatgcaagatctgaatggaaacaaccagtcagtgaccaggcagaagatgcagcagctggaacagatgctcactgcgctggaccagatgcggagaagcatcgtgagtgagctggcggggcttttgtcagcgatggagtacgtgcagaaaactctcacggacgaggagctggctgactggaagaggcggcaacagattgcctgcattggaggcccgcccaacatctgcctagatcggctagaaaactggataacgtcattagcagaatctcaacttcagacccgtcaacaaattaagaaactggaggagttgcagcaaaaagtttcctacaaaggggaccccattgtacagcaccggccgatgctggaggagagaatcgtggagctgtttagaaacttaatgaaaagtgcctttgtggtggagcggcagccctgcatgcccatgcatcctgaccggcccctcgtcatcaagaccggcgtccagttcactactaaagtcaggttgctggtcaaattccctgagttgaattatcagcttaaaattaaagtgtgcattgacaaagactctggggacgttgcagctctcagaggatcccggaaatttaacattctgggcacaaacacaaaagtgatgaacatggaagaatccaacaacggcagcctctctgcagaattcaaacacttgaccctgagggagcagagatgtgggaatgggggccgagccaattgtgatgcttccctgattgtgactgaggagctgcacctgatcacctttgagaccgaggtgtatcaccaaggcctcaagattgacctagagacccactccttgccagttgtggtgatctccaacatctgtcagatgccaaatgcctgggcgtccatcctgtggtacaacatgctgaccaacaatcccaagaatgtaaacttttttaccaagcccccaattggaacctgggatcaagtggccgaggtcctgagctggcagttctcctccaccaccaagcgaggactgagcatcgagcagctgactacactggcagagaaactcttgggacctggtgtgaattattcagggtgtcagatcacatgggctaaattttgcaaagaaaacatggctggcaagggcttctccttctgggtctggctggacaatatcattgaccttgtgaaaaagtacatcctggccctttggaacgaagggtacatcatgggctttatcagtaaggagcgggagcgggccatcttgagcactaagcctccaggcaccttcctgctaagattcagtgaaagcagcaaagaaggaggcgtcactttcacttgggtggagaaggacatcagcggtaagacccagatccagtccgtggaaccatacacaaagcagcagctgaacaacatgtcatttgctgaaatcatcatgggctataagatcatggatgctaccaatatcctggtgtctccactggtctatctctatcctgacattcccaaggaggaggcattcggaaagtattgtcggccagagagccaggagcatcctgaagctgacccaggtagcgctgccccatacctgaagaccaagtttatctgtgtgacaccaacgacctgcagcaataccattgacctgccgatgtccccccgcactttagattcattgatgcagtttggaaataatggtgaaggtgctgaaccctcagcaggagggcagtttgagtccctcacctttgacatggagttgacctcggagtgcgctacctcccccatgtgaggagctgagaacggaagctgcagaaagatacgactgaggcgcctacctgcattctgccacccctcacacagccaaaccccagatcatctgaaactactaactttgtggttccagattttttttaatctcctacttctgctatctttgagcaatctgggcacttttaaaaatagagaaatgagtgaatgtgggtgatctgcttttatctaaatgcaaataaggatgtgttctctgagacccatgatcaggggatgtggcggggggtggctagagggagaaaaaggaaatgtcttgtgttgttttgttcccctgccctcctttctcagcagctttttgttattgttgttgttgttcttagacaagtgcctcctggtgcctgcggcatccttctgcctgtttctgtaagcaaatgccacaggccacctatagctacatactcctggcattgcactttttaaccttgctgacatccaaatagaagataggactatctaagccctaggtttctttttaaattaagaaataataacaattaaagggcaaaaaacactgtatcagcatagcctttctgtatttaagaaacttaagcagccgggcatggtggctcacgcctgtaatcccagcactttgggaggccgaggcggatcataaggtcaggagatcaagaccatcctggctaacacggtgaaaccccgtctctactaaaagtacaaaaaattagctgggtgtggtggtgggcgcctgtagtcccagctactcgggaggctgaggcaggagaatcgcttgaacctgagaggcggaggttgcagtgagccaaaattgcaccactgcacactgcactccatcctgggcgacagtctgagactctgtctcaaaaaaaaaaaaaaaaaaaagaaacttcagttaacagcctccttggtgctttaagcattcagcttccttcaggctggtaatttatataatccctgaaacgggcttcaggtcaaacccttaagacatctgaagctgcaacctggcctttggtgttgaaataggaaggtttaaggagaatctaagcattttagacttttttttataaatagacttattttcctttgtaatgtattggccttttagtgagtaaggctgggcagagggtgcttacaaccttgactccctttctccctggacttgatctgctgtttcagaggctaggttgtttctgtgggtgccttatcagggctgggatacttctgattctggcttccttcctgccccaccctcccgaccccagtccccctgatcctgctagaggcatgtctccttgcgtgtctaaaggtccctcatcctgtttgttttaggaatcctggtctcaggacctcatggaagaagagggggagagagttacaggttggacatgatgcacactatggggccccagcgacgtgtctggttgagctcagggaatatggttcttagccagtttcttggtgatatccagtggcacttgtaatggcgtcttcattcagttcatgcagggcaaaggcttactgataaacttgagtctgccctcgtatgagggtgtatacctggcctccctctgaggctggtgactcctccctgctggggccccacaggtgaggcagaacagctagagggcctccccgcctgcccgccttggctggctagctcgcctctcctgtgcgtatgggaacacctagcacgtgctggatgggctgcctctgactcagaggcatggccggatttggcaactcaaaaccaccttgcctcagctgatcagagtttctgtggaattctgtttgttaaatcaaattagctggtctctgaattaagggggagacgaccttctctaagatgaacagggttcgccccagtcctcctgcctggagacagttgatgtgtcatgcagagctcttacttctccagcaacactcttcagtacataataagcttaactgataaacagaatatttagaaaggtgagacttgggcttaccattgggtttaaatcatagggacctagggcgagggttcagggcttctctggagcagatattgtcaagttcatggccttaggtagcatgtatctggtcttaactctgattgtagcaaaagttctgagaggagctgagccctgttgtggcccattaaagaacagggtcctcaggccctgcccgcttcctgtccactgccccctccccatccccagcccagccgagggaatcccgtgggttgcttacctacctataaggtggtttataagctgctgtcctggccactgcattcaaattccaatgtgtacttcatagtgtaaaaatttatattattgtgaggttttttgtcttttttttttttttttttttttggtatattgctgtatctactttaacttccagaaataaacgttatataggaaccgtaaaaa

SEQ ID NO: 24 - DNA, 4819 bp, Homo sapiens
cgctgtctctccccctcggctcggagaggcccttcggcctgagggagcctcgccgcccgtccccggcacacgcgcagccccggcctctcggcctctgccggagaaacagttgggacccctgattttagcaggatggcccaatggaatcagctacagcagcttgacacacggtacctggagcagctccatcagctctacagtgacagcttcccaatggagctgcggcagtttctggccccttggattgagagtcaagattgggcatatgcggccagcaaagaatcacatgccactttggtgtttcataatctcctgggagagattgaccagcagtatagccgcttcctgcaagagtcgaatgttctctatcagcacaatctacgaagaatcaagcagtttcttcagagcaggtatcttgagaagccaatggagattgcccggattgtggcccggtgcctgtgggaagaatcacgccttctacagactgcagccactgcggcccagcaagggggccaggccaaccaccccacagcagccgtggtgacggagaagcagcagatgctggagcagcaccttcaggatgtccggaagagagtgcaggatctagaacagaaaatgaaagtggtagagaatctccaggatgactttgatttcaactataaaaccctcaagagtcaaggagacatgcaagatctgaatggaaacaaccagtcagtgaccaggcagaagatgcagcagctggaacagatgctcactgcgctggaccagatgcggagaagcatcgtgagtgagctggcggggcttttgtcagcgatggagtacgtgcagaaaactctcacggacgaggagctggctgactggaagaggcggcaacagattgcctgcattggaggcccgcccaacatctgcctagatcggctagaaaactggataacgtcattagcagaatctcaacttcagacccgtcaacaaattaagaaactggaggagttgcagcaaaaagtttcctacaaaggggaccccattgtacagcaccggccgatgctggaggagagaatcgtggagctgtttagaaacttaatgaaaagtgcctttgtggtggagcggcagccctgcatgcccatgcatcctgaccggcccctcgtcatcaagaccggcgtccagttcactactaaagtcaggttgctggtcaaattccctgagttgaattatcagcttaaaattaaagtgtgcattgacaaagactctggggacgttgcagctctcagaggatcccggaaatttaacattctgggcacaaacacaaaagtgatgaacatggaagaatccaacaacggcagcctctctgcagaattcaaacacttgaccctgagggagcagagatgtgggaatgggggccgagccaattgtgatgcttccctgattgtgactgaggagctgcacctgatcacctttgagaccgaggtgtatcaccaaggcctcaagattgacctagagacccactccttgccagttgtggtgatctccaacatctgtcagatgccaaatgcctgggcgtccatcctgtggtacaacatgctgaccaacaatcccaagaatgtaaacttttttaccaagcccccaattggaacctgggatcaagtggccgaggtcctgagctggcagttctcctccaccaccaagcgaggactgagcatcgagcagctgactacactggcagagaaactcttgggacctggtgtgaattattcagggtgtcagatcacatgggctaaattttgcaaagaaaacatggctggcaagggcttctccttctgggtctggctggacaatatcattgaccttgtgaaaaagtacatcctggccctttggaacgaagggtacatcatgggctttatcagtaaggagcgggagcgggccatcttgagcactaagcctccaggcaccttcctgctaagattcagtgaaagcagcaaagaaggaggcgtcactttcacttgggtggagaaggacatcagcggtaagacccagatccagtccgtggaaccatacacaaagcagcagctgaacaacatgtcatttgctgaaatcatcatgggctataagatcatggatgctaccaatatcctggtgtctccactggtctatctctatcctgacattcccaaggaggaggcattcggaaagtattgtcggccagagagccaggagcatcctgaagctgacccaggtagcgctgccccatacctgaagaccaagtttatctgtgtgacaccattcattgatgcagtttggaaataatggtgaaggtgctgaaccctcagcaggagggcagtttgagtccctcacctttgacatggagttgacctcggagtgcgctacctcccccatgtgaggagctgagaacggaagctgcagaaagatacgactgaggcgcctacctgcattctgccacccctcacacagccaaaccccagatcatctgaaactactaactttgtggttccagattttttttaatctcctacttctgctatctttgagcaatctgggcacttttaaaaatagagaaatgagtgaatgtgggtgatctgcttttatctaaatgcaaataaggatgtgttctctgagacccatgatcaggggatgtggcggggggtggctagagggagaaaaaggaaatgtcttgtgttgttttgttcccctgccctcctttctcagcagctttttgttattgttgttgttgttcttagacaagtgcctcctggtgcctgcggcatccttctgcctgtttctgtaagcaaatgccacaggccacctatagctacatactcctggcattgcactttttaaccttgctgacatccaaatagaagataggactatctaagccctaggtttctttttaaattaagaaataataacaattaaagggcaaaaaacactgtatcagcatagcctttctgtatttaagaaacttaagcagccgggcatggtggctcacgcctgtaatcccagcactttgggaggccgaggcggatcataaggtcaggagatcaagaccatcctggctaacacggtgaaaccccgtctctactaaaagtacaaaaaattagctgggtgtggtggtgggcgcctgtagtcccagctactcgggaggctgaggcaggagaatcgcttgaacctgagaggcggaggttgcagtgagccaaaattgcaccactgcacactgcactccatcctgggcgacagtctgagactctgtctcaaaaaaaaaaaaaaaaaaaagaaacttcagttaacagcctccttggtgctttaagcattcagcttccttcaggctggtaatttatataatccctgaaacgggcttcaggtcaaacccttaagacatctgaagctgcaacctggcctttggtgttgaaataggaaggtttaaggagaatctaagcattttagacttttttttataaatagacttattttcctttgtaatgtattggccttttagtgagtaaggctgggcagagggtgcttacaaccttgactccctttctccctggacttgatctgctgtttcagaggctaggttgtttctgtgggtgccttatcagggctgggatacttctgattctggcttccttcctgccccaccctcccgaccccagtccccctgatcctgctagaggcatgtctccttgcgtgtctaaaggtccctcatcctgtttgttttaggaatcctggtctcaggacctcatggaagaagagggggagagagttacaggttggacatgatgcacactatggggccccagcgacgtgtctggttgagctcagggaatatggttcttagccagtttcttggtgatatccagtggcacttgtaatggcgtcttcattcagttcatgcagggcaaaggcttactgataaacttgagtctgccctcgtatgagggtgtatacctggcctccctctgaggctggtgactcctccctgctggggccccacaggtgaggcagaacagctagagggcctccccgcctgcccgccttggctggctagctcgcctctcctgtgcgtatgggaacacctagcacgtgctggatgggctgcctctgactcagaggcatggccggatttggcaactcaaaaccaccttgcctcagctgatcagagtttctgtggaattctgtttgttaaatcaaattagctggtctctgaattaagggggagacgaccttctctaagatgaacagggttcgccccagtcctcctgcctggagacagttgatgtgtcatgcagagctcttacttctccagcaacactcttcagtacataataagcttaactgataaacagaatatttagaaaggtgagacttgggcttaccattgggtttaaatcatagggacctagggcgagggttcagggcttctctggagcagatattgtcaagttcatggccttaggtagcatgtatctggtcttaactctgattgtagcaaaagttctgagaggagctgagccctgttgtggcccattaaagaacagggtcctcaggccctgcccgcttcctgtccactgccccctccccatccccagcccagccgagggaatcccgtgggttgcttacctacctataaggtggtttataagctgctgtcctggccactgcattcaaattccaatgtgtacttcatagtgtaaaaatttatattattgtgaggttttttgtcttttttttttttttttttttttggtatattgctgtatctactttaacttccagaaataaacgttatataggaaccgtaaaa

SEQ ID NO: 25 - DNA, 4437 bp, Mus musculus
tggcggctgggtggggattggctggaggggctgtaattcagcggtttccggagctgcagtgtagacagggagggggaacctggggttccgacgtcgcggcggagggaacgagccctaaccggatcgctgaggtacaaccccgctcggtgtcgcctgaccgcgtcggctaggagaggccaggcggccctcgggagcccagcagctcgcgcctggagtcagcgcaggccggccagtcgggcctcagccccggagacagtcgagacccctgactgcagcaggatggctcagtggaaccagctgcagcagctggacacacgctacctggagcagctgcaccagctgtacagcgacagcttccccatggagctgcggcagttcctggcaccttggattgagagtcaagactgggcatatgcagccagcaaagagtcacatgccacgttggtgtttcataatctcttgggtgaaattgaccagcaatatagccgattcctgcaagagtccaatgtcctctatcagcacaaccttcgaagaatcaagcagtttctgcagagcaggtatcttgagaagccaatggaaattgcccggatcgtggcccgatgcctgtgggaagagtctcgcctcctccagacggcagccacggcagcccagcaagggggccaggccaaccacccaacagccgccgtagtgacagagaagcagcagatgttggagcagcatcttcaggatgtccggaagcgagtgcaggatctagaacagaaaatgaaggtggtggagaacctccaggacgactttgatttcaactacaaaaccctcaagagccaaggagacatgcaggatctgaatggaaacaaccagtctgtgaccagacagaagatgcagcagctggaacagatgctcacagccctggaccagatgcggagaagcattgtgagtgagctggcggggctcttgtcagcaatggagtacgtgcagaagacactgactgatgaagagctggctgactggaagaggcggcagcagatcgcgtgcatcggaggccctcccaacatctgcctggaccgtctggaaaactggataacttcattagcagaatctcaacttcagacccgccaacaaattaagaaactggaggagctgcagcagaaagtgtcctacaagggcgaccctatcgtgcagcaccggcccatgctggaggagaggatcgtggagctgttcagaaacttaatgaagagtgccttcgtggtggagcggcagccctgcatgcccatgcacccggaccggcccttagtcatcaagactggtgtccagtttaccacgaaagtcaggttgctggtcaaatttcctgagttgaattatcagcttaaaattaaagtgtgcattgataaagactctggggatgttgctgccctcagagggtctcggaaatttaacattctgggcacgaacacaaaagtgatgaacatggaggagtctaacaacggcagcctgtctgcagagttcaagcacctgacccttagggagcagagatgtgggaatggaggccgtgccaattgtgatgcctccttgatcgtgactgaggagctgcacctgatcaccttcgagactgaggtgtaccaccaaggcctcaagattgacctagagacccactccttgccagttgtggtgatctccaacatctgtcagatgccaaatgcttgggcatcaatcctgtggtataacatgctgaccaataaccccaagaacgtgaacttcttcactaagccgccaattggaacctgggaccaagtggccgaggtgctcagctggcagttctcgtccaccaccaagcgggggctgagcatcgagcagctgacaacgctggctgagaagctcctagggcctggtgtgaactactcagggtgtcagatcacatgggctaaattctgcaaagaaaacatggctggcaagggcttctccttctgggtctggctagacaatatcatcgaccttgtgaaaaagtatatcttggccctttggaatgaagggtacatcatgggtttcatcagcaaggagcgggagcgggccatcctaagcacaaagcccccgggcaccttcctactgcgcttcagcgagagcagcaaagaaggaggggtcactttcacttgggtggaaaaggacatcagtggcaagacccagatccagtctgtagagccatacaccaagcagcagctgaacaacatgtcatttgctgaaatcatcatgggctataagatcatggatgcgaccaacatcctggtgtctccacttgtctacctctaccccgacattcccaaggaggaggcatttggaaagtactgtaggcccgagagccaggagcaccccgaagccgacccaggtagtgctgccccgtacctgaagaccaagttcatctgtgtgacaccattcattgatgcagtttggaaataacggtgaaggtgctgagccctcagcaggagggcagtttgagtcgctcacgtttgacatggatctgacctcggagtgtgctacctcccccatgtgaggagctgaaaccagaagctgcagagacgtgacttgagacacctgccccgtgctccacccctaagcagccgaaccccatatcgtctgaaactcctaactttgtggttccagatttttttttttaatttcctacttctgctatctttgggcaatctgggcactttttaaaatagagaaatgagtgagtgtgggtgataaactgttatgtaaagaggagagcacctctgagtctggggatggggctgagagcagaagggagcaaggggaacacctcctgtcctgcccgcctgccctcctttttcagcagctcggggttggttgttagacaagtgcctcctggtgcccatggcatcctgttgccccactctgtgagctgataccccaggctgggaactcctggctctgcactttcaaccttgctaatatccacatagaagctaggactaagcccagaggttcctctttaaattaaaaaaaaaaaaaataagaattaaagggcaaaacacactgacacagcatagcctttccatatcaaggaatactcagttaacagcctctccagcgctgtcttcaggctgatcatctatataaaccctggaatggttgcagatcaaatctgtaaaagagatccgagagctgtggcttggcctctggttcaaacacaaaggctagagagaacctagatatccctgggttttgtttacccagtatgcttgtcggttggaggtgtgaggtaggccaagggcactggaaagcctttgtcatcaccctactccctccccaacccagactccagaccctgtttcagggtcagcctgccctgtgggtgccttactgggcctagggtcaacctgccttcctttcccacttgaccttgctggtagtatgtccccttcccatgtccaaaggccctctgtcctgcttctattgggaatccctgcctcaggaccttgtgtcgagagggattgccttacaggtttgaacctgcctcagactacaggccctcagcaaagctcagggagtatggtccttattctatgcgcttggttcccagggatatctgtaaccacagggcaaaagctgacatatactccaggtctgccctcatatgagtggtgtattcttggcctcccctgagactggcaactgtctgctccccattgggtctcccaggtgaggtggaacacagttcctgcacctactgtggcctccatgtcgcttgcttgcttcgctcactcagcttactggaacactgagtgttcaaggcaagcctttcctgacagaggcatggctagattcagtgactcaaagccacctcattcagctgatcagtgtctgtggaattgtttccttccagttaaccagtgtctgaattaagggcagtgaggacattgtctccaagacgaactgcttgccttgaccaccccagccttctgcttcgagacagttactgctctcccaccccatcaatgttctttagttatacaataagctgaacttataaactgaaagggtatttaggaaggcaaggcttgggcatttttatggctttcaatcctggggacccaggaacaaggtgagggcttctctggggctggtgttgtacctcaggggctctgggaagtctgtgtgcctgggttaaccacccatagtgagcccctggaactgcccactttccctctccttggccccacttggccccagcctcacccagcctgcagactgcttagcctttcagtgcagtggcttgtgttctggccactgcactcagattccaatgtaaactttctagtgtaaaaatttatattattgtgggttgttttttgttgttgtttgtttttgtatattgctgtaactactttaacttccagaaataaagattatataggaactgtc

SEQ ID NO: 26 - DNA, 4487 bp, Mus musculus
tggcggctgggtggggattggctggaggggctgtaattcagcggtttccggagctgcagtgtagacagggagggggaacctggggttccgacgtcgcggcggagggaacgagccctaaccggatcgctgaggtacaaccccgctcggtgtcgcctgaccgcgtcggctaggagaggccaggcggccctcgggagcccagcagctcgcgcctggagtcagcgcaggccggccagtcgggcctcagccccggagacagtcgagacccctgactgcagcaggatggctcagtggaaccagctgcagcagctggacacacgctacctggagcagctgcaccagctgtacagcgacagcttccccatggagctgcggcagttcctggcaccttggattgagagtcaagactgggcatatgcagccagcaaagagtcacatgccacgttggtgtttcataatctcttgggtgaaattgaccagcaatatagccgattcctgcaagagtccaatgtcctctatcagcacaaccttcgaagaatcaagcagtttctgcagagcaggtatcttgagaagccaatggaaattgcccggatcgtggcccgatgcctgtgggaagagtctcgcctcctccagacggcagccacggcagcccagcaagggggccaggccaaccacccaacagccgccgtagtgacagagaagcagcagatgttggagcagcatcttcaggatgtccggaagcgagtgcaggatctagaacagaaaatgaaggtggtggagaacctccaggacgactttgatttcaactacaaaaccctcaagagccaaggagacatgcaggatctgaatggaaacaaccagtctgtgaccagacagaagatgcagcagctggaacagatgctcacagccctggaccagatgcggagaagcattgtgagtgagctggcggggctcttgtcagcaatggagtacgtgcagaagacactgactgatgaagagctggctgactggaagaggcggcagcagatcgcgtgcatcggaggccctcccaacatctgcctggaccgtctggaaaactggataacttcattagcagaatctcaacttcagacccgccaacaaattaagaaactggaggagctgcagcagaaagtgtcctacaagggcgaccctatcgtgcagcaccggcccatgctggaggagaggatcgtggagctgttcagaaacttaatgaagagtgccttcgtggtggagcggcagccctgcatgcccatgcacccggaccggcccttagtcatcaagactggtgtccagtttaccacgaaagtcaggttgctggtcaaatttcctgagttgaattatcagcttaaaattaaagtgtgcattgataaagactctggggatgttgctgccctcagagggtctcggaaatttaacattctgggcacgaacacaaaagtgatgaacatggaggagtctaacaacggcagcctgtctgcagagttcaagcacctgacccttagggagcagagatgtgggaatggaggccgtgccaattgtgatgcctccttgatcgtgactgaggagctgcacctgatcaccttcgagactgaggtgtaccaccaaggcctcaagattgacctagagacccactccttgccagttgtggtgatctccaacatctgtcagatgccaaatgcttgggcatcaatcctgtggtataacatgctgaccaataaccccaagaacgtgaacttcttcactaagccgccaattggaacctgggaccaagtggccgaggtgctcagctggcagttctcgtccaccaccaagcgggggctgagcatcgagcagctgacaacgctggctgagaagctcctagggcctggtgtgaactactcagggtgtcagatcacatgggctaaattctgcaaagaaaacatggctggcaagggcttctccttctgggtctggctagacaatatcatcgaccttgtgaaaaagtatatcttggccctttggaatgaagggtacatcatgggtttcatcagcaaggagcgggagcgggccatcctaagcacaaagcccccgggcaccttcctactgcgcttcagcgagagcagcaaagaaggaggggtcactttcacttgggtggaaaaggacatcagtggcaagacccagatccagtctgtagagccatacaccaagcagcagctgaacaacatgtcatttgctgaaatcatcatgggctataagatcatggatgcgaccaacatcctggtgtctccacttgtctacctctaccccgacattcccaaggaggaggcatttggaaagtactgtaggcccgagagccaggagcaccccgaagccgacccaggtagtgctgccccgtacctgaagaccaagttcatctgtgtgacaccaacgacctgcagcaataccattgacctgccgatgtccccccgcactttagattcattgatgcagtttggaaataacggtgaaggtgctgagccctcagcaggagggcagtttgagtcgctcacgtttgacatggatctgacctcggagtgtgctacctcccccatgtgaggagctgaaaccagaagctgcagagacgtgacttgagacacctgccccgtgctccacccctaagcagccgaaccccatatcgtctgaaactcctaactttgtggttccagatttttttttttaatttcctacttctgctatctttgggcaatctgggcactttttaaaatagagaaatgagtgagtgtgggtgataaactgttatgtaaagaggagagcacctctgagtctggggatggggctgagagcagaagggagcaaggggaacacctcctgtcctgcccgcctgccctcctttttcagcagctcggggttggttgttagacaagtgcctcctggtgcccatggcatcctgttgccccactctgtgagctgataccccaggctgggaactcctggctctgcactttcaaccttgctaatatccacatagaagctaggactaagcccagaggttcctctttaaattaaaaaaaaaaaaaataagaattaaagggcaaaacacactgacacagcatagcctttccatatcaaggaatactcagttaacagcctctccagcgctgtcttcaggctgatcatctatataaaccctggaatggttgcagatcaaatctgtaaaagagatccgagagctgtggcttggcctctggttcaaacacaaaggctagagagaacctagatatccctgggttttgtttacccagtatgcttgtcggttggaggtgtgaggtaggccaagggcactggaaagcctttgtcatcaccctactccctccccaacccagactccagaccctgtttcagggtcagcctgccctgtgggtgccttactgggcctagggtcaacctgccttcctttcccacttgaccttgctggtagtatgtccccttcccatgtccaaaggccctctgtcctgcttctattgggaatccctgcctcaggaccttgtgtcgagagggattgccttacaggtttgaacctgcctcagactacaggccctcagcaaagctcagggagtatggtccttattctatgcgcttggttcccagggatatctgtaaccacagggcaaaagctgacatatactccaggtctgccctcatatgagtggtgtattcttggcctcccctgagactggcaactgtctgctccccattgggtctcccaggtgaggtggaacacagttcctgcacctactgtggcctccatgtcgcttgcttgcttcgctcactcagcttactggaacactgagtgttcaaggcaagcctttcctgacagaggcatggctagattcagtgactcaaagccacctcattcagctgatcagtgtctgtggaattgtttccttccagttaaccagtgtctgaattaagggcagtgaggacattgtctccaagacgaactgcttgccttgaccaccccagccttctgcttcgagacagttactgctctcccaccccatcaatgttctttagttatacaataagctgaacttataaactgaaagggtatttaggaaggcaaggcttgggcatttttatggctttcaatcctggggacccaggaacaaggtgagggcttctctggggctggtgttgtacctcaggggctctgggaagtctgtgtgcctgggttaaccacccatagtgagcccctggaactgcccactttccctctccttggccccacttggccccagcctcacccagcctgcagactgcttagcctttcagtgcagtggcttgtgttctggccactgcactcagattccaatgtaaactttctagtgtaaaaatttatattattgtgggttgttttttgttgttgtttgtttttgtatattgctgtaactactttaacttccagaaataaagattatataggaactgtc

SEQ ID NO: 27 - DNA, 4484 bp, Mus musculus
tggcggctgggtggggattggctggaggggctgtaattcagcggtttccggagctgcagtgtagacagggagggggaacctggggttccgacgtcgcggcggagggaacgagccctaaccggatcgctgaggtacaaccccgctcggtgtcgcctgaccgcgtcggctaggagaggccaggcggccctcgggagcccagcagctcgcgcctggagtcagcgcaggccggccagtcgggcctcagccccggagacagtcgagacccctgactgcagcaggatggctcagtggaaccagctgcagcagctggacacacgctacctggagcagctgcaccagctgtacagcgacagcttccccatggagctgcggcagttcctggcaccttggattgagagtcaagactgggcatatgcagccagcaaagagtcacatgccacgttggtgtttcataatctcttgggtgaaattgaccagcaatatagccgattcctgcaagagtccaatgtcctctatcagcacaaccttcgaagaatcaagcagtttctgcagagcaggtatcttgagaagccaatggaaattgcccggatcgtggcccgatgcctgtgggaagagtctcgcctcctccagacggcagccacggcagcccagcaagggggccaggccaaccacccaacagccgccgtagtgacagagaagcagcagatgttggagcagcatcttcaggatgtccggaagcgagtgcaggatctagaacagaaaatgaaggtggtggagaacctccaggacgactttgatttcaactacaaaaccctcaagagccaaggagacatgcaggatctgaatggaaacaaccagtctgtgaccagacagaagatgcagcagctggaacagatgctcacagccctggaccagatgcggagaagcattgtgagtgagctggcggggctcttgtcagcaatggagtacgtgcagaagacactgactgatgaagagctggctgactggaagaggcggcagcagatcgcgtgcatcggaggccctcccaacatctgcctggaccgtctggaaaactggataacttcattagcagaatctcaacttcagacccgccaacaaattaagaaactggaggagctgcagcagaaagtgtcctacaagggcgaccctatcgtgcagcaccggcccatgctggaggagaggatcgtggagctgttcagaaacttaatgaagagtgccttcgtggtggagcggcagccctgcatgcccatgcacccggaccggcccttagtcatcaagactggtgtccagtttaccacgaaagtcaggttgctggtcaaatttcctgagttgaattatcagcttaaaattaaagtgtgcattgataaagactctggggatgttgctgccctcagagggtctcggaaatttaacattctgggcacgaacacaaaagtgatgaacatggaggagtctaacaacggcagcctgtctgcagagttcaagcacctgacccttagggagcagagatgtgggaatggaggccgtgccaattgtgatgcctccttgatcgtgactgaggagctgcacctgatcaccttcgagactgaggtgtaccaccaaggcctcaagattgacctagagacccactccttgccagttgtggtgatctccaacatctgtcagatgccaaatgcttgggcatcaatcctgtggtataacatgctgaccaataaccccaagaacgtgaacttcttcactaagccgccaattggaacctgggaccaagtggccgaggtgctcagctggcagttctcgtccaccaccaagcgggggctgagcatcgagcagctgacaacgctggctgagaagctcctagggcctggtgtgaactactcagggtgtcagatcacatgggctaaattctgcaaagaaaacatggctggcaagggcttctccttctgggtctggctagacaatatcatcgaccttgtgaaaaagtatatcttggccctttggaatgaagggtacatcatgggtttcatcagcaaggagcgggagcgggccatcctaagcacaaagcccccgggcaccttcctactgcgcttcagcgagagcagcaaagaaggaggggtcactttcacttgggtggaaaaggacatcagtggcaagacccagatccagtctgtagagccatacaccaagcagcagctgaacaacatgtcatttgctgaaatcatcatgggctataagatcatggatgcgaccaacatcctggtgtctccacttgtctacctctaccccgacattcccaaggaggaggcatttggaaagtactgtaggcccgagagccaggagcaccccgaagccgacccaggtgctgccccgtacctgaagaccaagttcatctgtgtgacaccaacgacctgcagcaataccattgacctgccgatgtccccccgcactttagattcattgatgcagtttggaaataacggtgaaggtgctgagccctcagcaggagggcagtttgagtcgctcacgtttgacatggatctgacctcggagtgtgctacctcccccatgtgaggagctgaaaccagaagctgcagagacgtgacttgagacacctgccccgtgctccacccctaagcagccgaaccccatatcgtctgaaactcctaactttgtggttccagatttttttttttaatttcctacttctgctatctttgggcaatctgggcactttttaaaatagagaaatgagtgagtgtgggtgataaactgttatgtaaagaggagagcacctctgagtctggggatggggctgagagcagaagggagcaaggggaacacctcctgtcctgcccgcctgccctcctttttcagcagctcggggttggttgttagacaagtgcctcctggtgcccatggcatcctgttgccccactctgtgagctgataccccaggctgggaactcctggctctgcactttcaaccttgctaatatccacatagaagctaggactaagcccagaggttcctctttaaattaaaaaaaaaaaaaataagaattaaagggcaaaacacactgacacagcatagcctttccatatcaaggaatactcagttaacagcctctccagcgctgtcttcaggctgatcatctatataaaccctggaatggttgcagatcaaatctgtaaaagagatccgagagctgtggcttggcctctggttcaaacacaaaggctagagagaacctagatatccctgggttttgtttacccagtatgcttgtcggttggaggtgtgaggtaggccaagggcactggaaagcctttgtcatcaccctactccctccccaacccagactccagaccctgtttcagggtcagcctgccctgtgggtgccttactgggcctagggtcaacctgccttcctttcccacttgaccttgctggtagtatgtccccttcccatgtccaaaggccctctgtcctgcttctattgggaatccctgcctcaggaccttgtgtcgagagggattgccttacaggtttgaacctgcctcagactacaggccctcagcaaagctcagggagtatggtccttattctatgcgcttggttcccagggatatctgtaaccacagggcaaaagctgacatatactccaggtctgccctcatatgagtggtgtattcttggcctcccctgagactggcaactgtctgctccccattgggtctcccaggtgaggtggaacacagttcctgcacctactgtggcctccatgtcgcttgcttgcttcgctcactcagcttactggaacactgagtgttcaaggcaagcctttcctgacagaggcatggctagattcagtgactcaaagccacctcattcagctgatcagtgtctgtggaattgtttccttccagttaaccagtgtctgaattaagggcagtgaggacattgtctccaagacgaactgcttgccttgaccaccccagccttctgcttcgagacagttactgctctcccaccccatcaatgttctttagttatacaataagctgaacttataaactgaaagggtatttaggaaggcaaggcttgggcatttttatggctttcaatcctggggacccaggaacaaggtgagggcttctctggggctggtgttgtacctcaggggctctgggaagtctgtgtgcctgggttaaccacccatagtgagcccctggaactgcccactttccctctccttggccccacttggccccagcctcacccagcctgcagactgcttagcctttcagtgcagtggcttgtgttctggccactgcactcagattccaatgtaaactttctagtgtaaaaatttatattattgtgggttgttttttgttgttgtttgtttttgtatattgctgtaactactttaacttccagaaataaagattatataggaactgtc

SEQ ID NO: 28 - DNA, 2476 bp, Sus scrofa
ttggcgctctccccgtgtctaataggggagactctgcggcctgagggagccctgcggcccgcaccccggcgcaagcgcagccccggccagtcgggcctcagccggagcagcagttgtgacccctgattccagcagaatggcccaatggaatcagctacagcagctggacacacggtacctggagcagcttcatcagctgtacagcgacagcttcccgatggagctccggcagtttctggccccttggattgagagtcaagattgggcatatgcagccagcaaagaatcccacgccactttggtgtttcataatctcttgggtgaaattgaccagcagtatagccgcttcctgcaggagtccaatgttctctatcagcacaacctgcgaagaatcaagcagttccttcagagcagatatcttgagaagccaatggagattgcccgaattgtggcccggtgcctgtgggaagagtcccgcctcctccagactgcagccactgcagcccagcaagggggccaggccaaccaccccacagccgctgtggtgacagagaagcagcagatgctggagcagcaccttcaggatgttcgcaagcgtgtccaggatctggaacagaaaatgaaagtggtggagaatctccaggatgactttgatttcaactacaaaaccctcaagagtcaaggagatatgcaagacctgaatggaaacaaccagtcagtgaccaggcagaagatgcagcagctggagcagatgctcactgcgctggaccagatgcggcgaagcatcgtgagtgagttggcggggcttttgtcagctatggagtatgtgcagaaaactctcacggatgaagagctggctgactggaagaggcggcagcagattgcgtgcattggaggccctcccaacatctgcctggatcggctagaaaactggataacctcattagcagaatctcaacttcagacccgccaacaaattaagaaactagaggagctgcagcagaaagtgagctacaaaggggaccccattgtacagcaccggcccatgctggaggagagaatcgtggagctgttcagaaacttgatgaaaagtgccttcgtggtggagcgacagccctgcatgcctatgcaccctgaccggccattagtcatcaagaccggtgtccagtttacaacgaaagtcagattgctggtcaaattccctgagctaaactatcagcttaaaattaaagtgtgcattgacaaagactccggggacgtcgcggctctcagaggatcccggaaatttaacatcctgggcaccaacacgaaagtgatgaatatggaagagtccaacaacggcagcctctccgcagagttcaaacacctgaccctgagagagcagagatgtgggaacgggggccgagccaattgcgacgcctccctcattgtgaccgaggagctgcacctgatcacctttgagaccgaggtgtatcatcaaggcctcaagattgacctggagacccactccttgccagtcgtggtcatctccaacatctgtcagatgcccaacgcctgggcctccatcctgtggtataacatgctgaccaacaaccccaagaacgtgaactttttcaccaagcccccgatcggaacgtgggatcaagtggccgaggtgctgagctggcagttctcttccaccaccaagcgagggctgagcatcgagcagttgagcacactggcagagaaactcctaggacctggtgtgaactattcagggtgtcagatcacgtgggctaagttttgcaaagaaaacatggctggcaagggcttctccttctgggtctggctggacaatatcattgaccttgtgaaaaagtacatcctggccctttggaatgaagggtacataatgggctttatcagtaaggagagggagcgggccatcttgagcaccaagcccccaggcaccttcctgctgagattcagtgaaagcagcaaagaaggaggagtcaccttcacttgggtggagaaggacatcagcggtaagacccagatccagtcggtggaaccgtacaccaagcagcagctcaacaacatgtcgtttgctgaaatcatcatgggctataagatcatggatgctaccaacatccttgtgtctccgctggtctacctctaccctgacattccaaaggaagaggcgtttggaaagtattgtcggccagagagccaggagcatcccgaagctgacccaggtagtgctgccccatacctgaagaccaagttcatctgtgtgacaccaacgacctgcagcaataccattgacctgccgatgtccccccgcactttagattcattgatgcagtttggaaataatggtgaaggtgctgagccctcagcaggagggcagtttgagtccctcaccttcgacatggagctgaactcggagtgcgctacctcccccatgtgaggagccgaggacaggagctgcagagag

SEQ ID NO: 29 - DNA, 3700 bp, Homo sapiens; identical to SEQ ID NO: 17

SEQ ID NO: 30 - DNA, 3399 bp, Homo sapiens
atgaatttacaaccaattttctggattggactgatcagttcagtttgctgtgtgtttgctcaaacagatgaaaatagatgtttaaaagcaaatgccaaatcatgtggagaatgtatacaagcagggccaaattgtgggtggtgcacaaattcaacatttttacaggaaggaatgcctacttctgcacgatgtgatgatttagaagccttaaaaaagaagggttgccctccagatgacatagaaaatcccagaggctccaaagatataaagaaaaataaaaatgtaaccaaccgtagcaaaggaacagcagagaagctcaagccagaggatattactcagatccaaccacagcagttggttttgcgattaagatcaggggagccacagacatttacattaaaattcaagagagctgaagactatcccattgacctctactaccttatggacctgtcttactcaatgaaagacgatttggagaatgtaaaaagtcttggaacagatctgatgaatgaaatgaggaggattacttcggacttcagaattggatttggctcatttgtggaaaagactgtgatgccttacattagcacaacaccagctaagctcaggaacccttgcacaagtgaacagaactgcaccagcccatttagctacaaaaatgtgctcagtcttactaataaaggagaagtatttaatgaacttgttggaaaacagcgcatatctggaaatttggattctccagaaggtggtttcgatgccatcatgcaagttgcagtttgtggatcactgattggctggaggaatgttacacggctgctggtgttttccacagatgccgggtttcactttgctggagatgggaaacttggtggcattgttttaccaaatgatggacaatgtcacctggaaaataatatgtacacaatgagccattattatgattatccttctattgctcaccttgtccagaaactgagtgaaaataatattcagacaatttttgcagttactgaagaatttcagcctgtttacaaggagctgaaaaacttgatccctaagtcagcagtaggaacattatctgcaaattctagcaatgtaattcagttgatcattgatgcatacaattccctttcctcagaagtcattttggaaaacggcaaattgtcagaaggagtaacaataagttacaaatcttactgcaagaacggggtgaatggaacaggggaaaatggaagaaaatgttccaatatttccattggagatgaggttcaatttgaaattagcataacttcaaataagtgtccaaaaaaggattctgacagctttaaaattaggcctctgggctttacggaggaagtagaggttattcttcagtacatctgtgaatgtgaatgccaaagcgaaggcatccctgaaagtcccaagtgtcatgaaggaaatgggacatttgagtgtggcgcgtgcaggtgcaatgaagggcgtgttggtagacattgtgaatgcagcacagatgaagttaacagtgaagacatggatgcttactgcaggaaagaaaacagttcagaaatctgcagtaacaatggagagtgcgtctgcggacagtgtgtttgtaggaagagggataatacaaatgaaatttattctggcaaattctgcgagtgtgataatttcaactgtgatagatccaatggcttaatttgtggaggaaatggtgtttgcaagtgtcgtgtgtgtgagtgcaaccccaactacactggcagtgcatgtgactgttctttggatactagtacttgtgaagccagcaacggacagatctgcaatggccggggcatctgcgagtgtggtgtctgtaagtgtacagatccgaagtttcaagggcaaacgtgtgagatgtgtcagacctgccttggtgtctgtgctgagcataaagaatgtgttcagtgcagagccttcaataaaggagaaaagaaagacacatgcacacaggaatgttcctattttaacattaccaaggtagaaagtcgggacaaattaccccagccggtccaacctgatcctgtgtcccattgtaaggagaaggatgttgacgactgttggttctattttacgtattcagtgaatgggaacaacgaggtcatggttcatgttgtggagaatccagagtgtcccactggtccagacatcattccaattgtagctggtgtggttgctggaattgttcttattggccttgcattactgctgatatggaagcttttaatgataattcatgacagaagggagtttgctaaatttgaaaaggagaaaatgaatgccaaatgggacacggtaagttacaaaacatccaaaaagcaaagtggcttataaagtaaatgtaatactcctaagacttatgtattagctgtcaggctgattattaaagtcctttctaagtattttattcccccaaaagtttcttactcaaggaatttgcatttagtgaaaaacagaaagcatcctaaatatatcccattgaaacaaaacattgattataagcatgtatattctggttcatgtggccgatatttttatttctttaatgattttgatcctaaatctgccttttcatctaatgtgaagtagaatcctaaataatgttatctgtgtagcaagctattcaatgggaaagctgcttctttctttaaaacaaacaaacaaaaaaaaccttcagtggaaagccaaattccaaaaggttatataccaagcttgtccaactcgcagctcgtcggccaggacatgcagcccagaatagctttgaatgtggccccaacacaaatttgtaaactttcttagaaattgtaattattattattatttttttttggtaactttttttaaagctcatcagctatcgttagtgtattttatgtgtggcccaagacagttcttcttcttgccagtgtggcccagggaagccaaaagattggacacccctgctatatactatatgattccatttagaggacattctggaaaagcaaaactgtaggggcaaaaatcagtggttgctaggggctggaatgggggaaagtgttgaccacagaggggcgtaagggatcttccttgggatgacttgattgtgggtggatttatgtatttgaaaactcacagaactatgtactttaaaaagatgtatgttcctctatgaaaattatatctcagtaaactttggcttataaaaatcttaaaagccctaagtgaccgaaaggttatgttagcattgagtgctttgaaatatggagtcagagggtggggtaaccaaatgttggcctttgtgtattcatcttttgatacaagaaagcaatgccaatcttcagtatttttaaattgtaaatgaattttgtagttcccgaattc

SEQ ID NO: 31 - DNA, 2516 bp, Homo sapiens
atgaatttacaaccaattttctggattggactgatcagttcagtttgctgtgtgtttgctcaaacagatgaaaatagatgtttaaaagcaaatgccaaatcatgtggagaatgtatacaagcagggccaaattgtgggtggtgcacaaattcaacatttttacaggaaggaatgcctacttctgcacgatgtgatgatttagaagccttaaaaaagaagggttgccctccagatgacatagaaaatcccagaggctccaaagatataaagaaaaataaaaatgtaaccaaccgtagcaaaggaacagcagagaagctcaagccagaggatattactcagatccaaccacagcagttggttttgcgattaagatcaggggagccacagacatttacattaaaattcaagagagctgaagactatcccattgacctctactaccttatggacctgtcttactcaatgaaagacgatttggagaatgtaaaaagtcttggaacagatctgatgaatgaaatgaggaggattacttcggacttcagaattggatttggctcatttgtggaaaagactgtgatgccttacattagcacaacaccagctaagctcaggaacccttgcacaagtgaacagaactgcaccagcccatttagctacaaaaatgtgctcagtcttactaataaaggagaagtatttaatgaacttgttggaaaacagcgcatatctggaaatttggattctccagaaggtggtttcgatgccatcatgcaagttgcagtttgtggatcactgattggctggaggaatgttacacggctgctggtgttttccacagatgccgggtttcactttgctggagatgggaaacttggtggcattgttttaccaaatgatggacaatgtcacctggaaaataatatgtacacaatgagccattattatgattatccttctattgctcaccttgtccagaaactgagtgaaaataatattcagacaatttttgcagttactgaagaatttcagcctgtttacaaggagctgaaaaacttgatccctaagtcagcagtaggaacattatctgcaaattctagcaatgtaattcagttgatcattgatgcatacaattccctttcctcagaagtcattttggaaaacggcaaattgtcagaaggagtaacaataagttacaaatcttactgcaagaacggggtgaatggaacaggggaaaatggaagaaaatgttccaatatttccattggagatgaggttcaatttgaaattagcataacttcaaataagtgtccaaaaaaggattctgacagctttaaaattaggcctctgggctttacggaggaagtagaggttattcttcagtacatctgtgaatgtgaatgccaaagcgaaggcatccctgaaagtcccaagtgtcatgaaggaaatgggacatttgagtgtggcgcgtgcaggtgcaatgaagggcgtgttggtagacattgtgaatgcagcacagatgaagttaacagtgaagacatggatgcttactgcaggaaagaaaacagttcagaaatctgcagtaacaatggagagtgcgtctgcggacagtgtgtttgtaggaagagggataatacaaatgaaatttattctggcaaattctgcgagtgtgataatttcaactgtgatagatccaatggcttaatttgtggaggaaatggtgtttgcaagtgtcgtgtgtgtgagtgcaaccccaactacactggcagtgcatgtgactgttctttggatactagtacttgtgaagccagcaacggacagatctgcaatggccggggcatctgcgagtgtggtgtctgtaagtgtacagatccgaagtttcaagggcaaacgtgtgagatgtgtcagacctgccttggtgtctgtgctgagcataaagaatgtgttcagtgcagagccttcaataaaggagaaaagaaagacacatgcacacaggaatgttcctattttaacattaccaaggtagaaagtcgggacaaattaccccagccggtccaacctgatcctgtgtcccattgtaaggagaaggatgttgacgactgttggttctattttacgtattcagtgaatgggaacaacgaggtcatggttcatgttgtggagaatccagagtgtcccactggtccagacatcattccaattgtagctggtgtggttgctggaattgttcttattggccttgcattactgctgatatggaagcttttaatgataattcatgacagaagggagtttgctaaatttgaaaaggagaaaatgaatgccaaatgggacacgtctctctctgtcgcccagcctggagtgcagtggtgtgatatcagctcactgcaacctctgacttccagattccagcaattctcctgcctcagcctcccgagtacctgggattacagggtgaaaatcctatttataagagtgccgtaacaactgtggtcaatccgaagtatgagggaaaatgagta

SEQ ID NO: 32 - DNA, 2481 bp, Homo sapiens
atgaatttacaaccaattttctggattggactgatcagttcagtttgctgtgtgtttgctcaaacagatgaaaatagatgtttaaaagcaaatgccaaatcatgtggagaatgtatacaagcagggccaaattgtgggtggtgcacaaattcaacatttttacaggaaggaatgcctacttctgcacgatgtgatgatttagaagccttaaaaaagaagggttgccctccagatgacatagaaaatcccagaggctccaaagatataaagaaaaataaaaatgtaaccaaccgtagcaaaggaacagcagagaagctcaagccagaggatattactcagatccaaccacagcagttggttttgcgattaagatcaggggagccacagacatttacattaaaattcaagagagctgaagactatcccattgacctctactaccttatggacctgtcttactcaatgaaagacgatttggagaatgtaaaaagtcttggaacagatctgatgaatgaaatgaggaggattacttcggacttcagaattggatttggctcatttgtggaaaagactgtgatgccttacattagcacaacaccagctaagctcaggaacccttgcacaagtgaacagaactgcaccagcccatttagctacaaaaatgtgctcagtcttactaataaaggagaagtatttaatgaacttgttggaaaacagcgcatatctggaaatttggattctccagaaggtggtttcgatgccatcatgcaagttgcagtttgtggatcactgattggctggaggaatgttacacggctgctggtgttttccacagatgccgggtttcactttgctggagatgggaaacttggtggcattgttttaccaaatgatggacaatgtcacctggaaaataatatgtacacaatgagccattattatgattatccttctattgctcaccttgtccagaaactgagtgaaaataatattcagacaatttttgcagttactgaagaatttcagcctgtttacaaggagctgaaaaacttgatccctaagtcagcagtaggaacattatctgcaaattctagcaatgtaattcagttgatcattgatgcatacaattccctttcctcagaagtcattttggaaaacggcaaattgtcagaaggagtaacaataagttacaaatcttactgcaagaacggggtgaatggaacaggggaaaatggaagaaaatgttccaatatttccattggagatgaggttcaatttgaaattagcataacttcaaataagtgtccaaaaaaggattctgacagctttaaaattaggcctctgggctttacggaggaagtagaggttattcttcagtacatctgtgaatgtgaatgccaaagcgaaggcatccctgaaagtcccaagtgtcatgaaggaaatgggacatttgagtgtggcgcgtgcaggtgcaatgaagggcgtgttggtagacattgtgaatgcagcacagatgaagttaacagtgaagacatggatgcttactgcaggaaagaaaacagttcagaaatctgcagtaacaatggagagtgcgtctgcggacagtgtgtttgtaggaagagggataatacaaatgaaatttattctggcaaattctgcgagtgtgataatttcaactgtgatagatccaatggcttaatttgtggaggaaatggtgtttgcaagtgtcgtgtgtgtgagtgcaaccccaactacactggcagtgcatgtgactgttctttggatactagtacttgtgaagccagcaacggacagatctgcaatggccggggcatctgcgagtgtggtgtctgtaagtgtacagatccgaagtttcaagggcaaacgtgtgagatgtgtcagacctgccttggtgtctgtgctgagcataaagaatgtgttcagtgcagagccttcaataaaggagaaaagaaagacacatgcacacaggaatgttcctattttaacattaccaaggtagaaagtcgggacaaattaccccagccggtccaacctgatcctgtgtcccattgtaaggagaaggatgttgacgactgttggttctattttacgtattcagtgaatgggaacaacgaggtcatggttcatgttgtggagaatccagagtgtcccactggtccagacatcattccaattgtagctggtgtggttgctggaattgttcttattggccttgcattactgctgatatggaagcttttaatgataattcatgacagaagggagtttgctaaatttgaaaaggagaaaatgaatgccaaatgggacacgcaagaaaatccgatttacaagagtcctattaataatttcaagaatccaaactacggacgtaaagctggtctctaaattgccggtgaaaatcctatttataagagtgccgtaacaactgtggtcaatccgaagtatgagggaaaatgagta

SEQ ID NO: 33 - DNA, 2498 bp, Homo sapiens
atgaatttacaaccaattttctggattggactgatcagttcagtttgctgtgtgtttgctcaaacagatgaaaatagatgtttaaaagcaaatgccaaatcatgtggagaatgtatacaagcagggccaaattgtgggtggtgcacaaattcaacatttttacaggaaggaatgcctacttctgcacgatgtgatgatttagaagccttaaaaaagaagggttgccctccagatgacatagaaaatcccagaggctccaaagatataaagaaaaataaaaatgtaaccaaccgtagcaaaggaacagcagagaagctcaagccagaggatattactcagatccaaccacagcagttggttttgcgattaagatcaggggagccacagacatttacattaaaattcaagagagctgaagactatcccattgacctctactaccttatggacctgtcttactcaatgaaagacgatttggagaatgtaaaaagtcttggaacagatctgatgaatgaaatgaggaggattacttcggacttcagaattggatttggctcatttgtggaaaagactgtgatgccttacattagcacaacaccagctaagctcaggaacccttgcacaagtgaacagaactgcaccagcccatttagctacaaaaatgtgctcagtcttactaataaaggagaagtatttaatgaacttgttggaaaacagcgcatatctggaaatttggattctccagaaggtggtttcgatgccatcatgcaagttgcagtttgtggatcactgattggctggaggaatgttacacggctgctggtgttttccacagatgccgggtttcactttgctggagatgggaaacttggtggcattgttttaccaaatgatggacaatgtcacctggaaaataatatgtacacaatgagccattattatgattatccttctattgctcaccttgtccagaaactgagtgaaaataatattcagacaatttttgcagttactgaagaatttcagcctgtttacaaggagctgaaaaacttgatccctaagtcagcagtaggaacattatctgcaaattctagcaatgtaattcagttgatcattgatgcatacaattccctttcctcagaagtcattttggaaaacggcaaattgtcagaaggagtaacaataagttacaaatcttactgcaagaacggggtgaatggaacaggggaaaatggaagaaaatgttccaatatttccattggagatgaggttcaatttgaaattagcataacttcaaataagtgtccaaaaaaggattctgacagctttaaaattaggcctctgggctttacggaggaagtagaggttattcttcagtacatctgtgaatgtgaatgccaaagcgaaggcatccctgaaagtcccaagtgtcatgaaggaaatgggacatttgagtgtggcgcgtgcaggtgcaatgaagggcgtgttggtagacattgtgaatgcagcacagatgaagttaacagtgaagacatggatgcttactgcaggaaagaaaacagttcagaaatctgcagtaacaatggagagtgcgtctgcggacagtgtgtttgtaggaagagggataatacaaatgaaatttattctggcaaattctgcgagtgtgataatttcaactgtgatagatccaatggcttaatttgtggaggaaatggtgtttgcaagtgtcgtgtgtgtgagtgcaaccccaactacactggcagtgcatgtgactgttctttggatactagtacttgtgaagccagcaacggacagatctgcaatggccggggcatctgcgagtgtggtgtctgtaagtgtacagatccgaagtttcaagggcaaacgtgtgagatgtgtcagacctgccttggtgtctgtgctgagcataaagaatgtgttcagtgcagagccttcaataaaggagaaaagaaagacacatgcacacaggaatgttcctattttaacattaccaaggtagaaagtcgggacaaattaccccagccggtccaacctgatcctgtgtcccattgtaaggagaaggatgttgacgactgttggttctattttacgtattcagtgaatgggaacaacgaggtcatggttcatgttgtggagaatccagagtgtcccactggtccagacatcattccaattgtagctggtgtggttgctggaattgttcttattggccttgcattactgctgatatggaagcttttaatgataattcatgacagaagggagtttgctaaatttgaaaaggagaaaatgaatgccaaatgggacacgcctggagtgcagtggtgtgatatcagctcactgcaacctctgacttccagattccagcaattctcctgcctcagcctcccgagtacctgggattacagggtgaaaatcctatttataagagtgccgtaacaactgtggtcaatccgaagtatgagggaaaatgagta

SEQ ID NO: 34 - DNA, 2533 bp, Homo sapiens
gagccagcccagccgcgttccgaacgcgagggtcgccggcctgggcgctgtcacgtcggggctgccggagctgcgggggaccgggcccgaacggcccctgacacctgcggtctcccgccgggctgggcaagcgcagatgaatttacaaccaattttctggattggactgatcagttcagtttgctgtgtgtttgctcaaacagatgaaaatagatgtttaaaagcaaatgccaaatcatgtggagaatgtatacaagcagggccaaattgtgggtggtgcacaaattcaacatttttacaggaaggaatgcctacttctgcacgatgtgatgatttagaagccttaaaaaagaagggttgccctccagatgacatagaaaatcccagaggctccaaagatataaagaaaaataaaaatgtaaccaaccgtagcaaaggaacagcagagaagctcaagccagaggatattactcagatccaaccacagcagttggttttgcgattaagatcaggggagccacagacatttacattaaaattcaagagagctgaagactatcccattgacctctactaccttatggacctgtcttactcaatgaaagacgatttggagaatgtaaaaagtcttggaacagatctgatgaatgaaatgaggaggattacttcggacttcagaattggatttggctcatttgtggaaaagactgtgatgccttacattagcacaacaccagctaagctcaggaacccttgcacaagtgaacagaactgcaccagcccatttagctacaaaaatgtgctcagtcttactaataaaggagaagtatttaatgaacttgttggaaaacagcgcatatctggaaatttggattctccagaaggtggtttcgatgccatcatgcaagttgcagtttgtggatcactgattggctggaggaatgttacacggctgctggtgttttccacagatgccgggtttcactttgctggagatgggaaacttggtggcattgttttaccaaatgatggacaatgtcacctggaaaataatatgtacacaatgagccattattatgattatccttctattgctcaccttgtccagaaactgagtgaaaataatattcagacaatttttgcagttactgaagaatttcagcctgtttacaaggagctgaaaaacttgatccctaagtcagcagtaggaacattatctgcaaattctagcaatgtaattcagttgatcattgatgcatacaattccctttcctcagaagtcattttggaaaacggcaaattgtcagaaggagtaacaataagttacaaatcttactgcaagaacggggtgaatggaacaggggaaaatggaagaaaatgttccaatatttccattggagatgaggttcaatttgaaattagcataacttcaaataagtgtccaaaaaaggattctgacagctttaaaattaggcctctgggctttacggaggaagtagaggttattcttcagtacatctgtgaatgtgaatgccaaagcgaaggcatccctgaaagtcccaagtgtcatgaaggaaatgggacatttgagtgtggcgcgtgcaggtgcaatgaagggcgtgttggtagacattgtgaatgcagcacagatgaagttaacagtgaagacatggatgcttactgcaggaaagaaaacagttcagaaatctgcagtaacaatggagagtgcgtctgcggacagtgtgtttgtaggaagagggataatacaaatgaaatttattctggcaaattctgcgagtgtgataatttcaactgtgatagatccaatggcttaatttgtggaggaaatggtgtttgcaagtgtcgtgtgtgtgagtgcaaccccaactacactggcagtgcatgtgactgttctttggatactagtacttgtgaagccagcaacggacagatctgcaatggccggggcatctgcgagtgtggtgtctgtaagtgtacagatccgaagtttcaagggcaaacgtgtgagatgtgtcagacctgccttggtgtctgtgctgagcataaagaatgtgttcagtgcagagccttcaataaaggagaaaagaaagacacatgcacacaggaatgttcctattttaacattaccaaggtagaaagtcgggacaaattaccccagccggtccaacctgatcctgtgtcccattgtaaggagaaggatgttgacgactgttggttctattttacgtattcagtgaatgggaacaacgaggtcatggttcatgttgtggagaatccagagtgtcccactggtccagacatcattccaattgtagctggtgtggttgctggaattgttcttattggccttgcattactgctgatatggaagcttttaatgataattcatgacagaagggagtttgctaaatttgaaaaggagaaaatgaatgccaaatgggacacgggtgaaaatcctatttataagagtgccgtaacaactgtggtcaatccgaagtatgagggaaaatga

SEQ ID NO: 35 - DNA, 3701 bp, Mus musculus
cgcggagcggccgaggcccgccgacgccacccgagaccaaccgagaagccgaggcccgccgacgccggcccgagaccctccgaaaggccgaggtccgccgacaccgacccgagaccctccgagaggcggaacgcggcggagcccgggccgcccgcgtgtcgggcggacgctgcgaaaagatgaatttgcaactggtttcctggattggattgatcagtttgatttgttctgtatttggccaaacagataaaaatagatgtttaaaagcaaatgccaaatcttgcggagaatgtatacaagcagggccaaattgtgggtggtgtacaaatacgacatttttgcaagaaggaatgcctacttctgcacgatgtgatgatttagaagctttgaaaaagaagggttgccagccaagtgacatagagaatcccagaggctctcaaactataaagaaaaataaaaatgtcaccaatcgcagcaaagggatggcagagaagctccggccagaagacattactcagatccaaccacaacagctgcttctaaaattgagatcaggagaaccacagaagtttacattaaaattcaagagggctgaagattaccctattgatctctactaccttatggatctctcctactctatgaaagatgatctggagaatgtgaaaagtcttggaacggatttgatgaatgaaatgaggaggattacttcagacttccgcattggctttggctcatttgtggagaaaactgtgatgccgtatattagcacaaccccagcaaagctaagaaatccttgtacaagtgaacaaaactgcaccagcccatttagctacaaaaatgtgcttagtcttactgacagaggagagtttttcaatgaacttgttggtcagcaacgcatatctggaaacttggattctccagaaggtggctttgatgcaatcatgcaggttgcggtttgtggatcgctgattggctggaggaatgtaacacgactgctggtgttttccacggatgctgggtttcactttgctggagatgggaaacttggtggtattgttttacccaatgatggacaatgtcacctggaaaataatgtatatacaatgagccattactatgattatccttcaattgctcaccttgttcagaaactaagtgaaaataatattcagacgatttttgcagttactgaagagttccaacctgtttacaaggaattgaagaatttgattcctaagtcagcagtgggcacactgtctggaaactctagtaatgtgatccagctaatcatcgatgcctacaactctctttcttcagaagtcattctggaaaatagcaaattgccagacggagtaacaataaattacaaatcctattgcaagaatggggtgaatgggacaggagaaaatggacgaaagtgttccaacatttctattggagatgaggttcaatttgaaattagcataactgctaataaatgtccaaataaggagtctgaaaccattaaaattaaacctctgggcttcactgaagaagtagaggtcgttcttcagttcatctgtaagtgcaattgtcaaagccatggcatcccagccagtcccaagtgccatgagggaaatgggacatttgagtgtggagcctgcaggtgcaatgaggggcgtgttgggaggcactgtgaatgtagcacagatgaagtgaacagtgaagacatggacgcttactgcaggaaagagaacagttcggaaatctgcagtaacaatggagaatgtgtctgtggacagtgtgtgtgtaggaagagagataatacaaatgaaatttactctggaaaattctgcgagtgtgataacttcaactgtgataggtctaatggcttaatttgtggaggcaatggcgtgtgcaggtgtcgtgtttgtgaatgctatcccaattacactggcagtgcatgtgactgttctttggacactggtccatgtctagcgtcaaatggtcagatctgcaatggccggggtatttgtgaatgtggtgcttgtaagtgcacagatcccaagtttcaagggccaacttgtgagacatgtcagacctgccttggcgtctgtgcagagcataaagaatgtgttcagtgcagagccttcaataaaggagaaaagaaagacacgtgtgcacaggagtgctcccacttcaatctcaccaaagtagaaagcagggagaagttgccccagccggtgcaggtcgatcctgtgacccattgcaaggagaaggacattgatgactgctggttctatttcacctattcagtgaatggcaacaatgaagctatcgtgcatgttgtggagactccagactgtcctactggtcccgacatcatcccaattgtagcaggcgtggttgctggaattgttcttattggccttgccttgctgctgatttggaaacttttaatgataattcatgacagaagggaatttgctaaatttgaaaaggagaaaatgaatgccaagtgggacacgggtgaaaatcctatttacaagagtgccgtgacaactgtggtcaatccgaagtatgagggaaaatgaatcctactcaggcggattttgcaacaccaagctcacagcagcagcatcttagtcacagtagggtagttttggggctctgtggccagggttttattcacatgcaggtttggaaaatgtacaatatgtataatttttaattttttattattttgaaaataatgttataatccatgccagggactgacagaagacttgagaaaggatctttatccttgtcagctgaggtcacagtgtgcctttttaacccttccttctggaccattggaatcaagctctcactagattgaatgacactgctagtgccaatgcagggcagagctgagcagcgtcgcagcatctgaaccatgactgattttcagcttcacagggtggccagggctggttatacagaatcaaagaacagtccttgctggtggctttgttctttgtttagcctgctggctgccctctgggaattcccagtgatgtatgtggaagacaagagtgttgacagtttccaattaaaataaaattgacacagttgatgttcaagtccattggaaaaattctgagaggtgggaaggaaatcttagctttaaaacctgtgtgccattgtgagttacttgatcctgtaactccgacgccttttctttataaatccaaccttggataaaagtactgggaacttctctgctaaaaagtccctgacttagcactattcacataaaggccataattttagtagcattgctgagtggggaccttttgggttgagcttattttaccttttttttttctttaattcctggtgctcctttatcaccttctctaatcttttaatgtgtctgtttgcaatatgggggttagactttttatcattaccttttcttttccttggctgtacatttacctttttcacaaatactgtaagctgtcctgctgcttgcaggactacagggcctgggcagggccccccagcaacaattcacccacagtgcacctgcacatgcctttcctacatgcttgctctgtctcgaactagtcacaatcttgttttaagtgccttttcattttgacagtgctattaactgaagttatttattaaaataaaaaggcctaaatacattaaaaaaaaaaaaaaaaaaa

SEQ ID NO: 36 - DNA, 3334 bp, Homo Sapiens
attcctgcctgggaggttgtggaagaaggaagatggccagagctttgtgtccactgcaagccctctggcttctggagtgggtgctgctgctcttgggaccttgtgctgcccctccagcctgggccttgaacctggacccagtgcagctcaccttctatgcaggccccaatggcagccagtttggattttcactggacttccacaaggacagccatgggagagtggccatcgtggtgggcgccccgcggaccctgggccccagccaggaggagacgggcggcgtgttcctgtgcccctggagggccgagggcggccagtgcccctcgctgctctttgacctccgtgatgagacccgaaatgtaggctcccaaactttacaaaccttcaaggcccgccaaggactgggggcgtcggtcgtcagctggagcgacgtcattgtggcctgcgccccctggcagcactggaacgtcctagaaaagactgaggaggctgagaagacgcccgtaggtagctgctttttggctcagccagagagcggccgccgcgccgagtactccccctgtcgcgggaacaccctgagccgcatttacgtggaaaatgattttagctgggacaagcgttactgtgaagcgggcttcagctccgtggtcactcaggccggagagctggtgcttggggctcctggcggctattatttcttaggtctcctggcccaggctccagttgcggatattttctcgagttaccgcccaggcatccttttgtggcacgtgtcctcccagagcctctcctttgactccagcaacccagagtacttcgacggctactgggggtactcggtggccgtgggcgagttcgacggggatctcaacactacagaatatgtcgtcggtgcccccacttggagctggaccctgggagcggtggaaattttggattcctactaccagaggctgcatcggctgcgcggagagcagatggcgtcgtattttgggcattcagtggctgtcactgacgtcaacggggatgggaggcatgatctgctggtgggcgctccactgtatatggagagccgggcagaccgaaaactggccgaagtggggcgtgtgtatttgttcctgcagccgcgaggcccccacgcgctgggtgcccccagcctcctgctgactggcacacagctctatgggcgattcggctctgccatcgcacccctgggcgacctcgaccgggatggctacaatgacattgcagtggctgccccctacgggggtcccagtggccggggccaagtgctggtgttcctgggtcagagtgaggggctgaggtcacgtccctcccaggtcctggacagccccttccccacaggctctgcctttggcttctcccttcgaggtgccgtagacatcgatgacaacggatacccagacctgatcgtgggagcttacggggccaaccaggtggctgtgtacagagctcagccagtggtgaaggcctctgtccagctactggtgcaagattcactgaatcctgctgtgaagagctgtgtcctacctcagaccaagacacccgtgagctgcttcaacatccagatgtgtgttggagccactgggcacaacattcctcagaagctatccctaaatgccgagctgcagctggaccggcagaagccccgccagggccggcgggtgctgctgctgggctctcaacaggcaggcaccaccctgaacctggatctgggcggaaagcacagccccatctgccacaccaccatggccttccttcgagatgaggcagacttccgggacaagctgagccccattgtgctcagcctcaatgtgtccctaccgcccacggaggctggaatggcccctgctgtcgtgctgcatggagacacccatgtgcaggagcagacacgaatcgtcctggactgtggggaagatgacgtatgtgtgccccagcttcagctcactgccagcgtgacgggctccccgctcctagttggggcagataatgtcctggagctgcagatggacgcagccaacgagggcgagggggcctatgaagcagagctggccgtgcacctgccccagggcgcccactacatgcgggccctaagcaatgtcgagggctttgagagactcatctgtaatcagaagaaggagaatgagaccagggtggtgctgtgtgagctgggcaaccccatgaagaagaacgcccagataggaatcgcgatgttggtgagcgtggggaatctggaagaggctggggagtctgtgtccttccagctgcagatacggagcaagaacagccagaatccaaacagcaagattgtgctgctggacgtgccggtccgggcagaggcccaagtggagctgcgagggaactcctttccagcctccctggtggtggcagcagaagaaggtgagagggagcagaacagcttggacagctggggacccaaagtggagcacacctatgagctccacaacaatggccctgggactgtgaatggtcttcacctcagcatccaccttccgggacagtcccagccctccgacctgctctacatcctggatatacagccccaggggggccttcagtgcttcccacagcctcctgtcaaccctctcaaggtggactgggggctgcccatccccagcccctcccccattcacccggcccatcacaagcgggatcgcagacagatcttcctgccagagcccgagcagccctcgaggcttcaggatccagttctcgtaagctgcgactcggcgccctgtactgtggtgcagtgtgacctgcaggagatggcgcgcgggcagcgggccatggtcacggtgctggccttcctgtggctgcccagcctctaccagaggcctctggatcagtttgtgctgcagtcgcacgcatggttcaacgtgtcctccctcccctatgcggtgcccccgctcagcctgccccgaggggaagctcaggtgtggacacagctgctccgggccttggaggagagggccattccaatctggtgggtgctggtgggtgtgctgggtggcctgctgctgctcaccatcctggtcctggccatgtggaaggtcggcttcttcaagcggaaccggccacccctggaagaagatgatgaagagggggagtgatggtgcagcctacactattctagcaggagggttgggcgtgctacctgcaccgccccttctccaacaagttgcctccaagctttgggttggagctgttccattgggtcctcttggtgtcgtttccctcccaacagagctgggctaccccccctcctgctgcctaataaagagactgagccctg

SEQ ID NO: 37 - DNA, 7878 bp, Homo Sapiens
ttttccctgctctcaccgggcgggggagagaagccctctggacagcttctagagtgtgcaggttctcgtatccctcggccaagggtatcctctgcaaacctctgcaaacccagcgcaactacggtcccccggtcagacccaggatggggccagaacggacaggggccgcgccgctgccgctgctgctggtgttagcgctcagtcaaggcattttaaattgttgtttggcctacaatgttggtctcccagaagcaaaaatattttccggtccttcaagtgaacagtttggctatgcagtgcagcagtttataaatccaaaaggcaactggttactggttggttcaccctggagtggctttcctgagaaccgaatgggagatgtgtataaatgtcctgttgacctatccactgccacatgtgaaaaactaaatttgcaaacttcaacaagcattccaaatgttactgagatgaaaaccaacatgagcctcggcttgatcctcaccaggaacatgggaactggaggttttctcacatgtggtcctctgtgggcacagcaatgtgggaatcagtattacacaacgggtgtgtgttctgacatcagtcctgattttcagctctcagccagcttctcacctgcaactcagccctgcccttccctcatagatgttgtggttgtgtgtgatgaatcaaatagtatttatccttgggatgcagtaaagaattttttggaaaaatttgtacaaggcctggatataggccccacaaagacacaggtggggttaattcagtatgccaataatccaagagttgtgtttaacttgaacacatataaaaccaaagaagaaatgattgtagcaacatcccagacatcccaatatggtggggacctcacaaacacattcggagcaattcaatatgcaagaaaatatgcttattcagcagcttctggtgggcgacgaagtgctacgaaagtaatggtagttgtaactgacggtgaatcacatgatggttcaatgttgaaagctgtgattgatcaatgcaaccatgacaatatactgaggtttggcatagcagttcttgggtacttaaacagaaacgcccttgatactaaaaatttaataaaagaaataaaagcaatcgctagtattccaacagaaagatactttttcaatgtgtctgatgaagcagctctactagaaaaggctgggacattaggagaacaaattttcagcattgaaggtactgttcaaggaggagacaactttcagatggaaatgtcacaagtgggattcagtgcagattactcttctcaaaatgatattctgatgctgggtgcagtgggagcttttggctggagtgggaccattgtccagaagacatctcatggccatttgatctttcctaaacaagcctttgaccaaattctgcaggacagaaatcacagttcatatttaggttactctgtggctgcaatttctactggagaaagcactcactttgttgctggtgctcctcgggcaaattataccggccagatagtgctatatagtgtgaatgagaatggcaatatcacggttattcaggctcaccgaggtgaccagattggctcctattttggtagtgtgctgtgttcagttgatgtggataaagacaccattacagacgtgctcttggtaggtgcaccaatgtacatgagtgacctaaagaaagaggaaggaagagtctacctgtttactatcaaagagggcattttgggtcagcaccaatttcttgaaggccccgagggcattgaaaacactcgatttggttcagcaattgcagctctttcagacatcaacatggatggctttaatgatgtgattgttggttcaccactagaaaatcagaattctggagctgtatacatttacaatggtcatcagggcactatccgcacaaagtattcccagaaaatcttgggatccgatggagcctttaggagccatctccagtactttgggaggtccttggatggctatggagatttaaatggggattccatcaccgatgtgtctattggtgcctttggacaagtggttcaactctggtcacaaagtattgctgatgtagctatagaagcttcattcacaccagaaaaaatcactttggtcaacaagaatgctcagataattctcaaactctgcttcagtgcaaagttcagacctactaagcaaaacaatcaagtggccattgtatataacatcacacttgatgcagatggattttcatccagagtaacctccagggggttatttaaagaaaacaatgaaaggtgcctgcagaagaatatggtagtaaatcaagcacagagttgccccgagcacatcatttatatacaggagccctctgatgttgtcaactctttggatttgcgtgtggacatcagtctggaaaaccctggcactagccctgcccttgaagcctattctgagactgccaaggtcttcagtattcctttccacaaagactgtggtgaggacggactttgcatttctgatctagtcctagatgtccgacaaataccagctgctcaagaacaaccctttattgtcagcaaccaaaacaaaaggttaacattttcagtaacgctgaaaaataaaagggaaagtgcatacaacactggaattgttgttgatttttcagaaaacttgttttttgcatcattctccctgccggttgatgggacagaagtaacatgccaggtggctgcatctcagaagtctgttgcctgcgatgtaggctaccctgctttaaagagagaacaacaggtgacttttactattaactttgacttcaatcttcaaaaccttcagaatcaggcgtctctcagtttccaagccttaagtgaaagccaagaagaaaacaaggctgataatttggtcaacctcaaaattcctctcctgtatgatgctgaaattcacttaacaagatctaccaacataaatttttatgaaatctcttcggatgggaatgttccttcaatcgtgcacagttttgaagatgttggtccaaaattcatcttctccctgaaggtaacaacaggaagtgttccagtaagcatggcaactgtaatcatccacatccctcagtataccaaagaaaagaacccactgatgtacctaactggggtgcaaacagacaaggctggtgacatcagttgtaatgcagatatcaatccactgaaaataggacaaacatcttcttctgtatctttcaaaagtgaaaatttcaggcacaccaaagaattgaactgcagaactgcttcctgtagtaatgttacctgctggttgaaagacgttcacatgaaaggagaatactttgttaatgtgactaccagaatttggaacgggactttcgcatcatcaacgttccagacagtacagctaacggcagctgcagaaatcaacacctataaccctgagatatatgtgattgaagataacactgttacgattcccctgatgataatgaaacctgatgagaaagccgaagtaccaacaggagttataataggaagtataattgctggaatccttttgctgttagctctggttgcaattttatggaagctcggcttcttcaaaagaaaatatgaaaagatgaccaaaaatccagatgagattgatgagaccacagagctcagtagctgaaccagcagacctacctgcagtgggaaccggcagcatcccagccagggtttgctgtttgcgtgaatggatttctttttaaatcccatattttttttatcatgtcgtaggtaaactaacctggtattttaagagaaaactgcaggtcagtttggaatgaagaaattgtggggggtgggggaggtgcggggggcaggtagggaaataatagggaaaatacctattttatatgatgggggaaaaaaagtaatctttaaactggctggcccagagtttacattctaatttgcattgtgtcagaaacatgaaatgcttccaagcatgacaacttttaaagaaaaatatgatactctcagattttaagggggaaaactgttctctttaaaatatttgtctttaaacagcaactacagaagtggaagtgcttgatatgtaagtacttccacttgtgtatattttaatgaatattgatgttaacaagaggggaaaacaaaacacaggttttttcaatttatgctgctcatccaaagttgccacagatgatacttccaagtgataattttatttataaactaggtaaaatttgttgttggttccttttagaccacggctgccccttccacaccccatcttgctctaatgatcaaaacatgcttgaataactgagcttagagtatacctcctatatgtccatttaagttaggagagggggcgatatagagaataaggcacaaaattttgtttaaaactcagaatataacatgtaaaatcccatctgctagaagcccatcctgtgccagaggaaggaaaaggaggaaatttcctttctcttttaggaggcacaacagttctcttctaggatttgtttggctgactggcagtaacctagtgaatttctgaaagatgagtaatttctttggcaaccttcctcctcccttactgaaccactctcccacctcctggtggtaccattattatagaagccctctacagcctgactttctctccagcggtccaaagttatcccctcctttacccctcatccaaagttcccactccttcaggacagctgctgtgcattagatattaggggggaaagtcatctgtttaatttacacacttgcatgaattactgtatataaactccttaacttcagggagctattttcatttagtgctaaacaagtaagaaaaataagctcgagtgaatttctaaatgttggaatgttatgggatgtaaacaatgtaaagtaagacatctcaggatttcaccagaagttacagatgaggcactggaagccaccaaattagcaggtgcaccttctgtggctgtcttgtttctgaagtacttaaacttccacaagagtgaatttgacctaggcaagtttgttcaaaaggtagatcctgagatgatttggtcagattgggataaggcccagcaatctgcattttaacaagcaccccagtcactaggatgcagatggaccacactttgagaaacaccacccatttctactttttgcaccttattttctctgttcctgagcccccacattctctaggagaaacttagaggaaaagggcacagacactacatatctaaagctttggacaagtccttgacctctataaacttcagagtcctcattataaaatgggaagactgagctggagttcagcagtgatgcttttagttttaaaagtctatgatctggacttcctataatacaaatacacaatcctccaagaatttgacttggaaaaaaatgtcaaaggaaaacaggttatctgcccatgtgcatatggacaaccttgactaccctggcctggcccgtggtggcagtccagggctatctgtactgtttacagaattactttgtagttgacaacacaaaacaaacaaaaaaggcataaaatgccagcggtttatagaaaaaacagcatggtattctccagttaggtatgccagagtccaattcttttaacagctgtgagaatttgctgcttcattccaacaaaattttatttaaaaaaaaaaaaaaaagactggagaaactagtcattagcttgataaagaatatttaacagctagtggtgctggtgtgtacctgaagctccagctacttgagagactgagacaggaagatcgcttgagcccaggagttcaagtccagcctaagcaacatagcaagaccctgtctcaaaaaaatgactatttaaaaagacaatgtggccaggcacggtggctcacacctgtaatcccaacactttgggaggctgaggccggtggatcacgaggtcaggagtttgagactagcctggccaacatggtgaaaccccatctctaataatataaaaattagctgggcgtagtagcaggtgcctgtaatcccagttactcgggaagctgaggcaggagaatcacttgaacccgggaggcagaggtttcagtgagccgagatcgcgccactgcactccagcctgggtgacagggcaagactctgtctcaaacaaacaaacaaaaaaaaagttagtactgtatatgtaaatactagcttttcaatgtgctatacaaacaattatagcacatccttccttttactctgtctcacctcctttaggtgagtacttccttaaataagtgctaaacatacatatacggaacttgaaagctttggttagccttgccttaggtaatcagcctagtttacactgtttccagggagtagttgaattactataaaccattagccacttgtctctgcaccatttatcacaccaggacagggtctctcaacctgggcgctactgtcatttggggccaggtgattcttccttgcaggggctgtcctgtaccttgtaggacagcagccctgtcctagaaggtatgtttagcagcattcctggcctctagctacccgatgccagagcatgctccccccgcagtcatgacaatcaaaaaatgtctccagacattgtcaaatgcctcctggggggcagtatttctcaagcacttttaagcaaaggtaagtattcatacaagaaatttagggggaaaaaacattgtttaaataaaagctatgtgttcctattcaacaatatttttgctttaaaagtaagtagagggcataaaagatgtcatattcaaatttccatttcataaatggtgtacagacaaggtctatagaatgtggtaaaaacttgactgcaacacaaggcttataaaatagtaagatagtaaaatagcttatgaagaaactacagagatttaaaattgtgcatgactcatttcagcagcaaaataagaactcctaactgaacagaaatttttctacctagcaatgttattcttgtaaaatagttacctattaaaactgtgaagagtaaaactaaagccaatttattatagtcacacaagtgattatactaaaaattattataaaggttataattttataatgtatttacctgtcctgatatatagctataacccaatatatgaaaatctcaaaaattaagacatcatcatacagaaggcaggattccttaaactgagatccctgatccatctttaatatttcaatttgcacacataaaacaatgcccttttgtgtacattcaggcatacccattttaatcaatttgaaaggttaatttaaacctctagaggtgaatgagaaacatgggggaaaagtatgaaataggtgaaaatcttaactatttctttgaactctaaagactgaaactgtagccattatgtaaataaagtttcatatgtacctgtttattttggcagattaagtcaaaatatgaatgtatatattgcataactatgttagaattgtatatattttaaagaaattgtcttggatattttcctttatacataatagataagtcttttttcaaatgtggtgtttgatgtttttgattaaatgtgttttgcctctttccacaaaaactgtaaaaataaatgcatgtttgtacaaaaagttgcagaattcatttgatttatgagaaacaaaaattaaattgtagtcaacagttagtagtttttctcatatccaagtataacaaacagaaaagtttcattattgtaacccacttttttcataccacattattgaatattgttacaattgttttgaaaataaagccattttctttgggcttttataagttaaaaaaaaaa

SEQ ID NO: 38 - DNA, 3437 bp, Mus musculus
ggggtgggccctcccccgtcctagtcgacgtctagaggctattgccctctgccatgcctgctgctcagccatgagctccagtctgataagctgaaacttccggtggtgggaacttcagggaaagggaaagagctggcccattcctgtttaggacgtttgggaagaaggaagatggccagagcctcctgcgcatggcattccctgtggcttctgcagtggacaccactgttcttgggtcctagtgctgttcctccagtctgggccttgaacctggactctgagaagttctccgtctatgcaggtcccaatggcagccactttggcttctcagtggactttcataaggacaaacatggaagcgtgtccatcgtggtgggcgcccccagggccctaaacgcaagtcaagaggagactggagccgtattcctgtgcccctggaaggccaatggtggcaagtgcaacccgttgctcttcgacctcagggatgagacacgaaacctaggcttccaaattttccaaaccttcaagaccgggcaaggacttggggcgtcggtcgtcagctggaatgacgtcattgtggcctgtgccccctggcagcactggaatgtcctggaaaagcgcgatgaggcagagaagactccggtaggaggctgcttcttggctcagctccaaagcgggggccgcgcagagtactcgccctgtcgcgccaacaccatgagctccgtttacgcagagagttttcgcggagacaagcgatactgtgaagcaggcttcagcttggcggtgacccaggctggggagctagtgcttggagcccctggaggctacttttttttaggtctcctggctcgcgttccaattgagaacatcatctccagctaccgcccgggtacccttttgtggcatgtttccaaccagcgcttcacctacgacaacagcaacccagtgtttttcgatggttaccggggatattcggtagccgtgggcgagtttgatggggatccgagcactacagagtacgtatcgggtgcccccacttggagctggaccttgggagcggtggaaattttggactcctactaccagcccctgcaccggcttcatggagaacagatggcttcatatttcgggcactcggtggcagtcactgacgtcaacggggacgggaggcatgacctactggtgggggctccattgtatatggagagcagggcagaccgcaagctggccgaggtgggccgcgtttatttgtttctgcagcctaagggcccccaagctctgagcacacccactctcctgctgactggcacccagctctatgggagatttggatctgccattgcacccctgggtgacctcaaccgagacggctataatgatattgctgtggctgccccctatgggggtcccagtggtcagggccaagtgctgatattcctgggtcagagtgaagggctgagtccacgcccctcccaggttctggacagccccttccccacaggctctggctttggcttctcccttcgtggtgctgtagacatcgatgacaatggatacccagacctgattgtgggagcatatggagctagcaaggtggctgtgtacagagctcagcctgtggtgatggccactgtccaactgatggttcaagactccctgaatccaacactgaagaactgtgtcctggatcagaccaagacaccagtcagctgcttcaacatccagatgtgtgtgggagccacaggacacaacattcctcagaagctgcatctaaaggcagagctgcagctggacttacagaagccccgtcagggccgccgggtgctcctgctggcatcccaacaggcgagcctcaccctgagcctggacctgggcggaagagacaagcctatctgccacaccacgggggccttccttcgggatgaggccgacttccgggacaagctgagcccaattgtgctaagcctcaacgtgtcgctgcccccagaagagactggaggagcccctgccgtggtattgcatggagaaacccatgtccaggagcagacacggatcatcctggattgtggggaagacgacctgtgtgtgccacagctccggctcacagctactgcgggggactccccactcctaatcggtgctgacaatgtgttggagctgaagattgaagcagccaatgatggtgagggagcctatgaagcggagctggctgtgcatctgcctccaggtgcccactacatgcgggctctcagcaacattgagggctttgagaggcttgtctgcactcagaagaaagagaatgagtccagggtggccctctgtgagctgggcaaccccatgaaaaaggacacccggataggaatcacaatgctggtgagtgtggagaacctggaagaagctggcgagtccgtgtccttccagctgcaggtcaggagcaagaacagccagaatccaaacagtaaggtcgtgatgttgcctgtggcaatccaagctgaagccacagtggagcttcgagggaattccttccctgcctccctggtggtggcagcagaagaaggtgacagggagcaggaagacttggacagatgggtctccaggctggagcacacctatgagctccacaacattggccctggcactgtgaatggcctcagacttctcatccacattcctggccagtcccagccctcggatctgctctacatcctggatgtgcagccgcagggaggtctcctttgctccacacagccatctcccaaggtggactggaaactatccacgcccagcccttcttccattcgccccgtccatcaccagcgtgagcgcagacaggcattcctgcaggggcccaagccagggcagcaggacccagttctggtgagctgcgacggctcagcgtcctgtacggtggtggagtgtgagctgcgggagatggtgcgcgggcagcgggccatggttactgtgcaggtcatgctggggctgtccagcctccgccagaggccgcaggagcagtttgtgctgcagtcgcacgcctggttcaacgtctcctccctaccttactcggtgccggtggtcagcttgcccagtgggcaagctcgggtgcagacacagctgcttcgggccttggaggagagggccattcctgtctggtgggtgctggtgggcgtgctgggcggtctgctgctgctgaccctgctagttttggccatgtggaaggctggcttcttcaagcggaatcgaccgcctctggaggaagatgaagaggaagagtgacagcagagggggcggggttcctggtgggaaagctgggtacctgcttcctgtttccagccagttgcctttgctcagaactccatgggggtcttccctttccagcagaccgggtgcatcccttttcctaatacctaataaagagcttgacagtgatatccgccatg

SEQ ID NO: 39 - DNA, 2222 bp, Homo Sapiens
attcggcacgagggaggaagcgagaggtgctgccctccccccggagttggaagcgcgttacccgggtccaaaatgcccaagaagaagccgacgcccatccagctgaacccggcccccgacggctctgcagttaacgggaccagctctgcggagaccaacttggaggccttgcagaagaagctggaggagctagagcttgatgagcagcagcgaaagcgccttgaggcctttcttacccagaagcagaaggtgggagaactgaaggatgacgactttgagaagatcagtgagctgggggctggcaatggcggtgtggtgttcaaggtctcccacaagccttctggcctggtcatggccagaaagctaattcatctggagatcaaacccgcaatccggaaccagatcataagggagctgcaggttctgcatgagtgcaactctccgtacatcgtgggcttctatggtgcgttctacagcgatggcgagatcagtatctgcatggagcacatggatggaggttctctggatcaagtcctgaagaaagctggaagaattcctgaacaaattttaggaaaagttagcattgctgtaataaaaggcctgacatatctgagggagaagcacaagatcatgcacagagatgtcaagccctccaacatcctagtcaactcccgtggggagatcaagctctgtgactttggggtcagcgggcagctcatcgactccatggccaactccttcgtgggcacaaggtcctacatgtcgccagaaagactccaggggactcattactctgtgcagtcagacatctggagcatgggactgtctctggtagagatggcggttgggaggtatcccatccctcctccagatgccaaggagctggagctgatgtttgggtgccaggtggaaggagatgcggctgagaccccacccaggccaaggacccccgggaggccccttagctcatacggaatggacagccgacctcccatggcaatttttgagttgttggattacatagtcaacgagcctcctccaaaactgcccagtggagtgttcagtctggaatttcaagattttgtgaataaatgcttaataaaaaaccccgcagagagagcagatttgaagcaactcatggttcatgcttttatcaagagatctgatgctgaggaagtggattttgcaggttggctctgctccaccatcggccttaaccagcccagcacaccaacccatgctgctggcgtctaagtgtttgggaagcaacaaagagcgagtcccctgcccggtggtttgccatgtcgcttttgggcctccttcccatgcctgtctctgttcagatgtgcatttcacctgtgacaaaggatgaagaacacagcatgtgccaagattctactcttgtcatttttaatattactgtctttattcttattactattattgttcccctaagtggattggctttgtgcttggggctatttgtgtgtatgctgatgatcaaaacctgtgccaggctgaattacagtgaaatttttggtgaatgtgggtagtcattcttacaattgcactgctgttcctgctccatgactggctgtctgcctgtattttcggactttgacatttgacatttggtggactttatcttgctgggcatactttctctctaggagggagccttgtgagatccttcacaggcagtgcatgtgaagcatgctttgctgctatgaaaatgagcatcagagagtgtacatcatgttattttattattattatttgcttttcatgtagaactcagcagttgacatccaaatctagccagagcccttcactgccatgatagctggggcttcaccagtctgtctactgtggtgatctgtagacttctggttgtatttctatatttattttcagtatactgtgtgggatacttagtggtatgtctctttaagttttgattaatgtttcttaaatggaattatttgaatgtcacaaattgatcaagatattaaaatgtcggatttatctttccccatatccaagtaccaatgctgttgtaaacaacgtgtatagtgcctaaaattgtatgaaaatccttttaaccattttaacctagatgtttaacaaatctaatctcttattctaataaatatactatgaaataaaaaaaaaaggagaaagctaaaaaaaaaaaaaaaaaaaaaaaaaaaaaaaaaaaaaaa

SEQ ID NO: 40 - DNA, 2387 bp, Mus musculus
agtccctcactgggacgtctgtgcgcggcgtctcggagcgccggagcagcggtggccgcactttctccaagctggggctgtagctgagctgtgggtagtgcgcagggagccgtccgagcccgaggaaccggtgtgctgaggcgagagttcccggccggcgagcgcgcgcagctggttctccgcgtgggttgggcggagggtcccaggagcgcggcgttgatcgagccgccccgactctgggcagagccgagggaggaagcgagaagcggccgcgcgctccctgctgagttgcaggctctttcccggctgcaagatgcccaagaagaagccgacgcccatccagctgaacccggcccccgatggctcggcggttaacgggaccagctcggccgagaccaacctggaggccttgcagaagaagctggaggagctggagcttgacgagcagcagcggaagcggctcgaggcctttctgacgcagaagcagaaggtgggggaactgaaggatgatgactttgagaagatcagcgaactgggagctggcaacggtggagtggtcttcaaggtctcccacaagccatctggcctggttatggctagaaagctgatccacctggagatcaaacccgcaatccggaaccagatcatccgggagctgcaggtactgcacgagtgcaactccccgtacatcgtgggcttctacggggccttctacagcgacggcgagatcagcatctgcatggagcacatggatggtgggtccttggatcaagttctgaagaaagctggaagaattcctgagcaaattttaggaaaagttagcattgctgtgataaaaggcctgacctatcttcgggagaagcacaagattatgcacagagatgtcaagccatccaacattctagtgaactcacgtggggagatcaaactctgtgattttggggtcagcgggcagctaattgactctatggccaactccttcgtgggcacgagatcctacatgtcgcctgagagactccaggggactcactactctgtgcagtcggacatctggagcatggggctctctctggtggagatggcagttgggagataccccattcctcctcctgatgccaaggagctggagctactgtttggatgccatgtggaaggagacgcagccgaaacaccacccaggccaaggacccctgggaggcctctcagctcatatggaatggacagccgacctcccatggcaatttttgagttgttggattacattgtcaatgagcctcctccaaaactgcccagtggagtattcagtctggagtttcaggattttgtgaataaatgcttaataaagaaccctgcagagagagcagatctgaagcagctcatggtacatgctttcatcaaaagatctgacgccgaggaggtagacttcgcaggctggctctgctccaccattgggcttaaccagcccagcacaccaacccacgctgccagcatctgagcctttaggaagcagcaaagaggaattctctgcccagtggcatgccatgttgctttcaggcctctcccatgcttgtctatgttcagacgtgcatctcatctgtgacaaaggatgaagaacacagcatgtgccaaattgtacttgtgtcatttttaatatcattgtctttatcactatggttactcccctaagtggattggctttgtgcttggggctatttgtctgttcatcaaacacatgccaggctgaactacagtgaaaccctagtgacctgggtggtcgttcttactgatgtttgcactgctgttcatcgtgactcactagctggctgcctgtattgtcaggattctcggaccttggtacttcactcttgctggtgacctctcagtctgagagggagccttgtgagacccttcacaggcagtgcatgcatggaaagcatgctttgctgctactgaaatgagcatcagaacgtgtacgtcatggtatttttattttttgcttttggtatagaactcagcaattcccatcaaaaaaacctaagcagagcccatcactgccatgatagctgggcttcagtctgtctactgtggtgatttttagacttctggttgtatttctatatttatttttaaatatacagtgtgggatacttagtggtgtgtgtctctaagtttggattagtgtttctaaattggtggttattttgaatgtcacaaatggattaaagcatcaatgtatcaagagttctatctttcttccagtctaagtaccaatgctattgtaaacaacgtgtatagtgcctacaaattgtatgaaaccccttttaaccactttaatcaagatgtttatcaaatctaatctcttattctaataaaaatactatcaagtt

SEQ ID NO: 41 - DNA, 3105 bp, Homo Sapiens
caactgggggcgccccggacgaccatgagagataaggactgagggccaggaaggggaagcgagcccgccgagaggtggcggggactgctcacgccaagggccacagcggccgcgctccggcctcgctccgccgctccacgcctcgcgggatccgcgggggcagcccggccgggcggggatgccggggctggggcggagggcgcagtggctgtgctggtggtgggggctgctgtgcagctgctgcgggcccccgccgctgcggccgcccttgcccgctgccgcggccgccgccgccggggggcagctgctgggggacggcgggagccccggccgcacggagcagccgccgccgtcgccgcagtcctcctcgggcttcctgtaccggcggctcaagacgcaggagaagcgggagatgcagaaggagatcttgtcggtgctggggctcccgcaccggccccggcccctgcacggcctccaacagccgcagcccccggcgctccggcagcaggaggagcagcagcagcagcagcagctgcctcgcggagagccccctcccgggcgactgaagtccgcgcccctcttcatgctggatctgtacaacgccctgtccgccgacaacgacgaggacggggcgtcggagggggagaggcagcagtcctggccccacgaagcagccagctcgtcccagcgtcggcagccgcccccgggcgccgcgcacccgctcaaccgcaagagccttctggcccccggatctggcagcggcggcgcgtccccactgaccagcgcgcaggacagcgccttcctcaacgacgcggacatggtcatgagctttgtgaacctggtggagtacgacaaggagttctcccctcgtcagcgacaccacaaagagttcaagttcaacttatcccagattcctgagggtgaggtggtgacggctgcagaattccgcatctacaaggactgtgttatggggagttttaaaaaccaaacttttcttatcagcatttatcaagtcttacaggagcatcagcacagagactctgacctgtttttgttggacacccgtgtagtatgggcctcagaagaaggctggctggaatttgacatcacggccactagcaatctgtgggttgtgactccacagcataacatggggcttcagctgagcgtggtgacaagggatggagtccacgtccacccccgagccgcaggcctggtgggcagagacggcccttacgacaagcagcccttcatggtggctttcttcaaagtgagtgaggtgcacgtgcgcaccaccaggtcagcctccagccggcgccgacaacagagtcgtaatcgctctacccagtcccaggacgtggcgcgggtctccagtgcttcagattacaacagcagtgaattgaaaacagcctgcaggaagcatgagctgtatgtgagtttccaagacctgggatggcaggactggatcattgcacccaagggctatgctgccaattactgtgatggagaatgctccttcccactcaacgcacacatgaatgcaaccaaccacgcgattgtgcagaccttggttcaccttatgaaccccgagtatgtccccaaaccgtgctgtgcgccaactaagctaaatgccatctcggttctttactttgatgacaactccaatgtcattctgaaaaaatacaggaatatggttgtaagagcttgtggatgccactaactcgaaaccagatgctggggacacacattctgccttggattcctagattacatctgccttaaaaaaacacggaagcacagttggaggtgggacgatgagactttgaaactatctcatgccagtgccttattacccaggaagattttaaaggacctcattaataatttgctcacttggtaaatgacgtgagtagttgttggtctgtagcaagctgagtttggatgtctgtagcataaggtctggtaactgcagaaacataaccgtgaagctcttcctaccctcctcccccaaaaacccaccaaaattagttttagctgtagatcaagctatttggggtgtttgttagtaaatagggaaaataatctcaaaggagttaaatgtattcttggctaaaggatcagctggttcagtactgtctatcaaaggtagattttacagagaacagaaatcggggaagtggggggaacgcctctgttcagttcattcccagaagtccacaggacgcacagcccaggccacagccagggctccacggggcgcccttgtctcagtcattgctgttgtatgttcgtgctggagttttgttggtgtgaaaatacacttatttcagccaaaacataccatttctacacctcaatcctccatttgctgtactctttgctagtaccaaaagtagactgattacactgaggtgaggctacaaggggtgtgtaaccgtgtaacacgtgaaggcaatgctcacctcttctttaccagaacggttctttgaccagcacattaacttctggactgccggctctagtaccttttcagtaaagtggttctctgcctttttactatacagcataccacgccacagggttagaaccaacgaagaaaataaaatgagggtgcccagcttataagaatggtgttagggggatgagcatgctgtttatgaacggaaatcatgatttcccttgtagaaagtgaggctcagattaaattttagaatattttctaaatgtctttttcacaatcatgtactgggaaggcaatttcatactaaactgattaaataatacatttataatctacaactgtttgcacttacagctttttttgtaaatataaactataatttattgtctattttatatctgttttgctgtaacattgaaggaaagaccagacttttaaaaaaaaagagtttatttagaaagtatcatagtgtaaacaaacaaattgtaccactttgattttcttggaatacaagactcgtgatgcaaagctgaagttgtgtgtacaagactcttgacagttgtgcttctctaggaggttgggtttttttaaaaaaagaattatctgtgaaccatacgtgattaataaagatttcctttaaggca

SEQ ID NO: 42 - DNA, 2410 bp, Mus musculus
gatcctggccgtcgccccgtcgtctcttctccacccgggcttctgggggcgccgcggatgaccatgagagataaggactgagtgccaggaccgggaagagagcccgccgagaggtggcgggggctgcccactccgagggccacagcctccgcgctccggcctcgctccgccgctcgacgcctcgcgggccccgcgggggcagccgggctgggcggcgatgcccgggctggggcggagggcgcagtggctgtgctggtggtgggggttgctgtgcagctgcggccccccgccactgcggccccctctgccggtagccgcggccgccgccggggggcagctgctgggagccggcgggagcccggtgcgcgctgagcagccaccgccacagtcctcttcttcgggcttcctctatcggcggctcaagacccacgagaagcgggagatgcaaaaggagatcctgtcggtgctggggctcccgcacaggccgcggcccctgcacggtctccagcagcctcagcccccggtgctcccgccacagcagcagcagcagcagcagcagcagcagacggcccgcgaggagccccctccagggcggctgaagtccgctccactcttcatgctggatctctacaacgccctgtccaatgacgacgaagaggatggggcatcggagggtgtggggcaagagcctgggtcccacggaggggccagctcgtcccagctcaggcagccgtctcccggcgctgcacactccttgaaccgcaagagtctcctggccccgggacccggtggcggtgcgtccccactgactagcgcgcaggacagcgctttcctcaacgacgcggacatggtcatgagctttgtgaacctggtggagtacgacaaggagttctccccacatcaacgacaccacaaagagttcaagttcaacctatcccagattcctgagggtgaggcggtgacggctgctgagttccgcgtctacaaggactgtgtggtggggagttttaaaaaccaaacctttcttatcagcatttaccaagtcttgcaggagcatcagcacagagactctgacctatttttgttggacacccgggtggtgtgggcctcagaagaaggttggctggaatttgacatcacagcaactagcaatctgtgggtggtgacaccgcagcacaacatggggctccagctgagtgtggtgactcgggatggactccacgtcaacccccgtgcggcgggcctggtgggcagagacggcccttacgacaagcagcccttcatggtggccttcttcaaggtgagcgaggtccacgtgcgcaccaccaggtcagcctccagtcggcggcggcagcagagtcgcaaccggtccacccagtcgcaggacgtgtcccggggctccggttcttcagactacaacggcagtgagttaaaaacagcttgcaagaagcatgagctctatgtgagcttccaggacctgggatggcaggactggatcattgcacccaaaggctacgctgccaactactgtgatggagagtgttccttcccactcaacgcacacatgaatgccaccaaccacgccattgtacagaccttggtccaccttatgaatcccgagtacgtccccaaaccatgctgcgcaccaaccaaactgaatgccatctcggttctttacttcgatgataactccaatgtcatcttgaaaaagtacaggaatatggtcgtgagagcttgtggttgccattaagttgaagctggtgtgtgtgtgtgggtgggggcatggttctgccttggattcctaacaacaacatctgccttaaaccacgaacaacagcacagcgaagcgggatggtgacacacagagggatcgtgacacgcagacacatctcccgctggtgccttacccacggaggcttttatgaggaccttgtcaagggctttcccagttcctaactgagcagttgctggtctgcaggaagctggaaggcttgtagtacaggcctggaaactgcagttacctaatgttcgcctcccccaaccccgcccggagtagttttagcttttagatctagctgcttgtggtgtaagtagagagtaaacttgaaggaatattaaatatccctgggttgaaagacccggtggtggctctacagcacccatcccagggagatttttgcagacatccgaatggaggggagaagggcactctttcaggttccattcccagcaagggcagctcacacaggacctgcagcctggccatcagcaggctctgtggaggtgccttctgtctactgttgtagttacgtgttttgtgttgactctcggtggtgtgagaatgtactaatctctgtcaagacaaactgtagcatttccaccccatcctcctccctccctcacagaattc

SEQ ID NO: 43 - DNA, 3665 bp, Homo Sapiens
ggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgtgtacgttggtgcccgctgctgtctaatgccctggagcctccctggcccccatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaaaaaaaaaaaa

SEQ ID NO: 44 - DNA, 3596 bp, Homo Sapiens
ggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccgtccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaaaaaaaaaaaa

SEQ ID NO: 45 - DNA, 3542 bp, Homo Sapiens
ggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaaaaaaaaaaaa

SEQ ID NO: 46 - DNA, 3507 bp, Homo Sapiens
ggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaaaaaaaaaaaa

SEQ ID NO: 47 - DNA, 3410 bp, Homo Sapiens
ggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaaaaaaaaaaaa

SEQ ID NO: 48 - DNA, 3476 bp, Homo Sapiens
ggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaaaaaaaaaaaa

SEQ ID NO: 49 - DNA, 3614 bp, Homo Sapiens
ggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgttccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaaaaaaaaaaaa

SEQ ID NO: 50 - DNA, 3547 bp, Mus musculus
agcgcagaggcttggggcagccgagctgcagcgaggccgcggcactgggggcgagctgagcggcggcagcggagctctgtcgcgagacgcagcgacaaggcagactattcagcggactcaccagcccgggagtctgtgctctgggatttgatattcaaacctcttaatttttttttcttaaactgtattgttttacgctttaatttatttttgcttcctattcccctcttaaatcgtgccaacggtttgaggaggttggttcttcactccctcaaatcacttcggattgtggaaatcagcagacgaaagaggtatcaagagctccagagagaagtcaaggaagagagagagagaccggtcagagagagcgcgctggcgagcgaacagagagagggacaggggcaaagtgactgacctgcttttgggggtgaccgccagagcgcggcgtgagccctcccccttgggatcttgcatcggaccagtcgcgctgacggacagacagacagacaccgcccccagccccagcgcccacctcctcgccggcgggctgccgacggtggacgcggcggcgagccgcgaggaaccgaagcccgcgcccggaggcggggtggagggggtcggggctcgcgggattgcacggaaacttttcgtccaacttctgggctcttctcgctccgtagtagccgtggtctgcgccgcaggagacaaaccgatcggagctgggagaagtgctagctcgggcctggagaagccggggcccgagaagagaggggaggaagagaaggaagaggagagggggccgcagtgggcgctcggctctcaggagccgagctcatggacgggtgaggcggccgtgtgcgcagacagtgctccagccgcgcgcgcgccccaggccccggcccgggcctcggttccagaagggagaggagcccgccaaggcgcgcaagagagcgggctgcctcgcagtccgagccggagagggagcgcgagccgcgccggccccggacgggcctccgaaaccatgaactttctgctctcttgggtgcactggaccctggctttactgctgtacctccaccatgccaagtggtcccaggctgcacccacgacagaaggagagcagaagtcccatgaagtgatcaagttcatggatgtctaccagcgaagctactgccgtccgattgagaccctggtggacatcttccaggagtaccccgacgagatagagtacatcttcaagccgtcctgtgtgccgctgatgcgctgtgcaggctgctgtaacgatgaagccctggagtgcgtgcccacgtcagagagcaacatcaccatgcagatcatgcggatcaaacctcaccaaagccagcacataggagagatgagcttcctacagcacagcagatgtgaatgcagaccaaagaaagacagaacaaagccagaaaaaaaatcagttcgaggaaagggaaagggtcaaaaacgaaagcgcaagaaatcccggtttaaatcctggagcgttcactgtgagccttgttcagagcggagaaagcatttgtttgtccaagatccgcagacgtgtaaatgttcctgcaaaaacacagactcgcgttgcaaggcgaggcagcttgagttaaacgaacgtacttgcagatgtgacaagccaaggcggtgagccaggctgcaggaaggagcctccctcagggtttcgggaaccagacctctcaccggaaagaccgattaaccatgtcaccaccacgccatcatcgtcaccgttgacagaacagtccttaatccagaaagcctgacatgaaggaagaggagactcttcgaggagcactttgggtccggagggcgagactccggcagacgcattcccgggcaggtgaccaagcacggtccctcgtgggactggattcgccattttcttatatctgctgctaaatcgccaagcccggaagattagggttgtttctgggattcctgtagacacacccacccacatacacacatatatatatattatatatataaataaatatatatgttttatatataaaatatatatatattcttttttttaaattaactctgctaatgttattggtgtcttcactggatatgtttgactgctgtggacttgtgttgggaggaggatgtcctcactcggatgccgacacgggagacaatgggatgaaaggcttcagtgtggtctgagagaggccgaagtccttttgcctgccggggagcaagcaaggccagggcacgggggcacattggctcacttccagaaacacgacaaacccattcctggccctgagtcaagaggacagagagacagatgatgacagagaaagagataaagatgccggttccaaccagaagtttggggagcctcaggacatggcatgctttgtggatccccatgatagtctacaaaagcaccccgcccctctgggcactgcctggaagaatcgggagcctggccagccttcagctcgctcctccacttctgaggggcctaggaggcctcccacaggtgtcccggcaagagaagacacggtggtggaagaagaggcctggtaatggcccctcctcctgggaccccttcgtcctctccttaccccacctcctgggtacagcccaggaggaccttgtgtgatcagaccattgaaaccactaattctgtccccaggagacttggctgtgtgtgtgagtggcttacccttcctcatcttcccttcccaaggcacagagcaatggggcaggacccgcaagcccctcacggaggcagagaaaagagaaagtgttttatatacggtacttatttaatagccctttttaattagaaattaaaacagttaatttaattaaagagtagggtttttttcagtattcttggttaatatttaatttcaactatttatgagatgtatctctcgctctctcttatttgtacttgtgtgtgtgtgtgtgtgtgtgtgtgtgtgtgtgtgtgtgtgtgtatgaaatctgtgtttccaatctctctctcccagatcggtgacagtcactagcttgtcctgagaagatatttaattttgctaacactcagctctgccctcccttgtccccaccacacattcctttgaaataaggtttcaatatacatttacatactatatatatatttggcaacttgtgtttgtatataaatatatatatatatatatatgtttatgtatatatgtgattctgataaaatagacattgctattctgttttttatatgtaaaaacaaaacaagaaaaatagagaattctacatactaaatctctctccttttttaattttaatatttgttatcatttatttattggtgctactgtttatccgtaataattgtgggggaaaaagatattaacatcacgtctttgtctctagagcagttttccgagatattccgtagtacatatttatttttaaacagcaacaaagaaatacagatatatcttaaaaaaaaaagcattttgtattaaagaattgaattctgatctcaaaaaaaaaaaaaaaaaaa

SEQ ID NO: 51 - DNA, 3343 bp, Mus musculus
agcgcagaggcttggggcagccgagctgcagcgaggccgcggcactgggggcgagctgagcggcggcagcggagctctgtcgcgagacgcagcgacaaggcagactattcagcggactcaccagcccgggagtctgtgctctgggatttgatattcaaacctcttaatttttttttcttaaactgtattgttttacgctttaatttatttttgcttcctattcccctcttaaatcgtgccaacggtttgaggaggttggttcttcactccctcaaatcacttcggattgtggaaatcagcagacgaaagaggtatcaagagctccagagagaagtcaaggaagagagagagagaccggtcagagagagcgcgctggcgagcgaacagagagagggacaggggcaaagtgactgacctgcttttgggggtgaccgccagagcgcggcgtgagccctcccccttgggatcttgcatcggaccagtcgcgctgacggacagacagacagacaccgcccccagccccagcgcccacctcctcgccggcgggctgccgacggtggacgcggcggcgagccgcgaggaaccgaagcccgcgcccggaggcggggtggagggggtcggggctcgcgggattgcacggaaacttttcgtccaacttctgggctcttctcgctccgtagtagccgtggtctgcgccgcaggagacaaaccgatcggagctgggagaagtgctagctcgggcctggagaagccggggcccgagaagagaggggaggaagagaaggaagaggagagggggccgcagtgggcgctcggctctcaggagccgagctcatggacgggtgaggcggccgtgtgcgcagacagtgctccagccgcgcgcgcgccccaggccccggcccgggcctcggttccagaagggagaggagcccgccaaggcgcgcaagagagcgggctgcctcgcagtccgagccggagagggagcgcgagccgcgccggccccggacgggcctccgaaaccatgaactttctgctctcttgggtgcactggaccctggctttactgctgtacctccaccatgccaagtggtcccaggctgcacccacgacagaaggagagcagaagtcccatgaagtgatcaagttcatggatgtctaccagcgaagctactgccgtccgattgagaccctggtggacatcttccaggagtaccccgacgagatagagtacatcttcaagccgtcctgtgtgccgctgatgcgctgtgcaggctgctgtaacgatgaagccctggagtgcgtgcccacgtcagagagcaacatcaccatgcagatcatgcggatcaaacctcaccaaagccagcacataggagagatgagcttcctacagcacagcagatgtgaatgcagaccaaagaaagacagaacaaagccagaaaaatgtgacaagccaaggcggtgagccaggctgcaggaaggagcctccctcagggtttcgggaaccagacctctcaccggaaagaccgattaaccatgtcaccaccacgccatcatcgtcaccgttgacagaacagtccttaatccagaaagcctgacatgaaggaagaggagactcttcgaggagcactttgggtccggagggcgagactccggcagacgcattcccgggcaggtgaccaagcacggtccctcgtgggactggattcgccattttcttatatctgctgctaaatcgccaagcccggaagattagggttgtttctgggattcctgtagacacacccacccacatacacacatatatatatattatatatataaataaatatatatgttttatatataaaatatatatatattcttttttttaaattaactctgctaatgttattggtgtcttcactggatatgtttgactgctgtggacttgtgttgggaggaggatgtcctcactcggatgccgacacgggagacaatgggatgaaaggcttcagtgtggtctgagagaggccgaagtccttttgcctgccggggagcaagcaaggccagggcacgggggcacattggctcacttccagaaacacgacaaacccattcctggccctgagtcaagaggacagagagacagatgatgacagagaaagagataaagatgccggttccaaccagaagtttggggagcctcaggacatggcatgctttgtggatccccatgatagtctacaaaagcaccccgcccctctgggcactgcctggaagaatcgggagcctggccagccttcagctcgctcctccacttctgaggggcctaggaggcctcccacaggtgtcccggcaagagaagacacggtggtggaagaagaggcctggtaatggcccctcctcctgggaccccttcgtcctctccttaccccacctcctgggtacagcccaggaggaccttgtgtgatcagaccattgaaaccactaattctgtccccaggagacttggctgtgtgtgtgagtggcttacccttcctcatcttcccttcccaaggcacagagcaatggggcaggacccgcaagcccctcacggaggcagagaaaagagaaagtgttttatatacggtacttatttaatagccctttttaattagaaattaaaacagttaatttaattaaagagtagggtttttttcagtattcttggttaatatttaatttcaactatttatgagatgtatctctcgctctctcttatttgtacttgtgtgtgtgtgtgtgtgtgtgtgtgtgtgtgtgtgtgtgtgtgtatgaaatctgtgtttccaatctctctctcccagatcggtgacagtcactagcttgtcctgagaagatatttaattttgctaacactcagctctgccctcccttgtccccaccacacattcctttgaaataaggtttcaatatacatttacatactatatatatatttggcaacttgtgtttgtatataaatatatatatatatatatatgtttatgtatatatgtgattctgataaaatagacattgctattctgttttttatatgtaaaaacaaaacaagaaaaatagagaattctacatactaaatctctctccttttttaattttaatatttgttatcatttatttattggtgctactgtttatccgtaataattgtgggggaaaaagatattaacatcacgtctttgtctctagagcagttttccgagatattccgtagtacatatttatttttaaacagcaacaaagaaatacagatatatcttaaaaaaaaaagcattttgtattaaagaattgaattctgatctcaaaaaaaaaaaaaaaaaaa

SEQ ID NO: 52 - DNA, 2857 bp, Mus musculus
gtctgctctgcgctctgtacttccctgccgaagctctccacgatttgaccatctgctttcgtgacctttgctccctgggctcgacaggggggccgctgcctgcaacaagtgttcttctctttctctgggagaacccaaatgctcccccaatcctcactcgagggttctattggggaagctggacgggagggactggtctatgcagagttaaaagccagtcttgaggtgctgttgcctagtgggtggatctgggcccagacggggtggagagtgggaggaagctcagcaacaggctggatgggtagctcttaggtagcatttagggggtcagggtgaagcttgggaggggtgcaggaggggaagggatgggggtggtaagaatccaggacctgaattcccagcctggccaacccttgcagctgtctgctctcaagaggagcaagagccccttgaggccagcagggttgggggagttggagtgacctgaggttcttttctgttagagccctggtcctcctatctccaccacctatccctgctcagtagaacccctgggtgctaaatggcaggagccccggggtgtcccataggggtatggctggctgggtcactaaccactgtgatctgctccctccctctacagatcatgcggatcaaacctcaccaaagccagcacataggagagatgagcttcctacagcacagcagatgtgaatgcagaccaaagaaagacagaacaaagccagaaaaaaaatcagttcgaggaaagggaaagggtcaaaaacgaaagcgcaagaaatcccggtttaaatcctggagcgttcactgtgagccttgttcagagcggagaaagcatttgtttgtccaagatccgcagacgtgtaaatgttcctgcaaaaacacagactcgcgttgcaaggcgaggcagcttgagttaaacgaacgtacttgcagatgtgacaagccaaggcggtgagccaggctgcaggaaggagcctccctcagggtttcgggaaccagacctctcaccggaaagaccgattaaccatgtcaccaccacgccatcatcgtcaccgttgacagaacagtccttaatccagaaagcctgacatgaaggaagaggagactcttcgaggagcactttgggtccggagggcgagactccggcagacgcattcccgggcaggtgaccaagcacggtccctcgtgggactggattcgccattttcttatatctgctgctaaatcgccaagcccggaagattagggttgtttctgggattcctgtagacacacccacccacatacacacatatatatatattatatatataaataaatatatatgttttatatataaaatatatatatattcttttttttaaattaactctgctaatgttattggtgtcttcactggatatgtttgactgctgtggacttgtgttgggaggaggatgtcctcactcggatgccgacacgggagacaatgggatgaaaggcttcagtgtggtctgagagaggccgaagtccttttgcctgccggggagcaagcaaggccagggcacgggggcacattggctcacttccagaaacacgacaaacccattcctggccctgagtcaagaggacagagagacagatgatgacagagaaagagataaagatgccggttccaaccagaagtttggggagcctcaggacatggcatgctttgtggatccccatgatagtctacaaaagcaccccgcccctctgggcactgcctggaagaatcgggagcctggccagccttcagctcgctcctccacttctgaggggcctaggaggcctcccacaggtgtcccggcaagagaagacacggtggtggaagaagaggcctggtaatggcccctcctcctgggaccccttcgtcctctccttaccccacctcctgggtacagcccaggaggaccttgtgtgatcagaccattgaaaccactaattctgtccccaggagacttggctgtgtgtgtgagtggcttacccttcctcatcttcccttcccaaggcacagagcaatggggcaggacccgcaagcccctcacggaggcagagaaaagagaaagtgttttatatacggtacttatttaatagccctttttaattagaaattaaaacagttaatttaattaaagagtagggtttttttcagtattcttggttaatatttaatttcaactatttatgagatgtatctctcgctctctcttatttgtacttgtgtgtgtgtgtgtgtgtgtgtgtgtgtgtgtgtgtgtgtgtgtatgaaatctgtgtttccaatctctctctcccagatcggtgacagtcactagcttgtcctgagaagatatttaattttgctaacactcagctctgccctcccttgtccccaccacacattcctttgaaataaggtttcaatatacatttacatactatatatatatttggcaacttgtgtttgtatataaatatatatatatatatatatgtttatgtatatatgtgattctgataaaatagacattgctattctgttttttatatgtaaaaacaaaacaagaaaaatagagaattctacatactaaatctctctccttttttaattttaatatttgttatcatttatttattggtgctactgtttatccgtaataattgtgggggaaaaagatattaacatcacgtctttgtctctagagcagttttccgagatattccgtagtacatatttatttttaaacagcaacaaagaaatacagatatatcttaaaaaaaaaagcattttgtattaaagaattgaattctgatctcaaaaaaaaaaaaaaaaaaa

SEQ ID NO: 53 - DNA, 2785 bp, Mus musculus
gtctgctctgcgctctgtacttccctgccgaagctctccacgatttgaccatctgctttcgtgacctttgctccctgggctcgacaggggggccgctgcctgcaacaagtgttcttctctttctctgggagaacccaaatgctcccccaatcctcactcgagggttctattggggaagctggacgggagggactggtctatgcagagttaaaagccagtcttgaggtgctgttgcctagtgggtggatctgggcccagacggggtggagagtgggaggaagctcagcaacaggctggatgggtagctcttaggtagcatttagggggtcagggtgaagcttgggaggggtgcaggaggggaagggatgggggtggtaagaatccaggacctgaattcccagcctggccaacccttgcagctgtctgctctcaagaggagcaagagccccttgaggccagcagggttgggggagttggagtgacctgaggttcttttctgttagagccctggtcctcctatctccaccacctatccctgctcagtagaacccctgggtgctaaatggcaggagccccggggtgtcccataggggtatggctggctgggtcactaaccactgtgatctgctccctccctctacagatcatgcggatcaaacctcaccaaagccagcacataggagagatgagcttcctacagcacagcagatgtgaatgcagaccaaagaaagacagaacaaagccagaaaatcactgtgagccttgttcagagcggagaaagcatttgtttgtccaagatccgcagacgtgtaaatgttcctgcaaaaacacagactcgcgttgcaaggcgaggcagcttgagttaaacgaacgtacttgcagatgtgacaagccaaggcggtgagccaggctgcaggaaggagcctccctcagggtttcgggaaccagacctctcaccggaaagaccgattaaccatgtcaccaccacgccatcatcgtcaccgttgacagaacagtccttaatccagaaagcctgacatgaaggaagaggagactcttcgaggagcactttgggtccggagggcgagactccggcagacgcattcccgggcaggtgaccaagcacggtccctcgtgggactggattcgccattttcttatatctgctgctaaatcgccaagcccggaagattagggttgtttctgggattcctgtagacacacccacccacatacacacatatatatatattatatatataaataaatatatatgttttatatataaaatatatatatattcttttttttaaattaactctgctaatgttattggtgtcttcactggatatgtttgactgctgtggacttgtgttgggaggaggatgtcctcactcggatgccgacacgggagacaatgggatgaaaggcttcagtgtggtctgagagaggccgaagtccttttgcctgccggggagcaagcaaggccagggcacgggggcacattggctcacttccagaaacacgacaaacccattcctggccctgagtcaagaggacagagagacagatgatgacagagaaagagataaagatgccggttccaaccagaagtttggggagcctcaggacatggcatgctttgtggatccccatgatagtctacaaaagcaccccgcccctctgggcactgcctggaagaatcgggagcctggccagccttcagctcgctcctccacttctgaggggcctaggaggcctcccacaggtgtcccggcaagagaagacacggtggtggaagaagaggcctggtaatggcccctcctcctgggaccccttcgtcctctccttaccccacctcctgggtacagcccaggaggaccttgtgtgatcagaccattgaaaccactaattctgtccccaggagacttggctgtgtgtgtgagtggcttacccttcctcatcttcccttcccaaggcacagagcaatggggcaggacccgcaagcccctcacggaggcagagaaaagagaaagtgttttatatacggtacttatttaatagccctttttaattagaaattaaaacagttaatttaattaaagagtagggtttttttcagtattcttggttaatatttaatttcaactatttatgagatgtatctctcgctctctcttatttgtacttgtgtgtgtgtgtgtgtgtgtgtgtgtgtgtgtgtgtgtgtgtgtatgaaatctgtgtttccaatctctctctcccagatcggtgacagtcactagcttgtcctgagaagatatttaattttgctaacactcagctctgccctcccttgtccccaccacacattcctttgaaataaggtttcaatatacatttacatactatatatatatttggcaacttgtgtttgtatataaatatatatatatatatatatgtttatgtatatatgtgattctgataaaatagacattgctattctgttttttatatgtaaaaacaaaacaagaaaaatagagaattctacatactaaatctctctccttttttaattttaatatttgttatcatttatttattggtgctactgtttatccgtaataattgtgggggaaaaagatattaacatcacgtctttgtctctagagcagttttccgagatattccgtagtacatatttatttttaaacagcaacaaagaaatacagatatatcttaaaaaaaaaagcattttgtattaaagaattgaattctgatctcaaaaaaaaaaaaaaaaaaa

SEQ ID NO: 54 - DNA, 2653 bp, Mus musculus
gtctgctctgcgctctgtacttccctgccgaagctctccacgatttgaccatctgctttcgtgacctttgctccctgggctcgacaggggggccgctgcctgcaacaagtgttcttctctttctctgggagaacccaaatgctcccccaatcctcactcgagggttctattggggaagctggacgggagggactggtctatgcagagttaaaagccagtcttgaggtgctgttgcctagtgggtggatctgggcccagacggggtggagagtgggaggaagctcagcaacaggctggatgggtagctcttaggtagcatttagggggtcagggtgaagcttgggaggggtgcaggaggggaagggatgggggtggtaagaatccaggacctgaattcccagcctggccaacccttgcagctgtctgctctcaagaggagcaagagccccttgaggccagcagggttgggggagttggagtgacctgaggttcttttctgttagagccctggtcctcctatctccaccacctatccctgctcagtagaacccctgggtgctaaatggcaggagccccggggtgtcccataggggtatggctggctgggtcactaaccactgtgatctgctccctccctctacagatcatgcggatcaaacctcaccaaagccagcacataggagagatgagcttcctacagcacagcagatgtgaatgcagaccaaagaaagacagaacaaagccagaaaaatgtgacaagccaaggcggtgagccaggctgcaggaaggagcctccctcagggtttcgggaaccagacctctcaccggaaagaccgattaaccatgtcaccaccacgccatcatcgtcaccgttgacagaacagtccttaatccagaaagcctgacatgaaggaagaggagactcttcgaggagcactttgggtccggagggcgagactccggcagacgcattcccgggcaggtgaccaagcacggtccctcgtgggactggattcgccattttcttatatctgctgctaaatcgccaagcccggaagattagggttgtttctgggattcctgtagacacacccacccacatacacacatatatatatattatatatataaataaatatatatgttttatatataaaatatatatatattcttttttttaaattaactctgctaatgttattggtgtcttcactggatatgtttgactgctgtggacttgtgttgggaggaggatgtcctcactcggatgccgacacgggagacaatgggatgaaaggcttcagtgtggtctgagagaggccgaagtccttttgcctgccggggagcaagcaaggccagggcacgggggcacattggctcacttccagaaacacgacaaacccattcctggccctgagtcaagaggacagagagacagatgatgacagagaaagagataaagatgccggttccaaccagaagtttggggagcctcaggacatggcatgctttgtggatccccatgatagtctacaaaagcaccccgcccctctgggcactgcctggaagaatcgggagcctggccagccttcagctcgctcctccacttctgaggggcctaggaggcctcccacaggtgtcccggcaagagaagacacggtggtggaagaagaggcctggtaatggcccctcctcctgggaccccttcgtcctctccttaccccacctcctgggtacagcccaggaggaccttgtgtgatcagaccattgaaaccactaattctgtccccaggagacttggctgtgtgtgtgagtggcttacccttcctcatcttcccttcccaaggcacagagcaatggggcaggacccgcaagcccctcacggaggcagagaaaagagaaagtgttttatatacggtacttatttaatagccctttttaattagaaattaaaacagttaatttaattaaagagtagggtttttttcagtattcttggttaatatttaatttcaactatttatgagatgtatctctcgctctctcttatttgtacttgtgtgtgtgtgtgtgtgtgtgtgtgtgtgtgtgtgtgtgtgtgtatgaaatctgtgtttccaatctctctctcccagatcggtgacagtcactagcttgtcctgagaagatatttaattttgctaacactcagctctgccctcccttgtccccaccacacattcctttgaaataaggtttcaatatacatttacatactatatatatatttggcaacttgtgtttgtatataaatatatatatatatatatatgtttatgtatatatgtgattctgataaaatagacattgctattctgttttttatatgtaaaaacaaaacaagaaaaatagagaattctacatactaaatctctctccttttttaattttaatatttgttatcatttatttattggtgctactgtttatccgtaataattgtgggggaaaaagatattaacatcacgtctttgtctctagagcagttttccgagatattccgtagtacatatttatttttaaacagcaacaaagaaatacagatatatcttaaaaaaaaaagcattttgtattaaagaattgaattctgatctcaaaaaaaaaaaaaaaaaaa

SEQ ID NO: 55 - DNA, 2346 bp, Homo Sapiens
ccttcgcgccctgggccatctccctcccacctccctccgcggagcagccagacagcgagggccccggccgggggcaggggggacgccccgtccggggcacccccccggctctgagccgcccgcggggccggcctcggcccggagcggaggaaggagtcgccgaggagcagcctgaggccccagagtctgagacgagccgccgccgcccccgccactgcggggaggagggggaggaggagcgggaggagggacgagctggtcgggagaagaggaaaaaaacttttgagacttttccgttgccgctgggagccggaggcgcggggacctcttggcgcgacgctgccccgcgaggaggcaggacttggggaccccagaccgcctccctttgccgccggggacgcttgctccctccctgccccctacacggcgtccctcaggcgcccccattccggaccagccctcgggagtcgccgacccggcctcccgcaaagacttttccccagacctcgggcgcaccccctgcacgccgccttcatccccggcctgtctcctgagcccccgcgcatcctagaccctttctcctccaggagacggatctctctccgacctgccacagatcccctattcaagaccacccaccttctggtaccagatcgcgcccatctaggttatttccgtgggatactgagacacccccggtccaagcctcccctccaccactgcgcccttctccctgaggacctcagctttccctcgaggccctcctaccttttgccgggagacccccagcccctgcaggggcggggcctccccaccacaccagccctgttcgcgctctcggcagtgccggggggcgccgcctcccccatgccgccctccgggctgcggctgctgccgctgctgctaccgctgctgtggctactggtgctgacgcctggccggccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagctgaggtcccgccccgccccgccccgccccggcaggcccggccccaccccgccccgcccccgctgccttgcccatgggggctgtatttaaggacacccgtgccccaagcccacctggggccccattaaagatggagagaggactgcggatctctgtgtcattgggcgcctgcctggggtctccatccctgacgttcccccactcccactccctctctctccctctctgcctcctcctgcctgtctgcactattcctttgcccggcatcaaggcacaggggaccagtggggaacactactgtagttagatc

SEQ ID NO: 56 - DNA, 2094 bp, Mus musculus
cgccgccgccgccgcccttcgcgccccaggccgtccccctcctcctcccgccgcggatcctccagacagccaggcccccggccggggcaggggggacgccccttcggggcacccccggctctgagccgcactcggagtcggcctccgctgggagccggcaaaggagcagccgaggagccgtccgaggccccagagtctgagaccagccgccgccgcagggaggagggggaggaggagtgggaggagggacgagctggttgagagaagaggaaaaaagttttgagacttttccgctgctactgcaagtcagagacgtggggacttcttggcactgcgctgtctcgcaaggaggcaggacctgaggactccagacagccctgctcaccgtcgtggacactcgatcgctacccggcgttcctcagacgcccctattccggaccagccctcgggagccacaaaccccgcctcccgcgaagacttcaccccaaagctggggcgcaccccttgcacgccgccctccccccagcctgcctcttgagtccctcgcatcccaggaccctctctcccccgagaggcagatctccctcggacctgctggcagtagctcccctatttaagaacacccacttttggatctcagagagcgctcatctcgatttttaccctggtggtatactgagacaccttggtgtcagagcctcaccgcgactcctgctgctttctccctcaacctcaaattattcaggactatcacctacctttccttgggagaccccaccccacaagccctgcaggggcggggcctccgcatcccacctttgccgagggttcccgctctccgaagtgccgtggggcgccgcctcccccatgccgccctcggggctgcggctactgccgcttctgctcccactcccgtggcttctagtgctgacgcccgggaggccagccgcgggactctccacctgcaagaccatcgacatggagctggtgaaacggaagcgcatcgaagccatccgtggccagatcctgtccaaactaaggctcgccagtcccccaagccagggggaggtaccgcccggcccgctgcccgaggcggtgctcgctttgtacaacagcacccgcgaccgggtggcaggcgagagcgccgacccagagccggagcccgaagcggactactatgctaaagaggtcacccgcgtgctaatggtggaccgcaacaacgccatctatgagaaaaccaaagacatctcacacagtatatatatgttcttcaatacgtcagacattcgggaagcagtgcccgaacccccattgctgtcccgtgcagagctgcgcttgcagagattaaaatcaagtgtggagcaacatgtggaactctaccagaaatatagcaacaattcctggcgttaccttggtaaccggctgctgacccccactgatacgcctgagtggctgtcttttgacgtcactggagttgtacggcagtggctgaaccaaggagacggaatacagggctttcgattcagcgctcactgctcttgtgacagcaaagataacaaactccacgtggaaatcaacgggatcagccccaaacgtcggggcgacctgggcaccatccatgacatgaaccggcccttcctgctcctcatggccacccccctggaaagggcccagcacctgcacagctcacggcaccggagagccctggataccaactattgcttcagctccacagagaagaactgctgtgtgcggcagctgtacattgactttaggaaggacctgggttggaagtggatccacgagcccaagggctaccatgccaacttctgtctgggaccctgcccctatatttggagcctggacacacagtacagcaaggtccttgccctctacaaccaacacaacccgggcgcttcggcgtcaccgtgctgcgtgccgcaggctttggagccactgcccatcgtctactacgtgggtcgcaagcccaaggtggagcagttgtccaacatgattgtgcgctcctgcaagtgcagctgaagccccgccccgccccgcccctcccggcaggcccggccccgcccccgccccgcc

SEQ ID NO: 57 - DNA, 2943 bp, Homo Sapiens
accaggcaacaccattgaaggctcatatgtaaaaatccatgccttcctttctcccaatctccattcccaaacttagccactggcttctggctgaggccttacgcatacctcccggggcttgcacacaccttcttctacagaagacacaccttgggcatatcctacagaagaccaggcttctctctggtccttggtagagggctactttactgtaacagggccagggtggagagttctctcctgaagctccatcccctctataggaaatgtgttgacaatattcagaagagtaagaggatcaagacttctttgtgctcaaataccactgttctcttctctaccctgccctaaccaggagcttgtcaccccaaactctgaggtgatttatgccttaatcaagcaaacttccctcttcagaaaagatggctcattttccctcaaaagttgccaggagctgccaagtattctgccaattcaccctggagcacaatcaacaaattcagccagaacacaactacagctactattagaactattattattaataaattcctctccaaatctagccccttgacttcggatttcacgatttctcccttcctcctagaaacttgataagtttcccgcgcttccctttttctaagactacatgtttgtcatcttataaagcaaaggggtgaataaatgaaccaaatcaataacttctggaatatctgcaaacaacaataatatcagctatgccatctttcactattttagccagtatcgagttgaatgaacatagaaaaatacaaaactgaattcttccctgtaaattccccgttttgacgacgcacttgtagccacgtagccacgcctacttaagacaattacaaaaggcgaagaagactgactcaggcttaagctgccagccagagagggagtcatttcattggcgtttgagtcagcaaagaagtcaagatggccaaagttccagacatgtttgaagacctgaagaactgttacagtgaaaatgaagaagacagttcctccattgatcatctgtctctgaatcagaaatccttctatcatgtaagctatggcccactccatgaaggctgcatggatcaatctgtgtctctgagtatctctgaaacctctaaaacatccaagcttaccttcaaggagagcatggtggtagtagcaaccaacgggaaggttctgaagaagagacggttgagtttaagccaatccatcactgatgatgacctggaggccatcgccaatgactcagaggaagaaatcatcaagcctaggtcagcaccttttagcttcctgagcaatgtgaaatacaactttatgaggatcatcaaatacgaattcatcctgaatgacgccctcaatcaaagtataattcgagccaatgatcagtacctcacggctgctgcattacataatctggatgaagcagtgaaatttgacatgggtgcttataagtcatcaaaggatgatgctaaaattaccgtgattctaagaatctcaaaaactcaattgtatgtgactgcccaagatgaagaccaaccagtgctgctgaaggagatgcctgagatacccaaaaccatcacaggtagtgagaccaacctcctcttcttctgggaaactcacggcactaagaactatttcacatcagttgcccatccaaacttgtttattgccacaaagcaagactactgggtgtgcttggcaggggggccaccctctatcactgactttcagatactggaaaaccaggcgtaggtctggagtctcacttgtctcacttgtgcagtgttgacagttcatatgtaccatgtacatgaagaagctaaatcctttactgttagtcatttgctgagcatgtactgagccttgtaattctaaatgaatgtttacactctttgtaagagtggaaccaacactaacatataatgttgttatttaaagaacaccctatattttgcatagtaccaatcattttaattattattcttcataacaattttaggaggaccagagctactgactatggctaccaaaaagactctacccatattacagatgggcaaattaaggcataagaaaactaagaaatatgcacaatagcagttgaaacaagaagccacagacctaggatttcatgatttcatttcaactgtttgccttctacttttaagttgctgatgaactcttaatcaaatagcataagtttctgggacctcagttttatcattttcaaaatggagggaataatacctaagccttcctgccgcaacagttttttatgctaatcagggaggtcattttggtaaaatacttcttgaagccgagcctcaagatgaaggcaaagcacgaaatgttattttttaattattatttatatatgtatttataaatatatttaagataattataatatactatatttatgggaaccccttcatcctctgagtgtgaccaggcatcctccacaatagcagacagtgttttctgggataagtaagtttgatttcattaatacagggcattttggtccaagttgtgcttatcccatagccaggaaactctgcattctagtacttgggagacctgtaatcatataataaatgtacattaattaccttgagccagtaattggtccgatctttgactcttttgccattaaacttacctgggcattcttgtttcaattccacctgcaatcaagtcctacaagctaaaattagatgaactcaactttgacaaccatgagaccactgttatcaaaactttcttttctggaatgtaatcaatgtttcttctaggttctaaaaattgtgatcagaccataatgttacattattatcaacaatagtgattgatagagtgttatcagtcataactaaataaagcttgcaacaaaattctctgacaaaaaaaaaaaaaaaa

SEQ ID NO: 58 - DNA, 1974 bp, Mus musculus
aagtctccagggcagagagggagtcaactcattggcgcttgagtcggcaaagaaatcaagatggccaaagttcctgacttgtttgaagacctaaagaactgttacagtgaaaacgaagactacagttctgccattgaccatctctctctgaatcagaaatccttctatgatgcaagctatggctcacttcatgagacttgcacagatcagtttgtatctctgagaacctctgaaacgtcaaagatgtccaacttcaccttcaaggagagccgggtgacagtatcagcaacgtcaagcaacgggaagattctgaagaagagacggctgagtttcagtgagaccttcactgaagatgacctgcagtccataacccatgatctggaagagaccatccaacccagatcagcaccttacacctaccagagtgatttgagatacaaactgatgaagctcgtcaggcagaagtttgtcatgaatgattccctcaaccaaactatatatcaggatgtggacaaacactatctcagcaccacttggttaaatgacctgcaacaggaagtaaaatttgacatgtatgcctactcgtcgggaggagacgactctaaatatcctgttactctaaaaatctcagattcacaactgttcgtgagcgctcaaggagaagaccagcccgtgttgctgaaggagttgccagaaacaccaaaactcatcacaggtagtgagaccgacctcattttcttctggaaaagtatcaactctaagaactacttcacatcagctgcttatccagagctgtttattgccaccaaagaacaaagtcgggtgcacctggcacggggactgccctctatgacagacttccagatatcataaaagcagccttatttcgggagtctattcacttgggaagtgctgacagtctgtatgtaccatgtacaggaaccttcctcaccctgagtcacttgcacagcatgtgctgagtctctgtaattctaaatgaatgtttaccctctttgtaagagaagagcaaaccctagtggagccaccccgacatatgatactatctgttattttaaagagtaccctatagtttgctcagtactaatcattttaattactattctgcatggcattcttaggaggatcaaaaagactctacacatattacagatgggttaacaaagggataaaacaactgaaaagcacactcaatgcatttggaatataaattcacagaccaatctcactgtgcaccttcggcttcaaaatgccagttgagtaggataaaggtataagaacttaatgctgtcattttcaaaaggaaggggacaatagctacatctttcctacctcagtgggttttactccagtgagatcatttggatgaaatcctcctgtaacagacctcaagaaggagacagactgttgaatgttatttttaagttattttatctatgtatttataaatatatttatgataattatattatttatggaacatccttaaatcctctgagcttgacggcaccctcgcagcagggttttctaggtggtcagttagatgtagtctcctctagagctccatgctacagacttttacactttttccacagccacgaagctctccgtacattcctgcacttgggagccctttcatcatgatcttaatctgtgctgtttactttgtgcatctaaaatgataattgagtcagtctttctccctcccgtccttaaagctgtctgggtattcttacatcattcagtctcacctgtaactaacaccaaccatctaaagatggaaagagcttaactgtgacaaccacatcactgatacctgaagtttcttttctagaatgtaatcagtgtttcccctggattccaatttttttttcaaaccacagtgtcatgtaactatcaacaataacaatcaactcattattattaatcataattaaataaaacaggtttgagctg

SEQ ID NO: 59 - DNA, 1695 bp, Homo sapiens
gttgccgctgcgcacctggctcaggtgagctgccccgcccccgcccggcgcgagccccaggtcctggcagcagcccctgacctgtccaggtgccctgtccagctgactgcaaggacagagaggagtcctgcccagctcttggatcagtctgctggccgaggagcccggtggagccaggggtgaccctggagcccagcctgccccgaggaggccccggctcagagccatgccaggtgtctgtgatagggcccctgacttcctctccccgtctgaagaccaggtgctgaggcctgccttgggcagctcagtggctctgaactgcacggcttgggtagtctctgggccccactgctccctgccttcagtccagtggctgaaagacgggcttccattgggaattgggggccactacagcctccacgagtactcctgggtcaaggccaacctgtcagaggtgcttgtgtccagtgtcctgggggtcaacgtgaccagcactgaagtctatggggccttcacctgctccatccagaacatcagcttctcctccttcactcttcagagagctggccctacaagccacgtggctgcggtgctggcctccctcctggtcctgctggccctgctgctggccgccctgctctatgtcaagtgccgtctcaacgtgctgctctggtaccaggacgcgtatggggaggtggagataaacgacgggaagctctacgacgcctacgtctcctacagcgactgccccgaggaccgcaagttcgtgaacttcatcctaaagccgcagctggagcggcgtcggggctacaagctcttcctggacgaccgcgacctcctgccgcgcgctgagccctccgccgacctcttggtgaacctgagccgctgccgacgcctcatcgtggtgctttcggacgccttcctgagccgggcctggtgcagccacagcttccgggagggcctgtgccggctgctggagctcacccgcagacccatcttcatcaccttcgagggccagaggcgcgaccccgcgcacccggcgctccgcctgctgcgccagcaccgccacctggtgaccttgctgctctggaggcccggctccgtgactccttcctccgatttttggaaagaagtgcagctggcgctgccgcggaaggtgcggtacaggccggtggaaggagacccccagacgcagctgcaggacgacaaggaccccatgctgattcttcgaggccgagtccctgagggccgggccctggactcagaggtggacccggaccctgagggcgacctgggtgtccgggggcctgtttttggagagccatcagctccaccgcacaccagtggggtctcgctgggagagagccggagcagcgaagtggacgtctcggatctcggctcgcgaaactacagtgcccgcacagacttctactgcctggtgtccaaggatgatatgtagctcccaccccagagtgcaggatcatagggacagcgggggccagggcagcggcgtcgctcctctgctcaacaggaccacaacccctgccagcagccctgggaccctgccagcagccctgggaaaaggctgtggcctcagggcgcctcccagtgccagaaaataaagtccttttggattctgaaaaaaaaaaaaaaaaaaaaaaaaaaaaaaaaaaaaaaaaaaaaaaaaaaaaaaaa

SEQ ID NO: 60 - DNA, 4909 bp, Homo sapiens
tagacgcaccctctgaagatggtgactccctcctgagaagctggaccccttggtaaaagacaaggccttctccaagaagaatatgaaagtgttactcagacttatttgtttcatagctctactgatttcttctctggaggctgataaatgcaaggaacgtgaagaaaaaataattttagtgtcatctgcaaatgaaattgatgttcgtccctgtcctcttaacccaaatgaacacaaaggcactataacttggtataaagatgacagcaagacacctgtatctacagaacaagcctccaggattcatcaacacaaagagaaactttggtttgttcctgctaaggtggaggattcaggacattactattgcgtggtaagaaattcatcttactgcctcagaattaaaataagtgcaaaatttgtggagaatgagcctaacttatgttataatgcacaagccatatttaagcagaaactacccgttgcaggagacggaggacttgtgtgcccttatatggagttttttaaaaatgaaaataatgagttacctaaattacagtggtataaggattgcaaacctctacttcttgacaatatacactttagtggagtcaaagataggctcatcgtgatgaatgtggctgaaaagcatagagggaactatacttgtcatgcatcctacacatacttgggcaagcaatatcctattacccgggtaatagaatttattactctagaggaaaacaaacccacaaggcctgtgattgtgagcccagctaatgagacaatggaagtagacttgggatcccagatacaattgatctgtaatgtcaccggccagttgagtgacattgcttactggaagtggaatgggtcagtaattgatgaagatgacccagtgctaggggaagactattacagtgtggaaaatcctgcaaacaaaagaaggagtaccctcatcacagtgcttaatatatcggaaattgaaagtagattttataaacatccatttacctgttttgccaagaatacacatggtatagatgcagcatatatccagttaatatatccagtcactaatttccagaagcacatgattggtatatgtgtcacgttgacagtcataattgtgtgttctgttttcatctataaaatcttcaagattgacattgtgctttggtacagggattcctgctatgattttctcccaataaaagcttcagatggaaagacctatgacgcatatatactgtatccaaagactgttggggaagggtctacctctgactgtgatatttttgtgtttaaagtcttgcctgaggtcttggaaaaacagtgtggatataagctgttcatttatggaagggatgactacgttggggaagacattgttgaggtcattaatgaaaacgtaaagaaaagcagaagactgattatcattttagtcagagaaacatcaggcttcagctggctgggtggttcatctgaagagcaaatagccatgtataatgctcttgttcaggatggaattaaagttgtcctgcttgagctggagaaaatccaagactatgagaaaatgccagaatcgattaaattcattaagcagaaacatggggctatccgctggtcaggggactttacacagggaccacagtctgcaaagacaaggttctggaagaatgtcaggtaccacatgccagtccagcgacggtcaccttcatctaaacaccagttactgtcaccagccactaaggagaaactgcaaagagaggctcacgtgcctctcgggtagcatggagaagttgccaagagttctttaggtgcctcctgtcttatggcgttgcaggccaggttatgcctcatgctgacttgcagagttcatggaatgtaactatatcatcctttatccctgaggtcacctggaatcagattattaagggaataagccatgacgtcaatagcagcccagggcacttcagagtagagggcttgggaagatcttttaaaaaggcagtaggcccggtgtggtggctcacgcctataatcccagcactttgggaggctgaagtgggtggatcaccagaggtcaggagttcgagaccagcccagccaacatggcaaaaccccatctctactaaaaatacaaaaatgagctaggcatggtggcacacgcctgtaatcccagctacacctgaggctgaggcaggagaattgcttgaaccggggagacggaggttgcagtgagccgagtttgggccactgcactctagcctggcaacagagcaagactccgtctcaaaaaaagggcaataaatgccctctctgaatgtttgaactgccaagaaaaggcatggagacagcgaactagaagaaagggcaagaaggaaatagccaccgtctacagatggcttagttaagtcatccacagcccaagggcggggctatgccttgtctggggaccctgtagagtcactgaccctggagcggctctcctgagaggtgctgcaggcaaagtgagactgacacctcactgaggaagggagacatattcttggagaactttccatctgcttgtattttccatacacatccccagccagaagttagtgtccgaagaccgaattttattttacagagcttgaaaactcacttcaatgaacaaagggattctccaggattccaaagttttgaagtcatcttagctttccacaggagggagagaacttaaaaaagcaacagtagcagggaattgatccacttcttaatgctttcctccctggcatgaccatcctgtcctttgttattatcctgcattttacgtctttggaggaacagctccctagtggcttcctccgtctgcaatgtcccttgcacagcccacacatgaaccatccttcccatgatgccgctcttctgtcatcccgctcctgctgaaacacctcccaggggctccacctgttcaggagctgaagcccatgctttcccaccagcatgtcactcccagaccacctccctgccctgtcctccagcttcccctcgctgtcctgctgtgtgaattcccaggttggcctggtggccatgtcgcctgcccccagcactcctctgtctctgctcttgcctcgacccttcctcctcctttgcctaggaggccttctcgcattttctctagctgatcagaattttaccaaaattcagaacatcctccaattccacagtctctgggagactttccctaagaggcgacttcctctccagccttctctctctggtcaggcccactgcagagatggtggtgagcacatctgggaggctggtctccctccagctggaattgctgctctctgagggagaggctgtggtggctgtctctgtccctcactgccttccaggagcaatttgcacatgtaacatagatttatgtaatgctttatgtttaaaaacattccccaattatcttatttaatttttgcaattattctaattttatatatagagaaagtgacctattttttaaaaaaatcacactctaagttctattgaacctaggacttgagcctccatttctggcttctagtctggtgttctgagtacttgatttcaggtcaataacggtcccccctcactccacactggcacgtttgtgagaagaaatgacattttgctaggaagtgaccgagtctaggaatgcttttattcaagacaccaaattccaaacttctaaatgttggaattttcaaaaattgtgtttagattttatgaaaaactcttctactttcatctattctttccctagaggcaaacatttcttaaaatgtttcattttcattaaaaatgaaagccaaatttatatgccaccgattgcaggacacaagcacagttttaagagttgtatgaacatggagaggacttttggtttttatatttctcgtatttaatatgggtgaacaccaacttttatttggaataataattttcctcctaaacaaaaacacattgagtttaagtctctgactcttgcctttccacctgctttctcctgggcccgctttgcctgcttgaaggaacagtgctgttctggagctgctgttccaacagacagggcctagctttcatttgacacacagactacagccagaagcccatggagcagggatgtcacgtcttgaaaagcctattagatgttttacaaatttaattttgcagattattttagtctgtcatccagaaaatgtgtcagcatgcatagtgctaagaaagcaagccaatttggaaacttaggttagtgacaaaattggccagagagtgggggtgatgatgaccaagaattacaagtagaatggcagctggaatttaaggagggacaagaatcaatggataagcgtgggtggaggaagatccaaacagaaaagtgcaaagttattccccatcttccaagggttgaattctggaggaagaagacacattcctagttccccgtgaacttcctttgacttattgtccccactaaaacaaaacaaaaaacttttaatgccttccacattaattagattttcttgcagtttttttatggcatttttttaaagatgccctaagtgttgaagaagagtttgcaaatgcaacaaaatatttaattaccggttgttaaaactggtttagcacaatttatattttccctctcttgcctttcttatttgcaataaaaggtattgagccattttttaaatgacatttttgataaattatgtttgtactagttgatgaaggagttttttttaacctgtttatataattttgcagcagaagccaaattttttgtatattaaagcaccaaattcatgtacagcatgcatcacggatcaatagactgtacttattttccaataaaattttcaaactttgtactgttaaa

SEQ ID NO: 61 - DNA, 1990 bp, Mus musculus
ggacaggtaaactgtcagcttccgcgcgagtctcaggtgggtggcagtcgtgactcagacacgcggaggtcctggccaggtgacaagcacccatgcacccacctgcatacagcaatccatccattacaggggacagccaggaatgaggctaactgctctctcctgaggtcagatccttcttggagcatgtctgctgtggccctgagtcaccacagtggccacaggacatgtgaaggtggtccatgggcctgcccccttccaaaccgtgtgaggcagaatctgctctgtgcttgtgagatctgggagctcacccagaactccgagactggccaaggttgcaaggcaagctctctggtgtgcagccagtactgctggtggctgacagttactggttaactcatcctgttcctgtttaacttgtcccctggggatggtggcagcctctgacctccaggacctcctgtccgtctggctgaatctacagaaaggagcccttctcagcctgcagtggaccccatctgctcggttaaggagccacggcggtgccagggagggaaaccagcgtttggcctgccgtgaagaggtcccagaagagccatggcaggtgtctgtgacatggcccctaatttcctttccccatctgaagaccaggccttgggtcttgcccttggcagagaagttgctttgaattgcacagcttgggtgttctctaggccccagtgtccccagccatcagtgcagtggctgaaagatggtctggcattgggcaatggaagccacttcagcctccatgaggacttctgggtcagcgccaacttctcagagattgtgtccagtgtcctggtgctcaacttgaccaatgcagaggactatggaaccttcacctgttctgtctggaatgtcagctcccattccttcactctttggcgagctggccctgctggccatgtggctgcagtactggcttccctcctggtcctggtggttctgctgctggtggccctgctctatgttaagtgtcggctgaacatgctgctttggtaccaagacacttacggggaggtggagatgaacgatgggaagttatacgatgcctacgtgtcctatagcgactgcccagaggaccgcaaatttgtaaattttattctgaagcctcagttggagcggcgtcggggatacaaactcttcctagaggaccgcgacctcttgcctcgcgcggagccctctgccgaccttttggtgaacctgagtcgctgtcggcgtctcatcgtggttctttcagatgccttcctaagccggccctggtgtagccagagcttccgggagggactgtgccgcctactggagctcacccgcagacctatcttcatcacctttgagggccagaggcgtgagcccatacaccctgctctccggctcctgcgccagcaccgccacctcgtgaccctggtgctttggaagcctggctccgtgactccttcctctgatttttggaaagagctacagctagcactgccacggaaggtgcagtacaggccggtggagggagaccctcaaacccgacttcaggatgacaaagatcccatgctaatcgtgagaggacgtgctgcccagggccggggcatggagtcagagctggatccagaccctgagggagacctgggtgtccgtggacctgtctttggggagccaccaactccactgcaggaaaccaggatctgcataggagagagccacggcagtgaaatggatgtctctgacctcggctctcgaaactacagtgcacggacagacttctactgcctcgtgtctgaggatgatgtgtagcccatatcccagcagcccagaccatgagatcacggtggcagcttccagggtagaggcagcaggcactccttcctaggatcacaacccttgcctctatccctgggcccctcaggaaaggagtgtggccccagggtgtcacaaaataaaatcctgttggttcctg

SEQ ID NO: 62 - DNA, 4739 bp, Mus musculus
tgggtcgtctgactagaagtgagctgtctgtcattcttgtgcacgccagcccagtaatcatttggaggcaaagcaaactgtaagtaatgctgtcctgggctgacttgaggaggcagttttcgttttaacagccagtgtttatttgctcagcaaacgttgtctcggggagaaatgtcgctggatgtcatcagagttcccagtgccccgaaccgtgaacaacacaaatggagaatatgaaagtgctactggggctcatttgtctcatggtgcctctgctgtcgctggagattgacgtatgtacagaatatccaaatcagatcgttttgtttttatctgtaaatgaaattgatattcgcaagtgtcctcttactccaaataaaatgcacggcgacaccataatttggtacaagaatgacagcaagacccccatatcagcggaccgggactccaggattcatcagcagaatgaacatctttggtttgtacctgccaaggtggaggactcaggatattactattgtatagtaagaaactcaacttactgcctcaaaactaaagtaaccgtaactgtgttagagaatgaccctggcttgtgttacagcacacaggccaccttcccacagcggctccacattgccggggatggaagtcttgtgtgcccttatgtgagttattttaaagatgaaaataatgagttacccgaggtccagtggtataagaactgtaaacctctgcttcttgacaacgtgagcttcttcggagtaaaagataaactgttggtgaggaatgtggctgaagagcacagaggggactatatatgccgtatgtcctatacgttccgggggaagcaatatccggtcacacgagtaatacaatttatcacaatagatgaaaacaagagggacagacctgttatcctgagccctcggaatgagacgatcgaagctgacccaggatcaatgatacaactgatctgcaacgtcacgggccagttctcagaccttgtctactggaagtggaatggatcagaaattgaatggaatgatccatttctagctgaagactatcaatttgtggaacatccttcaaccaaaagaaaatacacactcattacaacacttaacatttcagaagttaaaagccagttttatcgctatccgtttatctgtgttgttaagaacacaaatatttttgagtcggcgcatgtgcagttaatatacccagtccctgacttcaagaattacctcatcgggggctttatcatcctcacggctacaattgtatgctgtgtgtgcatctataaagtcttcaaggttgacatagtgctttggtacagggactcctgctctggttttcttccttcaaaagcttcagatggaaagacatacgatgcctatattctttatcccaagaccctgggagaggggtccttctcagacttagatacttttgtttttaaactgttgcctgaggtcttggagggacagtttggatacaagctgttcatttatggaagggatgactatgttggagaagataccatcgaggttactaatgaaaatgtaaagaaaagcaggaggctgattatcattctagtgagagatatgggaggcttcagctggctgggccagtcatctgaagagcaaatagccatatacaatgctctcatccaggaaggaattaaaatcgtcctgcttgagttggagaaaatccaagactatgagaaaatgccagattctattcagttcattaagcagaaacacggagtcatttgctggtcaggagactttcaagaaagaccacagtctgcaaagaccaggttctggaaaaacttaagataccagatgccagcccaacggagatcaccattgtctaaacaccgcttactaaccctggatcctgtgcgggacactaaggagaaactgccggcagcaacacacttaccactcggctagcatggcaaaagtgggcaggccaagaacttcggaatatctcccatcataagaggctgcagctgggctgtgcctcccagtaaaacagtcacgaaccaaacctgtgcagtcccttgttccagatcacctggaactggattgggaagagaacaggacttggtggccaggaccgctcagagagccatggttgctcagggatgctgctccgggatgcttgactaacagtcgaggcagtgaactgggtgtagaaagcgtcaggaaatggccacatgtgtggatggtttaattagattctgtggagtctcacagtgggattgtggttgtctgaggacactttggggggtcgctgtccaagaagtggctccccaaagtataagtgcgggtgaggtttactgataccccacagtggaaggcatttcttccagatattttccagataccttggccgccaaacttcgttttcaaacactgtgttttattgtgtagaccttgaaaaccacggtttcagtgaagaaggattctgtagggttcactcttctgcagtcactttagctttctgcagaagtctagattttaaattaactgtagaaggaagtcagacacccacaggtttcttctaatcaagcttccaccctgcaatggctgtcgtgccatcttccttctgtgttttactcggctcattgcttctctggagacaaatgcaatgcttcctacccccgtcctgtcacaggcagtctgaggactcaccttcacacgtgagacttcgctcttaccccagcccccaccgctggaggggtttcaatcccccaccctggggctcttctgcgcagaagctgaagtctacgctcctcccaatctttggtcccagtgccgctgttcgccttcctgctggttgattgcagaaccacctctgagacctctgtacctgctcttggaatgtctctgtctttgccctgccgcccacttcccgtgcctatgatttgctctagcatttctgccacctgacaaggagaggagcttgctaaagtttaaagtttatttcaacgctaagatcccaaccttccgatctctgcctactctcagacttggccagccctcttgcctgtcatcccaaccctgctgcttctctgtaaaaggttgtttctttactagcagaaatcagtctgcagggaggggtagtgcgtgtatctcactgttttccgtattgggaacagatggtatagatttgccaaggcctttattttaaagtgttcttggaaaatcgtatttaaatttgcaattattctaatttgatatagagagaaactgacctatttaacaaatggactctcttaattctactggacccaggccttgaactttctggtttctgtctctgtgctctgactttttgatttcaggtctagccctgtgcacttacctcagtactcagtacttactcagtactaactcagtactcgtgggaggacacccgacccgggtgttctttgagaagcagctgagtgaggcatgttttgacaggaggaacccagttcttttcactgttggtatttatagcacctaccttcagatttttcagtatgatgtttctattttccaatactggttccctaacggtcaacatttcttctcccaactaaaaatgtaagaagaaaaaaaaatttttttctttttctctttttttttttttttgtggtacagaagttttttgtttggttatttgtttgtttgattttaactagatgactgtcttcagaataatcctcacagttctggaagattgaaaaaccaagttcacatgacaagaacaaacccggttttaagggttgaatgtagggggataccattctgtcttttagactttcaacatctgctatggtggagcacaaatgttatttaaaagcatcttctcacctacatagaagtaaacaaaacacacaatttgggattttgactcttcagttttgacctcacttctcctggatccaacctgacaggagatgactggattgctggggtagagaaggaaaggctgtccccgacagcagccagaagtctgtgggagtgaggtcattcctgacccgtgatgctttcaaggcttggtttttctgataattatttttgcctgtcatcccaaaaacgtattgacatgcgtgtatattacctgtgagtgagagttcttagaaacttggccagagaggataaaggggagagcagtgacagagaaccgccgatcttgtgacaccggagtctgaggtggaacagagccagtgtcaggaaagcccctcagcaagaaagaagagtcattgctggttcccaggggcagtcacagaggatgaaataccacatcaaggcagtgggaacttcctgcgacttctcctggtcctccccagaagtatctggtgtcagctgaagaccatgtgtcccccaatacttagatttcaccgtactttctgatggtgtttttaaaaggccaagtgttgcaaaagtttgcacgtgcaaccgtatattaatcgccagtgtttaaaactggtttatcagaatttatatttttgtctctcctgccttttgaattttacttatgacagagaaagtattgagccatgactttttaaaaataatttatatttatactggtcaataatgaaggtttttttttattattaaagaacttgattttataattctgcagcagatgccaaatgttatctatcacaaaccacaaaatttatgtacagtgcatattattgatcaataaactaaacttgttttccaataaattgttaa

SEQ ID NO: 63 - DNA, 5561 bp, Homo sapiens
aaagcagcagagacgctgcagagggcttttcttagacatcaactgcagacggctggcaggatagaagcagcggctcacttggactttttcaccagggaaatcagagacaatgatggggctcttccccagaactacaggggctctggccatcttcgtggtggtcatattggttcatggagaattgcgaatagagactaaaggtcaatatgatgaagaagagatgactatgcaacaagctaaaagaaggcaaaaacgtgaatgggtgaaatttgccaaaccctgcagagaaggagaagataactcaaaaagaaacccaattgccaagattacttcagattaccaagcaacccagaaaatcacctaccgaatctctggagtgggaatcgatcagccgccttttggaatctttgttgttgacaaaaacactggagatattaacataacagctatagtcgaccgggaggaaactccaagcttcctgatcacatgtcgggctctaaatgcccaaggactagatgtagagaaaccacttatactaacggttaaaattttggatattaatgataatcctccagtattttcacaacaaattttcatgggtgaaattgaagaaaatagtgcctcaaactcactggtgatgatactaaatgccacagatgcagatgaaccaaaccacttgaattctaaaattgccttcaaaattgtctctcaggaaccagcaggcacacccatgttcctcctaagcagaaacactggggaagtccgtactttgaccaattctcttgaccgagagcaagctagcagctatcgtctggttgtgagtggtgcagacaaagatggagaaggactatcaactcaatgtgaatgtaatattaaagtgaaagatgtcaacgataacttcccaatgtttagagactctcagtattcagcacgtattgaagaaaatattttaagttctgaattacttcgatttcaagtaacagatttggatgaagagtacacagataattggcttgcagtatatttctttacctctgggaatgaaggaaattggtttgaaatacaaactgatcctagaactaatgaaggcatcctgaaagtggtgaaggctctagattatgaacaactacaaagcgtgaaacttagtattgctgtcaaaaacaaagctgaatttcaccaatcagttatctctcgataccgagttcagtcaaccccagtcacaattcaggtaataaatgtaagagaaggaattgcattccgtcctgcttccaagacatttactgtgcaaaaaggcataagtagcaaaaaattggtggattatatcctgggaacatatcaagccatcgatgaggacactaacaaagctgcctcaaatgtcaaatatgtcatgggacgtaacgatggtggatacctaatgattgattcaaaaactgctgaaatcaaatttgtcaaaaatatgaaccgagattctactttcatagttaacaaaacaatcacagctgaggttctggccatagatgaatacacgggtaaaacttctacaggcacggtatatgttagagtacccgatttcaatgacaattgtccaacagctgtcctcgaaaaagatgcagtttgcagttcttcaccttccgtggttgtctccgctagaacactgaataatagatacactggcccctatacatttgcactggaagatcaacctgtaaagttgcctgccgtatggagtatcacaaccctcaatgctacctcggccctcctcagagcccaggaacagatacctcctggagtataccacatctccctggtacttacagacagtcagaacaatcggtgtgagatgccacgcagcttgacactggaagtctgtcagtgtgacaacaggggcatctgtggaacttcttacccaaccacaagccctgggaccaggtatggcaggccgcactcagggaggctggggcctgccgccatcggcctgctgctccttggtctcctgctgctgctgttggccccccttctgctgttgacctgtgactgtggggcaggttctactgggggagtgacaggtggttttatcccagttcctgatggctcagaaggaacaattcatcagtggggaattgaaggagcccatcctgaagacaaggaaatcacaaatatttgtgtgcctcctgtaacagccaatggagccgatttcatggaaagttctgaagtttgtacaaatacgtatgccagaggcacagcggtggaaggcacttcaggaatggaaatgaccactaagcttggagcagccactgaatctggaggtgctgcaggctttgcaacagggacagtgtcaggagctgcttcaggattcggagcagccactggagttggcatctgttcctcagggcagtctggaaccatgagaacaaggcattccactggaggaaccaataaggactacgctgatggggcgataagcatgaattttctggactcctacttttctcagaaagcatttgcctgtgcggaggaagacgatggccaggaagcaaatgactgcttgttgatctatgataatgaaggcgcagatgccactggttctcctgtgggctccgtgggttgttgcagttttattgctgatgacctggatgacagcttcttggactcacttggacccaaatttaaaaaacttgcagagataagccttggtgttgatggtgaaggcaaagaagttcagccaccctctaaagacagcggttatgggattgaatcctgtggccatcccatagaagtccagcagacaggatttgttaagtgccagactttgtcaggaagtcaaggagcttctgctttgtccacctctgggtctgtccagccagctgtttccatccctgaccctctgcagcatggtaactatttagtaacggagacttactcggcttctggttccctcgtgcaaccttccactgcaggctttgatccacttctcacacaaaatgtgatagtgacagaaagggtgatctgtcccatttccagtgttcctggcaacctagctggcccaacgcagctacgagggtcacatactatgctctgtacagaggatccttgctcccgtctaatatgaccagaatgagctggaataccacactgaccaaatctggatctttggactaaagtattcaaaatagcatagcaaagctcactgtattgggctaataatttggcacttattagcttctctcataaactgatcacgattataaattaaatgtttgggttcataccccaaaagcaatatgttgtcactcctaattctcaagtactattcaaattgtagtaaatcttaaagtttttcaaaaccctaaaatcatattcgccaggaaattttcctaaacattcttaagcttctatttttcccctgccaaaggaaggtgtttatcattttaaaatgcaatgtgatttagtggattaagcaggagcgctggttcttgtctccattgccttttcttatatcattgataatgatgtaagaatcacaaggggccgggcgcggtggctcacgcctgtaatcccagcactttgggaggccgaggcaggtggatcatgaggtcaggagatcgagaccatcctggctaacaaggtgaaaccccgtctctactaaaaatacaaaaaattagccgggcgcagtggcgggcgcctgtagtcccagctactcgggaggctgaggcaggagaatggcatgaacccgggaagcggagcttgcagtgagccgagattgcgccactgcagtccgcagtccggcctgggcgacagagcgagactccgtctcaaaaaaaaaaaaaaaaaaagaatcacaaggtatttgctaaagcattttgagctgcttggaaaaagggaagtagttgcagtagagtttcttccatcttcttggtgctgggaagccatatatgtgtcttttactcaagctaaggggtataagcttatgtgttgaatttgctacatctatatttcacatattctcacaataagagaattttgaaatagaaatatcatagaacatttaagaaagtttagtataaataatattttgtgtgttttaatccctttgaagggatctatccaaagaaaatattttacactgagctccttcctacacgtctcagtaacagatcctgtgttagtctttgaaaatagctcattttttaaatgtcagtgagtagatgtagcatacatatgatgtataatgacgtgtattatgttaacaatgtctgcagattttgtaggaatacaaaacatggccttttttataagcaaaacgggccaatgactagaataacacatagggcaatctgtgaatatgtattataagcagcattccagaaaagtagttggtgaaataattttcaagtcaaaaagggatatggaaagggaattatgagtaacctctattttttaagccttgcttttaaattaaacagctacagccatttaagccttgaggataataaagcttgagagtaataatgttaggttagcaaaggtttagatgtatcacttcatgcatgctaccatgatagtaatgcagctcttcgagtcatttctggtcattcaagatattcacccttttgcccatagaaagcaccctacctcacctgcttactgacattgtcttagctgatcacaagatcattatcagcctccattattccttactgtatataaaatacagagttttatattttcctttcttcgtttttcaccatattcaaaacctaaatttgtttttgcagatggaatgcaaagtaatcaagtgtttgtgctttcacctagaagggtgtggtcctgaaggaaagaggtcccctaaatatcccccaccctggtgctcctccctctccctggtaccctgactaccaggaagtcaggtgctagagcagctggagaagtgcaggcagcctgtgcttccacagatgggggtgctgctgcaacaaggctttcaatgtgcccatcttaggtgggagaagctagatcctgtgcagcagcctggtaagtcctgaggaggttccattgctcttcctgctgctgtcctttgcttctcaacggtggctcgctctacagtctagagcacatgcagctaacttgtgcctctgcttatgcatgagggttaaattaacaaccataaccttcatttgaagttcaaaggtgtattcaggatcctcaaagcattttaaccttgccgcttaaaacccaatttaccgtgaaatgggaattttgctgcattgttaaactgtagtggaaaccatgctatagtaataaaggttatataagagagaaattgaaattaaatgtgtttttaaatttcaaaaaaaaatcaatctttaggatgacttaaaaattgatttgccatgtaaaatgtatctgcattttttacacaaaacttgttttaagcataaaattttaaaactgtactacttgatgtattatacattttgaaccatatgtattaaaccataaacagtataatgttgttataataaaacaggcaataaatttataaataaaagctgaaaaaaaaaaaa

SEQ ID NO: 64 - DNA, 4108 bp, Mus musculus
agatatacagacacccaggtgaggagctgcagcaagaagcttggatttattcaccagacacaccagcaacaatgacctgcctcttccctagagccttggggtctctggccctcctgatggtggtgctgctggtacagggggaactgcatgtgaagccgggaggacagcacagagaagatgggacagcattgcagctggccaaaagaaggtacaaacgtgaatgggtgaaatttgcaaaaccctgcagagaaagagaagacaactcaagacgaaacccaattgccaagatcacttcagatttccaaaaaaaccagaaaattacctaccgcatttctggagtgggaattgatcagcctccctttgggatctttgttgttgacccaaacaatggtgatatcaacataacagccatagttgatcgtgaggaaaccccaagcttcctgatcacatgccgtgcactaaatgccctgggacaggatgtagagagaccacttatattaacagtgaaaattttagacgtcaatgataaccctccaatattttcacaaaccatattcaagggtgaaattgaagaaaatagtgcttcaaattccctggtaatgatcctaaatgccacggatgcagatgaaccaaaccatatgaactctaaaattgccttcaaaattgtctcccaggagcccgcaggcatgtccatgttcctcatcagcagaaacaccggagaagtccgcactttgaccagttctcttgatcgagagcaaattagtagctaccatctggttgtgagcggtgcagacaacgatggaacaggactgtcaactcagtgtgaatgtagtattaaaattaaagatgtcaatgacaacttcccagtgctcagagagtctcagtattcagcacgtattgaagaaaatactttgaatgcagagctactccgatttcaagtgacagactgggatgaagaatacacagataattggttggctgtgtatttcttcacctctgggaatgaggggaattggtttgaaattgaaacagatcctagaaccaatgaaggcatcctgaaggtggttaaggctctggattatgaacaagtgcagagtatgcagtttagtattgctgtcagaaacaaagctgagttccaccagtcagtgatctctcagtaccgagtgcagtcaacgccagtcacaattcaagtcattgatgtccgagagggaatctcattccgtcctccttccaagacatttactgtgcaaaggggtgtaagcactaacaaattagttggttacatcctggggacataccaagctactgatgaggacacgggtaaagctgcctcctctgtcagatatgtcctgggtcgtaatgatggtggtttgcttgttattgattcaaagactgctcagatcaaatttgtcaaaaacatcgaccgagattctactttcatagttaacaagacaatttctgctgaggttctggccatagacgaaaacactggtaagacctccacaggcacaatatatgtggaagtgcccagttttaatgaaaactgcccgtcagttgtcctcgagaagaaagacatctgtacttcctcaccttctgtgaccctctcagtgagaaccctggacaggggtaaatacactggcccctacacagtgtccctggaggagcagccactgaagctgccagtcatgtggaccatcacgacgctgaatgctacatcagctcttctacaagcccagcagcaagtgtctccaggagtgtacaatgtcccagtcatcgtgaaggacaatcaggatgggctgtgtgacacaccagagagcttgaccctgacagtgtgtcaatgtgatgaccggagcatgtgcagagcccctatcccgagcagagagcctaacacatatggggagtcatcctggaggctggggcctgctgccattggcttgatcctgctaggactcctgatgttgctattggccccacttctgcttctgacctgtgactgtgggtcaggtcccattggaggagcagcgacaggtggctttatcccagtgcccgatggttcggaaggtacaattcatcagtggggaatcgaaggagctcagcccgaagacaaggaaatcacaaatatttgtgttcctccagttacaaccaatggagccgattttatggaaagctctgaagtttgcacaaatacatatgctggtgggaccatggtagaaggtgcttcaggaatggaaatgattaccaagctgggaggagctactggagctacagcggcacttggtccctgttccttgggttattcaggaacaatgagaacaaggcattccactggagggaccttgaaggactatgctgcgccagtcaacatgactttcctaggctcctatttctctcagaaatcactcgcctatgccgaggaagaggatgagcgcgaagtgaacgactgtttgctgatctacgatgatgaaggcgaggatgctgccccccactcccccaccctcagctcctgcagcatttttgccgatgacctagacgacaacttcttggattcccttggccccaaatttaaaaagcttgcagagatctgtctcggtattgatgacgaagccaagcaagcaaagccaggccctaaagacagtggttctggggctgatacctgcgctcgctctatggaagtccctcagtcaggttctaacaggtaccagactttgcctggcagtctggaagtcacccaaacgggttctaaaatatgccacacgctgtcaggcaaccaagaaacttctgttatgtccacttctgggtctgtccaccctgctgttgccatccctgaccctctacagcttgggaactacttgctcacagaaacttactcaacttcaggttcctttgcacaacccaccactgttacctttgacccccatgtcacacaaaatgtcactgtaacagaaagggtcatttgtccccttcctagtgcctccagcagtatagtggctcccacagaactgcgaggctcatacaatatgctctatacaaaagaaacctgttcccatctatgaccaaagcaaactggaaaaacataccaggcaaatttggatgtgtgtacctacatatctttttttaatatagcaatgctagcactgttggactaatttggctcttagtggataatctctgatcataagaataattcaaattttctgattcatatatgaaagctgggtatgttgtcatttctaattatctgattttttcaagcagagtaaacctgtatatatatatatatttttaattatctaagatgttagacaggtatatttcccaagcatcttcacatttctacattatcgacaacaaagggagatgctgttgccttaaaatgcagtgtgggttagcagattggctgccttcttaaagttcacatttctgtttctaccacccattctgtttctaccatccatgaataaggatatgagagtattattaataacaatgggaatttgaggtgctcaatgtagcttacagggcaacttggaataaagacattaaatgcaactggggatttttggtttggggatttttggtttggtttggtttggtttggtttggtttggtttggtttggtttggtttggtttggtttggtttctcttcttggtgatggaactcaaagcatatcttttgctcaagtccagggttgtgagtaaacctgtttaatttgcaatatctatatgctgtgtatcttcaaaataaggcaattttaaaatgcaagtatcataaaacatgtaagaaaatttagtatggcataaataatacgcagtgtgtactttgatctagcccaagaaaaatacaatgaacttcctcattgacactcctctgaaactgatcctgtgtcagtctttaacaaagaaatccttattttgtaagtgccagggagtagaaatgactcaaaagatgtgcagtgaggttcgttatgttaaccgtgtctaacagggtttgtgggaatagatagcatgccttctcttctaagcaaaaaggtctatggcctaaaataacatggagagaagtgtgtgaatgtacatcattttaggaaaatgatgaagtaaatttcaagt

SEQ ID NO: 65 - DNA, 1927 bp, Homo sapiens
tgccagcccaagtcggaacttggatcacatcagatcctctcgagctccagcaggagaggcccttcctcgcctggcagcccctgagcggctcagcagggcaccatggcaagatcccttctcctgcccctgcagatcttactgctatccttagccttggaaactgcaggagaagaagcccagggtgacaagattattgatggcgccccatgtgcaagaggctcccacccatggcaggtggccctgctcagtggcaatcagctccactgcggaggcgtcctggtcaatgagcgctgggtgctcactgccgcccactgcaagatgaatgagtacaccgtgcacctgggcagtgatacgctgggcgacaggagagctcagaggatcaaggcctcgaagtcattccgccaccccggctactccacacagacccatgttaatgacctcatgctcgtgaagctcaatagccaggccaggctgtcatccatggtgaagaaagtcaggctgccctcccgctgcgaaccccctggaaccacctgtactgtctccggctggggcactaccacgagcccagatgtgacctttccctctgacctcatgtgcgtggatgtcaagctcatctccccccaggactgcacgaaggtttacaaggacttactggaaaattccatgctgtgcgctggcatccccgactccaagaaaaacgcctgcaatggtgactcagggggaccgttggtgtgcagaggtaccctgcaaggtctggtgtcctggggaactttcccttgcggccaacccaatgacccaggagtctacactcaagtgtgcaagttcaccaagtggataaatgacaccatgaaaaagcatcgctaacgccacactgagttaattaactgtgtgcttccaacagaaaatgcacaggagtgaggacgccgatgacctatgaagtcaaatttgactttacctttcctcaaagatatatttaaaccaacctcatgccctgttgataaaccaatcaaattggtaaagacctaaaaccaaaacaaataaagaaacacaaaaccctcagtgctggagaagagtcagtgagaccagcactctcaaacactggaactggacgttcgtacagtctttacggaagacacttggtcaacgtacaccgagacccttattcaccacctttgacccagtaactctaatcttaggaagaacctactgaaacaaaaaaaatccaaaatgtagaacaagacttgaatttaccatgatattatttatcacagaaatgaagtgaaaccatcaaacatgttccaaaagtaccagatggcttaaataatagtctggcttggcacaacgatgttttttttctttgagacagagtctctgttgcttgggctgcaatgcagtgatgcaatcttggctcactgcaacctccgcctcctgggttcaagtgattctcgtgcttcagcctcccaagtacctgggactacaggtgtgcaccaccacaccaggctaattttttgtgtatttttactagagacagggtttcaccatgttggccagcgtggtcttgaacgcctgacctcagatgatccacccaccttggcctcccaaagtgctgggattacaggcatgagccaccacggccagcccacaatgatattacaaacctattaaaaatgatacttagacagaattgtcagtattattcaagaacatttaggctataggatgttaaatgacaaaaggaaggacaaaaatatatatgtatgtgaccctacccataaaaaatgaaatattcacagaatcagatctgaaaacacatgtcccagactgcatactggggtcgtcatgaggtgtctccttccttctgtgtacttttccttgaatgtgcacttttataacatgaaaaataaaggtggggaaaaaagtctgaaga

SEQ ID NO: 66 - DNA, 1927 bp, Homo sapiens
ggagagggtgatgctggctccaagcctgactctgctctcgagagctccagcaggagaggcccttcctcgcctggcagcccctgagcggctcagcagggcaccatggcaagatcccttctcctgcccctgcagatcttactgctatccttagccttggaaactgcaggagaagaagcccagggtgacaagattattgatggcgccccatgtgcaagaggctcccacccatggcaggtggccctgctcagtggcaatcagctccactgcggaggcgtcctggtcaatgagcgctgggtgctcactgccgcccactgcaagatgaatgagtacaccgtgcacctgggcagtgatacgctgggcgacaggagagctcagaggatcaaggcctcgaagtcattccgccaccccggctactccacacagacccatgttaatgacctcatgctcgtgaagctcaatagccaggccaggctgtcatccatggtgaagaaagtcaggctgccctcccgctgcgaaccccctggaaccacctgtactgtctccggctggggcactaccacgagcccagatgtgacctttccctctgacctcatgtgcgtggatgtcaagctcatctccccccaggactgcacgaaggtttacaaggacttactggaaaattccatgctgtgcgctggcatccccgactccaagaaaaacgcctgcaatggtgactcagggggaccgttggtgtgcagaggtaccctgcaaggtctggtgtcctggggaactttcccttgcggccaacccaatgacccaggagtctacactcaagtgtgcaagttcaccaagtggataaatgacaccatgaaaaagcatcgctaacgccacactgagttaattaactgtgtgcttccaacagaaaatgcacaggagtgaggacgccgatgacctatgaagtcaaatttgactttacctttcctcaaagatatatttaaaccaacctcatgccctgttgataaaccaatcaaattggtaaagacctaaaaccaaaacaaataaagaaacacaaaaccctcagtgctggagaagagtcagtgagaccagcactctcaaacactggaactggacgttcgtacagtctttacggaagacacttggtcaacgtacaccgagacccttattcaccacctttgacccagtaactctaatcttaggaagaacctactgaaacaaaaaaaatccaaaatgtagaacaagacttgaatttaccatgatattatttatcacagaaatgaagtgaaaccatcaaacatgttccaaaagtaccagatggcttaaataatagtctggcttggcacaacgatgttttttttctttgagacagagtctctgttgcttgggctgcaatgcagtgatgcaatcttggctcactgcaacctccgcctcctgggttcaagtgattctcgtgcttcagcctcccaagtacctgggactacaggtgtgcaccaccacaccaggctaattttttgtgtatttttactagagacagggtttcaccatgttggccagcgtggtcttgaacgcctgacctcagatgatccacccaccttggcctcccaaagtgctgggattacaggcatgagccaccacggccagcccacaatgatattacaaacctattaaaaatgatacttagacagaattgtcagtattattcaagaacatttaggctataggatgttaaatgacaaaaggaaggacaaaaatatatatgtatgtgaccctacccataaaaaatgaaatattcacagaatcagatctgaaaacacatgtcccagactgcatactggggtcgtcatgaggtgtctccttccttctgtgtacttttccttgaatgtgcacttttataacatgaaaaataaaggtggggaaaaaagtctgaaga

SEQ ID NO: 67 - DNA, 1745 bp, Mus musculus
gagacactggtctccggatcaaggacagagcctcgccaagtcactcccagctctctcatctggcagtccctgagccactgtcgccgagatgggagtctggctcctttccctgataactgtgctgctgtctttagccctggaaaccgctggacaaggagaaaggattatagatggctacaaatgtaaagaaggctcgcacccatggcaggtggctctgctcaaaggcaatcagcttcactgtggaggggtgctggtggacaaatactgggtgctcacagcagcccactgcaaaatgggtcagtaccaagtacagctgggcagtgataaaataggggaccagagtgcccagaagatcaaggctacaaagtcattccggcaccctggctactccacaaagacccacgtcaatgacatcatgcttgtgaggttggatgagccagtcaagatgtcatccaaagtggaggcagtccagcttcctgaacactgtgaacccccagggacgtcatgtaccgtctctggatggggcaccacaaccagcccagacgtgacctttccctcggatcttatgtgctcggatgtgaagctcatctcctccagggagtgcaagaaggtgtacaaggacctgctggggaaaaccatgctgtgtgctggcattcctgactctaagaccaacacgtgcaatggtgactcaggggggcccttggtgtgcaacgacaccctccaaggtctggtgtcctggggtacctacccttgtggccagcccaacgacccaggcgtctacactcaagtctgcaagtacaaacggtgggtgatggagaccatgaaaacccatcgctaacatgccaggagccgagctgtgagcctttgaggttatgatcatggagtaaggacatcgtggcatccctgtaaagtcctgcctggctttacctttcctccaggacatagccaaacctcaacactaggttaagtttataaaccaatcagaaccccagagacctaaaagcaaatcaaggcaagagactaagaaacctgagggctgtggaagatgccagaaggctaaagctttccaatacaagagccagggattcctacagtcctttattggaaagtggttggtaactgtgagaccttcatccactgccttggactcaggtcatccatttctgcagagttctttctcaaaaaaaaaaaaaaaaaagattgaaccaagtttggattcattacagtacttatcatagaggaagaaaagaatggaaacggttaacatgttcaaaatagtagattatttacatgggagttggactgctctcattatgaagttgtgaagttgcttaaggtgtactgcttagccttatgcaagagagactgttgttgtgtggtgtttaatggggacgtgggctgaacatgccaaatgaatactagtgagacagaataaaatctaagtgatattgatgtgtgtgctcataacctggaaggtaacgttggggaagctgggagccacgatggtggagggaggagatctatgaagaagagttggctaagaaattgtagctaggccagcaagatggctcagtggataaaggtacttgctgccaatcctgacatggcttaagtttcatctctagagcctacacagtggaagggaacaactgacgcacgcttgttatcctctgacctccaagtgtgctggggcacaagtgtccctgcacacaaatatggacgccaaaccagattaaaatgcaaat

SEQ ID NO: 68 - DNA, 1669 bp, Homo sapiens
ctccctcagcaaggacagcagaggaccagctaagagggagagaagcaactacagaccccccctgaaaacaaccctcagacgccacatcccctgacaagctgccaggcaggttctcttcctctcacatactgacccacggctccaccctctctcccctggaaaggacaccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaggaggacgaacatccaaccttcccaaacgcctcccctgccccaatccctttattaccccctccttcagacaccctcaacctcttctggctcaaaaagagaattgggggcttagggtcggaacccaagcttagaactttaagcaacaagaccaccacttcgaaacctgggattcaggaatgtgtggcctgcacagtgaagtgctggcaaccactaagaattcaaactggggcctccagaactcactggggcctacagctttgatccctgacatctggaatctggagaccagggagcctttggttctggccagaatgctgcaggacttgagaagacctcacctagaaattgacacaagtggaccttaggccttcctctctccagatgtttccagacttccttgagacacggagcccagccctccccatggagccagctccctctatttatgtttgcacttgtgattatttattatttatttattatttatttatttacagatgaatgtatttatttgggagaccggggtatcctgggggacccaatgtaggagctgccttggctcagacatgttttccgtgaaaacggagctgaacaataggctgttcccatgtagccccctggcctctgtgccttcttttgattatgttttttaaaatatttatctgattaagttgtctaaacaatgctgatttggtgaccaactgtcactcattgctgagcctctgctccccaggggagttgtgtctgtaatcgccctactattcagtggcgagaaataaagtttgcttagaaaagaa

SEQ ID NO: 69 - DNA, 1619 bp, Mus musculus
cctcagcgaggacagcaagggactagccaggagggagaacagaaactccagaacatcttggaaatagctcccagaaaagcaagcagccaaccaggcaggttctgtccctttcactcactggcccaaggcgccacatctccctccagaaaagacaccatgagcacagaaagcatgatccgcgacgtggaactggcagaagaggcactcccccaaaagatggggggcttccagaactccaggcggtgcctatgtctcagcctcttctcattcctgcttgtggcaggggccaccacgctcttctgtctactgaacttcggggtgatcggtccccaaagggatgagaagttcccaaatggcctccctctcatcagttctatggcccagaccctcacactcagatcatcttctcaaaattcgagtgacaagcctgtagcccacgtcgtagcaaaccaccaagtggaggagcagctggagtggctgagccagcgcgccaacgccctcctggccaacggcatggatctcaaagacaaccaactagtggtgccagccgatgggttgtaccttgtctactcccaggttctcttcaagggacaaggctgccccgactacgtgctcctcacccacaccgtcagccgatttgctatctcataccaggagaaagtcaacctcctctctgccgtcaagagcccctgccccaaggacacccctgagggggctgagctcaaaccctggtatgagcccatatacctgggaggagtcttccagctggagaagggggaccaactcagcgctgaggtcaatctgcccaagtacttagactttgcggagtccgggcaggtctactttggagtcattgctctgtgaagggaatgggtgttcatccattctctacccagcccccactctgacccctttactctgacccctttattgtctactcctcagagcccccagtctgtatccttctaacttagaaaggggattatggctcagggtccaactctgtgctcagagctttcaacaactactcagaaacacaagatgctgggacagtgacctggactgtgggcctctcatgcaccaccatcaaggactcaaatgggctttccgaattcactggagcctcgaatgtccattcctgagttctgcaaagggagagtggtcaggttgcctctgtctcagaatgaggctggataagatctcaggccttcctaccttcagacctttccagattcttccctgaggtgcaatgcacagccttcctcacagagccagcccccctctatttatatttgcacttattatttattatttatttattatttatttatttgcttatgaatgtatttatttggaaggccggggtgtcctggaggacccagtgtgggaagctgtcttcagacagacatgttttctgtgaaaacggagctgagctgtccccacctggcctctctaccttgttgcctcctcttttgcttatgtttaaaacaaaatatttatctaacccaattgtcttaataacgctgatttggtgaccaggctgtcgctacatcactgaacctctgctccccacgggagccgtgactgtaatcgccctacgggtcattgagagaaataaa

SEQ ID NO: 70 - DNA, 4261 bp, Homo sapiens
agccgccttcctatttccgcccggcgggcagcgctgcggggcgagtgccagcagagaggcgctcggtcctccctccgccctcccgcgccgggggcaggccctgcctagtctgcgtctttttcccccgcaccgcggcgccgctccgccactcgggcaccgcaggtagggcaggaggctggagagcctgctgcccgcccgcccgtaaaatggtcccctcggctggacagctcgccctgttcgctctgggtattgtgttggctgcgtgccaggccttggagaacagcacgtccccgctgagtgacccgcccgtggctgcagcagtggtgtcccattttaatgactgcccagattcccacactcagttctgcttccatggaacctgcaggtttttggtgcaggaggacaagccagcatgtgtctgccattctgggtacgttggtgcacgctgtgagcatgcggacctcctggccgtggtggctgccagccagaagaagcaggccatcaccgccttggtggtggtctccatcgtggccctggctgtccttatcatcacatgtgtgctgatacactgctgccaggtccgaaaacactgtgagtggtgccgggccctcatctgccggcacgagaagcccagcgccctcctgaagggaagaaccgcttgctgccactcagaaacagtggtctgaagagcccagaggaggagtttggccaggtggactgtggcagatcaataaagaaaggcttcttcaggacagcactgccagagatgcctgggtgtgccacagaccttcctacttggcctgtaatcacctgtgcagccttttgtgggccttcaaaactctgtcaagaactccgtctgcttggggttattcagtgtgacctagagaagaaatcagcggaccacgatttcaagacttgttaaaaaagaactgcaaagagacggactcctgttcacctaggtgaggtgtgtgcagcagttggtgtctgagtccacatgtgtgcagttgtcttctgccagccatggattccaggctatatatttctttttaatgggccacctccccacaacagaattctgcccaacacaggagatttctatagttattgttttctgtcatttgcctactggggaagaaagtgaaggaggggaaactgtttaatatcacatgaagaccctagctttaagagaagctgtatcctctaaccacgagaccctcaaccagcccaacatcttccatggacacatgacattgaagaccatcccaagctatcgccacccttggagatgatgtcttatttattagatggataatggttttatttttaatctcttaagtcaatgtaaaaagtataaaaccccttcagacttctacattaatgatgtatgtgttgctgactgaaaagctatactgattagaaatgtctggcctcttcaagacagctaaggcttgggaaaagtcttccagggtgcggagatggaaccagaggctgggttactggtaggaataaaggtaggggttcagaaatggtgccattgaagccacaaagccggtaaatgcctcaatacgttctgggagaaaacttagcaaatccatcagcagggatctgtcccctctgttggggagagaggaagagtgtgtgtgtctacacaggataaacccaatacatattgtactgctcagtgattaaatgggttcacttcctcgtgagccctcggtaagtatgtttagaaatagaacattagccacgagccataggcatttcaggccaaatccatgaaagggggaccagtcatttattttccattttgttgcttggttggtttgttgctttatttttaaaaggagaagtttaactttgctatttattttcgagcactaggaaaactattccagtaatttttttttcctcatttccattcaggatgccggctttattaacaaaaactctaacaagtcacctccactatgtgggtcttcctttcccctcaagagaaggagcaattgttcccctgagcatctgggtccatctgacccatggggcctgcctgtgagaaacagtgggtcccttcaaatacatagtggatagctcatccctaggaattttcattaaaatttggaaacagagtaatgaagaaataatatataaactccttatgtgaggaaatgctactaatatctgaaaagtgaaagatttctatgtattaactcttaagtgcacctagcttattacatcgtgaaaggtacatttaaaatatgttaaattggcttgaaattttcagagaattttgtcttcccctaattcttcttccttggtctggaagaacaatttctatgaattttctctttatttttttttataattcagacaattctatgacccgtgtcttcatttttggcactcttatttaacaatgccacacctgaagcacttggatctgttcagagctgaccccctagcaacgtagttgacacagctccaggtttttaaattactaaaataagttcaagtttacatcccttgggccagatatgtgggttgaggcttgactgtagcatcctgcttagagaccaatcaacggacactggtttttagacctctatcaatcagtagttagcatccaagagactttgcagaggcgtaggaatgaggctggacagatggcggaagcagaggttccctgcgaagacttgagatttagtgtctgtgaatgttctagttcctaggtccagcaagtcacacctgccagtgccctcatccttatgcctgtaacacacatgcagtgagaggcctcacatatacgcctccctagaagtgccttccaagtcagtcctttggaaaccagcaggtctgaaaaagaggctgcatcaatgcaagcctggttggaccattgtccatgcctcaggatagaacagcctggcttatttggggatttttcttctagaaatcaaatgactgataagcattggatccctctgccatttaatggcaatggtagtctttggttagctgcaaaaatactccatttcaagttaaaaatgcatcttctaatccatctctgcaagctccctgtgtttccttgccctttagaaaatgaattgttcactacaattagagaatcatttaacatcctgacctggtaagctgccacacacctggcagtggggagcatcgctgtttccaatggctcaggagacaatgaaaagcccccatttaaaaaaataacaaacattttttaaaaggcctccaatactcttatggagcctggatttttcccactgctctacaggctgtgactttttttaagcatcctgacaggaaatgttttcttctacatggaaagatagacagcagccaaccctgatctggaagacagggccccggctggacacacgtggaaccaagccagggatgggctggccattgtgtccccgcaggagagatgggcagaatggccctagagttcttttccctgagaaaggagaaaaagatgggattgccactcacccacccacactggtaagggaggagaatttgtgcttctggagcttctcaagggattgtgttttgcaggtacagaaaactgcctgttatcttcaagccaggttttcgagggcacatgggtcaccagttgctttttcagtcaatttggccgggatggactaatgaggctctaacactgctcaggagacccctgccctctagttggttctgggctttgatctcttccaacctgcccagtcacagaaggaggaatgactcaaatgcccaaaaccaagaacacattgcagaagtaagacaaacatgtatatttttaaatgttctaacataagacctgttctctctagccattgatttaccaggctttctgaaagatctagtggttcacacagagagagagagagtactgaaaaagcaactcctcttcttagtcttaataatttactaaaatggtcaacttttcattatctttattataataaacctgatgcttttttttagaactccttactctgatgtctgtatatgttgcactgaaaaggttaatatttaatgttttaatttattttgtgtggtaagttaattttgatttctgtaatgtgttaatgtgattagcagttattttccttaatatctgaattatacttaaagagtagtgagcaatataagacgcaattgtgtttttcagtaatgtgcattgttattgagttgtactgtaccttatttggaaggatgaaggaatgaatctttttttcctaaa

SEQ ID NO: 71 - DNA, 4264 bp, Homo sapiens
agccgccttcctatttccgcccggcgggcagcgctgcggggcgagtgccagcagagaggcgctcggtcctccctccgccctcccgcgccgggggcaggccctgcctagtctgcgtctttttcccccgcaccgcggcgccgctccgccactcgggcaccgcaggtagggcaggaggctggagagcctgctgcccgcccgcccgtaaaatggtcccctcggctggacagctcgccctgttcgctctgggtattgtgttggctgcgtgccaggccttggagaacagcacgtccccgctgagtgcagacccgcccgtggctgcagcagtggtgtcccattttaatgactgcccagattcccacactcagttctgcttccatggaacctgcaggtttttggtgcaggaggacaagccagcatgtgtctgccattctgggtacgttggtgcacgctgtgagcatgcggacctcctggccgtggtggctgccagccagaagaagcaggccatcaccgccttggtggtggtctccatcgtggccctggctgtccttatcatcacatgtgtgctgatacactgctgccaggtccgaaaacactgtgagtggtgccgggccctcatctgccggcacgagaagcccagcgccctcctgaagggaagaaccgcttgctgccactcagaaacagtggtctgaagagcccagaggaggagtttggccaggtggactgtggcagatcaataaagaaaggcttcttcaggacagcactgccagagatgcctgggtgtgccacagaccttcctacttggcctgtaatcacctgtgcagccttttgtgggccttcaaaactctgtcaagaactccgtctgcttggggttattcagtgtgacctagagaagaaatcagcggaccacgatttcaagacttgttaaaaaagaactgcaaagagacggactcctgttcacctaggtgaggtgtgtgcagcagttggtgtctgagtccacatgtgtgcagttgtcttctgccagccatggattccaggctatatatttctttttaatgggccacctccccacaacagaattctgcccaacacaggagatttctatagttattgttttctgtcatttgcctactggggaagaaagtgaaggaggggaaactgtttaatatcacatgaagaccctagctttaagagaagctgtatcctctaaccacgagaccctcaaccagcccaacatcttccatggacacatgacattgaagaccatcccaagctatcgccacccttggagatgatgtcttatttattagatggataatggttttatttttaatctcttaagtcaatgtaaaaagtataaaaccccttcagacttctacattaatgatgtatgtgttgctgactgaaaagctatactgattagaaatgtctggcctcttcaagacagctaaggcttgggaaaagtcttccagggtgcggagatggaaccagaggctgggttactggtaggaataaaggtaggggttcagaaatggtgccattgaagccacaaagccggtaaatgcctcaatacgttctgggagaaaacttagcaaatccatcagcagggatctgtcccctctgttggggagagaggaagagtgtgtgtgtctacacaggataaacccaatacatattgtactgctcagtgattaaatgggttcacttcctcgtgagccctcggtaagtatgtttagaaatagaacattagccacgagccataggcatttcaggccaaatccatgaaagggggaccagtcatttattttccattttgttgcttggttggtttgttgctttatttttaaaaggagaagtttaactttgctatttattttcgagcactaggaaaactattccagtaatttttttttcctcatttccattcaggatgccggctttattaacaaaaactctaacaagtcacctccactatgtgggtcttcctttcccctcaagagaaggagcaattgttcccctgagcatctgggtccatctgacccatggggcctgcctgtgagaaacagtgggtcccttcaaatacatagtggatagctcatccctaggaattttcattaaaatttggaaacagagtaatgaagaaataatatataaactccttatgtgaggaaatgctactaatatctgaaaagtgaaagatttctatgtattaactcttaagtgcacctagcttattacatcgtgaaaggtacatttaaaatatgttaaattggcttgaaattttcagagaattttgtcttcccctaattcttcttccttggtctggaagaacaatttctatgaattttctctttatttttttttataattcagacaattctatgacccgtgtcttcatttttggcactcttatttaacaatgccacacctgaagcacttggatctgttcagagctgaccccctagcaacgtagttgacacagctccaggtttttaaattactaaaataagttcaagtttacatcccttgggccagatatgtgggttgaggcttgactgtagcatcctgcttagagaccaatcaacggacactggtttttagacctctatcaatcagtagttagcatccaagagactttgcagaggcgtaggaatgaggctggacagatggcggaagcagaggttccctgcgaagacttgagatttagtgtctgtgaatgttctagttcctaggtccagcaagtcacacctgccagtgccctcatccttatgcctgtaacacacatgcagtgagaggcctcacatatacgcctccctagaagtgccttccaagtcagtcctttggaaaccagcaggtctgaaaaagaggctgcatcaatgcaagcctggttggaccattgtccatgcctcaggatagaacagcctggcttatttggggatttttcttctagaaatcaaatgactgataagcattggatccctctgccatttaatggcaatggtagtctttggttagctgcaaaaatactccatttcaagttaaaaatgcatcttctaatccatctctgcaagctccctgtgtttccttgccctttagaaaatgaattgttcactacaattagagaatcatttaacatcctgacctggtaagctgccacacacctggcagtggggagcatcgctgtttccaatggctcaggagacaatgaaaagcccccatttaaaaaaataacaaacattttttaaaaggcctccaatactcttatggagcctggatttttcccactgctctacaggctgtgactttttttaagcatcctgacaggaaatgttttcttctacatggaaagatagacagcagccaaccctgatctggaagacagggccccggctggacacacgtggaaccaagccagggatgggctggccattgtgtccccgcaggagagatgggcagaatggccctagagttcttttccctgagaaaggagaaaaagatgggattgccactcacccacccacactggtaagggaggagaatttgtgcttctggagcttctcaagggattgtgttttgcaggtacagaaaactgcctgttatcttcaagccaggttttcgagggcacatgggtcaccagttgctttttcagtcaatttggccgggatggactaatgaggctctaacactgctcaggagacccctgccctctagttggttctgggctttgatctcttccaacctgcccagtcacagaaggaggaatgactcaaatgcccaaaaccaagaacacattgcagaagtaagacaaacatgtatatttttaaatgttctaacataagacctgttctctctagccattgatttaccaggctttctgaaagatctagtggttcacacagagagagagagagtactgaaaaagcaactcctcttcttagtcttaataatttactaaaatggtcaacttttcattatctttattataataaacctgatgcttttttttagaactccttactctgatgtctgtatatgttgcactgaaaaggttaatatttaatgttttaatttattttgtgtggtaagttaattttgatttctgtaatgtgttaatgtgattagcagttattttccttaatatctgaattatacttaaagagtagtgagcaatataagacgcaattgtgtttttcagtaatgtgcattgttattgagttgtactgtaccttatttggaaggatgaaggaatgaatctttttttcctaaa

SEQ ID NO: 72 - DNA, 3772 bp, Mus musculus
gcgcctttttcccccgcgcacaccgcggcggcgcgcggctactcgccaaccgcagggagcgcggtggctgcagcaccctgcgctcggaagatggtccccgcgaccggacagctcgctctgctagcgctgggtatcctgttagctgtgtgccaggctctggagaacagcacatcccccctgagtgactcacccgtggcggctgcagtggtgtctcacttcaacaagtgcccagattcccacactcagtactgcttccatggaacctgccggtttttggtgcaggaagagaagccagcatgtgtctgccactctgggtacgtgggtgttcgctgtgagcatgcagacctcctggctgtggtggctgccagccagaagaagcaagccatcactgccctggtggtggtctccattgtggccctggctgtcctcattatcacctgtgtgctgatccactgctgtcagctccgcaaacactgtgagtggtgccgtgccctcgtctgcagacatgagaagcccagcgccctcctgaagggaaggactgcttgctgccactctgagacagtggtctgaagatcccagaggaggaatttggccaggtggcctatgacagcccaaccaagaaaaggcatcttgggacaacacccctggcagtgcccaggcccatgggacatgctgggagaccttccccctcagtgcacaactgcctgggcaggcttcttccttgagagtcttcaaaactgtgtgataaagctgcctgctgggctcgctcagtacacccagagaagaggccagtggaccacattttaaagacaagttgaacacgaacctcaaagggttggccttcttgctaacccacaccgagaatgagctggggctgctgtcccctgccagccatgacttccagactgtttcttctctatgggccatctacccccccgtcccgagactccatggttggtgtacaaaatggacaaggggaaacgtctattgttctttgaagacaccatggcgtcccatgcgccctgacatctcctcaggctgtcgtcaggatgcgtgtcttatttattagatggataacatggttttatttgtaatctctttatgtcaatgtcaggcgtccatgctagcgatgcatgagtggcgggccgtgaagttacaccaaatagaaatgtccagccactttgataaagtaaggctgcagggaggggagcctccggggggccgtagagacagaattcagatgtgcatcctgctgggaaccaacctgctatcccaaggctgtgccccctcaggcaagaaagtccctggatacatgctgtagcaaacttagcaaggccaatggcagagagtgtcccccttgtggggacaagaggacaaaagagcctgtgtgggcacctaggaaggatgcacaccccaacccttgattaagtggattagcgtcctcgtgagcccagctccagcatcagcaatgagctgtgagccaaatccatgaaggggaccagccctttattggcttttgcttatttttagaaggagaaatttgcctttcctatttattttcaagcattaagaaaaatagcccactgttttgttttgtttcccctttaataatgttggcattattaacaaaatcgctaccaagcctctccacccctctctggggaaggaaatgtgttccctgagcacccgaagatgctctgacccattgagcccacccttgttcctgggctgcttgggactgtcaggagacagaatacattcagttgcatattaaagagaccattctccaactcagaagtgaaaagtctgccccctcggccctccagggcctctgaagtaaggcatgagggaaggggtactgggcataccaagttggtttgaagcttctgtcagaagctgcctgttcctcattcttctcctttatgaatttttctcctctcatatgatctaggcattgatctgcccaggtcttaaccctttgttcacaaggcctgaagccctgaaagaagaacctgaaactgttcagtgctgtcctctaactgcttagctgacacagctccaggcagaagttgtcattcttgctattattttggtttgatgctttatcttgttttgtgagacaaggtcttgctgtgtagcccaggttgacctttaagttaagatcctgcttcagcaacctggaagctgggcttacagatgtgcaccaccacgcccagcactttttgattactagagaaaataggttaagtttgctttccttgggtcatacaagtgggtcaaggccaagtgtcacctgctgctcagacaccaatcaacctcactggacttcagccctctattggcccccagtgctctcagtagaccccgcctgaagtactggctcactggagggcagggagcaacacaaatggaggagatgtggggcttgttttagtttagttcacactgttagcttctgatgacaggtcttttctggtctgctggaggctcctgggtgtctgacttcttttaaagagtttgaactgatatcaaaatatcattgggaacttcctcacttaatatcccatcctgaagatgttgtcatccaagactaagcagaggtggtaaacgcttagcacgagcactcgatggtgaaggcagaggatctcgggaggacctcagctttagtgtccaaacggtcctgttctcaggtccagccagtcgcagcagccagtaccatcccagtgcctgtacacagatgcagctggctcaaacacctcccagaagtgccttcccatcagtctaccctttgcagagtcagaggctgagactaggttgagacaagtccggttggacatcgtccctgccccgacgtatgacagccctgctgcgttggtgactttgattttggaagtcagtcagacaaacgctgtgtaccttctcctgtgccatgtcacagcacagatgggcctggttagctgtaccagcgccctcctgtcagttcacactggccctctcttgatatttagtacagctacttaatcaggagcactcttagcagctaggcaggtcagagggcagtacagttgattcagtgactcaggggactagggccattcccattaaatgagctgaaattaattctagagacattgagacagctcagttagcaatggcacttaccaaaatcccagataacctgaattcaatcccctgggatcccatctggtggaaggagagaaccggcttgcccaggatgtcttctgatttctacgtacacgcacacgcacacacacgcacgcgcacacacacgcacacacacgcacatatgagtacacacaaagtctaagtttttaaaataaaaattctagaaagacctctacacctctagctataactttaaacacaccatgggacatgttttctccacggaaaagcacaattggagacccagtctgaaatgaaagatagattccaggtggacatacacagaaccaaaccatgagttagtttgtcatgtgccccagaaaaaggtgggaacaataatcccaacatccctttcctctccccccccaaaaaagggggggaggaggcatggttggtacccacccacctgcactggtgagggaagtgactatgtccctgagtcctagatggtgacgttggtcccttcaagtccccaggccaggtttcctagtgcctatgaggcactggatagaccagcaacactctgacactgcctacaggccccagtcctgtaatgagtaataaacttgtctctctcccaaggcca

SEQ ID NO: 73 - DNA, 522 bp, Sus scrofa
gcccgtaaaatggtcccctcggctggacagttcgccctcttcgcgctgggtatcttgttggccgtgtgccaggccctggagaacagcacgtccgccctgagtgcagacccgcccatagctgcagctgtggtgtcccattttaatgactgcccagattcccacagtcagttctgcttccatggaacctgcaggtttttggtgcaggaggacaagccagcatgtgtctgccactctgggtacgtcggcgcacgctgtgaacacgcagacctcctggcggtggtggccgccagccagaagaagcaggccattaccgccttggtggtggtctccatagtggccctggctgtcctcatcatcacatgtgtgctgatacactgctgccaggtccgaaaacactgtgagtggtgccgggccctcatctgccggcacgagaagcccagtgccctcctgaagggaaggaccgcttgctgccactcagaaacagtggtctgaggagcccagaggaggagtttggccaggtgg

SEQ ID NO: 74 - DNA, 1252 bp, Homo sapiens
ctttgaattcctagctcctgtggtctccagatttcaggcctaagatgaaagcctctagtcttgccttcagccttctctctgctgcgttttatctcctatggactccttccactggactgaagacactcaatttgggaagctgtgtgatcgccacaaaccttcaggaaatacgaaatggattttctgagatacggggcagtgtgcaagccaaagatggaaacattgacatcagaatcttaaggaggactgagtctttgcaagacacaaagcctgcgaatcgatgctgcctcctgcgccatttgctaagactctatctggacagggtatttaaaaactaccagacccctgaccattatactctccggaagatcagcagcctcgccaattcctttcttaccatcaagaaggacctccggctctgtcatgcccacatgacatgccattgtggggaggaagcaatgaagaaatacagccagattctgagtcactttgaaaagctggaacctcaggcagcagttgtgaaggctttgggggaactagacattcttctgcaatggatggaggagacagaataggaggaaagtgatgctgctgctaagaatattcgaggtcaagagctccagtcttcaatacctgcagaggaggcatgaccccaaaccaccatctctttactgtactagtcttgtgctggtcacagtgtatcttatttatgcattacttgcttccttgcatgattgtctttatgcatccccaatcttaattgagaccatacttgtataagatttttgtaatatctttctgctattggatatatttattagttaatatatttatttattttttgctatttaatgtatttatttttttacttggacatgaaactttaaaaaaattcacagattatatttataacctgactagagcaggtgatgtatttttatacagtaaaaaaaaaaaaccttgtaaattctagaagagtggctaggggggttattcatttgtattcaactaaggacatatttactcatgctgatgctctgtgagatatttgaaattgaaccaatgactacttaggatgggttgtggaataagttttgatgtggaattgcacatctaccttacaattactgaccatccccagtagactccccagtcccataattgtgtatcttccagccaggaatcctacacggccagcatgtatttctacaaataaagttttctttgcataacaaaaaaaaaaaaaaaaaaa

SEQ ID NO: 75 - DNA, 824 bp, Mus musculus
tgggagacatcgatagccctgattgatctctttgaattttcgcttctggtctccaggatctaggtgtaagatgaaaggctttggtcttgcctttggactgttctccgctgtgggttttcttctctggactcctttaactgggctcaagaccctccatttgggaagctgtgtgattactgcaaacctacaggcaatacaaaaggaattttctgagattcgggatagtgtgcaagctgaagatacaaatattgacatcagaattttaaggacgactgagtctttgaaagacataaagtctttggataggtgctgcttccttcgtcatctagtgagattctatctggacagggtattcaaagtctaccagacccctgaccaccataccctgagaaagatcagcagcctcgccaactcctttcttatcatcaagaaggacctctcagtctgtcattctcacatggcatgtcattgtggggaagaagcaatggagaaatacaaccaaattctgagtcacttcatagagttggaacttcaggcagcggtggtaaaggctttgggagaactaggcattcttctgagatggatggaggagatgctatagatgaaagtggagaggctgctgagaacactcctgtccaagaatctcagacctcagcaccatgaagacatggccccaggtgctggcatttctactcaagagttccagtcctcagcaccacgaagatggcctcaaaccaccacccctttgtgatataacttagtgctagctatgtgtatattatttctacattattggctcccttatgtgaatgccttcatgtgtc

SEQ ID NO: 76 - DNA, 1240 bp, Homo sapiens
cacattgttctgatcatctgaagatcagctattagaagagaaagatcagttaagtcctttggacctgatcagcttgatacaagaactactgatttcaacttctttggcttaattctctcggaaacgatgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaatggttgtcctgcctgcaatatttgaattttaaatctaaatctatttattaatatttaacattatttatatggggaatatatttttagactcatcaatcaaataagtatttataatagcaacttttgtgtaatgaaaatgaatatctattaatatatgtattatttataattcctatatcctgtgactgtctcacttaatcctttgttttctgactaattaggcaaggctatgtgattacaaggctttatctcaggggccaactaggcagccaacctaagcaagatcccatgggttgtgtgtttatttcacttgatgatacaatgaacacttataagtgaagtgatactatccagttactgccggtttgaaaatatgcctgcaatctgagccagtgctttaatggcatgtcagacagaacttgaatgtgtcaggtgaccctgatgaaaacatagcatctcaggagatttcatgcctggtgcttccaaatattgttgacaactgtgactgtacccaaatggaaagtaactcatttgttaaaattatcaatatctaatatatatgaataaagtgtaagttcacaacaaaaaaaaaaaaaaaaaaaaaaaaaaaaaa

SEQ ID NO: 77 - DNA, 1207 bp, Mus musculus
atagctgccatcggctgacctagagaagacacatcagctgatcctttggaccctctgacttgagacagaagttctgggcttctcctcctgcggcctagctctgagacaatgaacgctacacactgcatcttggctttgcagctcttcctcatggctgtttctggctgttactgccacggcacagtcattgaaagcctagaaagtctgaataactattttaactcaagtggcatagatgtggaagaaaagagtctcttcttggatatctggaggaactggcaaaaggatggtgacatgaaaatcctgcagagccagattatctctttctacctcagactctttgaagtcttgaaagacaatcaggccatcagcaacaacataagcgtcattgaatcacacctgattactaccttcttcagcaacagcaaggcgaaaaaggatgcattcatgagtattgccaagtttgaggtcaacaacccacaggtccagcgccaagcattcaatgagctcatccgagtggtccaccagctgttgccggaatccagcctcaggaagcggaaaaggagtcgctgctgattcggggtggggaagagattgtcccaataagaataattctgccagcactatttgaatttttaaatctaaacctatttattaatatttaaaactatttatatggagaatctattttagatgcatcaaccaaagaagtatttatagtaacaacttatatgtgataagagtgaattcctattaatatatgtgttatttataatttctgtctcctcaactatttctctttgaccaattaattattctttctgactaattagccaagactgtgattgcggggttgtatctgggggtgggggacagccaagcggctgactgaactcagattgtagcttgtacctttacttcactgaccaataagaaacattcagagctgcagtgaccccgggaggtgctgctgatgggaggagatgtctacactccgggccagcgctttaacagcaggccagacagcactcgaatgtgtcaggtagtaacaggctgtccctgaaagaaagcagtgtctcaagagacttgacacctggtgcttccctatacagctgaaaactgtgactacacccgaatgacaaataactcgctcatttatagtttatcactgtctaattgcatatgaataaagtatacctttgcaacc

SEQ ID NO: 78 - DNA, 717 bp, Sus scrofa
cagaagctaactctctccgaaacaatgagttatacaacttatttcttagcttttcagctttgcgtgactttgtgtttttctggctcttactgccaggcgcccttttttaaagaaataacgatcctaaaggactattttaatgcaagtacctcagatgtacctaatggtggacctcttttcttagaaattttgaagaattggaaagaggagagtgacaaaaaaataattcagagccaaattgtctccttctacttcaaattctttgaaatcttcaaagataaccaggccattcaaaggagcatggatgtgatcaagcaagacatgtttcagaggttcctaaatggtagctctgggaaactgaatgacttcgaaaagctgattaaaattccggtagataatctgcagatccagcgcaaagccatcagtgaactcatcaaagtgatgaatgatctgtcaccaagatctaacctaagaaagcggaagagaagtcagactatgttccaaggccagagagcatcaaaataattgtcatcctgcctgcaatatttgaatttttaaatctcaatctatttattaatatttaatattttacattatttatatggggaatattgattcattgatcaaagtatttataatgtaacttttatgtgatgaaaatgggtatctattaacatatgtgttatttatgattcctgtatcctgtgactatttcac

SEQ ID NO: 79 - DNA, 3853 bp, Homo sapiens
acgcgctcacacacagagagaaaatccttctgcctgttgatttatggaaacaattatgattctgctggagaacttttcagctgagaaatagtttgtagctacagtagaaaggctcaagttgcaccaggcagacaacagacatggaattcttatatatccagctgttagcaacaaaacaaaagtcaaatagcaaacagcgtcacagcaactgaacttactacgaactgtttttatgaggatttatcaacagagttatttaaggaggaatcctgtgttgttatcaggaactaaaaggataaggctaacaatttggaaagagcaactactctttcttaaatcaatctacaattcacagataggaagaggtcaatgacctaggagtaacaatcaactcaagattcattttcattatgttattcatgaacacccggagcactacactataatgcacaaatggatactgacatggatcctgccaactttgctctacagatcatgctttcacattatctgtctagtgggtactatatctttagcttgcaatgacatgactccagagcaaatggctacaaatgtgaactgttccagccctgagcgacacacaagaagttatgattacatggaaggaggggatataagagtgagaagactcttctgtcgaacacagtggtacctgaggatcgataaaagaggcaaagtaaaagggacccaagagatgaagaataattacaatatcatggaaatcaggacagtggcagttggaattgtggcaatcaaaggggtggaaagtgaattctatcttgcaatgaacaaggaaggaaaactctatgcaaagaaagaatgcaatgaagattgtaacttcaaagaactaattctggaaaaccattacaacacatatgcatcagctaaatggacacacaacggaggggaaatgtttgttgccttaaatcaaaaggggattcctgtaagaggaaaaaaaacgaagaaagaacaaaaaacagcccactttcttcctatggcaataacttaattgcatatggtatataaagaacccagttccagcagggagatttctttaagtggactgttttctttcttctcaaaattttctttccttttattttttagtaatcaagaaaggctggaaaaactactgaaaaactgatcaagctggacttgtgcatttatgtttgttttaagacactgcattaaagaaagatttgaaaagtatacacaaaaatcagatttagtaactaaaggttgtaaaaaattgtaaaactggttgtacaatcatgatgttagtaacagtaatttttttcttaaattaatttacccttaagagtatgttagatttgattatctgataatgattatttaaatattcctatctgcttataaaatggctgctataataataataatacagatgttgttatataaggtatatcagacctacaggcttctggcaggatttgtcagataatcaagccacactaactatggaaaatgagcagcattttaaatgctttctagtgaaaaattataatctacttaaactctaatcagaaaaaaaattctcaaaaaaactattatgaaagtcaataaaatagataatttaacaaaagtacaggattagaacatgcttatacctataaataagaacaaaatttctaatgctgctcaagtggaaagggtattgctaaaaggatgtttccaaaaatcttgtatataagatagcaacagtgattgatgataatactgtacttcatcttacttgccacaaaataacattttataaatcctcaaagtaaaattgagaaatctttaagtttttttcaagtaacataatctatctttgtataattcatatttgggaatatggcttttaataatgttcttcccacaaataatcatgcttttttcctatggttacagcattaaactctattttaagttgtttttgaactttattgttttgttatttaagtttatgttatttataaaaaaaaaaccttaataagctgtatctgtttcatatgcttttaattttaaaggaataacaaaactgtctggctcaacggcaagtttccctcccttttctgactgacactaagtctagcacacagcacttgggccagcaaatcctggaagcagacaaaaataagagcctgaagcaatgcttacaatagatgtctcacacagaacaatacaaatatgtaaaaactctttcaccacatattcttgccaattaattggatcatataagtaaaatcattacaaatataagtatttacaggattttaaagttagaatatatttgaatgcatgggtagaaaatatcatattttaaaactatgtatatttaaatttagtaattttctaatctctagaaatctctgctgttcaaaaggtggcagcactgaaagttgttttcctgttagatggcaagagcacaatgcccaaaatagaagatgcagttaagaataaggggccctgaatgtcatgaaggcttgaggtcagcctacagataacaggattattacaaggatgaatttccacttcaaaagtctttcattggcagatcttggtagcactttatatgttcaccaatgggaggtcaatatttatctaatttaaaaggtatgctaaccactgtggttttaatttcaaaatatttgtcattcaagtccctttacataaatagtatttggtaatacatttatagatgagagttatatgaaaaggctaggtcaacaaaaacaatagattcatttaattttcctgtggttgacctatacgaccaggatgtagaaaactagaaagaactgcccttcctcagatatactcttgggagagagcatgaatggtattctgaactatcacctgattcaaggactttgctagctaggttttgaggtcaggcttcagtaactgtagtcttgtgagcatattgagggcagaggaggacttagtttttcatatgtgtttccttagtgcctagcagactatctgttcataatcagttttcagtgtgaattcactgaatgtttatagacaaaagaaaatacacactaaaactaatcttcattttaaaagggtaaaacatgactatacagaaatttaaatagaaatagtgtatatacatataaaatacaagctatgttaggaccaaatgctctttgtctatggagttatacttccatcaaattacatagcaatgctgaattaggcaaaaccaacatttagtggtaaatccattcctggtagtataagtcacctaaaaaagacttctagaaatatgtactttaattatttgtttttctcctatttttaaatttattatgcaaattttagaaaataaaatttgctctagttacacacctttagaattctagaatattaaaactgtaaggggcctccatccctcttactcatttgtagtctaggaaattgagattttgatacacctaaggtcacgcagctgggtagatatacagctgtcacaagagtctagatcagttagcacatgctttctactcttcgattattagtattattagctaatggtctttggcatgtttttgttttttatttctgttgagatatagcctttacatttgtacacaaatgtgactatgtcttggcaatgcacttcatacacaatgactaatctatactgtgatgatttgactcaaaaggagaaaagaaattatgtagttttcaattctgattcctattcaccttttgtttatgaatggaaagctttgtgcaaaatatacatataagcagagtaagccttttaaaaatgttctttgaaagataaaattaaatacatgagtttctaacaattaga

SEQ ID NO: 80 - DNA, 1925 bp, Mus musculus
aagaggtaatcctcaagttgcacgaggcagacagcagacacggaactcttgtgtacccagctgttccaaacagaacaaaagtcaaggagcaaccgcactgcagcaactggccttgtcacgacctgtttctataaggatacctatcagctgtgttcctgaaggaggaatcgtgttattggtatcagaagctaaaagataaggttaacagtttggaaagagcgacgactttttctctcatcaatctccagttcacaaataggaggaggcccatgacctaggagtagcaatcaacgcaagattcaattctcattatgttattcatgaacacccggggcactacactataatgcgcaaatggatactgacacggatcctgccaactctgctctacaggtcatgcttccacctcgtctgtctagtgggcactatatctctagcttgcaatgacatgagtccggagcaaacggctacgagtgtgaactgttccagccccgagcgacacaccagaagttatgactacatggaaggaggggatataagggtgagaagactgttctgtcgcacccagtggtacctgaggattgacaaacgaggcaaagtgaaagggacccaggagatgaagaacagctacaacatcatggaaatcaggaccgtggcagttggaattgtggcaatcaaaggggtggaaagtgaatactatcttgccatgaacaaggaagggaaactctatgcaaagaaagaatgcaatgaggattgcaacttcaaagaactgattctggaaaaccattataacacctatgcatcagctaaatggacacacagcggaggggaaatgttcgttgccttaaatcaaaaggggattcctgtcaaagggaagaaaacgaagaaagaacaaaaaacagcccattttcttcctatggcaataacctaagtgtacatggtatttgagaaaccagtttcattcagcagggagatttctttccagtggacttttcttctctctctctctctctctctctctctctctctctctctctctctctctctctcagtaaccaagaaaggctggaaaactactgaaaactgatcaagctggacttgcgcatttttgtttattttaaaagactgcattaaagaaagatttgaagaatatacacaaaaatcagatttagtaaaggttgtaaaaagttgtaaaagtggttgtacaatcatgatgttagtgacgtgatatttttcttaaattaatttacccttaagagtatgttagatttgactatctgataatgaatatttaatatcactatctgcttataaaatggctgctataataatattaataatgcagatgatgttatataaggtatgtcagacctaaaggctgctgggatgctttgtcagatgatcaagccccacacaaactacagacgatgagcagtgtttgaaagctttccagtgagaactataatccggaaaaatgaatgttttcaacattttattatagaatagaatctacaggtggttttacaaaatcacaggcttggaacatgcctgtactgactaatatgaacaaaaatccactcaaattgaaaaggtattgctaacaggatgttttaaaaatctgtatataaaatagcaataatgatgataatattgtatttcatctcaccacaaaataatattttataatcccttaaaataaatttcagaaatctttacatttttttcaagtaaatctatctgttgtataaatcacatttgggaacacaacttttagtaaatgctcttcccacaaataatcatgcttatttccctctgtgattacagcattaaactctactttaagttgtatttgaattttattgttttgttatttaagtttatgttatttataaagaaaaaaaccttaaaaaaaaaaaaaaaaaaaa

SEQ ID NO: 81 - DNA, 1131 bp, Homo sapiens
cattctgccctcgagcccaccgggaacgaaagagaagctctatctcccctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattcagca

SEQ ID NO: 82 - DNA, 1087 bp, Mus musculus
ccaagaacgatagtcaattccagaaaccgctatgaagttcctctctgcaagagacttccatccagttgccttcttgggactgatgctggtgacaaccacggccttccctacttcacaagtccggagaggagacttcacagaggataccactcccaacagacctgtctataccacttcacaagtcggaggcttaattacacatgttctctgggaaatcgtggaaatgagaaaagagttgtgcaatggcaattctgattgtatgaacaacgatgatgcacttgcagaaaacaatctgaaacttccagagatacaaagaaatgatggatgctaccaaactggatataatcaggaaatttgcctattgaaaatttcctctggtcttctggagtaccatagctacctggagtacatgaagaacaacttaaaagataacaagaaagacaaagccagagtccttcagagagatacagaaactctaattcatatcttcaaccaagaggtaaaagatttacataaaatagtccttcctaccccaatttccaatgctctcctaacagataagctggagtcacagaaggagtggctaaggaccaagaccatccaattcatcttgaaatcacttgaagaatttctaaaagtcactttgagatctactcggcaaacctagtgcgttatgcctaagcatatcagtttgtggacattcctcactgtggtcagaaaatatatcctgttgtcaggtatctgacttatgttgttctctacgaagaactgacaatatgaatgttgggacactattttaattatttttaatttattgataatttaaataagtaaactttaagttaatttatgattgatatttattatttttatgaagtgtcacttgaaatgttatatgttatagttttgaaatgataacctaaaaatctatttgatataaatattctgttacctagccagatggtttcttggaatgtataagtttacctcaatgaattgctaatttaaatatgtttttaaagaaatctttgtgatgtatttttataatgtttagactgtcttcaaacaaataaattatattatattt

SEQ ID NO: 83 - DNA, 1123 bp, Sus scrofa
cagtctgccctcgagcccaccaggaacgaaagagagctccatctgccctccaggaacccagctatgaactccctctccacaagcgccttcagtccagtcgccttctccctggggctgcttctggtgatggctactgccttccctaccccgggacgcctggaagaagatgccaaaggtgatgccacctcagacaaaatgctcttcacctctccggacaaaactgaagaactcattaagtacatcctcggcaaaatctctgcaatgagaaaggagatgtgtgagaagtatgagaagtgtgaaaacagcaaggaggtactggcagaaaacaacctgaaccttccaaaaatggcagaaaaagacggatgcttccaatctgggttcaatcaggagacctgcttgatgagaatcaccaccggtcttgtggagtttcagatatacctggactacctccagaaagagtatgagagcaataagggaaatgtcgaggctgtgcagattagtaccaaagcactgatccagaccctgaggcaaaagggaaagaatccagacaaagccaccacccctaaccccaccacaaatgccggcctgctggataagctgcagtcacagaacgagtggatgaagaacacaaagatcattctcatcctgcgcagccttgaggatttcctgcagttcagcctgagggccattcggataatgtagctggggcacctgagattgatgccgtccacgggcattccctcctctggtcagaaacctgtccactgggcacataacttatgttgttctctatgaagaactaaaagtatgagcgttaggacactattttaattattttaatttattgatatttaaatatgtgatgtcgagttaatttatataagtgatagatatttatatttttattaagtgccacttgaaatattttatgtatttgttttgaaaaagtaacgtaaaatggctacacggcttgaagatccttgttgtttcagagccaggttgtttcttggagtgtgtaggcttacctcaaataaattgctaacttatacgtatttttaaaagaaatatttatattgtatttatataaagtttaagttgtttttataccaataaacaccttttt

SEQ ID NO: 84 - DNA, 1822 bp, Homo sapiens
ttgccagacagcatagaggctgaggctggggccaggacccagacagagacacacggtcactgcagctgaagccgctgcccctgctacaggcaccaccaggaccagctgatcattccagcccacagcaatggagccacatgactcctcccacatggactctgagttccgatacactctcttcccgattgtttacagcatcatctttgtgctcggggtcattgctaatggctacgtgctgtgggtctttgcccgcctgtacccttgcaagaaattcaatgagataaagatcttcatggtgaacctcaccatggcggacatgctcttcttgatcaccctgccactttggattgtctactaccaaaaccagggcaactggatactccccaaattcctgtgcaacgtggctggctgccttttcttcatcaacacctactgctctgtggccttcctgggcgtcatcacttataaccgcttccaggcagtaactcggcccatcaagactgctcaggccaacacccgcaagcgtggcatctctttgtccttggtcatctgggtggccattgtgggagctgcatcctacttcctcatcctggactccaccaacacagtgcccgacagtgctggctcaggcaacgtcactcgctgctttgagcattacgagaagggcagcgtgccagtcctcatcatccacatcttcatcgtgttcagcttcttcctggtcttcctcatcatcctcttctgcaacctggtcatcatccgtaccttgctcatgcagccggtgcagcagcagcgcaacgctgaagtcaagcgccgggcgctgtggatggtgtgcacggtcttggcggtgttcatcatctgcttcgtgccccaccacgtggtgcagctgccctggacccttgctgagctgggcttccaggacagcaaattccaccaggccattaatgatgcacatcaggtcaccctctgcctccttagcaccaactgtgtcttagaccctgttatctactgtttcctcaccaagaagttccgcaagcacctcaccgaaaagttctacagcatgcgcagtagccggaaatgctcccgggccaccacggatacggtcactgaagtggttgtgccattcaaccagatccctggcaattccctcaaaaattagtccctgcttccaggcctgaagtcttctcctccatgaacatcatggactgagctgggggaagaagggatatctactgtggtctgggcaccacctctgtgggcactggtgggccattagatttggaggctacctcacctgggcagggatgatggcagagccaggctgttggaaaatccagaactcaaatgagccccttcatccgcctgtgggcgcatactacagtaactgtgactgatgactttatcctgagtcccttaatcttatggggccggaaggaatgtcagggccaggtgcagaccttgggggaagactttaaaccacctagttctccccgatggggcatcggtctaaagctttgggggagtgggcgcagtggctcacacctgtaatcccagcactttgggaggccgaggtgggcagatcatgggtcaagagatcgagaccatcctggccaacattgtaaaaccccatctctactaaaacatacaaaaattagccgggcatggtggcacacgcctgtagtcccagctactcaggaggctgaggcaggagaatcgcttgaacctgggaggcagaggttgcagtgaacctagattgcaccattgcactctagcctggcaacagagcgagattccatctcaaaaaaaaaaaaaaaaaaaaaaaaaa

SEQ ID NO: 85 - DNA, 1820 bp, Sus scrofa
agtactgagtctggctggctcagtccgtgtggaggttcctgtggcatctggtggaggggccctgggtcactgtgtttcctcccaggggtgacttggcagtgctttgctctgtttcctcacttctgcaattgccaggccacaacacagaggctcgagctgaggcttggaccaggagggagcagagcctacggctgagctcctcctacaggcatattcagcgctccatgaccaggtgacccttcctgcccagagcaatggagcacaatggctcctttcgtgtggattctgagtttcgatacacgctctttccgattgtttacagtgtcatctttatactgggggtggttgccaacggctatgtgctatgggtctttgctaacttgtacccttccaagaaactaaatgagataaagatctttatggtgaatctcactatggctgacctgctcttcctgatcaccctcccactgtggattgtctactactacaacgagggcgactggattctacccaacttcctgtgcaacgtggctggctgcctcttcttcatcaatacctactgcagtgtggcctttttgggtgtcatcacttataaccgctaccaggcagtagcctatcccatcaagactgcacaggccaccacccgcaagcgtggcatctctttgtccctgatcatttgggtatccattgtggctactgcatcctatttcctggccacagactccaccaacctagtgcccaataaggatggctcaggcaacatcacccgctgctttgagcattatgagccatacagtgtgcccatccttgttgttcatgtcttcatcgccttctgcttcttcctcgtcttcttccttatcttctactgcaacttggtcatcatccacacgctgctcacgcagcccatgaggcagcagcgcaaagcgggggtgaagcggagggcgctgtggatggtctgcacggtcttggcggtattcatcatctgttttgtgccccatcacgtggtccagctgccctggaccctagcagagttgggctaccagaccaacttccatcaggctattaatgatgcccatcaaatcaccctctgcctcctcagcaccaactgtgtcttagatcccgttatctattgctttcttaccaagaagttccgaaagcacctcagtgagaagttttacagcatgcgcagtagccggaagtgctccagagccacgagtgacacgtgcaccgaggtgatagtgccagccaaccagactcctattgtgtcgctgaaaaattaatctctgcttattaaagccagatccagagccttctcttcaatggacctcacagacggagctaggaggtggactgaccctggtggactcaagtgcggcagttacttcctcgcagagagccggttgctggaagatacagaacctggatgctccttttcactagtctttgggtcaacgctagaggactgtggctgatggactcacccagagcttcagattgtgacatccagctagggtcagaccttggggagaaacgctaagcagctcagttcatccaactggggcatcaacttaaggtttggtcagtgacttccaggggctacaggaagaggggagatgggttgtggggattcatcactgattgtgtggaagatttttggggaagcaattaagtttcagatggtgccttccttggtccatctataggtacaaggtccttgtgacccttcctagaggaagtggtttggtggtatctcttgactgacccaagtacctggcctcaaatgtaaccagaggaataaacacagaagttgaccagaagct

SEQ ID NO: 86 - DNA, 2347 bp, Homo sapiens
ctgtttcagggccattggactctccgtcctgcccagagcaagatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttaggttctgatccaggatgaaaatttggaggaaaagtggaagatattaagcaaaatgtttaaagacacaacggaatagacccaaaaagataatttctatctgatttgctttaaaacgtttttttaggatcacaatgatatctttgctgtatttgtatagttagatgctaaatgctcattgaaacaatcagctaatttatgtatagattttccagctctcaagttgccatgggccttcatgctatttaaatatttaagtaatttatgtatttattagtatattactgttatttaacgtttgtctgccaggatgtatggaatgtttcatactcttatgacctgatccatcaggatcagtccctattatgcaaaatgtgaatttaattttatttgtactgacaacttttcaagcaaggctgcaagtacatcagttttatgacaatcaggaagaatgcagtgttctgataccagtgccatcatacacttgtgatggatgggaacgcaagagatacttacatggaaacctgacaatgcaaacctgttgagaagatccaggagaacaagatgctagttcccatgtctgtgaagacttcctggagatggtgttgataaagcaatttagggccacttacacttctaagcaagtttaatctttggatgcctgaattttaaaagggctagaaaaaaatgattgaccagcctgggaaacataacaagaccccgtctctacaaaaaaaatttaaaattagccaggcgtggtggctcatgcttgtggtcccagctgttcaggaggatgaggcaggaggatctcttgagcccaggaggtcaaggctatggtgagccgtgattgtgccactgcataccagcctaggtgacagaatgagaccctgtctcaaaaaaaaaaatgattgaaattaaaattcagctttagcttccatggcagtcctcacccccacctctctaaaagacacaggaggatgacacagaaacaccgtaagtgtctggaaggcaaaaagatcttaagattcaagagagaggacaagtagttatggctaaggacatgaaattgtcagaatggcaggtggcttcttaacagccctgtgagaagcagacagatgcaaagaaaatctggaatccctttctcattagcatgaatgaacctgatacacaattatgaccagaaaatatggctccatgaaggtgctacttttaagtaatgtatgtgcgctctgtaaagtgattacatttgtttcctgtttgtttatttatttatttatttttgcattctgaggctgaactaataaaaactcttctttgtaatc

SEQ ID NO: 87 - DNA, 1951 bp, Mus musculus
agaaggaacagtgggtgtccaggcacatcagaccaggcagctcgcagcaaagcaaggtaagttctctcctcttccctgtcgctaactccctgcatctagaggctgtccagattcagactccaggggacaggctacccctgaaccaggcagcgtgggagtgggatgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatcctaggatgcaacgttggaaaggaaagaaaagtggaagacattaaggaagaaaaatttaaactcaggatggaagagtcccccaaaagctgtcttctgcttggttggctttttccagttttcctaagttcatcatgacacctttgctgatttctacatgtaaatgttaaatgcccgcagagccagggagctaatgtatgcatagatattctagcattccacttggccttatgctgttgaaatatttaagtaatttatgtatttattaatttatttctgcatttcacatttgtataccaagatgtattgaatatttcatgtgctcgtggcctgatccactgggaccaggccctattatgcaaattgtgagcttgttatcttcttcaacagctcttcaatcagggctgcgtaggtacattagcttttgtgacaaccaataagaacataatattctgacacaagcagtgttacatatttgtgaccagtaaagacataggtggtatttggagacatgaagaagctgtaaagttgactctgaagagtttagcactagtttcaacaccaagaaagactttttagaagtgatattgataagaaaccagggccttctttagaagggtacctaaatttaaaagaattttgaaaggctgggtatcggtggtatatgcttttaattccagcactcaggagaccaaggcaggcagatctctgtgagtttgaggacagcctggtgtacagagggagttccagcacagccagtgccacacagaaattctgtctcaaaaaca

SEQ ID NO: 88 - DNA, 2090 bp, Sus scrofa
gtgagaagcaactacggtttcagacccgacgaactctgcagcctgaccagagcaagatgcaccttcagcagctggttgtctcctggttttccctggtttggctggcatctcccattgtggccatatgggaactggagaaaaatgtttatgtcgtagagttggactggtaccccaatgcccctggagaaatggtggtcctcacctgcaacacccctgaagaagacggcatcacgtggacctcagaccagagcagtgaggtcttgggcactggcaaaaccctgaccatccacgtcaaagagtttggagatgctggccagtacacctgtcgcaaaggaggcgcagttctgagccagtcactcctgctgcttcacaaaaaggaagatggaatttggtccactgatattttaaaagaccagaaagagcccaaaaacaagagctttctaaaatgtgaggcaaagaattactccggacgtttcacctgctggtggctgacagcaatcagtactgatttgaaattcagtgtcaaaagcagcagaggctccactgacccccgtggcgtgacatgtggcacggcaacgctctcagaggacctcggggagtataagaagtacagagtggagtgtcaggagggcagtgcctgcccagccgctgaggagagcctgcccattgaggtcgtgctggaagctgttcacaagctcaagtatgaaaactataccagcagcttcttcatcagggacatcatcaaaccagaccctcccaagaatctgcagctgaatccattaaagaattctcgacacgtggagatcagctgggagtaccctgacacctggagcaccccacattcctacttttccctgatgtttggtgttcaagttcagggcaagaacaaaagagaaaagaaagataaactcttcacggaccaaatctcagccaaggttacatgccacaaggatgccaacatccgcgtgcaagcccgggaccgctactacagctcctcctggagtgaatgggcatctgtgtcctgcaattaggttctaatcttgggacaaaaccatggaggaaaagtggattatgctaaattgttaaaagacatgatggagcagacccctcccccaaagatattttctaccaacttgcttttttttaggattataaagatgtctttgctgtattttattatttaaatgttaaatgtccactgaaacaatcaactaatttatttataggctttccagctagcagatcacagctgacttttatgttatttaaatatttaagtaatttatgtatttattaatttattactattatttaacatgtgtgtgccaagatgtatgtttcatatactcatgacctgatttatgaggaatgggccttattatgcaaaatgtgaatgtatgtgttatttatactgacaatttttcagtctagcctgcaagtgccctcagtaagagtaagaacagtaagaacataatgtcctaattccagtgccacaatacatttttgatgggtgggaacacaagagatagttaaatgaggcatggagacccaaactctttgaggaggtcctggagagtacatgttagttcctgtgtccatgaaaacttccttgcaggggtggtggtaattcagggccactggtacctctaggcaagtttagcctttagatacctgaacgtagagctaaaaaaatgatgattgaaattgaaattcaacttcactccccacccccttctatctgtaagacataggagagacatgataagtctctggaaagtaaaaagatcttatgattcaagacagaagacaagtgtagttatggtcaaggatataaaattgtcagaattgctggttgctccttaatgccacataagaagcagacagatgtggaggaaaaaggccaagaatttcttacttgtcagcatgggggaacatgagaagcagatgtgaccagaagtgcctagccattaaaatgctacttttaagtaatgaatgtgcttcctgtaaagtgattccatttgttttctgtttacttatttatttttatattctgatggtatacaaataaaatataattcttctttgcaata

SEQ ID NO: 89 - DNA, 1270 bp, Homo sapiens
agacgttcgcacacctgggtgccagcgccccagaggtcccgggacagcccgaggcgccgcgcccgccgccccgagctccccaagccttcgagagcggcgcacactcccggtctccactcgctcttccaacacccgctcgttttggcggcagctcgtgtcccagagaccgagttgccccagagaccgagacgccgccgctgcgaaggaccaatgagagccccgctgctaccgccggcgccggtggtgctgtcgctcttgatactcggctcaggccattatgctgctggattggacctcaatgacacctactctgggaagcgtgaaccattttctggggaccacagtgctgatggatttgaggttacctcaagaagtgagatgtcttcagggagtgagatttcccctgtgagtgaaatgccttctagtagtgaaccgtcctcgggagccgactatgactactcagaagagtatgataacgaaccacaaatacctggctatattgtcgatgattcagtcagagttgaacaggtagttaagcccccccaaaacaagacggaaagtgaaaatacttcagataaacccaaaagaaagaaaaagggaggcaaaaatggaaaaaatagaagaaacagaaagaagaaaaatccatgtaatgcagaatttcaaaatttctgcattcacggagaatgcaaatatatagagcacctggaagcagtaacatgcaaatgtcagcaagaatatttcggtgaacggtgtggggaaaagtccatgaaaactcacagcatgattgacagtagtttatcaaaaattgcattagcagccatagctgcctttatgtctgctgtgatcctcacagctgttgctgttattacagtccagcttagaagacaatacgtcaggaaatatgaaggagaagctgaggaacgaaagaaacttcgacaagagaatggaaatgtacatgctatagcataactgaagataaaattacaggatatcacattggagtcactgccaagtcatagccataaatgatgagtcggtcctctttccagtggatcataagacaatggaccctttttgttatgatggttttaaactttcaattgtcactttttatgctatttctgtatataaaggtgcacgaaggtaaaaagtattttttcaagttgtaaataatttatttaatatttaatggaagtgtatttattttacagctcattaaacttttttaaccaaacagaaaaaaaaaaaaaaaaaaaaaaaaaaaaaaaaa

SEQ ID NO: 90 - DNA, 1199 bp, Mus musculus
ctcccttctgacccgcggccaccgcccggacatctccagctgagcctgaggcgctggccgctccagccgtccggagcccggcgggctgggcttccctgcgccggtgctcgcccgctgctccgtggttccgctgctcccgccgcccggagccgctgtcgtgttgctgcagagaccgagacgctgccggccggtggaaccaatgagaactccgctgctaccgctggcgcgctcagtgctgttgctgctggtcttaggctcaggccattatgcagctgctttggagctcaatgaccccagctcagggaaaggcgaatcgctttctggggaccacagtgccggtggacttgagctttctgtgggaagagaggtttccaccataagcgaaatgccttctggcagtgaactctccacaggggactacgactactcagaggagtatgataatgaaccacaaatatccggctatattatagatgattcagtcagagttgaacaggtgattaagcccaagaaaaacaagacagaaggagaaaagtctacagaaaaacccaaaaggaagaaaaagggaggcaaaaatggaaaaggcagaaggaataagaagaaaaagaatccatgcactgccaagtttcagaacttttgcattcatggcgaatgcagatacatcgagaacctggaggtggtgacatgcaattgtcatcaagattactttggtgaacggtgtggagaaaaatccatgaagactcacagcgaggatgacaaggacctatccaagattgcagtagtagctgtcactatctttgtctctgccatcatcctcgcagctattggcatcggcatcgttatcacagtgcacctttggaaacgatacttcagggaatatgaaggagaaacagaagaaagaaggaggcttcgacaagaaaacgggactgtgcatgccattgcctagctgaggacaatgcagggtaaaagttgaatcattgccaagccacaccggaaatgacattggtccttctttcagaaaaggaagtggagctttcggatggttccagatgcccagttgtcactttttatgatagtcttacttctgtacataaagagatgtgtgaagataaaatatttttttttcatgttgtaaataatttatttaatatttaagtgttatttattttatagctcattaaactttttttaaacaaaca

SEQ ID NO: 91 - DNA, 1221 bp, Sus scrofa
ccttgtgcgggagccggagcccagggaatcccgaggcgagctccaggcgctgcctccagcgcccgagcccggccgggcctctgagcacagctgctcggcccccctggtccccggtcacctcctcccgcccgcccgcagctcccggccgagttgccccagagaccgcgacgccacagctccgcgggaccaatgagagctccgctgctgccgccggcgcccgtggtgctgtcactcttgatcctctgctcagcccattatgctgctggactggacgtcaacggcacctcctctgggaaaggagaaccattttctggggaccatggtgctgaggcatttgaggtgacctccagaagtgagatgtcctcgggaagtgaggctccccctgctagcgaaatgccttctggtagcgactatgactatgctgaagagtacgataacgaaccgcacatatctggctatattgtagatgattcagtcagagttgaacaggtagttaagcccaagagaaacagaacagaaagtgaaaatacttcagataaacccaaaagaaagaaaaagggaggcaaaagtggaaaaaatagaagaaacagaaagaagaaaaatccatgtgatgcagaattccaaaacttctgcattcacggagattgcaaatatatagagcacctggaagcagtaacctgcaaatgttaccaggattactttggtgaacgatgtggggaaaagtccatgaagactcacaccatggtccacagcgatttatcaaaaattgctttagcagccattgctgcttttgtctctgccatgagcttcacagctattgctgttgttattacaatctaccttcgaaaacgatacttcagggaatatgaaggtgcagctgaagaacgaaagaaacttcgacaagaaaatgcaaatgcacatgccatagcatgactgaagataatattacagggtagcagatcggggtcactgccaagtcataaccatgactgatgagttggttcctctttgcagtggatcataagaagacagaacctttttgttctggtggttttaaacttccaactgtcactttttttaatgctaagtcttatttctgtacataaagatgcacggagataaaaagtattttttcaagttgtaaataatttatttaatatttaatggaagtgtatttattttacagttcattaaacttttttaatcaaaaaaaaaaaaaaa

SEQ ID NO: 92 - DNA, 4678 bp, Homo sapiens
tgaattgcgagatggatagggcttgagtgcccccagccctgctgataccaaatgcctttaagatacagcctttcccatcctaatctacaaaggaaacaggaaaaaggaacttaaaactccctgtgctcagacagaaatgagactgttacagcctgcttctgtgctgttccttcttgcctctaacttgtaaacaagacgtagtaggacgatgctaatggaaagtcacaaaccgctgggtttttgaaaggatccttgggacctcatgcacatttgtggaaactggatggagagatttggggaagcatggactctttagccagcttagttctctgtggagtcagcttgctcctttctggaactgtggaaggtgccatggacttgatcttgatcaattccctacctcttgtatctgatgctgaaacatctctcacctgcattgcctctgggtggcgcccccatgagcccatcaccataggaagggactttgaagccttaatgaaccagcaccaggatccgctggaagttactcaagatgtgaccagagaatgggctaaaaaagttgtttggaagagagaaaaggctagtaagatcaatggtgcttatttctgtgaagggcgagttcgaggagaggcaatcaggatacgaaccatgaagatgcgtcaacaagcttccttcctaccagctactttaactatgactgtggacaagggagataacgtgaacatatctttcaaaaaggtattgattaaagaagaagatgcagtgatttacaaaaatggttccttcatccattcagtgccccggcatgaagtacctgatattctagaagtacacctgcctcatgctcagccccaggatgctggagtgtactcggccaggtatataggaggaaacctcttcacctcggccttcaccaggctgatagtccggagatgtgaagcccagaagtggggacctgaatgcaaccatctctgtactgcttgtatgaacaatggtgtctgccatgaagatactggagaatgcatttgccctcctgggtttatgggaaggacgtgtgagaaggcttgtgaactgcacacgtttggcagaacttgtaaagaaaggtgcagtggacaagagggatgcaagtcttatgtgttctgtctccctgacccctatgggtgttcctgtgccacaggctggaagggtctgcagtgcaatgaagcatgccaccctggtttttacgggccagattgtaagcttaggtgcagctgcaacaatggggagatgtgtgatcgcttccaaggatgtctctgctctccaggatggcaggggctccagtgtgagagagaaggcataccgaggatgaccccaaagatagtggatttgccagatcatatagaagtaaacagtggtaaatttaatcccatttgcaaagcttctggctggccgctacctactaatgaagaaatgaccctggtgaagccggatgggacagtgctccatccaaaagactttaaccatacggatcatttctcagtagccatattcaccatccaccggatcctcccccctgactcaggagtttgggtctgcagtgtgaacacagtggctgggatggtggaaaagcccttcaacatttctgttaaagttcttccaaagcccctgaatgccccaaacgtgattgacactggacataactttgctgtcatcaacatcagctctgagccttactttggggatggaccaatcaaatccaagaagcttctatacaaacccgttaatcactatgaggcttggcaacatattcaagtgacaaatgagattgttacactcaactatttggaacctcggacagaatatgaactctgtgtgcaactggtccgtcgtggagagggtggggaagggcatcctggacctgtgagacgcttcacaacagcttctatcggactccctcctccaagaggtctaaatctcctgcctaaaagtcagaccactctaaatttgacctggcaaccaatatttccaagctcggaagatgacttttatgttgaagtggagagaaggtctgtgcaaaaaagtgatcagcagaatattaaagttccaggcaacttgacttcggtgctacttaacaacttacatcccagggagcagtacgtggtccgagctagagtcaacaccaaggcccagggggaatggagtgaagatctcactgcttggacccttagtgacattcttcctcctcaaccagaaaacatcaagatttccaacattacacactcctcagctgtgatttcttggacaatattggatggctattctatttcttctattactatccgttacaaggttcaaggcaagaatgaagaccagcacgttgatgtgaagataaagaatgccaccatcactcagtatcagctcaagggcctagagcctgaaacagcataccaggtggacatttttgcagagaacaacatagggtcaagcaacccagccttttctcatgaactggtgaccctcccagaatctcaagcaccagcggacctcggaggggggaagatgctgcttatagccatccttggctctgctggaatgacctgcctgactgtgctgttggcctttctgatcatattgcaattgaagagggcaaatgtgcaaaggagaatggcccaagccttccaaaacgtgagggaagaaccagctgtgcagttcaactcagggactctggccctaaacaggaaggtcaaaaacaacccagatcctacaatttatccagtgcttgactggaatgacatcaaatttcaagatgtgattggggagggcaattttggccaagttcttaaggcgcgcatcaagaaggatgggttacggatggatgctgccatcaaaagaatgaaagaatatgcctccaaagatgatcacagggactttgcaggagaactggaagttctttgtaaacttggacaccatccaaacatcatcaatctcttaggagcatgtgaacatcgaggctacttgtacctggccattgagtacgcgccccatggaaaccttctggacttccttcgcaagagccgtgtgctggagacggacccagcatttgccattgccaatagcaccgcgtccacactgtcctcccagcagctccttcacttcgctgccgacgtggcccggggcatggactacttgagccaaaaacagtttatccacagggatctggctgccagaaacattttagttggtgaaaactatgtggcaaaaatagcagattttggattgtcccgaggtcaagaggtgtatgtgaaaaagacaatgggaaggctcccagtgcgctggatggccatcgagtcactgaattacagtgtgtacacaaccaacagtgatgtatggtcctatggtgtgttactatgggagattgttagcttaggaggcacaccctactgcgggatgacttgtgcagaactctacgagaagctgccccagggctacagactggagaagcccctgaactgtgatgatgaggtgtatgatctaatgagacaatgctggcgggagaagccttatgagaggccatcatttgcccagatattggtgtccttaaacagaatgttagaggagcgaaagacctacgtgaataccacgctttatgagaagtttacttatgcaggaattgactgttctgctgaagaagcggcctaggacagaacatctgtataccctctgtttccctttcactggcatgggagacccttgacacctgctgagaaaacatgcctctgccaaaggatgtgatatataagtgtacatatgtgctgtacacctgggaccttcaccactgtagatcccatgcatggatctatgtagtatgctctgactctaataggactgtatatactgttttaagaatgggctgaaatcagaatgcctgtttgtggtttcatatgcaataatatatttttttaaaaatgtggacttcataggaaggcgtgagtacaattagtataatgcataactcattgttgtcctagatattttgatatttacctttatgttgaatgctattaaatgttttcctgtgtcaaagtaaaatattgttaataaacctaacaatgaccctgatagtacaggttaagtgagagaactatatgaattctaacaagtcataggttaatatttaagacactgaaaaatctaagtgatataaatcagattcttctctctcaattttatccctcacctgtagcagccagtcccgtttcatttagtcatgtgaccactctgtcttgtgtttccacagcctgcaagtcagtccaggatgctaacatctaaaaatagacttaaatctcattgcttacaagcctaagaatctttagagaagtatacataagtttaggataaaataatgggattttcttttcttttctctggtaatattgacttgtatattttaagaaataacagaaagcctgggtgacatttgggagacatgtgacatttatatattgaattaatatccctacatgtattgcacattgtaaaaagttttagttttgatgagttgtgagtttaccttgtatactgtaggcacactttgcactgatatatcatgagtgaataaatgtcttgcctactcacgtctcaaaaaaaaaaaaaaaaaaaaaaaaaaaaaaaaaaaaaaaaaaaaaaaaaaaaaaaaaaa

SEQ ID NO: 93 - DNA, 4654 bp, Mus musculus
gagcaggagccggagcaggagcagaagataagccttggatgaagggcaagatggatagggctcgctctgccccaagccctgctgataccaagtgcctttaagatacagcctttcccatcctaatctgcaaaggaaacaggaaaaaggaacttaaccctccctgtgctcagacagaaatgagactgttaccgcctgcttctgtggtgtttctccttgccgccaacttgtaaacaagagcgagtggaccatgcgagcgggaagtcgcaaagttgtgagttgttgaaagcttcccagggactcatgctcatctgtggacgctggatggggagatctggggaagtatggactctttagccggcttagttctctgtggagtcagcttgctcctttatggagtagtagaaggcgccatggacctgatcttgatcaattccctacctcttgtgtctgatgccgaaacatccctcacctgcattgcctctgggtggcacccccatgagcccatcaccataggaagggactttgaagccttaatgaaccagcaccaagatccactggaggttactcaagatgtgaccagagaatgggcgaaaaaagttgtttggaagagagaaaaggccagtaagattaatggtgcttatttctgtgaaggtcgagttcgaggacaggctataaggatacggaccatgaagatgcgtcaacaagcatccttcctacctgctactttaactatgaccgtggacaggggagataatgtgaacatatctttcaaaaaggtgttaattaaagaagaagatgcagtgatttacaaaaatggctccttcatccactcagtgccccggcatgaagtacctgatattttagaagttcacttgccgcatgctcagccccaggatgctggtgtgtactcggccaggtacataggaggaaacctgttcacctcagccttcaccaggctgattgttcggagatgtgaagctcagaagtgggggcccgactgtagccgtccttgtactacttgcaagaacaatggagtctgccatgaagataccggggaatgcatttgccctcctgggtttatggggagaacatgtgagaaagcttgtgagccgcacacatttggcaggacctgtaaagaaaggtgtagtggaccagaaggatgcaagtcttatgtgttctgtctcccagacccttacgggtgttcctgtgccacaggctggagggggttgcagtgcaatgaagcatgcccatctggttactacggaccagactgtaagctcaggtgccactgtaccaatgaagagatatgtgatcggttccaaggatgcctctgctctcaaggatggcaagggctgcagtgtgagaaagaaggcaggccaaggatgactccacagatagaggatttgccagatcacattgaagtaaacagtggaaaatttaaccccatctgcaaagcctctgggtggccactacctactagtgaagaaatgaccctagtgaagccagatgggacagtgctccaaccaaatgacttcaactatacagatcgtttctcagtggccatattcactgtcaaccgagtcttacctcctgactcaggagtctgggtctgcagtgtgaacacagtggctgggatggtggaaaagcctttcaacatttccgtcaaagttcttccagagcccctgcacgccccaaatgtgattgacactggacataactttgctatcatcaatatcagctctgagccttactttggggatggacccatcaaatccaagaagcttttctataaacctgtcaatcaggcctggaaatacattgaagtgacgaatgagattttcactctcaactacttggagccgcggactgactacgagctgtgtgtgcagctggcccgtcctggagagggtggagaagggcatcctgggcctgtgagacgatttacaacagcgtctatcggactccctcctccaagaggtctcagtctcctgccaaaaagccagacagctctaaatttgacttggcaaccgatatttacaaactcagaagatgaattttatgtggaagtcgagaggcgatccctgcaaacaacaagtgatcagcagaacatcaaagtgcctgggaacctgacctcggtgctactgagcaacttagtccccagggagcagtacacagtccgagctagagtcaacaccaaggcgcagggggagtggagtgaagaactcagggcctggacccttagtgacattctccctcctcaaccagaaaacatcaagatctccaacatcactgactccacagctatggtttcttggacaatagtggatggctattcgatttcttccatcatcatccggtataaggttcagggcaaaaatgaagaccagcacattgatgtgaagatcaagaatgctaccgttactcagtaccagctcaagggcctagagccagagactacataccatgtggatatttttgctgagaacaacataggatcaagcaacccagccttttctcatgaactgaggacgcttccacattccccagcctctgcagacctcggagggggaaagatgctactcatagccatccttgggtcggctggaatgacttgcatcaccgtgctgttggcgtttctgattatgttgcaactgaagagagcaaatgtccaaaggagaatggctcaggcattccagaacgtgagagaagaaccagctgtgcagtttaactcaggaactctggcccttaacaggaaggccaaaaacaatccggatcccacaatttatcctgtgcttgactggaatgacatcaagtttcaagacgtgatcggagagggcaactttggccaggttctgaaggcacgcatcaagaaggatgggttacggatggatgccgccatcaagaggatgaaagagtatgcctccaaagatgatcacagggacttcgcaggagaactggaggttctttgtaaacttggacaccatccaaacatcattaatctcttgggagcatgtgaacaccgaggctatttgtacctagctattgagtatgccccgcatggaaacctcctggacttcctgcgtaagagcagagtgctagagacagaccctgcttttgccatcgccaacagtacagcttccacactgtcctcccaacagcttcttcattttgctgcagatgtggcccgggggatggactacttgagccagaaacagtttatccacagggacctggctgccagaaacattttagttggtgaaaactacatagccaaaatagcagattttggattgtcacgaggtcaagaagtgtatgtgaaaaagacaatgggaaggctcccagtgcgttggatggcaatcgaatcactgaactatagtgtctatacaaccaacagtgatgtctggtcctatggtgtattgctctgggagattgttagcttaggaggcaccccctactgcggcatgacgtgcgcggagctctatgagaagctaccccagggctacaggctggagaagcccctgaactgtgatgatgaggtgtatgatctaatgagacagtgctggagggagaagccttatgagagaccatcatttgcccagatattggtgtccttaaacaggatgctggaagaacggaagacatacgtgaacaccacactgtatgagaagtttacctatgcaggaattgactgctctgcggaagaagcagcctagagcagaactcttcatgtacaacggccatttctcctcactggcgcgagagcgccttgacacctgtaccaagcaagccacccactgccaagagatgtgatatataagtgtatatattgtgctgtgtttgggaccctcctcatacagctcgtgcggatctgcagtgtgttctgactctaatgtgactgtatatactgctcggagtaagaatgtgctaagatcagaatgcctgttcgtggtttcatataatatatttttctaaaagcatagattgcacaggaaggtatgagtacaaatactgtaatgcataacttgttattgtcctagatgtgtttgatatttttcctttacaactgaatgctataaaagtgttttgctgtgtacacataagatactgttcgttaaaataagcattcccttgacagcacaggaagaaaagcgagggaaatgtatggattatattaaatgtgggttactacacaagaggccgaacattccaagtagcagaagagagggtctctcaactctgctcctcacctgcagaagccagtttgtttggccatgtgacaattgtcctgtgtttttatagcacccaaatcattctaaaatatgaacatctaaaaactttgctaggagactaagaacctttggagagatagatataagtacggtcaaaaaacaaaactgtgggacttacatttattttctatagtaatctgttgtacattttaagaagtaaaactaggaatttaggagtgatgtgtgacatttctgacatggagttaccatccccacatgtatcacatactgtcatattcccacatgtatcacacatgtattgtaaaattttgtagttttgatcacttgtgaatttactgttgatgtggtagccacctgctgcaatggttcctcttgtaggtgaataaatgtcttgtctacccaca

SEQ ID NO: 94 - DNA, 2887 bp, Homo sapiens
ggagctgagaggaacaggaagtgtcaggactttacgacccgcgcctccagctgaggtttctagacgtgacccagggcagactggtagcaaagcccccacgcccagccaggagcaccgccgaggactccagcacaccgagggacatgctgggcctgcgccccccactgctcgccctggtggggctgctctccctcgggtgcgtcctctctcaggagtgcacgaagttcaaggtcagcagctgccgggaatgcatcgagtcggggcccggctgcacctggtgccagaagctgaacttcacagggccgggggatcctgactccattcgctgcgacacccggccacagctgctcatgaggggctgtgcggctgacgacatcatggaccccacaagcctcgctgaaacccaggaagaccacaatgggggccagaagcagctgtccccacaaaaagtgacgctttacctgcgaccaggccaggcagcagcgttcaacgtgaccttccggcgggccaagggctaccccatcgacctgtactatctgatggacctctcctactccatgcttgatgacctcaggaatgtcaagaagctaggtggcgacctgctccgggccctcaacgagatcaccgagtccggccgcattggcttcgggtccttcgtggacaagaccgtgctgccgttcgtgaacacgcaccctgataagctgcgaaacccatgccccaacaaggagaaagagtgccagcccccgtttgccttcaggcacgtgctgaagctgaccaacaactccaaccagtttcagaccgaggtcgggaagcagctgatttccggaaacctggatgcacccgagggtgggctggacgccatgatgcaggtcgccgcctgcccggaggaaatcggctggcgcaacgtcacgcggctgctggtgtttgccactgatgacggcttccatttcgcgggcgacgggaagctgggcgccatcctgacccccaacgacggccgctgtcacctggaggacaacttgtacaagaggagcaacgaattcgactacccatcggtgggccagctggcgcacaagctggctgaaaacaacatccagcccatcttcgcggtgaccagtaggatggtgaagacctacgagaaactcaccgagatcatccccaagtcagccgtgggggagctgtctgaggactccagcaatgtggtccaactcattaagaatgcttacaataaactctcctccagggtcttcctggatcacaacgccctccccgacaccctgaaagtcacctacgactccttctgcagcaatggagtgacgcacaggaaccagcccagaggtgactgtgatggcgtgcagatcaatgtcccgatcaccttccaggtgaaggtcacggccacagagtgcatccaggagcagtcgtttgtcatccgggcgctgggcttcacggacatagtgaccgtgcaggttcttccccagtgtgagtgccggtgccgggaccagagcagagaccgcagcctctgccatggcaagggcttcttggagtgcggcatctgcaggtgtgacactggctacattgggaaaaactgtgagtgccagacacagggccggagcagccaggagctggaaggaagctgccggaaggacaacaactccatcatctgctcagggctgggggactgtgtctgcgggcagtgcctgtgccacaccagcgacgtccccggcaagctgatatacgggcagtactgcgagtgtgacaccatcaactgtgagcgctacaacggccaggtctgcggcggcccggggagggggctctgcttctgcgggaagtgccgctgccacccgggctttgagggctcagcgtgccagtgcgagaggaccactgagggctgcctgaacccgcggcgtgttgagtgtagtggtcgtggccggtgccgctgcaacgtatgcgagtgccattcaggctaccagctgcctctgtgccaggagtgccccggctgcccctcaccctgtggcaagtacatctcctgcgccgagtgcctgaagttcgaaaagggcccctttgggaagaactgcagcgcggcgtgtccgggcctgcagctgtcgaacaaccccgtgaagggcaggacctgcaaggagagggactcagagggctgctgggtggcctacacgctggagcagcaggacgggatggaccgctacctcatctatgtggatgagagccgagagtgtgtggcaggccccaacatcgccgccatcgtcgggggcaccgtggcaggcatcgtgctgatcggcattctcctgctggtcatctggaaggctctgatccacctgagcgacctccgggagtacaggcgctttgagaaggagaagctcaagtcccagtggaacaatgataatccccttttcaagagcgccaccacgacggtcatgaaccccaagtttgctgagagttaggagcacttggtgaagacaaggccgtcaggacccaccatgtctgccccatcacgcggccgagacatggcttgccacagctcttgaggatgtcaccaattaaccagaaatccagttattttccgccctcaaaatgacagccatggccggccgggtgcttctgggggctcgtcggggggacagctccactctgactggcacagtctttgcatggagacttgaggagggagggcttgaggttggtgaggttaggtgcgtgtttcctgtgcaagtcaggacatcagtctgattaaaggtggtgccaatttatttacatttaaacttgtcagggtataaaatgacatcccattaattatattgttaatcaatcacgtgtatagaaaaaaaataaaacttcaatacaggctgtccatggaaaaaaaaaaaaaaaaaaaaaa

SEQ ID NO: 95 - DNA, 2862 bp, Mus musculus
ggcctcagcggctgtgttctttagacacaaccacacagccagctggcaagaagtaggcaaagacatctccagtcagattctcggagtggaggcttcctgggactgagagggggacatgctgggcccacactcactgctgcttgccctagctggactgttcttcctgggatctgctgtgtcccaggaatgcaccaagtacaaagtcagcagttgccgggactgtatccagtcggggcctggctgttcctggtgccagaagctgaacttcactggaccaggagaacctgactccttgcgctgtgacacacgggcacagctgctgctgaagggttgtccagccgatgatatcatggaccccaggagcatcgctaatcctgagttcgaccaacgggggcaacggaaacagctatctccacaaaaagtgacactttacttgcgaccaggacaggctgccgcattcaatgtgactttccggcgggccaagggataccccattgatctgtactacctcatggatctctcctactccatgcttgatgacctcaacaacgtcaagaagctgggcggggacttgctgcaggccctcaacgagatcaccgagtctggccgcatcggctttgggtcgtttgtggacaagacggtgctgccttttgttaacacccatcctgagaagctgaggaacccatgtcccaacaaggagaaggcctgccagcccccatttgcctttcggcacgtgctcaagttaaccgacaactccaaccagtttcagacagaggtcggcaagcaactgatttccggaaacctggacgcccctgagggtgggctggatgccataatgcaagttgctgcatgtccggaggaaattggctggcgcaatgtcacgaggctgctggtgtttgccacagacgatggcttccactttgctggtgatggcaaactgggtgccatcctgacccccaatgatggccgctgccacctggaggataacatgtacaagaggagcaatgagttcgactacccatccgtgggtcagctggcacacaaactttccgagagcaacatccagcccatctttgcggtgacaaagaagatggtgaaaacgtatgagaaactcacggagatcatccccaagtcagcagtgggggaactgtctgacgactccagcaacgtggtgcagctcatcaagaatgcctactataaactctcctctagagtcttcctggaccacagcaccctcccggacaccctgaaagtcacctatgactccttctgcagtaatggagcatcgagtataggcaaatcccgtggggactgtgatggcgtacagatcaacaacccggtcaccttccaggtaaaggtcatggcttccgagtgtatccaggagcagtcctttgtcatccgggcactgggtttcacggatacagtgaccgtgcaggtccgtccccagtgtgagtgtcagtgccgggaccagagtcgggagcagagtctctgtggaggcaagggagtcatggagtgtggtatctgcaggtgtgagtctggctacattgggaaaaactgtgagtgccagactcagggtcggagcagccaggagctggagagaaactgtcggaaggacaatagttccatcgtgtgctcagggcttggggactgcatctgtgggcagtgtgtatgccataccagtgacgtccccaacaaagagatctttgggcaatactgcgagtgtgacaatgtcaactgtgagagatataacagccaagtctgcggtggctcagatcggggttcctgcaactgtggcaaatgtagttgcaagcccggttacgagggctcggcctgccagtgtcagaggtccaccacgggctgtctgaatgcacggctggtagagtgcagtggccgtggccactgccaatgcaacaggtgcatatgtgacgaaggctaccagccaccgatgtgtgaggattgtcccagctgtggctcgcactgcagggacaaccacacctcttgtgccgagtgcctgaagtttgataagggcccttttgagaagaactgtagtgttcagtgtgctggtatgacgctgcagactatccctttgaagaaaaagccctgcaaggagagggactcggaaggctgttggataacttacactttgcagcagaaggacggaaggaacatttacaacatccatgtggaggacagtctagagtgtgtgaagggccccaatgtggctgccatcgtagggggcaccgtggtaggtgtcgtactgattggtgtcctcctcctggtcatctggaaggccctgacccacctgactgacctcagggagtacaggcgctttgagaaggagaaactcaagtcccaatggaacaatgacaaccccctcttcaagagtgctacgacaacggtcatgaaccccaagtttgctgaaagctagagcatgagttatcataatcaagcagatgtgaccccctcagaccacgcctcctcccctctgcaaacacaacgtggcttacagctcaccccagtgctgccaaggatccaaaagcctgctcggtttctttccgccattatatcaagtctgccagggtttccagggacttgtcttccgacctgcacaatcttgccgcagagccctaagaattgtcccgagtcccaagaggttccacccacattttcttgcataaaggaagacagcagtctcagtaaaggtggccccaacttatttatatttaaacttgtcagagtataaaactcctattatattgttaacatcccatctgttgtattatatgtgagtataaaaactatatccaacgtattatttcataatcatgtatgaaaaataataaagcttccatccatgctgtc

SEQ ID NO: 96 - DNA, 2929 bp, Sus scrofa
ggccttcgccagcagggcctctgcgggcgggtgtcttcccagacgcgcacgagacccagcctgccaaagggtggaactgaagccccccagcccagccgggaccccctgcggaggtctccaggacatcaagcaggacatgctgtgccggtgctccccgctgctcctcctggtgggcctgctcaccctccggtccgccctctcccaggagtgtgccaagtacaaggtcagcacctgccgggactgcattgagtcgggacccggctgtgcctggtgccagaagctgaacttctctgggcaaggggagcccgactccgtccgctgtgacacgcgggagcagctgcttgcaaagggctgtgtcgcggatgacatcgtggatcccaggagcctggccgagacccaggaggaccaggcagggggccagaagcagctgtccccacagaaagtgacactctacctgagaccaggtcaggcggccacgttcaacgtgaccttccggcgcgccaagggctaccccatcgacctgtactacctgatggacctgtcctactccatgctcgacgacctcatcaacgtgaagaagctggggggcgacctgctcagggctctcaacgagatcaccgagtctggccgcatcggctttgggtctttcgtggacaagacggtgcttcccttcgtcaacacgcaccccgagaagctgcggaacccctgccccaacaaagagaaggagtgccaggccccgttcgccttccgacacgtgctcaagctcacggacaactccaaccagttccagacggaggtcgggaagcagctgatctcggggaacctggacgcccccgagggcgggctggatgccatgatgcaggtggccgcgtgcccggaggagatcggctggcgcaacgtcaccaggctgctggtgttcgccacggacgatggcttccactttgcgggcgacgggaagctgggcgccatcctgacccccaatgacggccgctgccacctggaagacaacttatacaaaagcagcaatgaattcgactacccatcagtgggacagctggcacacaaactggccgaaagcaacatccagcccatctttgccgtgaccaagaaaatggtgaaaacgtatgagaagctcacagacatcatccccaagtccgccgtcggggagctgtcggaggattccagcaacgtcctggagctcattaagaacgcctacaataaactgtcctccagagtgtttttggatcacaacgccctccctgacaccctgaaggtcacgtacgactccttctgcagcaacggggtgtcgcaggtgaaccagcccagaggggactgcgacggcgtccagatcaacgtcccgatcaccttccaggtgaaggtcaccgcctccgagtgcatccaggagcagtcgttcgtcatccgggcgctgggcttcaccgacacggtgaccgtgcgggtgctcccccagtgtgagtgccgctgcggggacagcagcaaggagcgcacgctctgcggcaacaagggctccatggagtgcggggtctgcaggtgcgatgccggctacatcgggaagcactgcgagtgccagacgcagggccggagcagccaggagctggaaggaagctgccgcaaggacaacagctccatcatctgctcggggctgggcgactgcatctgcgggcagtgcgtgtgccacacgagcgacgtgcccaacaagaagatttacggccagttctgcgagtgtgacaacatgaactgcgagcgcttcgatggccaagtctgcgggggcgagaagcggggcctctgcttctgcagcacctgcaggtgccaagaaggtttcgagggctcggcgtgccagtgcctcaagtccacgcagggctgcctcaacctgcagggcgtcgagtgcagcggccgcggccggtgccgctgcaacgtgtgccagtgtgactttggctaccagccgcccctgtgcaccgactgccccagctgccaggtgccctgcgcccgctacgccaaatgcgccgagtgcctgaagttcgacaccggccccttcgccaaaaactgcagcgcggagtgcgggaccactaagctgctgcccagccggatgtcgggccgcaagtgcaatgagcgggactccgagggctgctggatgacctacttcctggtgcagcgcgacggccgggacaactacgacctgcacgtggaggagacgcgcgagtgtgtgaaaggccccaacatcgccgccatcgtggggggcaccgtggggggagtcgtgctcgtgggcatcttcctgctggtcatctggaaggtcctgacccacctgagtgacctcagggagtacaagcgcttcgagaaggagaagctcaagtcccagtggaacaacgataacccccttttcaagagcgccaccacgacagttatgaaccccaagtttgctgagcgctaggggtgcttggtgaagacaaggtcttctgctccgtccagacggggccgcctcccctccggcaggctgaccgtgaccctgtctggcggacccagctgacgtccccccacccccgaccccctcaacatcagctgaaaacctgctcttttccctgcgccccaaatgacggatctgttcagatgcttcaatggactcatcggaagggacaatctcccgcttctgagaggtgtgacttctggtagccacttgaaaaaggactcgcttgtgtctcgtaaggtcagacacctgtcttcctttgtaaaaattagcacagcagtcagatggatggagattccgatttatttatatgtgaacttatagggaatagaattgcattcctgtgattatgctgtttccaatcatgtgcacggaaaaaaatcgaataaatctgcagaggctcgctgggggctgggctctgcgccccatctcagctgtgcccacactccc

SEQ ID NO: 97 - DNA, 1802 bp, Homo sapiens
gggcagctccaccctgggagggactgtggcccaggtactgcccgggtgctactttatgggcagcagctcagttgagttagagtctggaagacctcagaagacctcctgtcctatgaggccctccccatggctttagagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagcggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagtactgcccaggcctgcctgttcccattcttgcatggcaaggactgcagggactgccagtccccctgccccagggctcccggctatgggggcactgaggaccagccattgaggggtggaccctcagaaggcgtcacaagaacctggtcacaggactctgcctcctcttcaactgaccagcctccatgctgcctccagaatggtctttctaatgtgtgaatcagagcacagcagcccctgcacaaagcccttccatgtcgcctctgcattcaggatcaaaccccgaccacctgcccaacctgctctcctcttgccactgcctcttcctccctcattccaccttcccatgccctggatccatcaggccacttgatgacccccaaccaagtggctcccacaccctgttttacaaaaaagaaaagaccagtccatgagggaggtttttaagggtttgtggaaaatgaaaattaggatttcatgatttttttttttcagtccccgtgaaggagagcccttcatttggagattatgttctttcggggagaggctgaggacttaaaatattcctgcatttgtgaaatgatggtgaaagtaagtggtagcttttcccttctttttcttctttttttgtgatgtcccaacttgtaaaaattaaaagttatggtactatgttagccccataattttttttttccttttaaaacacttccataatctggactcctctgtccaggcactgctgcccagcctccaagctccatctccactccagattttttacagctgcctgcagtactttacctcctatcagaagtttctcagctcccaaggctctgagcaaatgtggctcctgggggttctttcttcctctgctgaaggaataaattgctccttgacattgtagagcttctggcacttggagacttgtatgaaagatggctgtgcctctgcctgtctcccccaccgggctgggagctctgcagagcaggaaacatgactcgtatatgtctcaggtccctgcagggccaagcacctagcctcgctcttggcaggtactcagcgaatgaatgctgtatatgttgggtgcaaagttccctacttcctgtgacttcagctctgttttacaataaaatcttgaaaatgcctaaaaaaaaaaaaaaaaaaaaaaaaaaaaaaaaaaaaaaaaaaaaaaaaaaaa

SEQ ID NO: 98 - DNA, 1865 bp, Homo sapiens
gggcagctccaccctgggagggactgtggcccaggtactgcccgggtgctactttatgggcagcagctcagttgagttagagtctggaagacctcagaagacctcctgtcctatgaggccctccccatggctttagctgacttgtatgaagaaggaggtggaggaggaggagaaggtgaagacaatgctgactcaaaggagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagcggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagtactgcccaggcctgcctgttcccattcttgcatggcaaggactgcagggactgccagtccccctgccccagggctcccggctatgggggcactgaggaccagccattgaggggtggaccctcagaaggcgtcacaagaacctggtcacaggactctgcctcctcttcaactgaccagcctccatgctgcctccagaatggtctttctaatgtgtgaatcagagcacagcagcccctgcacaaagcccttccatgtcgcctctgcattcaggatcaaaccccgaccacctgcccaacctgctctcctcttgccactgcctcttcctccctcattccaccttcccatgccctggatccatcaggccacttgatgacccccaaccaagtggctcccacaccctgttttacaaaaaagaaaagaccagtccatgagggaggtttttaagggtttgtggaaaatgaaaattaggatttcatgatttttttttttcagtccccgtgaaggagagcccttcatttggagattatgttctttcggggagaggctgaggacttaaaatattcctgcatttgtgaaatgatggtgaaagtaagtggtagcttttcccttctttttcttctttttttgtgatgtcccaacttgtaaaaattaaaagttatggtactatgttagccccataattttttttttccttttaaaacacttccataatctggactcctctgtccaggcactgctgcccagcctccaagctccatctccactccagattttttacagctgcctgcagtactttacctcctatcagaagtttctcagctcccaaggctctgagcaaatgtggctcctgggggttctttcttcctctgctgaaggaataaattgctccttgacattgtagagcttctggcacttggagacttgtatgaaagatggctgtgcctctgcctgtctcccccaccgggctgggagctctgcagagcaggaaacatgactcgtatatgtctcaggtccctgcagggccaagcacctagcctcgctcttggcaggtactcagcgaatgaatgctgtatatgttgggtgcaaagttccctacttcctgtgacttcagctctgttttacaataaaatcttgaaaatgcctaaaaaaaaaaaaaaaaaaaaaaaaaaaaaaaaaaaaaaaaaaaaaaaaaaaa

SEQ ID NO: 99 - DNA, 1760 bp, Homo sapiens
atttctttataaaccacaactctgggcccgcaatggcagtccactgccttgctgcagtcacagaatggaaatctgcagaggcctccgcagtcacctaatcactctcctcctcttcctgttccattcagagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagcggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagtactgcccaggcctgcctgttcccattcttgcatggcaaggactgcagggactgccagtccccctgccccagggctcccggctatgggggcactgaggaccagccattgaggggtggaccctcagaaggcgtcacaagaacctggtcacaggactctgcctcctcttcaactgaccagcctccatgctgcctccagaatggtctttctaatgtgtgaatcagagcacagcagcccctgcacaaagcccttccatgtcgcctctgcattcaggatcaaaccccgaccacctgcccaacctgctctcctcttgccactgcctcttcctccctcattccaccttcccatgccctggatccatcaggccacttgatgacccccaaccaagtggctcccacaccctgttttacaaaaaagaaaagaccagtccatgagggaggtttttaagggtttgtggaaaatgaaaattaggatttcatgatttttttttttcagtccccgtgaaggagagcccttcatttggagattatgttctttcggggagaggctgaggacttaaaatattcctgcatttgtgaaatgatggtgaaagtaagtggtagcttttcccttctttttcttctttttttgtgatgtcccaacttgtaaaaattaaaagttatggtactatgttagccccataattttttttttccttttaaaacacttccataatctggactcctctgtccaggcactgctgcccagcctccaagctccatctccactccagattttttacagctgcctgcagtactttacctcctatcagaagtttctcagctcccaaggctctgagcaaatgtggctcctgggggttctttcttcctctgctgaaggaataaattgctccttgacattgtagagcttctggcacttggagacttgtatgaaagatggctgtgcctctgcctgtctcccccaccgggctgggagctctgcagagcaggaaacatgactcgtatatgtctcaggtccctgcagggccaagcacctagcctcgctcttggcaggtactcagcgaatgaatgctgtatatgttgggtgcaaagttccctacttcctgtgacttcagctctgttttacaataaaatcttgaaaatgcctaaaaaaaaaaaaaaaaaa

SEQ ID NO: 100 - DNA, 1973 bp, Homo sapiens
gggcagctccaccctgggagggactgtggcccaggtactgcccgggtgctactttatgggcagcagctcagttgagttagagtctggaagacctcagaagacctcctgtcctatgaggccctccccatggctttagggggattataaaactaatcatcaaagccaagaaggcaagagcaagcatgtaccgctgaaaacacaagataactgcataagtaatgactttcagtgcagattcatagctaacccataaactgctggggcaaaaatcatcttggaaggctctgaacctcagaaaggattcacaagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagcggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagtactgcccaggcctgcctgttcccattcttgcatggcaaggactgcagggactgccagtccccctgccccagggctcccggctatgggggcactgaggaccagccattgaggggtggaccctcagaaggcgtcacaagaacctggtcacaggactctgcctcctcttcaactgaccagcctccatgctgcctccagaatggtctttctaatgtgtgaatcagagcacagcagcccctgcacaaagcccttccatgtcgcctctgcattcaggatcaaaccccgaccacctgcccaacctgctctcctcttgccactgcctcttcctccctcattccaccttcccatgccctggatccatcaggccacttgatgacccccaaccaagtggctcccacaccctgttttacaaaaaagaaaagaccagtccatgagggaggtttttaagggtttgtggaaaatgaaaattaggatttcatgatttttttttttcagtccccgtgaaggagagcccttcatttggagattatgttctttcggggagaggctgaggacttaaaatattcctgcatttgtgaaatgatggtgaaagtaagtggtagcttttcccttctttttcttctttttttgtgatgtcccaacttgtaaaaattaaaagttatggtactatgttagccccataattttttttttccttttaaaacacttccataatctggactcctctgtccaggcactgctgcccagcctccaagctccatctccactccagattttttacagctgcctgcagtactttacctcctatcagaagtttctcagctcccaaggctctgagcaaatgtggctcctgggggttctttcttcctctgctgaaggaataaattgctccttgacattgtagagcttctggcacttggagacttgtatgaaagatggctgtgcctctgcctgtctcccccaccgggctgggagctctgcagagcaggaaacatgactcgtatatgtctcaggtccctgcagggccaagcacctagcctcgctcttggcaggtactcagcgaatgaatgctgtatatgttgggtgcaaagttccctacttcctgtgacttcagctctgttttacaataaaatcttgaaaatgcctaaaaaaaaaaaaaaaaaaaaaaaaaaaaaaaaaaaaaaaaaaaaaaaaaaaa

SEQ ID NO: 101 - DNA, 2316 bp, Homo sapiens
gtcgctagtctctattgccttgctgtggcctcgggatggaaatctgctggggaccctacagtcacctaatctctctccttctcatccttctgtttcattcagaggcagcctgccgcccttctgggaaaagaccctgcaagatgcaagccttcagaatctgggatactaaccagaagaccttttacctgagaaacaaccagctcattgctgggtacttacaaggaccaaatatcaaactagaagaaaagatagacatggtgcctattgaccttcatagtgtgttcttgggcatccacgggggcaagctgtgcctgtcttgtgccaagtctggagatgatatcaagctccagctggaggaagttaacatcactgatctgagcaagaacaaagaagaagacaagcgctttaccttcatccgctctgagaaaggccccaccaccagctttgagtcagctgcctgtccaggatggttcctctgcacaacactagaggctgaccgtcctgtgagcctcaccaacacaccggaagagccccttatagtcacgaagttctacttccaggaagaccaatagtactgccgaggcctgtaataatcaccaactgcctgatcactctggccatcattggggcctgaggaacaacttttgcagggtgtatgtacagtagaaggagacagaagagttctgatgatagatctctgcctcagtctgttggctggcctaatccccatgatgattccagaataatcttgcaaattggatcatggcaggtgcttgttcaaagccctttcttgttgcctctgccatctgggtgaagtctagaccacttgcttggcctaggtgtcttctgctctaccacccaccctacccctgccacaaacacacactttttttgtttttgttttttccattgttctgcacttccacagtccagaccaatcaagtcacttgacaatatgccccaagtgactcccttaccctgttttataaacctgtgcctgtctatggagaaggttttaattctccttgttattcattttgggctttttgatgaaaccaccagggcatcacatatactaagcatgtgctctaccatcatgctatgcttccagctcaggggggcacttttaaggatctagaaaacagaaattaaggatctcatagttattttattaggccagccttattccatgtcggcaagaggtttcttgtggaaattatgtcctttctgagaggagctggggattagatgctcctgcatttgtgaaatggttataagcatagaaaaataggtggtaagctttccttctttccttattttgtgtgatgccttaaactgaaaagttaaaaattgatggattgtagcattcccataatctcccccttctttttttttcctttggaaatgtccaatagtctatattcctctgtcccgcccaaacaccatcttcactccaagcctaccacagatgcctgaagaagttcctcactatctgcaaatgtggctctcaggcccttcctgatgtgatgaatgaatctactaatcatttcttgaccattcattttatcacttctaaccttgaaacatgtggaagtagctatgttcctgactgtttcctctgccagacaatgaactctggagatcagggagcttcgtgtgtgtgtgtgtgtgtgtgtgtgtgtgtgtgtgtgtgtgtgcgtgcgcgcgcgcgtgcgcacgcacgtgcatgcacatgctatgtattgggtccctccaaggatgaaccctctctttggcttagaaggcactcagagaatatgtgttattcgtgctcacggaaagtttcttattcatccctgtgactttggctttattttacaataaaacactgaaaatgtccactttgttagttgtgaacatgagcccaggcctaaggtgctgggaaacagaaagggcgggagatttttctttattctatggctagaaaatagttacctcctctctgaaagtcttcttcctcatttctgggtaacagaatatcaaacaccttgcttataagttataaagtagtgttgtccaccatgaacccaccaagtaaaaacaacccaaatacctatcatggatgaataatcatgcaagtatcagatctgcactcaatgccacacaatgacaaagatagcaaatgagccacagacggctccacccaacccaatagatgaacacttggttcaaaatcactaaagctcaaatactcccaggtcaaacaccaggtaacaagttaatactcaacaaagggggaaaacaaatgttccactgaatcctgtgaccctgtgggcg

SEQ ID NO: 102 - DNA, 2455 bp, Homo sapiens
agttccaccctgggaaggtctgtgccatagacactgcctgggtgctcctttatacacagcaagtctctctggagtgagacgttggaaggcagtggaagaccttgtgtcctgtttagctcacccatggcttcagaggcagcctgccgcccttctgggaaaagaccctgcaagatgcaagccttcagaatctgggatactaaccagaagaccttttacctgagaaacaaccagctcattgctgggtacttacaaggaccaaatatcaaactagaagaaaagatagacatggtgcctattgaccttcatagtgtgttcttgggcatccacgggggcaagctgtgcctgtcttgtgccaagtctggagatgatatcaagctccagctggaggaagttaacatcactgatctgagcaagaacaaagaagaagacaagcgctttaccttcatccgctctgagaaaggccccaccaccagctttgagtcagctgcctgtccaggatggttcctctgcacaacactagaggctgaccgtcctgtgagcctcaccaacacaccggaagagccccttatagtcacgaagttctacttccaggaagaccaatagtactgccgaggcctgtaataatcaccaactgcctgatcactctggccatcattggggcctgaggaacaacttttgcagggtgtatgtacagtagaaggagacagaagagttctgatgatagatctctgcctcagtctgttggctggcctaatccccatgatgattccagaataatcttgcaaattggatcatggcaggtgcttgttcaaagccctttcttgttgcctctgccatctgggtgaagtctagaccacttgcttggcctaggtgtcttctgctctaccacccaccctacccctgccacaaacacacactttttttgtttttgttttttccattgttctgcacttccacagtccagaccaatcaagtcacttgacaatatgccccaagtgactcccttaccctgttttataaacctgtgcctgtctatggagaaggttttaattctccttgttattcattttgggctttttgatgaaaccaccagggcatcacatatactaggcatgtgctctaccatcatgctatgcttccagctcaggggggcacttttaaggatctagaaaacagaaattaaggatctcatagttattttattaggccagccttattccatgtcggcaagaggtttcttgtggaaattatgtcctttctgagaggagctggggattagatgctcctgcatttgtgaaatggttataagcatagaaaaataggtggtaagctttccttctttccttattttgtgtgatgccttaagctgaaaagttaaaaattgatggattgtagcattcccataatctcccccttctttttttttcctttggaaatgtccaatagtctatattcctctgtcccgcccaaacaccatcttcactccaagcctaccacagatgcctgaagaagttcctcactatctgcaaatgtggctctcaggcccttcctgatgtgatgaatgaatctactaatcatttcttgaccattcattttatcacttctaaccttgaaacatgtggaagtagctatgttcctgactgtttcctctgccagacaatgaactctggagatcagggagcttcgtgtgtgtgtgtgtgtgtgtgtgtgtgtgtgtgtgtgtgtgtgcgtgcgcgcgcgcgtgcgcacgcacgtgcatgcacatgctatgtattgggtccctccaaggatgaaccctctctttggcttagaaggcactcagagaatatgtgttattcgtgctcacggaaagtttcttactcatccctgtgactttggctttattttacaataaaacactgaaaatgtccactttgttagttgtgaacatgagcccaggcctaaggtgctgggaaacagaaaggggcgggagatttttctttattctatggctagaaaatagttacctcctctctgaaagtcttcttcctcatttctgggtaacagaatatcaaacaccttgcttataagttataaagtagtgttgtccaccatgaacccaccaagtaaaaacaacccaaatacctatcatggatgaataatcatgcaagtatcagatctgcactcaatgccacacaatgacaaagatagcaaatgagccacagacggctccacccaacccaatagatgaacacttggttcaaaatcactaaagctcaaatactcccaggtcaaacaccaggtaacaagttaatactcaacaaagggggaaaacaaatgttccactgaatcctgtgaccctgtgggcgtggttcacctcctgtgttgtttgccatgtgtgctcaggatgagctgattaaagctcttctcaggggttcagttttcccatctctgcttgtaaataaaaagcttatgc

SEQ ID NO: 103 - DNA, 563 bp, Mus musculus
gagtgtcctgttgttgcatggtcacaacgatggaagttagcaggtatctgtgcagttacctaatctctttcctccttttcctgttccactcagagactgcctgccaccccttgggaaagagaccttgcaggatgcaagccttcagaatctgggatgtcaaccagaagactttctacctgaggaataaccaattagttgctggatacttgcaaggaccaaatactaaactggaagagaagatagatgtggtgcctgttgagcctcattttgtgttcctggggatccatggagggaagctgtgcctgtcctgtgtcaagtctggtgatgagatgaagctccagttggacgcagttaacatcacagacctgagaaagaacagcgagcaggacaagcgcttcaccttcatccgctccgacagtggccccaccaccagctttgagtcagccgcctgtcctggctggttcctctgcactgcactagaggcagaccagcctgttggcctcaccaacacgcccaaagcagccgtcaaggtcaccaagttctacttccagcaggaccagtaa

SEQ ID NO: 104 - DNA, 3916 bp, Homo sapiens
gagcaggaagtgtttgaggaagtcgcgccgcgctgcccgcgttaagattcccgcattttaatgttttcaggggggtgtcatagccccgggtttggccgccccagccccgccttccccgccccggggagcccgccccctgccccgcgtccctgccgacagagttagcacgacatcagtatgagctggtcaccttccctgacaacgcagacatgtggggcctgggaaatgaaagagcgccttgggacagggggatttggaaatgtcatccgatggcacaatcaggaaacaggtgagcagattgccatcaagcagtgccggcaggagctcagcccccggaaccgagagcggtggtgcctggagatccagatcatgagaaggctgacccaccccaatgtggtggctgcccgagatgtccctgaggggatgcagaacttggcgcccaatgacctgcccctgctggccatggagtactgccaaggaggagatctccggaagtacctgaaccagtttgagaactgctgtggtctgcgggaaggtgccatcctcaccttgctgagtgacattgcctctgcgcttagataccttcatgaaaacagaatcatccatcgggatctaaagccagaaaacatcgtcctgcagcaaggagaacagaggttaatacacaaaattattgacctaggatatgccaaggagctggatcagggcagtctttgcacatcattcgtggggaccctgcagtacctggccccagagctactggagcagcagaagtacacagtgaccgtcgactactggagcttcggcaccctggcctttgagtgcatcacgggcttccggcccttcctccccaactggcagcccgtgcagtggcattcaaaagtgcggcagaagagtgaggtggacattgttgttagcgaagacttgaatggaacggtgaagttttcaagctctttaccctaccccaataatcttaacagtgtcctggctgagcgactggagaagtggctgcaactgatgctgatgtggcacccccgacagaggggcacggatcccacgtatgggcccaatggctgcttcaaggccctggatgacatcttaaacttaaagctggttcatatcttgaacatggtcacgggcaccatccacacctaccctgtgacagaggatgagagtctgcagagcttgaaggccagaatccaacaggacacgggcatcccagaggaggaccaggagctgctgcaggaagcgggcctggcgttgatccccgataagcctgccactcagtgtatttcagacggcaagttaaatgagggccacacattggacatggatcttgtttttctctttgacaacagtaaaatcacctatgagactcagatctccccacggccccaacctgaaagtgtcagctgtatccttcaagagcccaagaggaatctcgccttcttccagctgaggaaggtgtggggccaggtctggcacagcatccagaccctgaaggaagattgcaaccggctgcagcagggacagcgagccgccatgatgaatctcctccgaaacaacagctgcctctccaaaatgaagaattccatggcttccatgtctcagcagctcaaggccaagttggatttcttcaaaaccagcatccagattgacctggagaagtacagcgagcaaaccgagtttgggatcacatcagataaactgctgctggcctggagggaaatggagcaggctgtggagctctgtgggcgggagaacgaagtgaaactcctggtagaacggatgatggctctgcagaccgacattgtggacttacagaggagccccatgggccggaagcaggggggaacgctggacgacctagaggagcaagcaagggagctgtacaggagactaagggaaaaacctcgagaccagcgaactgagggtgacagtcaggaaatggtacggctgctgcttcaggcaattcagagcttcgagaagaaagtgcgagtgatctatacgcagctcagtaaaactgtggtttgcaagcagaaggcgctggaactgttgcccaaggtggaagaggtggtgagcttaatgaatgaggatgagaagactgttgtccggctgcaggagaagcggcagaaggagctctggaatctcctgaagattgcttgtagcaaggtccgtggtcctgtcagtggaagcccggatagcatgaatgcctctcgacttagccagcctgggcagctgatgtctcagccctccacggcctccaacagcttacctgagccagccaagaagagtgaagaactggtggctgaagcacataacctctgcaccctgctagaaaatgccatacaggacactgtgagggaacaagaccagagtttcacggccctagactggagctggttacagacggaagaagaagagcacagctgcctggagcaggcctcatgatgtggggggactcgaccccctgacatggggcagcccatagcaggccttgtgcagtggggggactcgaccccctgacatggggctgcctggagcaggccgcgtgacgtggggctgcctggccgcggctctcacatggtggttcctgctgcactgatggcccaggggtctctggtatccagatggagctctcgcttcctcagcagctgtgactttcacccaggacccaggacgcagccctccgtgggcactgccggcgccttgtctgcacactggaggtcctccattacagaggcccagcgcacatcgctggccccacaaacgttcaggggtacagccatggcagctccttcctctgccgtgagaaaagtgcttggagtacggtttgccacacacgtgactggacagtgtccaattcaaatctttcagggcagagtccgagcagcgcttggtgacagcctgtcctctcctgctctccaaaggccctgctccctgtcctctctcactttacagcttgtgtttcttctggattcagcttctcctaaacagacagtttaattatagttgcggcctggccccatcctcacttcctctttttatttcactgctgctaaaattgtgtttttacctactactttggtggttgtcctcttttcggcaaagttggagcgagtgccaagctctccatctgtggtcctttctgccaagagcgactcatagtaaccaggatgggagagcagctgccttattctgaatcccaaaaattacttgggggtgattgtcacagaggagggacagaaagggtatctgctgaccaccagcctgcctacccatgcccatgtctccattcctgctcaagcgtgtgtgctgggccggggagtccctgtctctcacagcatctagcagtattattaaatggattcattttaaaaatagctcctatattttgtaacatgtctcaaacactcatactgggttccacaatccactgttagaatacctatggttagggcttctgaactaaaataatggaaaattttaacaatttgtatagtgcctggatcattactagtgccataaccctgcttcttcaacatttcacagaacttctcttttatataaaggcaagagcacaaaatgagttcagatgatcacaaacaggtgagttttgttggagaagaaagttggagtaggagactttcacaagtggtttccatggagatagaatgaagcattctgtggtcaagtaagtttagggagctattcatgtttcacttgctttgtggagattcacactatgcactgggaaagtatctgaaaagtcttataataaagaaacaggcttaactttgtgtaagaacactgtttatcaatgtcatttggctatagaaacattttctcctgctgattgtgtgtgtgaaacatgtattaacattccaatgaactagcatttaataaagcacaattttgg

SEQ ID NO: 105 - DNA, 3973 bp, Mus musculus
gcggccgcgtcgacgtttgaggaagtggcgctgggctggctgcgtggggaccccgagttttcatgtcctcagcgggtgtcgctggcggccccgcggcccccggggcagagttagcagggcatcgatatgagctggtcaccgtccctcccaacccagacatgtggagcctgggaaatgaaagaacgcctggggaccgggggatttggaaacgtcatccggtggcacaatcaggcgacaggtgaacagatcgccatcaagcaatgccgacaggagctcagcccaaagaacagagaccgctggtgcctcgaaatccagatcatgagaaggctgaaccatcccaatgtggtggctgcccgggatgtcccagaggggatgcagaacctggcacccaatgatttgccactgctggccatggagtactgccaaggaggagatctccgaagatacttgaaccagttcgagaactgctgtggcctgcgggaaggagctgtccttaccctgctgagtgacatcgcatcggctcttagataccttcacgaaaacagaatcatccatcgagacctgaagccagaaaacatcgttctgcagcaaggagagaaaagattaatacacaaaattattgatctaggatatgccaaggagctggatcagggcagtctgtgcacgtcatttgtggggactctgcaatacctggcgccagagcttctggagcagcagaagtacaccgtgaccgttgactactggagcttcggcaccctggccttcgagtgcatcactggcttccggcccttcctccctaactggcagcctgtgcagtggcactccaaagtccggcagaagagcgaagtggacatcgttgttagtgaagacttgaatggagcagtgaagttttcaagttcgctacccttccccaataatcttaacagtgtcttggctgaacggctggagaagtggctgcagctgatgcttatgtggcaccctcggcaaaggggcacggatccccagtatggccccaacggctgcttcagagccctggatgacatcttgaacttgaagctggttcatgtcttgaacatggtcacaggcaccgttcacacataccccgtgacggaggatgagagtctgcagagcttaaaaaccagaatccaggaagacacggggatcctggagacagaccaggagctgctgcaagaggcagggctggtgctgctccctgacaagcctgctactcagtgcatctcagacagcaagacaaacgagggcctcacgttggacatggatcttgtttttctctttgacaacagtaaaatcaactatgagactcagatcaccccccgaccccaaccggaaagtgtcagctgtatccttcaggagcccaagcggaacctctccttcttccagctgaggaaagtgtggggccaagtctggcacagcatccagacgctgaaggaagactgtaaccggctgcagcagggacagcgagcagccatgatgagtctcctccggaataacagctgcctctctaagatgaagaacgccatggcctccacggcccagcagctcaaggccaagctggacttcttcaaaaccagcatccagatcgacctggagaagtataaagagcagaccgagtttgggatcacctcagataaattgctgctggcttggcgggagatggagcaggctgtggagcagtgtgggcgggagaatgacgtgaagcatctagtagagcggatgatggcactgcagactgacattgtggacctgcagaggagcccgatgggtcggaagcaggggggcaccctggatgacctagaggaacaagcgagggagctctaccgaagactcagggagaagccaagagaccaaaggacagaaggtgacagccaggagatggtacggctgctgcttcaggcaatccaaagctttgagaagaaagttcgggtgatttatacacagctcagtaagaccgtggtttgtaagcagaaggcactggagttgctgcccaaggtagaagaggtagtgagccttatgaacgaggacgagaggaccgtggtccggcttcaggagaagcggcagaaggaactctggaacctcctgaagatcgcctgtagcaaagtccgaggtcccgtgagtggaagcccagacagcatgaatgtgtctcgactcagtcaccctggtcagctaatgtcccagccttccagtgcctgtgacagcttacctgaatcagacaagaaaagtgaagaactggtggccgaagcccacgccctctgctcccggctagaaagtgcgctgcaggacactgtgaaggagcaagacagaagcttcacggtaaccgcctgagggagcccggcttgtccgtcccttcctctctgcagcaaaagtgcaaacgcagtttggcatcagccttggctcagcagagactcatggttggccctgcggttttcaggtttttgtatttatttcatgcttaggactgccaggtgcattcattatcttaatcccttatgacacgtaatcctaaggaggtgacagcagtaccaaaagccccttctttctctgtgggaccattgcagaacatctgtatgtgctgggcactgtgctgggctctgtgacaaaagagaaacctgtgtgtgtctcaaggactcagtttagccacaagcagtatcagtgtcagagaaactgacctgaaggaaatctagccacgatccaggcactttcagacagagcttaaacagttgttggtgtctaaatggccttttcctgctaatgcaagcatccactaactactcttgcatctaactttggggagtcagaagaaaccccattcagttgccgtcctcgctaacctgctcactggtgagcctcctaactcctgctgcttctcacttgtctcctggtgctgtttcttccatctcactacttgcttgcactttagccagagaaaagggcagccttctggttggcatggaaacggataactgagatgattcccacctatctgcagtgagtatgtaaatgagagctctatgctacaggccgtactaaaatgaccatccacattccacaggcacagagttccaggaacttcagctagtccaaaggaaatctgctgcacatttgaatctgtaactcacttggctcaagggtgccctgtagtagcaggactgacgctgataaggcttaaacgtacttctagattgtgttgcttgcctcctgaagaggaacgatggccccgccacttgagtatttaatgtgcctgttgactttcagactctagactggagctggttacagatggaggatgaagaaaggtgtagcctggagcaggcctgtgactgaggtgcccatgagctggcccgtggctctgcatgtgacgatgttctggtatctccaggtggacatctcactctgccagcagctgtgatacttgcccgcagcagcctaggctggccctgtccacacactggattctcgcccacagaagcacaacaatgcaggtacaagcacttgcagcagcgacgcttcatggctgctgtacaaagaaagcgacacagagaaatgacaagtgtctatcttgagtgtttccagacagcagacagcctgtctgctttggactcctgggggaggaagctctgccctgcccacccctcctctccttacagctggacgtcatccggatccagctctcttagacagttaatcctaagtttgactatcctatatctctcttcctcttcacttctcccctggtaaatgtttcaaccttctgtgctggtcgctgtggcaaacgacctttgactatgctgtagtgatggcgatgccaaactccctgctgtttctggtcctttaagccacaaagtgacttacaaagccaggcagaacagaaatgcagctacatatcttgagtcccaaagattacctagaatcaattgtcacagagaaaaaaggaagatgaaggtgttctcgtgcc

SEQ ID NO: 106 - DNA, 1832 bp, Homo sapiens
gagcggccaggccagcctcggagccagcagggagctgggagctgggggaaacgacgccaggaaagctatcgcgccagagagggcgacgggggctcgggaagcctgacagggcttttgcgcacagctgccggctggctgctacccgcccgcgccagcccccgagaacgcgcgaccaggcacccagtccggtcaccgcagcggagagctcgccgctcgctgcagcgaggcccggagcggccccgcagggaccctccccagaccgcctgggccgcccggatgtgcactaaaatggaacagcccttctaccacgacgactcatacacagctacgggatacggccgggcccctggtggcctctctctacacgactacaaactcctgaaaccgagcctggcggtcaacctggccgacccctaccggagtctcaaagcgcctggggctcgcggacccggcccagagggcggcggtggcggcagctacttttctggtcagggctcggacaccggcgcgtctctcaagctcgcctcttcggagctggaacgcctgattgtccccaacagcaacggcgtgatcacgacgacgcctacacccccgggacagtacttttacccccgcgggggtggcagcggtggaggtgcagggggcgcagggggcggcgtcaccgaggagcaggagggcttcgccgacggctttgtcaaagccctggacgatctgcacaagatgaaccacgtgacaccccccaacgtgtccctgggcgctaccggggggcccccggctgggcccgggggcgtctacgccggcccggagccacctcccgtttacaccaacctcagcagctactccccagcctctgcgtcctcgggaggcgccggggctgccgtcgggaccgggagctcgtacccgacgaccaccatcagctacctcccacacgcgccgcccttcgccggtggccacccggcgcagctgggcttgggccgcggcgcctccaccttcaaggaggaaccgcagaccgtgccggaggcgcgcagccgggacgccacgccgccggtgtcccccatcaacatggaagaccaagagcgcatcaaagtggagcgcaagcggctgcggaaccggctggcggccaccaagtgccggaagcggaagctggagcgcatcgcgcgcctggaggacaaggtgaagacgctcaaggccgagaacgcggggctgtcgagtaccgccggcctcctccgggagcaggtggcccagctcaaacagaaggtcatgacccacgtcagcaacggctgtcagctgctgcttggggtcaagggacacgccttctgaacgtcccctgcccctttacggacaccccctcgcttggacggctgggcacacgcctcccactggggtccagggagcaggcggtgggcacccaccctgggacctaggggcgccgcaaaccacactggactccggccctcctaccctgcgcccagtccttccacctcgacgtttacaagcccccccttccacttttttttgtatgttttttttctgctggaaacagactcgattcatattgaatataatatatttgtgtatttaacagggaggggaagagggggcgatcgcggcggagctggccccgccgcctggtactcaagcccgcggggacattgggaaggggacccccgccccctgccctcccctctctgcaccgtactgtggaaaagaaacacgcacttagtctctaaagagtttattttaagacgtgtttgtgtttgtgtgtgtttgttctttttattgaatctatttaagtaaaaaaaaaattggttctttaaaaaaaaaaaaaaaaa

SEQ ID NO: 107 - DNA, 1817 bp, Mus musculus
tataaaagcttggggctggggccgagcactggggactttgagggtggccaggccagcgtaggatcctgctgggagcggggaactgagggaagcgacgccgagaaagcaggcgtaccacggagggagagaaaagctccggaagcccagcagcgcctttacgcacagctgccaactggccgctgccgaccgtctccagctcccgaggacgcgcgaccggacaccgggtcctgccacagccgaggacagctcgccgctcgccgcagcgaggcccggggcggcccttcagggggacctttcccagatcgcccaggccgcccggatgtgcacgaaaatggaacagcctttctatcacgacgactcttacgcagcggcgggatacggtcggagccctggcagcctgtctctacacgactacaaactcctgaaacccaccttggcgctcaacctggcggatccctatcggggtctcaagggtcctggggcgcggggtccaggcccggagggcagtggggcaggcagctacttttcgggtcagggatcagacacaggcgcatctctgaagctagcctccacggaactggagcgcttgatcgtccccaacagcaacggcgtgatcacgacgacgcccacgcctccgggacagtacttttacccccgtgggggtggcagcggtggaggtacagggggcggcgtcaccgaggagcaggagggctttgcggacggttttgtcaaagccctggacgacctgcacaagatgaaccacgtgacgccccccaacgtgtccctgggcgccagcgggggtccccaggccggcccagggggcgtctatgctggtccggagccgcctcccgtctacaccaacctcagcagttactctccagcctctgcaccctctggaggctccgggaccgccgtcgggactgggagctcatacccgacggccaccatcagctacctcccacatgcaccaccctttgcgggcggccacccggcacagctgggtttgagtcgtggcgcttccgcctttaaagaggaaccgcagaccgtaccggaggcacgcagccgcgacgccacgccgcctgtgtcccccatcaacatggaagaccaggagcgcatcaaagtggagcgaaagcggctgcggaacaggctggcggccaccaagtgccggaagcggaagctggagcgcatcgcgcgcctggaggacaaggtgaagacactcaaggctgagaacgcggggctgtcgagtgctgccggtctcctaagggagcaagtggcgcagctcaagcagaaggtcatgacccatgtcagcaacggctgccagttgctgctaggggtcaagggacacgccttctgagagcctcccttgccccatacggacacccccagccttgaaggctgggcgcctgccccccactggggtgaggggggcaggcgatgggcacccgccaaaaggcctggggcgcagctcacacactggactccggcccgcccgcctgcgcccagtccttccacctcgaggtttacatggcccccttccagcgtattttgtatgttttttttttctgcaaagagactgaattcatattgaatataatatatttgtgtatttaacagggaggggagaagggggctgtcgcggcggagctggccccgccgcttggtactcagcctgcggggatactagggagggacctccgccccctgccctccccctctgcatagtactgtggagaagaaacacgcacttcgtgtctaaagtctattttaagatgtgtttgtgtgtgtgtgtttgactttttattgaatctatttaagta

SEQ ID NO: 108 - DNA, 3083 bp, Homo sapiens
cagaggcgcgcctggcggatctgagtgtgttgcccgggcagcggcgcgcgggaccaacgcaaggagcagctgacagacgaagaaaagtgctggacaggaagggagaattctgacgccaacatgcagcgaagtatcatgtcatttttccaccccaagaaagagggtaaagcaaagaagcctgagaaggaggcatccaatagcagcagagagacggagccccctccaaaggcggcactgaaggagtggaatggagtggtgtccgagagtgactctccggtgaagaggccagggaggaaggcggcccgggtcctgggcagcgaaggggaagaggaggatgaagcccttagccctgctaaaggccagaagcctgccctggactgctcacaggtctccccgccccgtcctgccacatctcctgagaacaatgcttccctctctgacacctctcccatggacagttccccatcagggattccgaagcgtcgcacagctcggaagcagctcccgaaacggaccattcaggaagtcctggaagagcagagtgaggacgaggacagagaagccaagaggaagaaggaggaggaagaagaggagaccccgaaagaaagcctcacagaggctgaagtggcaacagagaaggaaggagaagacggggaccagcccaccacgcctcccaagcccctaaagacctccaaagcagagaccccgacggaaagcgtttcagagcctgaggtggccacgaagcaggaactgcaggaggaggaagagcagaccaagcctccccgcagagctcccaagacgctcagcagcttcttcaccccccggaagccagcagtcaaaaaagaagtgaaggaagaggagccaggggctccaggaaaggagggagctgctgagggacccctggatccatctggttacaatcctgccaagaacaactatcatcccgtggaagatgcctgctggaaaccgggccagaaggttccttacctggctgtggcccggacgtttgagaagatcgaggaggtgtctgctcggctccggatggtggagacgctgagcaacttgctgcgctccgtggtggccctgtcgcctccagacctcctccctgtcctctacctcagcctcaaccaccttgggccaccccagcagggcctggagcttggcgtgggtgatggtgtccttctcaaggcagtggcccaggccacaggtcggcagctggagtccgtccgggctgaggcagccgagaaaggcgacgtggggctggtggccgagaacagccgcagcacccagaggctcatgctgccaccacctccgctcactgcctccggggtcttcagcaagttccgcgacatcgccaggctcactggcagtgcttccacagccaagaagatagacatcatcaaaggcctctttgtggcctgccgccactcagaagcccggttcatcgctaggtccctgagcggacggctgcgccttgggctggcagagcagtcggtgctggctgccctctcccaggcagtgagcctcacgcccccgggccaagaattcccaccagccatggtggatgctgggaagggcaagacagcagaggccagaaagacgtggctggaggagcaaggcatgatcctgaagcagacgttctgcgaggttcccgacctggaccgaattatccccgtgctgctggagcacggcctggaacgtctcccggagcactgcaagctgagcccagggattcccctgaaaccaatgttggcccatcccacccggggcatcagcgaggtcctgaaacgctttgaggaggcagctttcacctgcgaatacaaatatgacgggcagagggcacagatccacgccctggaaggcggggaggtgaagatcttcagcaggaatcaggaagacaacactgggaagtacccggacatcatcagccgcatccccaagattaaactcccatcggtcacatccttcatcctggacaccgaagccgtggcttgggaccgggaaaagaagcagatccagccattccaagtgctcaccacccgcaaacgcaaggaggtggatgcgtctgagatccaggtgcaggtgtgtttgtacgccttcgacctcatctacctcaatggagagtccctggtacgtgagcccctttcccggcgccggcagctgctccgggagaactttgtggagacagagggcgagtttgtcttcgccacctccctggacaccaaggacatcgagcagatcgccgagttcctggagcagtcagtgaaagactcctgcgaggggctgatggtgaagaccctggatgttgatgccacctacgagatcgccaagagatcgcacaactggctcaagctgaagaaggactaccttgatggcgtgggtgacaccctggacctggtggtgatcggcgcctacctgggccgggggaagcgggccggccggtacgggggcttcctgctggcctcctacgacgaggacagtgaggagctgcaggccatatgcaagcttggaactggcttcagtgatgaggagctggaggagcatcaccagagcctcaaggcgctggtgctgcccagcccacgcccttacgtgcggatagatggcgctgtgattcccgaccactggctggaccccagcgctgtgtgggaggtgaagtgcgctgacctctccctctctcccatctaccctgctgcgcggggcctggtggatagtgacaagggcatctcccttcgcttccctcggtttattcgagtccgtgaagacaagcagccggagcaggccaccaccagtgctcaggtggcctgtttgtaccggaagcaaagtcagattcagaaccaacaaggcgaggactcaggctctgaccctgaagatacctactaagccctcgccctcctagggcctgggtacagggcatgagttggacggaccccagggttattattgcctttgctttttagcaaatctgctgtggcaggctgtggattttgagagtcaggggaggggtgtgtgtgtgagggggtggcttactccggagtctgggattcatcccgtcatttctttcaataaataattattggatagct

SEQ ID NO: 109 - DNA, 3186 bp, Mus musculus
gtggcgccaagagaagaggggcggcctggggccggaaaggaaggcttgagtgtggtgtttcaccctggacgcctgctatcaatcggtgtgggatgcttggtgttatgggacgcttgtaatgaaagcaaggtgacgtcattggtcagatagtggtccgatttgaagactagagaagcttggcgcgatgagtgtcaggagccagggaaaaggtacaactgatgagaaaaaaagagcaagagaggaaaggagagacttctgctgccaacatgcagagaagtattatgtcattttttcaacccacaaaagaaggtaaagcgaagaagccagagaaggagacacccagcagcatcagagagaaggaaccccctccaaaggtggcgctgaaggagaggaatcaagtggtgcccgagagtgattctccagtgaagaggacaggaaggaaggtagcccaggttctgagctgtgaaggggaggacgaagatgaagcccctggcacccccaaagtccagaagcctgtgtcagactctgaacagagctctcctcccagccctgacacatgtcctgagaacagtcctgtcttcaactgcagctcccccatggacatctccccatcaggattcccaaagcgtcgaactgcgcggaagcagctccccaaacggacaattcaagacactcttgaggagcagaatgaggacaaaaccaaaacagctaagaaaaggaagaaagaagaagagaccccaaaagaaagcctcgcagaggctgaagacgtaaaacagaaggaagaaaaggagggggaccagctcatagtcccctctgagcccacaaagtcccctgagtcagtaaccctgacaaagacagagaacattccagtgtgtaaggcaggagtgaaactgaagcctcaggaagaggaacagagcaaacctcctgctagaggtgccaagacactgagcagtttcttcacaccccgaaaaccagcagtgaaaactgaagtcaaacaagaagagtcaggcactctaaggaaggaagagaccaagggaaccctggatccagcaaattataatccttccaagaataactatcatcccattgaagatgcctgctggaaacatggccagaaggtcccttttctcgccgtggcccggacatttgagaagattgaggaggtttctgctaggctcaagatggtggagacattgagcaacctgctgcgctccgtggtggccctgtcccctccagacttgctccctgttctctatctcagcctcaaccgcctcgggccacctcagcagggactagagctgggtgttggtgatggtgtcctcctcaaggccgttgcccaggccacaggtcgtcagctggagtccatccgggctgaggtagctgagaagggggatgtggggctggtggccgagaacagccgcagcactcagagactcatgctgcctccacctccacttactatttctggagtcttcaccaaattctgtgacattgcccggctcactggcagtgcttccatggccaagaagatggacattatcaagggcctttttgttgcctgccgccactcagaagccaggtacattgccaggtccctaagcgggcggctgcgccttggcctagctgagcagtcggtactggctgctcttgcccaggctgtgagcctcacaccccctggccaagaatttcccacggcggtggtggatgctgggaagggcaagacagcagaggccagaaagatgtggttggaagaacaaggcatgatcttgaagcagaccttctgtgaggttcctgacctggatcgaatcatccctgtgctgctggaacatggcctggaacgtctcccagagcactgcaagctgagcccaggggtccctcttaaaccaatgctggctcatcccactcggggtgtcagtgaggtcctaaaacgctttgaggaggtcgacttcacctgtgaatataaatacgatgggcagcgagcacagatacatgttctggaaggtggagaagtgaagatcttcagcaggaaccaggaagacaacacaggaaagtatcctgacattatcagccgcatccccaagattaaacacccctcagtcacatctttcattctggacactgaggctgtggcctgggaccgggaaaagaagcagatccaaccattccaagtactcaccacgcgcaagcgcaaggaggttgatgcatcggagatacaggtgcaagtgtgtctgtatgcttttgacctcatctacctcaacggagagtccctggttcgccagcccctgtctcgacgccggcagctgctccgtgagaacttcgtggagacagagggtgagtttgtcttcaccacctctttggacaccaaggacaccgagcagattgctgagttcttggagcagtcagtgaaggactcctgtgagggcctgatggtgaagaccttggatgttgatgccacttatgagattgccaagaggtcacacaactggctcaagctgaagaaggactaccttgacggtgtgggtgacactctggaccttgtggtgattggtgcctacctgggccgggggaagcgggctggccggtatggaggcttcctgttggctgcctacgatgaggagagtgaagagcttcaggctatatgcaagctgggaactggattcagcgatgaggagctggaggaacaccaccagagcctgcaggccctggtattgcctaccccacgcccctatgtgaggattgatggggcagtggccccagaccattggctggacccaagcattgtgtgggaggtgaagtgtgctgatctttccctgtcccccatctaccctgctgcacggggcctggtggacaaagaaaaagggatctctcttcgttttcctcggttcattcgtgtccgtaaagacaagcagccagagcaggccactaccagtaaccaggttgcctctttgtaccggaagcagagtcagattcagaaccaacaaagctcagacttggactccgacgttgaagactattaacctcctgctctcctgggcctggggtggaggttgcgagaacatgggcactgttggattggtggtgttgattggtgtgtgtggagtcacaggagtgagatcctggggctggaatgctttattttcttcaataaatgacttcttagatgcctacaaaaaaaaaaaaaaaaaa

SEQ ID NO: 110 - DNA, 3111 bp, Mus musculus
gtctccgccccctcccgccaaagtgcgcgcgaaatctgtgtggcgcgaatttggggatttcgggtttgcgtctctgagcgagcgagcgagcgagtgagcgagtgtggcggcgtccggcaaagtgatttggagaggtacaactgatgagaaaaaaagagcaagagaggaaaggagagacttctgctgccaacatgcagagaagtattatgtcattttttcaacccacaaaagaaggtaaagcgaagaagccagagaaggagacacccagcagcatcagagagaaggaaccccctccaaaggtggcgctgaaggagaggaatcaagtggtgcccgagagtgattctccagtgaagaggacaggaaggaaggtagcccaggttctgagctgtgaaggggaggacgaagatgaagcccctggcacccccaaagtccagaagcctgtgtcagactctgaacagagctctcctcccagccctgacacatgtcctgagaacagtcctgtcttcaactgcagctcccccatggacatctccccatcaggattcccaaagcgtcgaactgcgcggaagcagctccccaaacggacaattcaagacactcttgaggagcagaatgaggacaaaaccaaaacagctaagaaaaggaagaaagaagaagagaccccaaaagaaagcctcgcagaggctgaagacgtaaaacagaaggaagaaaaggagggggaccagctcatagtcccctctgagcccacaaagtcccctgagtcagtaaccctgacaaagacagagaacattccagtgtgtaaggcaggagtgaaactgaagcctcaggaagaggaacagagcaaacctcctgctagaggtgccaagacactgagcagtttcttcacaccccgaaaaccagcagtgaaaactgaagtcaaacaagaagagtcaggcactctaaggaaggaagagaccaagggaaccctggatccagcaaattataatccttccaagaataactatcatcccattgaagatgcctgctggaaacatggccagaaggtcccttttctcgccgtggcccggacatttgagaagattgaggaggtttctgctaggctcaagatggtggagacattgagcaacctgctgcgctccgtggtggccctgtcccctccagacttgctccctgttctctatctcagcctcaaccgcctcgggccacctcagcagggactagagctgggtgttggtgatggtgtcctcctcaaggccgttgcccaggccacaggtcgtcagctggagtccatccgggctgaggtagctgagaagggggatgtggggctggtggccgagaacagccgcagcactcagagactcatgctgcctccacctccacttactatttctggagtcttcaccaaattctgtgacattgcccggctcactggcagtgcttccatggccaagaagatggacattatcaagggcctttttgttgcctgccgccactcagaagccaggtacattgccaggtccctaagcgggcggctgcgccttggcctagctgagcagtcggtactggctgctcttgcccaggctgtgagcctcacaccccctggccaagaatttcccacggcggtggtggatgctgggaagggcaagacagcagaggccagaaagatgtggttggaagaacaaggcatgatcttgaagcagaccttctgtgaggttcctgacctggatcgaatcatccctgtgctgctggaacatggcctggaacgtctcccagagcactgcaagctgagcccaggggtccctcttaaaccaatgctggctcatcccactcggggtgtcagtgaggtcctaaaacgctttgaggaggtcgacttcacctgtgaatataaatacgatgggcagcgagcacagatacatgttctggaaggtggagaagtgaagatcttcagcaggaaccaggaagacaacacaggaaagtatcctgacattatcagccgcatccccaagattaaacacccctcagtcacatctttcattctggacactgaggctgtggcctgggaccgggaaaagaagcagatccaaccattccaagtactcaccacgcgcaagcgcaaggaggttgatgcatcggagatacaggtgcaagtgtgtctgtatgcttttgacctcatctacctcaacggagagtccctggttcgccagcccctgtctcgacgccggcagctgctccgtgagaacttcgtggagacagagggtgagtttgtcttcaccacctctttggacaccaaggacaccgagcagattgctgagttcttggagcagtcagtgaaggactcctgtgagggcctgatggtgaagaccttggatgttgatgccacttatgagattgccaagaggtcacacaactggctcaagctgaagaaggactaccttgacggtgtgggtgacactctggaccttgtggtgattggtgcctacctgggccgggggaagcgggctggccggtatggaggcttcctgttggctgcctacgatgaggagagtgaagagcttcaggctatatgcaagctgggaactggattcagcgatgaggagctggaggaacaccaccagagcctgcaggccctggtattgcctaccccacgcccctatgtgaggattgatggggcagtggccccagaccattggctggacccaagcattgtgtgggaggtgaagtgtgctgatctttccctgtcccccatctaccctgctgcacggggcctggtggacaaagaaaaagggatctctcttcgttttcctcggttcattcgtgtccgtaaagacaagcagccagagcaggccactaccagtaaccaggttgcctctttgtaccggaagcagagtcagattcagaaccaacaaagctcagacttggactccgacgttgaagactattaacctcctgctctcctgggcctggggtggaggttgcgagaacatgggcactgttggattggtggtgttgattggtgtgtgtggagtcacaggagtgagatcctggggctggaatgctttattttcttcaataaatgacttcttagatgcctacaaaaaaaaaaaaaaaaaa

(19) United States
(12) Patent Application Publication
(10) Pub. No.: US 2010/0122356 A1
(43) Pub. Date: May 13, 2010

(54) PIG MODEL FOR PSORIASIS

(75) Inventors: Peter Michael Kragh, Trondheim (NO); Lars Axel Bolund, Skodstrup (DK); Karsten Kristiansen, Broby (DK); Charlotte Brandt Sørensen, Hojbjerg (DK); Jacob Giehm Mikkelsen, Silkeborg (DK); Nicklas Heine Staunstrup, Aarhus N (DK); Thomas Kongstad Petersen, Stenlose (DK); Lars Svensson, Lund (SE)

Correspondence Address: WEINGARTEN, SCHURGIN, GAGNEBIN & LEBOVICI LLP, TEN POST OFFICE SQUARE, BOSTON, MA, 02109, US

(73) Assignees: AARHUS UNIVERSITET, Aarhus C (DK); LEO PHARMA A/S, Ballerup (DK); SYDDANSK UNIVERSITET, Odense M (DK)

(21) Appl. No.: 12/529,812

(22) PCT Filed: Mar. 7, 2008
(86) PCT No.: PCT/DK2008/050059

§ 371 Date: Jan. 20, 2010

(30) Foreign Application Priority Data
Mar. 7, 2007 (DK) PA 2007 00343
Mar. 8, 2007 (DK) PA 2007 00362

(51) Int. Cl. A61K 49/00; A01K 67/027; C12N 5/10; C12N 15/63; A61P 17/06

(52) U.S. Cl. 800/3; 800/9; 435/325; 800/24

(57) ABSTRACT

The present invention relates to a genetically modified pig as a model for studying psoriasis. The modified pig model displays one or more phenotypes associated with psoriasis. Disclosed is also a modified pig comprising a mutation in the endogenous ILK-I Ra, JunB/cJun, CD18, IKK2, and/or LIG1 gene, and/or a human, porcine and/or murine PPARs, PPAR-δ, lκB-α, STAT3c, Integrin beta 1, Integrin alpha 2, MEK1, Amphiregulin, BMP-6, VEGF, JunBΔec-JunΔep, IL-I a, TGF.beta 1, CD18 hypo, Cre/lkk2FL/FL, Dsg1, SCCE, TGF-a, TNF-a, IL-20, IFN-g, LIG1 KO, KGF, IL-6, PAFR1 Cre/lkk2FL/FL, IL1 R, Dsg3, IFN-gamma, p40, ILI Ra, IKK2, JunB/c-Jun, and/or LIG1 gene, transcriptional and/or translational product or part thereof. The invention further relates to methods for producing the modified pig; and methods for evaluating the effect of a therapeutical treatment of psoriasis, for screening the efficacy of a pharmaceutical composition, and a method for treatment of human being suffering from psoriasis are disclosed.

### FIELD OF INVENTION

The present invention relates to a genetically modified pig as a model for studying psoriasis, wherein the pig model expresses at least one phenotype associated with said disease. The invention further relates to methods by which the genetically modified pig is produced. In addition, methods for evaluating the response of a therapeutical treatment of psoriasis, for screening the efficacy of a pharmaceutical composition, and a method for treatment of human being suffering from psoriasis are disclosed.

### BACKGROUND OF INVENTION

Transgenic, non-human animals can be used to understand the action of a single gene or genes in the context of the whole animal and the interrelated phenomena of gene activation, expression, and interaction. The technology has also led to the production of models for various diseases in humans and other animals which contributes significantly to an increased understanding of genetic mechanisms and of genes associated with specific diseases.

Traditionally, smaller animals such as mice have been used as disease models for human diseases and have been found to be suitable as models for certain diseases. However, their value as animal models for many human diseases is quite limited due to differences in mice compared to humans. Larger transgenic animals are much more suitable than mice for the study of many of the effects and treatments of most human diseases because of their greater similarity to humans in many aspects. Particularly, pigs are believed to be valuable as disease models for human diseases.

It is estimated that 2-3 percent of the Western population suffers from psoriasis. Psoriasis affects both sexes equally and can occur at any age, although it most commonly appears for the first time between the ages of 15 and 25 years. Psoriasis is a chronic skin condition characterized by inflamed, red, raised areas covered with white scales. Scaling occurs when cells in the outer layer of skin reproduce faster than normal and pile up on the skin's surface. Consequently, the skin sheds every three to four days. Most often, the skin on the elbows, knees, in the scalp or in the genital region is attacked by psoriasis. Furthermore, nail changes are common and include pitting and a yellowish discoloration that resembles a fungal infection. Psoriasis may also cause hair loss.

Psoriasis is a chronic condition in which outbreaks of psoriasis recur varying in severity from minor localised areas of the body to complete body coverage. In addition, psoriatic arthritis is also observed in 10 to 15 percent of the patients suffering from psoriasis. Psoriatic arthritis is caused by inflammation of the joints due to psoriasis.

After outbreak, psoriasis will often reoccur with varying severity. The cause of psoriasis is not fully understood. It is generally considered to be an auto-immune disease, in which the body has an immune response against one of its own tissues or types of cells. Psoriasis is not contagious, but the condition appears to be hereditary.

Psoriasis can manifest itself in a variety of forms, including plaque, pustular, guttate and flexural psoriasis. Each individual may experience symptoms differently, as psoriasis comes in several forms and severities.

Discoid psoriasis is also called plaque psoriasis and is the most common form. Symptoms may include patches of red, raised skin on the trunk, arms, legs, knees, elbows, genitals, and scalp. Nails may also thicken, become pitted, and separate from the nail beds. Plaque psoriasis affects 80 to 90% of people with psoriasis.

Guttate psoriasis is a moderate level of psoriasis, which mostly affects children. Symptoms may include many small patches of red, raised skin. A sore throat associated with streptococcal infection usually precedes the onset of this type of psoriasis. Guttate psoriasis is characterized by numerous small oval spots, appearing over large areas of the body, for example the trunk, limbs, and scalp.

Flexural psoriasis is smooth inflamed patches of skin, occurring in skin folds, for example in the armpits, under the breasts and particularly around the genitals. Flexural psoriasis is often subject to fungal infections and the condition seems to become worse by friction and sweat.

In severe cases erythrodermic psoriasis is observed particularly following abrupt withdrawal of a systemic treatment. Erythrodermic psoriasis involves the widespread inflammation and exfoliation of the skin over most of the body surface, often accompanied by itching, swelling and pain. The extreme inflammation and exfoliation of the skin may even disrupt the body's ability to regulate temperature and for the skin to perform barrier functions which may in turn be fatal.

Finally, in Pustular psoriasis, symptoms may include small pustules (non-infectious pus-containing blisters) all over the body or just on the palms, soles, and other small areas. The symptoms of psoriasis may resemble other skin conditions. When the skin condition progresses to the development of white scales, the physician can usually diagnose psoriasis with a medical examination of the nails and skin. Confirmation of diagnosis may be done with a skin biopsy, in which a small skin specimen is examined under a microscope.

Unfortunately, the cause of psoriasis is not elucidated. Two main theories about the process that occurs in the development of the disease seem to exist. According to one theory, psoriasis is considered to be primarily a disorder of excessive growth and reproduction of skin cells, involving dysfunction of the epidermis and its keratinocytes. According to another theory, psoriasis is believed to be an immune-mediated disorder. the symptoms of which occur in the skin cells due to factors produced by the immune system. T cells have been suggested to become activated, migrate to the dermis and here trigger the release of cytokines. Subsequently, the cytokines cause inflammation and the rapid production of skin cells. The latter theory has been supported by the observation that immunosuppressant medications can alleviate psoriasis plaques. However, an animal model of psoriasis can be triggered in mice lacking T cells (Zenz R, Eferl R, Kenner L, Florin L, Hummerich L, Mehic D, Scheuch H, Angel P, Tschachler E, Wagner E. Psoriasis-like skin disease and arthritis caused by inducible epidermal deletion of Jun proteins. Nature. 2005; 437(7057):369-75.

Existing animal models, display only a few aspects that resembles human psoriasis. Thus, a need exists for an efficient animal model which displays aspects that resemble human psoriasis. Such an animal model will allow for further studying the causes of psoriasis and to test drugs that will alleviate the symptoms of a large number of people suffering from psoriasis.

Even though the genes responsible for psoriasis or involved in the development of disease have been identified in humans it does not follow that animals transgenic for such mutations display a phenotype comparable to that of the human disease.

However, the present invention has surprisingly shown that the genetically modified pig models according of the present invention display the psoriasis phenotype.

### SUMMARY OF INVENTION

The present invention concerns a genetically modified pig model which allows for the study of psoriasis.

Thus, one aspect of the present invention relates to a genetically modified pig as a model for studying psoriasis, wherein the pig model expresses at least one phenotype associated with said disease, and/or a modified pig comprising at least one mutation in the endogenous ILK-1 Ra, JunB/cJun, CD18, IKK2, and/or LIG1 gene or part thereof, transcriptional and/or translational product or part thereof, and/or a modified pig comprising at least one human, porcine and/or murine PPARs, PPAR-δ, IκB-α, STAT3c, Integrin beta 1, Integrin alpha 2, MEK1, Amphiregulin, BMP-6, VEGF, JunBΔec-JunΔep, IL-1a, TGF.beta 1, CD18 hypo, Cre-IIKK2 fl7fl, Dsg1, SCCE, TGF-a, TNF-a, IL-20, IFN-g, LIG1 KO, KGF, IL-6, PAFR, Cre/Ikk2FL/FL, ID R, Dsg3, IFN-gamma, p40, IL1Ra, IKK2, JunB/c-Jun, and/or LIG1 gene or part thereof, transcriptional and/or translational product or part thereof. Embodiments for the present invention comprises, mini-pigs for example selected from the group consisting of Goettingen, Yucatan, Bama Xiang Zhu, Wuzhishan and Xi Shuang Banna, including any combination thereof. However, another embodiment relates to pigs that are not a mini-pig, such as the species of Sus domesticus, for example where the pig is selected from the group consisting of Landrace, Yorkshire, Hampshire, Duroc, Chinese Meishan, Berkshire and Piêtrain, including any combination thereof. In a preferred embodiment the pig, embryo, fetus, blastocyst, donor cell and/or cell nucleus is a Goettingen minipig or from a Goettingen minipig.

Embodiments of the present invention comprise the genetically modified pig, wherein the pig is transgenic due to insertion of at least a porcine PPAR-δ gene or part thereof, or due to insertion of at least a human PPAR-δ gene or part thereof, or due to insertion of at least a human PPAR-δ cDNA or part thereof, or due to insertion of at least a porcine PPAR-δ cDNA or part thereof, or due to insertion of at least a porcine IκB-α gene or part thereof, or due to insertion of at least a human IκB-α gene or part thereof, or due to insertion of at least a human IκB-a cDNA or part thereof, or due to insertion of at least a porcine IκB-a cDNA or part thereof, or due to insertion of at least a porcine NFκB gene or part thereof, or due to insertion of at least a human NFκB gene or part thereof, or due to insertion of at least a human NFκB cDNA or part thereof, or due to insertion of at least a porcine NFκB cDNA or part thereof.

A second aspect of the present invention relates to genetically modified porcine blastocyst derived from the genetically modified pig model as disclosed herein and/or

a modified porcine blastocyst comprising at least one mutation in the endogenous ILK-1Ra, JunB/cJun, CD18, IKK2, and/or LIG1 gene or part thereof, transcriptional and/or translational product or part thereof,
and/or
a modified porcine blastocyst comprising at least one human, porcine and/or murine PPARs, PPAR-δ, IκB-α, STAT3c, Integrin beta 1, Integrin alpha 2, MEK1, Amphiregulin, BMP-6, VEGF, JunBΔec-JunΔep, IL-1a, TGF.beta 1, CD18 hypo, Cre-IIKK2 fl7fl, Dsg1, SCCE, TGF-a, TNF-a, IL-20, IFN-g, LIG1 KO, KGF, IL-6, PAFR, Cre/Ikk2FL/FL, IL1R, Dsg3, IFN-gamma, p40, IL1Ra, IKK2, JunB/c-Jun, and/or LIG1 gene or part thereof, transcriptional and/or translational product or part thereof.

A third aspect of the present invention pertains to a genetically modified porcine embryo derived from the genetically modified pig model as disclosed herein and/or

a modified porcine embryo comprising at least one mutation in the endogenous ILK-1Ra, JunB/cJun, CD18, IKK2, and/or LIG1 gene or part thereof, transcriptional and/or translational product or part thereof,
and/or
a modified porcine embryo comprising at least one human, porcine and/or murine PPARs, PPAR-δ, IκB-α, STAT3c, Integrin beta 1, Integrin alpha 2, MEK1, Amphiregulin, BMP-6, VEGF, JunBΔec-JunΔec, IL-1a, TGF.beta 1, CD18 hypo, Cre-IIKK2 fl7fl, Dsg1, SCCE, TGF-a, TNF-a, IL-20, IFN-g, LIG1 KO, KGF, IL-6, PAFR, Cre/Ikk2FL/FL, IL1R, Dsg3, IFN-gamma, p40, IL1Ra, IKK2, JunB/c-Jun, and/or LIG1 gene or part thereof, transcriptional and/or translational product or part thereof.

A fourth aspect of the present invention concerns a genetically modified porcine fetus derived from the genetically modified pig model as disclosed herein

and/or
a modified porcine fetus comprising at least one mutation in the endogenous ILK-1Ra, JunB/cJun, CD18, IKK2, and/or LIG1 gene or part thereof, transcriptional and/or translational product or part thereof,
and/or
a modified porcine fetus comprising at least one human, porcine and/or murine PPARs, PPAR-δ, IκB-α, STAT3c, Integrin beta 1, Integrin alpha 2, MEK1, Amphiregulin, BMP-6, VEGF, JunBΔec-JunΔep, IL-1a, TGF.beta 1, CD18 hypo, Cre-IIKK2 fl7fl, Dsg1, SCCE, TGF-a, TNF-a, IL-20, IFN-g, LIG1 KO, KGF, IL-6, PAFR, Cre/Ikk2FL/FL, IL1R, Dsg3, IFN-gamma, p40, IL1Ra, IKK2, JunB/c-Jun, and/or LIG1 gene or part thereof, transcriptional and/or translational product or part thereof.

A fifth aspect of the present invention relates to a genetically modified porcine donor cell and/or cell nucleus derived from the genetically modified pig model as disclosed herein

and/or
a modified porcine donor cell and/or cell nucleus comprising at least one mutation in the endogenous ILK-1Ra, JunB/cJun, CD18, IKK2, and/or LIG1 gene or part thereof, transcriptional and/or translational product or part thereof,
and/or
a modified porcine donor cell and/or cell nucleus comprising at least one human, porcine and/or murine PPARs, PPAR-δ, IκB-α, STAT3c, Integrin beta 1, Integrin alpha 2, MEK1, Amphiregulin, BMP-6, VEGF, JunBΔec-JunΔep, IL-1a, TGF.beta 1, CD18 hypo, Cre-IIKK2 fl7fl, Dsg1, SCCE, TGF-a, TNF-a, IL-20, IFN-g, LIG1 KO, KGF, IL-6, PAFR, Cre/Ikk2FL/FL, IL1R, Dsg3, IFN-gamma, p40, IL1Ra, IKK2, JunB/c-Jun, and/or LIG1 gene or part thereof, transcriptional and/or translational product or part thereof.

Also, the present invention relates to the genetically modified pig model, porcine blastocyst, embryo, fetus, and/or donor cell as described above obtainable by nuclear transfer comprising the steps of

i) establishing at least one oocyte having at least a part of a modified zona pellucida,
ii) separating the oocyte into at least two parts obtaining an oocyte having a nucleus and at least one cytoplast,
iii) establishing a donor cell or cell nucleus with desired genetic properties,
iv) fusing at least one cytoplast with the donor cell or membrane surrounded cell nucleus,
v) obtaining a reconstructed embryo,
vi) activating the reconstructed embryo to form an embryo; culturing said embryo; and
vii) transferring said cultured embryo to a host mammal such that the embryo develops into a genetically modified fetus,
wherein said genetically modified embryo obtainable by nuclear transfer comprises steps i) to v) and/or vi),
wherein said genetically modified blastocyst obtainable by nuclear transfer comprises steps i) to vi) and/or vii),
wherein said genetically modified fetus obtainable by nuclear transfer comprises steps i) to vii).

Furthermore, a sixth aspect pertains to a method for producing a transgenic pig, porcine blastocyst, embryo, fetus and/or donor cell as a model for psoriasis comprising:

i) establishing at least one oocyte
ii) separating the oocyte into at least three parts obtaining at least one cytoplast,
iii) establishing a donor cell or cell nucleus having desired genetic properties,
iv) fusing at least one cytoplast with the donor cell or membrane surrounded cell nucleus,
v) obtaining a reconstructed embryo,
vi) activating the reconstructed embryo to form an embryo; culturing said embryo; and
vii) transferring said cultured embryo to a host mammal such that the embryo develops into a genetically modified foetus,
wherein said transgenic embryo comprises steps i) to v) and/or vi),
wherein said transgenic blastocyst comprises steps i) to vi) and/or vii),
wherein said transgenic fetus comprises steps i) to vii.).

Embodiments of the aspects comprise one or more of the features as defined in any of the preceding claims, wherein the method for activation of the reconstructed embryo is selected from the group of methods consisting of electric pulse, chemically induced shock, increasing intracellular levels of divalent cations and reducing phosphorylation. Further embodiments of the sixth aspects comprise one or more of the features as defined above, wherein steps iv) and vi) are performed sequentially or simultaneously, and embodiments comprising one or more of the features, wherein the embryo is cultured in vitro. Such embryo may be cultured in sequential culture. The embryo, for example at the blastocyst stage, is cryopreserved prior to transfer to a host mammal.

For the methods of the present invention embodiments cover pigs, mini-pigs for example selected from the group consisting of Goettingen, Yucatan, Bama Xiang Zhu, Wuzhishan and Xi Shuang Banna, including any combination thereof. However, another embodiment relates to pigs that are not a mini-pig, such as the species of Sus domesticus, for example where the pig is selected from the group consisting of Landrace, Yorkshire, Hampshire, Duroc, Chinese Meishan, Berkshire and Piêtrain, including any combination thereof.

A seventh aspect of the present invention relates to a method for evaluating the effect of a therapeutical treatment of psoriasis, said method comprising the steps of

i) providing the pig model the present invention
ii) treating said pig model with a pharmaceutical composition exerting an effect on said phenotype, and

iii) evaluating the effect observed.

An eighth aspect pertains to a method for screening the efficacy of a pharmaceutical composition, said method comprising the steps of
i) providing the pig model of the present invention,
ii) expressing in said pig model said genetic determinant and exerting said phenotype for said disease,
iii) administering to said pig model a pharmaceutical composition the efficacy of which is to be evaluated, and
   iv) evaluating the effect, if any, of the pharmaceutical composition on the phenotype exerted by the genetic determinant when expressed in the pig model.

Finally, a ninth aspect of the present invention relates to a method for treatment of a human being suffering from psoriasis, said method comprising the initial steps of

i) providing the pig model of the present invention,
ii) expressing in said pig model said genetic determinant and exerting said phenotype for said disease,
iii) administering to said pig model a pharmaceutical composition the efficacy of which is to be evaluated, and
iv) evaluating the effect observed, and
v) treating said human being suffering from psoriasis based on the effects observed in the pig model.

### DESCRIPTION OF DRAWINGS

FIG. 1 shows the bi-phased technology of the present invention in which an integrating SB vector, carrying a reporter gene and a selective marker gene, serves as a reporter for continuous gene expression and hence as a target for gene insertion. In a second modification step this vector may serve as a target for insertion of one or more gene expression cassettes in a well-characterized locus.

FIG. 2 shows a schematic representation of pSBT/RSV-GFIP.

FIG. 3 shows transposition of SB vectors in porcine fibroblasts. A standard transposon encoding a puromycin resistance gene (SBT/PGK-puro) was employed and varying levels of transposition were detected, resulting in about 75 drug-resistant colonies in cultures of fibroblasts co-transfected with pSBT/PGK-puro and pCMV-SB, less than 3 colonies appeared after transfection with pSBT/PGK-puro and pCMV-mSB, the latter which encodes an inactive version of the transposase. Interestingly, a mean of almost 140 colonies was obtained using the hyperactive transposase variant HSB3, indicating that HSB3 also in porcine cells mediates higher levels of transposition compared to the original SB transposase.

FIG. 4 shows efficient insertion of a FRT-tagged SB vector in pig fibroblasts SB-tagged cell clones containing a Flp recombination target site for site-specific gene insertion were co-transfected the pSBT/loxP.SV40-lopP257 plasmid with pCMV-mSB, pCMV-SB, and pCMV-HSB3, respectively. HSB3 again showed the highest activity, resulting in about 30 drug-resistant colonies after transfection of 3H 10⁴ fibroblasts.

FIG. 5 shows clone analysis by fluorescence microscopy of isolated and expanded puromycin-resistant colonies demonstrates efficient FRTeGFP expression

FIG. 6. (a) Oocytes trisection; (b) couplets of fibroblast-oocyte fragment for the first fusion; (c) embryos reconstructed with triplets (note elongation under the AC currency); (d) triplets fusion. Scale bar=50 μm.

FIG. 7. (a) In vitro matured oocytes after partial zona digestion. (b) Delipated oocytes after centrifugation. (c) Bisection of delipated oocytes. (d) Couplets of fibroblast-oocyte fragment for the first fusion. (e) Four-cell stage reconstructed embryos developed from delipated oocytes. (f) Four-cell stage reconstructed embryos developed from intact oocytes. (g) Re-expanded blastocysts from delipated embryos after warming. (h) Hoechst staining and UV illumination of re-expanded blastocysts from delipated embryos after warming. Bar represents 100 μm.

FIG. 8. Bisection at chemically assisted enucleation. Note the extrusion cone or polar body connected to the smaller part (putative karyoplast). Stereomicroscopic picture. Bar represents 50 μm.

FIG. 9. Hoechst staining and UV illumination of the absence and presence of chromatin. UV light, inverted fluorescent microscopic picture. Bar represents 50 μm. (a) The absence of chromatin in putative cytoplasts (b) The presence of chromatin in putative karyoplasts.

FIG. 10. Stereomicroscopic picture of Day 7 blastocysts produced with chemically assisted handmade enucleation (CAHE). Bar represents 50 μm.

FIG. 11. Hoechst staining and UV illumination of blastocyst developed after chemically assisted handmade enucleation (CAHE). Bar represents 50 μm.

FIG. 12 shows porcine PPAR δ cDNA (Sus scrofa; Landrace) expressed in the skin of the pig model.

FIG. 13 shows human IκB-a cDNA to be expressed in the skin of the pig model.

FIG. 14 shows human PPAR δ cDNA expressed in the skin of the pig model.

FIG. 15 shows porcine IκB-a cDNA (Sus scrofa; Landrace) to be expressed in the skin of the pig model.

FIG. 16 is a schematic representation of a Transposon vector (pT2 vector) construct, which may be used for insertion of a transgene, preferably integrin, according to the present invention.

### DETAILED DESCRIPTION OF THE INVENTION

Abnormal epidermal proliferation and differentiation characterize the inflammatory skin disease psoriasis. Psoriatic human epidermis is unbalanced with respect to the gene regulators PPAR-δ and NFκB. Down-regulating NFκB by expression of a dominant negative variant of IκB-α and up-regulating PPAR-δ in pig cause the development of a primitive pig epidermal tissue in which psoriatic-like dysregulation can be studied.

The present invention pertains to a genetically modified pig model for studying psoriasis, wherein the pig model expresses at least one phenotype associated with psoriasis.

It will be appreciated that the invention does not comprise processes for modifying the genetic identity of pigs which are likely to cause them suffering without any substantial medical benefit to man or animal, or animals resulting from such processes.

The present invention also relates to genetically modified pig embryos, blastocyst, fetus, donor cells and/or cell nucleus obtainable by the methods described herein.

The methods for producing the pig model for studying psoriasis described herein do not encompass a surgical step performed on the pig.

The term “genetic determinant” is used herein to refer to a single-stranded or double-stranded “polynucleotide molecule” or “nucleic acid” comprising a structural gene of interest. The “genetic determinant” encodes a protein not ordinarily made in appreciable amounts in the target cells. Thus, “genetic determinants” include nucleic acids which are not ordinarily found in the genome of the target cell. “Genetic determinants” also include nucleic acids which are ordinarily found within the genome of the target cell, but is in a form which allows for the expression of proteins which are not ordinarily expressed in the target cells in appreciable amounts. Alternatively, “genetic determinants” may encode a variant or mutant form of a naturally-occurring protein.

The terms “polynucleotide” and “nucleic acid” are used interchangeably, and, when used in singular or plural, generally refers to any polyribonucleotide or polydeoxyribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs. The term includes DNAs (including cDNAs) and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

### Pigs

The terms ‘transgenic’ pig and ‘genetically modified’ pig are used in identical meaning herein.

The present invention relates to a modified pig as a model for studying psoriasis, wherein the pig model expresses at least one phenotype associated with psoriasis. The pig of the present invention may be any pig. The pig is evolutionary close to humans as compared to for example rodentia. Furthermore, the pig has been widely used in bio-medical research because of the similarities between human and porcine physiology (Douglas, 1972; Book & Bustad, 1974).

In one embodiment of the present invention, the pig is a wild pig. In another embodiment the pig is the domestic pig, Sus scrofa, such as S. domesticus. In yet another embodiment the invention relates to mini pigs, as well as to inbred pigs. The pig can be selected e.g. from the group consisting of Landrace, Yorkshire, Hampshire, Duroc, Chinese Meishan, Berkshire and Piêtrain, such as the group consisting of Landrace, Yorkshire, Hampshire and Duroc, for example the group consisting of Landrace, Duroc and Chinese Meishan, such as the group consisting of Berkshire, Pietrain, Landrace and Chinese Meishan, for example the group consisting of Landrace and Chinese Meishan. In one embodiment, the pig is not a mini-pig. In another embodiment the pig of the present invention is an inbred pig.

In another embodiment of the present invention the pig is a mini-pig and the mini-pig is preferably selected from the group consisting of Goettingen, Yucatan, Bama Xiang Zhu, Wuzhishan and Xi Shuang Banna. Thus, the present invention relates to any of Goettingen, Yucatan, Bama Xiang Zhu, Wuzhishan and Xi Shuang Banna separately, or in any combination.

Due to its size and weight of about 200 kg the domestic pig is not easily handled in a laboratory setting. A preferred alternative to the domestic pig is the Goettingen (Göttingen) mini-pig that weighs about 30 kg. The Goettingen minipig has a brain with almost the same brain size and identical morphology to the domestic pig, although differences may exist in the postnatal development (Jelsing et al. J. Exp. Biol. 2006). Thus, the Göttingen minipig is increasingly used in neuroscience and has served as experimental models for functional imaging studies, and a volumetric screening procedure and a magnetic resonance-based stereotaxic atlas has been developed (Jelsing et al. Exp Brain Res 2005; Watanabe et al. NeuroImage 2001). Therefore, a preferred embodiment the pig of the present invention is the Goettingen mini pig.

### Genetically Modified

The modifications are introduced in the somatic cell prior to cell nuclear transfer. However, the modification may in another embodiment be introduced during the cell nuclear transfer process, for example by addition of transgenes at different steps of the hand made cloning (HMC) procedure that will then find their way to the genome of the embryo.

The genetic modifications comprise random integration of a disease causing gene, mutated gene, into the genome of the somatic cell. It could also be random integration of a normal non-mutated gene that will cause a disease when expressed in a specific tissue or at a specific expression level.

However, the invention also pertains to modified pig embryos, blastocyst, fetus, donor cells and/or cell nucleus obtained by transfer of mRNA and/or protein of the genes disclosed herein. Thus, the modification of the pig embryos, blastocyst, fetus, donor cells and/or cell nucleus is in one embodiment does not lead to integration of a transgene into the genome of the pig, embryo, blastocyst and/or fetus. The introduced gene or transgene, transcriptional and/or translational product or part thereof, may originate from any species, including bacteria, pig, human, mouse, rat, yeast, invertebrates, or plants. Regulatory sequences of the transgene may drive ubiquitous or inducible or tissue- and/or time-specific expression and may also originate from any species including pig, human, mouse, rat, yeast, invertebrates, or plants.

Importantly, the genetic modification in the somatic cell may be targeted to a specific region in the porcine genome by homologous recombination of a targeting construct or by gene editing procedures. This could be inactivation (e.g. knock-out) of specific genes that will cause a disease or phenotype, or it could be integration (knock-in) of specific mutations to specific genes that will then cause disease. Also, disease causing transgenes can be integrated into specific regulatory regions of the porcine genome by homologous recombination methods.

Homologous recombination occurs between two homologous DNA molecules. It is also called DNA crossover. By homologous recombination, one DNA segment can replace another DNA segment with a similar sequence. The process involve breakage and reunion between the homologous regions of DNA, which is mediated by specialized enzymes. The technique allows replacing one allele with an engineered construct without affecting any other locus in the genome. Using homologous recombination it is possible to direct the insertion of a transgene to a specific known locus of the host cells genom. Knowing the DNA sequence of the target locus, it is possible to replace any gene with a genetically modified DNA construct, thereby either replacing or deleting the target sequence. The technique comprises discovering and isolating the normal gene and then determining its function by replacing it in vivo with a defective copy. This procedure is known as ‘gene knock-out’, which allows for specific gene targeting by taking advantage of homologous recombination. Cloned copies of the target gene are altered to make them nonfunctional and are then introduced into ES cells where they recombine with the homologous gene in the cell's genome, replacing the normal gene with a nonfunctional copy.

Homologous recombination can similarly be exploited to generate fusion genes or insertion of point mutations in a ‘knock-in’ strategy, in which a targeting vector, comprising a relevant exon of the target locus fused with the cDNA sequence of chromosomal translocation-fusion partner, is transfected into embryonic stem cells, whereby the recombinant sequence is fused to an endogenous gene to generate fusion a gene.

Another applicable technique to exploits the phenomenon called RNA interference (RNAi), in which 21 nucleotide small interfering RNAs (siRNA) can elicit an effective degradation of specific mRNAs. RNA interference constitutes a new level of gene regulation in eukaryotic cells. It is based on the fact that presence of double stranded RNA in a cell eliminates the expression of a gene of the same sequence, whereas expression of other unrelated genes is left undisturbed. The siRNA stimulates the cellular machinery to cut up other single-stranded RNA having the same sequence as the siRNA.

The genetic modifications introduced into the porcine genome prior or during the HMC procedure could also be epigenetic modifications (e.g. methylation of DNA or methylation or acetylation/deacetylation of histones) by incubating somatic cells, oocytes or reconstructed HMC embryos with chemical components such as Tricostatin or compounds with similar effect.

The present invention relates to a modified pig embryos, blastocyst, fetus, donor cells and/or cell nucleus, comprising a genetic determinant as described in detail herein. The present invention also relates to porcine embryos, blastocysts and/or fetuses derived from a modified pig expressing at least one phenotype associated with psoriasis.

In one embodiment of the present invention the transgenic pig embryos, blastocyst, fetus, donor cells and/or cell nucleus is transgenic for at least one gene selected from the porcine PPAR δ gene (SEQ ID NO: 1) or part thereof, human PPAR δ gene (SEQ ID NO: 2) or part thereof, the porcine IκB-α gene (SEQ ID NO: 3) or part thereof or human IκB-α gene (SEQ ID NO: 4) or part thereof. However, in another embodiment the transgenic pig is transgenic for a combination of genes, for example the porcine PPAR δ gene or part thereof and the human IκB-α gene or part thereof, or the transgenic pig is transgenic for the combination of the porcine PPAR δ gene or part thereof and the porcine IκB-α gene or part thereof; or the transgenic pig is transgenic for the combination of the human PPAR δ gene or part thereof and the human IκB-α gene or part thereof, or the transgenic pig is transgenic for the combination of the human PPAR δ gene or part thereof and the porcine IκB-a gene or part thereof. It is appreciated that the cDNA or part thereof of the porcine PPAR δ gene and/or the cDNA or part thereof of the human PPAR δ gene and/or the cDNA or part thereof of the porcine IκB-α gene and/or the cDNA or part thereof of the human IκB-α gene, and combinations as outlined herein is within the scope of the present invention. Furthermore in another embodiment, the genetically modified pig comprises the transcriptional product or part thereof and/or the translational product or part thereof of the porcine and/or human PPAR delta gene. In yet a further embodiment the genetically modified pig comprises the transcriptional product or part thereof and/or the translational product or part thereof of the porcine and/or human IκB-a gene, or combination thereof as described herein.

It is appreciated that the genes (transgenes) may be driven by promoters that direct expression of the transgene in the skin of the pig according to the present invention. A number of skin-specific promoters are known that are suitable for skin-specific expression, for example keratin 1 (K1), keratin 5 (K5) promoter, keratin 10 (K10) promoter, keratin 14 (K14) promoter and the involucrine promoter. It is also within the scope of the present invention that the transgene is expressed constitutively or by induction.

Genetic determinants of psoriasis according to the present invention also comprise overexpression of transgenes. Overexpression of transgenes described herein lead to a psoriasis phenotype in the pig according to the present invention. Embodiments relate to transgenes such as PPARs, such as PPAR-δ, IκB-α, STAT3c, Integrin beta 1, Integrin alpha 2, MEK1, Amphiregulin, BMP-6, VEGF, JunBΔec-JunΔep, IL-1a, TGF.beta 1, CD18 hypo, Cre-IIKK2 fl7fl, Dsg1, SCCE, TGF-a, TNF-a, IL-20, IFN-g, LIG1 KO, KGF, IL-6, PAFR, Cre/Ikk2FL/FL, IL1R, Dsg3, IFN-gamma, p40, IL1Ra, IKK2, JunB/c-Jun, and/or LIG1.

The cDNA or part thereof of the genes listed herein, and combinations of the transgenes is within the scope of the present invention. Furthermore in another embodiment, the genetically modified pig comprises the transcriptional product or part thereof and/or the translational product or part thereof of the porcine, human or murine genes.

In another embodiment, genetic determinants of psoriasis according to the present invention also comprise deletion, mutation and/or suppression of transgenes. Thus, in other embodiments, deletion, mutation and/or suppression of transgenes described herein lead to a psoriasis phenotype in the pig according to the present invention. Such embodiments comprise transgenes such as JunB/c-Jun, IL-1Ra, ILK-1Ra, CD18, and/or LIG2.

Embodiments of the present invention in relation to the combination of promoter and transgene are for example K5—STAT3c (Sano et al Nat Immunol 2005), Involucrine—Integrin beta 1 (Caroll et al Cell 1995), Involucrine—Integrin alpha 2(Carrol et al Cell 1995), Involucrine—MEK1(Hobbs et al J Invest derm 2004), K14—Amphiregulin (Cook et al J Clin Invest 1997), K10—BMP-6 (Blessing et al J Cell biol 1996; Kaiser et al J Invest Dermatol 1998), K14—VEGF (Kunstfeldt et al Blood 2004, Xia et al Blood 2003), K5—JunBΔec-JunΔep (Zenz et al 2005), K14—IL-1a (Groves et al J Clin Invest 1996; Groves et al PNAS1995), K5—TGF.beta 1 (Li et al Derm Symp Proc 2005; Li et al EMBO 2004), CD18 hypo (Bullard et al PNAS 1996; Barlow et al Am J pathol 2003), K14—Cre-IIKK2 fl7 μl (Pasparakis et al Nature 2002), K1—Dsg1 or K1—Dsg3 (Merrit et al Mol Cell Biol 2002), SCCE (Ny et al Act Derm Venerol 2004), K14—TGF-a (Vassar et al Genes devel 1991), K14—TNF-a (Genes Dev. 1992 August; 6(8):1444-56), K14—IL-20 (Blumberg et al Cell 2001), Involucrine—IFN-gamma (Carroll et al J Invest dermatol 1997), LIG1 KO (Suzuki et al FEBS 2002), K14—KGF (Guo et al EMBO 1993), K14—IL-6 (Turksen et al PNAS 1992), PAFR (sato et al Arch Dermatol Res 1999), K14—Cre/Ikk2FL/FL, K14-p40 (Kopp et al, J Invest Dermatol. 2001 September; 117(3):618-26), K14—Tie2 (Voskas et al, Am J. Pathol. 2005 March; 166(3):843-55), K14—IL-1Ra (Shepherd et al, J Invest Dermatol. 2004 March; 122(3):665-9), K14—IKK2 (M. Pasparakis et al., Nature 417(6891), 2002, pp. 861-866), or K14—LIG-1 (Y. Suzuki et al., FEBS Lett. 521(1-3), 2002, pp. 67-71).

### Sequence Identity

Functional equivalents and variants are used interchangeably herein. In one preferred embodiment of the invention there is also provided variants of the human and/or porcine PPAR delta gene and/or IκB-a gene and variants of fragments thereof, and/or any other transgene described herein. When being polypeptides, variants are determined on the basis of their degree of identity or their homology with a predetermined amino acid sequence, said predetermined amino acid sequence being SEQ ID NO: 4, and/or SEQ ID NO: 6, or, when the variant is a fragment, a fragment of any of the aforementioned amino acid sequences, respectively.

Similarly, functional equivalents and variants of PPARs, such as PPAR-δ, IκB-α, STAT3c, Integrin beta 1, Integrin alpha 2, MEK1, Amphiregulin, BMP-6, VEGF, JunBΔec-JunΔep, IL-1a, TGF.beta 1, CD18 hypo, Cre-IIKK2 fl7fl, Dsg1, SCCE, TGF-a, TNF-a, IL-20, IFN-g, LIG1 KO, KGF, IL-6, PAFR, Cre/Ikk2FL/FL, ID R, Dsg3, IFN-gamma, p40, IL1Ra, IKK2, JunB/c-Jun, and/or LIG1 are within the scope of the present invention.

Accordingly, variants preferably have at least 91% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with the predetermined sequence.

The following terms are used to describe the sequence relationships between two or more polynucleotides: “predetermined sequence”, “comparison window”, “sequence identity”, “percentage of sequence identity”, and “substantial identity”.

A “predetermined sequence” is a defined sequence used as a basis for a sequence comparison; a predetermined sequence may be a subset of a larger sequence, for example, as a segment of a full-length DNA or gene sequence given in a sequence listing, such as a polynucleotide sequence of SEQ ID NO:1, SEQ ID NO:2, SEQ ID NO:3, SEQ ID NO:4 or may comprise a complete DNA or gene sequence. Generally, a predetermined sequence is at least 20 nucleotides in length, frequently at least 25 nucleotides in length, and often at least 50 nucleotides in length.

Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window”, as used herein, refers to a conceptual segment of at least 20 contiguous nucleotide positions wherein a polynucleotide sequence may be compared to a predetermined sequence of at least 20 contiguous nucleotides and wherein the portion of the polynucleotide sequence in the comparison window may comprise additions or deletions (i.e., gaps) of 20 percent or less as compared to the predetermined sequence (which does not comprise additions or deletions) for optimal alignment of the two sequences.

Optimal alignment of sequences for aligning a comparison window may be conducted by the local homology algorithm of Smith and Waterman (1981) Adv. Appl. Math. 2: 482, by the homology alignment algorithm of Needleman and Wunsch (1970) J. Mol. Biol. 48: 443, by the search for similarity method of Pearson and Lipman (1988) Proc. Natl. Acad. Sci. (U.S.A.) 85: 2444, by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected.

The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a predetermined sequence over a comparison window of at least 20 nucleotide positions, frequently over a window of at least 25-50 nucleotides, wherein the percentage of sequence identity is calculated by comparing the predetermined sequence to the polynucleotide sequence which may include deletions or additions which total 20 percent or less of the predetermined sequence over the window of comparison. The predetermined sequence may be a subset of a larger sequence, for example, as a segment of the full-length PPAR and/or IκB-a polynucleotide sequence illustrated herein.

By the term “transcriptional or translational products” is meant herein products of gene transcription, such as a RNA transcript, for example an unspliced RNA transcript, a mRNA transcript and said mRNA transcript splicing products, and products of gene translation, such as polypeptide(s) translated from any of the gene mRNA transcripts and various products of post-translational processing of said polypeptides, such as the products of post-translational proteolytic processing of the polypeptide(s) or products of various post-translational modifications of said polypeptide(s).

As used herein, the term “transcriptional product of the gene” refers to a pre-messenger RNA molecule, pre-mRNA, that contains the same sequence information (albeit that U nucleotides replace T nucleotides) as the gene, or mature messenger RNA molecule, mRNA, which was produced due to splicing of the pre-mRNA, and is a template for translation of genetic information of the gene into a protein.

### Phenotypes

The phenotypes associated with psoriasis are many. It is appreciated that the pig model of the present invention expresses at least one phenotype associated with psoriasis, such as three, for example four, five, six, seven, eight, nine, ten, eleven, 12, 13, 14, 15, 16, 17, 18, 19 or 20 phenotypes associated with psoriasis.

The phenotypes associated with psoriasis comprise the disease appearance selected from plaque psoriasis, guttate psoriasis, flexural psoriasis, erythrodermic psoriasis, pustular psoriasis or psoriatic arthritis. Thus, it is appreciated that any one of the phenotypes plaque psoriasis, guttate psoriasis, flexural psoriasis, erythrodermic psoriasis, pustular psoriasis or psoriatic arthritis is displayed in the pig model separately or in combination. However, one or more of the phenotypes may be displayed in the pig model such as a combination of plaque psoriasis and psoriatic arthritis, or a combination of guttate psoriasis and psoriatic arthritis, or a combination of erythrodermic psoriasis and psoriatic arthritis, or a combination of pustular psoriasis and psoriatic arthritis, or a combination of flexural psoriasis and psoriatic arthritis, or a combination of plaque psoriasis and flexural psoriasis, or a combination of pustular psoriasis and plaque psoriasis, or a combination of plaque psoriasis and flexural psoriasis, or a combination of plaque psoriasis and erythrodermic psoriasis, or a combination of guttate psoriasis and erythrodermic psoriasis, or a combination of pustular psoriasis and erythrodermic psoriasis, or a combination of flexural psoriasis and erythrodermic psoriasis.

One phenotype indicative of psoriasis is inflamed, red, raised areas covered with white scales. Scaling occurs when cells in the outer layer of skin reproduce faster than normal and pile up on the skin's surface. Consequently, the skin sheds every three to four days. Most often, the skin on the elbows, knees, in the scalp or in the genital region is attacked by psoriasis. Furthermore, nail changes are common and include pitting and a yellowish discoloration that resembles a fungal infection. Psoriasis may also cause hair loss.

Psoriasis can manifest itself in a variety of forms, including plaque, pustular, guttate and flexural psoriasis. Each individual may experience symptoms differently, as psoriasis comes in several forms and severities.

Discoid psoriasis is also called plaque psoriasis and is the most common form.

Symptoms may include patches of red, raised skin on the trunk, arms, legs, knees, elbows, genitals, and scalp. Nails may also thicken, become pitted, and separate from the nail beds. Plaque psoriasis affects 80 to 90% of people with psoriasis.

Guttate psoriasis is a moderate level of psoriasis, which mostly affects children. Symptoms may include many small patches of red, raised skin. A sore throat associated with streptococcal infection usually precedes the onset of this type of psoriasis. Guttate psoriasis is characterized by numerous small oval spots, appearing over large areas of the body, for example the trunk, limbs, and scalp.

Flexural psoriasis is smooth inflamed patches of skin, occurring in skin folds, for example in the armpits, under the breasts and particularly around the genitals. Flexural psoriasis is often subject to fungal infections and the condition seems to become worse by friction and sweat.

In severe cases erythrodermic psoriasis is observed particularly following abrupt withdrawal of a systemic treatment. Erythrodermic psoriasis involves the widespread inflammation and exfoliation of the skin over most of the body surface, often accompanied by itching, swelling and pain. The extreme inflammation and exfoliation of of the skin may even disrupt the body's ability to regulate temperature and for the skin to perform barrier functions which may in turn be fatal.

Finally, in Pustular psoriasis, symptoms may include small pustules (non-infectious pus-containing blisters) all over the body or just on the palms, soles, and other small areas. The symptoms of psoriasis may resemble other skin conditions. When the skin condition progresses to the development of white scales, the physician can usually diagnose psoriasis with a medical examination of the nails and skin. Confirmation of diagnosis may be done with a skin biopsy, in which a small skin specimen is examined under a microscope.

In one embodiment, the phenotype of the present invention is selected from the group consisting of plaque psoriasis, guttate psoriasis, flexural psoriasis, erythrodermic psoriasis, pustular psoriasis or psoriatic arthritis. In another embodiment, the phenotype of the present invention is selected from the group consisting of white scales, skin inflammation, raised skin, red skin, skin shedding, nail changing, yellowish discoloration of nails, and hair loss. In a further embodiment, the phenotype of the present invention is skin shedding. In yet another embodiment, the phenotype of the present invention is patches of red, raised skin on the trunk, arms, legs, knees, elbows, genitals, and scalp. In another embodiment, the phenotype of the present invention is selected the group consisting of small patches of red skin, raised skin, numerous small oval spots appearing over large areas of the body. In a further embodiment, the phenotype of the present invention is selected from the group consisting of smooth inflamed patches of skin, occurring in skin folds, for example in the armpits, under the breasts and particularly around the genitals. In another one embodiment, the phenotype of the present invention is selected from the group consisting of widespread inflammation and exfoliation of the skin over most of the body surface, itching, swelling and pain, disruption of the body's ability to regulate temperature, and death. In yet another embodiment, the phenotype of the present invention is small pustules all over the body or just on the palms, soles, and other small areas.

The diagnosis is made primarily on the basis of clinical observation and microscopic examination of skin tissue, for example in the form of biopsies.

The phenotype may be studied at various ages of the pig, for example age 6, 12, 18, 24 months of age, or 2.5, 3, 3.5, 4, 4.5, 5, 5.5, 6, 6.5 or 7 years of age.

It is appreciated that the modified porcine embryo, blastocyst and/or fetus derivable from the modified pig model for studying psoriasis, expressing at least one phenotype associated with psoriasis may be the result of the crossing of for example a pig overexpressing transgenes one or more of for example PPARs, such as PPAR-δ, IκB-α, STAT3c, Integrin beta 1, Integrin alpha 2, MEK1, Amphiregulin, BMP-6, VEGF, JunBΔec-JunΔep, IL-1a, TGF.beta 1, CD18 hypo, Cre-IIKK2 fl7fl, Dsg1, SCCE, TGF-a, TNF-a, IL-20, IFN-g, LIG1 KO, KGF, IL-6, PAFR, Cre/Ikk2FL/FL, ID R, Dsg3, IFN-gamma, p40, IL1Ra, IKK2, JunB/c-Jun, and/or LIG1 with a different transgene of the same group. However, a pig overexpressing one or more of for example PPARs, such as PPAR-δ, IκB-α, STAT3c, Integrin beta 1, Integrin alpha 2, MEK1, Amphiregulin, BMP-6, VEGF, JunBΔec-JunΔep, IL-1a, TGF.beta 1, CD18 hypo, Cre-IIKK2 fl7fl, Dsg1, SCCE, TGF-a, TNF-a, IL-20, IFN-g, LIG1 KO, KGF, IL-6, PAFR, Cre/Ikk2FL/FL, IL1R, Dsg3, IFN-gamma, p40, IL1Ra, IKK2, JunB/c-Jun, and/or LIG may be crossed with a pig harbouring at least one deletion, mutation and/or suppression of transgenes, such as JunB/c-Jun, IL-1Ra, ILK-1Ra, CD18, and/or LIG2.

### Methods for Producing Pig Model for Studying Psoriasis

The modified pig embryos, blastocyst, fetus, donor cells and/or cell nucleus of the present invention may be produced using any technique in which modified genetic material, transcriptional product and/or translational product or part thereof, is transferred from at donor cell to a host cell, such as an enucleated oocyte. A number of techniques exist such as introducing genetic material from a genetically modified somatic cell into an enucleated oocyte by for example microinjection or by nuclear transfer. The present invention provides improved procedures for cloning pigs by nuclear transfer which refers to the introduction of a full complement of nuclear DNA from one cell to an enucleated cell.

In cloning, the transfer of the nucleus of a somatic (body) cell or somatic cell into an egg cell (oocyte) which has had its own nucleus removed (denucleated or enucleated) is called somatic cell nuclear transfer. The new individual will develop from this reconstructed embryo and be genetically identical to the donor of the somatic cell. In the present invention the modified pig model, porcine embryo, blastocyst and/or fetus is obtainable by somatic cell nuclear transfer comprising the steps of a) establishing at least one oocyte having at least a part of a modified zona pellucida, b) separating the oocyte into at least two parts obtaining at least one cytoplast, c) establishing a donor cell or cell nucleus having desired genetic properties, d) fusing at least one cytoplast with the donor cell or membrane surrounded cell nucleus, e) obtaining a reconstructed embryo. However, the present invention also relates to a method for producing a transgenic pig as a model for psoriasis comprising the steps of a) establishing at least one oocyte, b) separating the oocyte into at least three parts obtaining at least two cytoplasts, c) establishing a donor cell or cell nucleus having desired genetic properties, d) fusing at least one cytoplast with the donor cell or membrane surrounded cell nucleus, e) obtaining a reconstructed embryo, f) activating the reconstructed embryo to form an embryo; and g) transferring said cultured embryo to a host mammal such that the embryo develops into a genetically modified fetus, wherein said genetically modified embryo obtainable by nuclear transfer comprises steps a) to e) and/or f),

wherein said genetically modified blastocyst obtainable by nuclear transfer comprises steps a) to e) and/or f), wherein said genetically modified fetus obtainable by nuclear transfer comprises steps a) to g)

It is appreciated that the donor cell or cell nucleus of c) harbours genetic determinants for psoriasis, for example in the form of modified human or porcine PPAR and/or Integrin gene or part thereof and/or transcriptional and/or translational products thereof. The host mammal of g) is in one embodiment a pig, preferably a Goettingen mini pig.

However, the present invention also relates to a method for producing a transgenic pig, porcine blastocyst, embryo and/or fetus as a model for psoriasis comprising the steps of a) establishing at least one oocyte, b) separating the oocyte into at least three parts obtaining at least one cytoplasts, c) establishing a donor cell or cell nucleus having desired genetic properties, d) fusing at least one cytoplast with the donor cell or membrane surrounded cell nucleus, e) obtaining a reconstructed embryo, f) activating the reconstructed embryo to form an embryo; and g) transferring said cultured embryo to a host mammal such that the embryo develops into a genetically modified fetus, wherein said genetically modified embryo obtainable by nuclear transfer comprises steps a) to e) and/or f), wherein said genetically modified blastocyst obtainable by nuclear transfer comprises steps a) to e) and/or f), wherein said genetically modified fetus obtainable by nuclear transfer comprises steps a) to g).

The oocyte of b) may in another embodiment be separated into at least three parts obtaining at least two cytoplasts. It is appreciated that the donor cell or cell nucleus of c) harbours genetic determinants for psoriasis, for example in the form of modified human or porcine PPAR and/or Integrin gene or part thereof and/or transcriptional and/or translational products thereof. The host mammal of g) is in one embodiment a pig, preferably a Goettingen mini pig.

The various parameters are described in detail below.

### Oocyte

The term ‘oocyte’ according to the present invention means an immature female reproductive cell, one that has not completed the maturing process to form an ovum (gamete). In the present invention an enucleated oocyte is the recipient cell in the nuclear transfer process.

The oocytes according to the present invention are isolated from oviducts and/or ovaries of a mammal. Normally, oocytes are retrieved from deceased pigs, although they may be isolated also from either oviducts and/or ovaries of live pigs. In one embodiment the oocytes are isolated by oviductal recovery procedures or transvaginal recovery methods. In a preferred embodiment the oocytes are isolated by aspiration. Oocytes are typically matured in a variety of media known to a person skilled in the art prior to enucleation. The oocytes can also be isolated from the ovaries of a recently sacrificed animal or when the ovary has been frozen and/or thawed. Preferably, the oocytes are freshly isolated from the oviducts.

Oocytes or cytoplasts may also be cryopreserved before use. While it will be appreciated by those skilled in the art that freshly isolated and matured oocytes are preferred, it will also be appreciated that it is possible to cryopreserve the oocytes after harvesting or after maturation. If cryopreserved oocytes are utilised then these must be initially thawed before placing the oocytes in maturation medium. Methods of thawing cryopreserved materials such that they are active after the thawing process are well-known to those of ordinary skill in the art. However, in general, cryopreservation of oocytes and cytoplasts is a very demanding procedure, and it is especially difficult in pigs, because of the above mentioned general fragility of pig oocytes and cytoplasts, and because of the high lipid content that makes them very sensitive to chilling injury (i.e. injury that occurs between +15 and +5° C. during the cooling and warming procedure).

In another embodiment, mature (metaphase II) oocytes that have been matured in vivo, may be harvested and used in the nuclear transfer methods disclosed herein. Essentially, mature metaphase II oocytes are collected surgically from either nonsuperovulated or superovulated pigs 35 to 48 hours past the onset of estrus or past the injection of human chorionic gonadotropin (hCG) or similar hormone.

Where oocytes have been cultured in vitro, cumulus cells that are surrounding the oocytes in vivo may have accumulated may be removed to provide oocytes that are at a more suitable stage of maturation for enucleation. Cumulus cells may be removed by pipetting or vortexing, for example, in the presence of in the range of 0.1 to 5% hyaluronidase, such as in the range of 0.2 to 5% hyaluronidase, for example in the range of 0.5 to 5% hyaluronidase, such as in the range of 0.2 to 3% hyaluronidase, for example in the range of 0.5 to 3% hyaluronidase, such as in the range of 0.5 to 2% hyaluronidase, for example in the range of 0.5 to 1% hyaluronidase, such as 0.5% hyaluronidase.

The first step in the preferred methods involves the isolation of a recipient oocyte from a suitable pig. In this regard, the oocyte may be obtained from any pig source and at any stage of maturation.

The stage of maturation of the oocyte at enucleation and nuclear transfer has been reported to be of significance for the success of nuclear transfer methods. Immature (prophase I) oocytes from pig ovaries are often harvested by aspiration. In order to employ techniques such as genetic engineering, nuclear transfer and cloning, such harvested oocytes are preferably matured in vitro before the oocyte cells may be used as recipient cells for nuclear transfer.

Preferably, successful pig embryo cloning uses the metaphase II stage oocyte as the recipient oocyte because it is believed that at this stage of maturation the oocyte can be or is sufficiently activated to treat the introduced nucleus as if it were a fertilising sperm. However, the present invention relates to any maturation stage of the oocyte which is suitable for carrying out somatic cell nuclear transfer, embryos, blastocysts, and/or transgenic pigs obtainable by the method of somatic cell nuclear transfer of the present invention.

The in vitro maturation of oocytes usually takes place in a maturation medium until the oocyte has reached the metaphase II stage or has extruded the first polar body. The time it takes for an immature oocyte to reach maturation is called the maturation period.

In a preferred embodiment of the present invention the oocyte is from sow or gilt, preferably from a sow.

The donor (somatic cell or nucleus of somatic cell) and recipient (cytoplast) involved in the cell nuclear transfer method according to the present invention is a pig. Likewise, reconstructed embryos may be implanted in a pig according to the present invention. The different pigs suitable as donor, recipient or foster mother are described elsewhere herein.

The donor pig according to the present invention may be female, or male. The age of the pig can be any age such as an adult, or for example a fetus.

### Embryo

According to the present invention a reconstructed embryo (i.e. single cell embryo) contains the genetic material of the donor cell. Subsequently, the reconstructed embryo divides progressively into a multi-cell embryo after the onset of mitosis. In vitro the onset of mitosis is typically induced by activation as described herein.

In the present invention the term ‘embryo’ also refers to reconstructed embryos which are embryos formed after the process of nuclear transfer after the onset of mitosis by activation. Reconstructed embryos are cultured in vitro.

When the embryo contains about 12-16 cells, it is called a “morula”. Subsequently, the embryo divides further and many cells are formed, and a fluid-filled cystic cavity within its center, blastocoele cavity. At this stage, the embryo is called a “blastocyst”. The developmental stage of the “fertilized” oocyte at the time it is ready to implant; formed from the morula and consists of an inner cell mass, an internal cavity, and an outer layer of cells called trophectodermal cells.

The blastocyst according to the present invention may be implanted into the uterus of a host mammal, in particular a pig, preferably a Goettingen minipig, and continues to grow into a fetus and then an animal.

In the methods provided herein for producing genetically modified or transgenic non-human mammal, for cloning a non-human mammal, for culturing a reconstructed embryo, and/or for cryopreservation of a pig embryo, the embryo may be cultured in vitro. The embryo may for example be cultured in sequential culture. It will be appreciated that the embryo may be a normal embryo, or a reconstructed embryo as defined elsewhere herein.

The present invention thus relates to a modified porcine embryo, blastocyst and/or fetus derived from the genetically modified pig model as disclosed herein and/or the modified porcine embryo comprising at least one mutation in the endogenous ILK-1Ra, JunB/cJun, CD18, IKK2, and/or LIG1 gene or part thereof, transcriptional and/or translational product or part thereof, and/or a modified pig comprising at least one human, porcine and/or murine PPARs, PPAR-δ, IκB-α, STAT3c, Integrin beta 1, Integrin alpha 2, MEK1, Amphiregulin, BMP-6, VEGF, JunBΔec-JunΔep, IL-1a, TGF.beta 1, CD18 hypo, Cre-IIKK2 fl7fl, Dsg1, SCCE, TGF-a, TNF-a, IL-20, IFN-g, LIG1 KO, KGF, IL-6, PAFR, Cre/Ikk2FL/FL, IL1R, Dsg3, IFN-gamma, p40, IL1Ra, IKK2, JunB/c-Jun, or LIG1 gene or part thereof, transcriptional and/or translational product or part thereof.

It is appreciated that the modified porcine embryo, blastocyst and/or fetus derivable from the modified pig model for studying psoriasis, expressing at least one phenotype associated with psoriasis may have been the result of the crossing of a pig transgenic for any of the genetic determinants for psoriasis as defined herein, in particular a pig comprising at least one human or porcine PPAR gene or part thereof and/or a pig comprising at least one modified Integrin gene or part thereof.

### Cytoplast

An oocyte or a part of an oocyte from which the nucleus has been removed.

### Donor Cell

By the term ‘donor cell’ of the present invention is meant somatic cell and/or cells derived from the germ line.

By the term ‘somatic cell’ of the present invention is meant any (body) cell from an animal at any stage of development. For example somatic cells may originate from fetal, neonatal or adult tissue. Especially preferred somatic cells are those of foetal or, neonatal origin. However, cells from a germ line may also be used. According to the present invention a donor cell is a somatic cell. In another embodiment of the present invention the donor cell is a cell derived from a germ cell line.

In a preferred embodiment of the present invention the donor cell harbours desired genetic properties. However, the donor cell may harbour desired genetic properties which have been gained by genetic manipulation as described elsewhere herein.

Somatic cells are selected from the group consisting of epithelial cells, neural cells, epidermal cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, and other muscle cells.

These may be obtained from different organs, e.g., skin, lung, pancreas, liver, stomach, intestine, heart, reproductive organs, bladder, kidney, urethra and other urinary organs.

The pigs from which the somatic cells may be derived are described elsewhere herein. A preferred embodiment of the invention is the use of somatic cells originating from the same species as the recipient oocyte (cytoplast).

Preferably, the somatic cells are fibroblast cells as the can be obtained from both developing foetuses, newborn piglets and adult animals in large quantities. Fibroblasts may furthermore be easily propagated in vitro. Most preferably, the somatic cells are in vitro cultured fibroblasts of foetal or neonatal origin.

In a preferred embodiment the somatic cells are genetically modified. In yet a further preferred embodiment of the present invention the somatic cells are preferably of foetal or neonatal origin, or for example from adults.

One aspect of the present invention relates to a modified porcine donor cell and/or cell nucleus derived from the modified pig model as disclosed herein and/or a modified porcine donor cell and/or cell nucleus comprising at least one mutation in the endogenous ILK-1Ra, JunB/cJun, CD18, IKK2, and/or LIG1 gene or part thereof, transcriptional and/or translational product or part thereof, and/or a modified porcine donor cell and/or cell nucleus comprising at least one human, porcine and/or murine PPARs, PPAR-δ, I□B-□, STAT3c, Integrin beta 1, Integrin alpha 2, MEK1, Amphiregulin, BMP-6, VEGF, JunBΔec-JunΔep, IL-1a, TGF.beta 1, CD18 hypo, Cre-IIKK2 fl7fl, Dsg1, SCCE, TGF-a, TNF-a, IL-20, IFN-g, LIG1 KO, KGF, IL-6, PAFR, Cre/Ikk2FL/FL, IL1R, Dsg3, IFN-gamma, p40, IL1Ra, IKK2, JunB/c-Jun, and/or LIG1 gene or part thereof, transcriptional and/or translational product or part thereof. It is appreciated that the modified donor cell may be any type of tissue as described elsewhere herein, however, the preferred donor cell is a porcine fibroblast cell.

It is appreciated that the modified porcine donor cell or cell nucleus derivable from the modified pig model for studying psoriasis, expressing at least one phenotype associated with psoriasis may have been the result of the crossing of a pig transgenic for any of the genetic determinants for psoriasis as defined herein, in particular a pig comprising at least one human or porcine PPAR gene or part thereof and/or a pig comprising at least one modified Integrin gene or part thereof.

### Type of Genetic Modification

The donor cells may be genetically modified by any of standard method known in the art. The genetic modification may be a modification of the genomic DNA by deletion, insertion, duplication and/or other forms of mutation, including point mutation. The modification may be made in coding sequences and/or non-coding sequences. DNA constructs for insertion may harbour a gene of interest and/or regulatory sequences such as promoters, insulators, enhancers, repressors or ribosomal entry sites. In some embodiments, only one genetic modification is introduced in the genome. In other embodiments, however, the genome may be modified at more than one site. Suitable techniques for genetic modification of mammalian cells, such as fibroblasts, include techniques such as gene addition by nonhomologous recombination, gene replacement by homologous recombination, and gene editing. This may include the use of retroviral insertion, transposon transfer and/or artificial chromosome techniques. Nonhomologous DNA recombination may e.g. be carried out as described in Kragh et al. (2004) Reprod. Fert. Dev. 16:290 or Kragh et al. (2004) Reprod. Fert. Dev. 16:315, Transposon-based gene transfer may be carried out as described in Izsvak et al. (1997) Cell 91:501. Gene replacement by homologous recombination may e.g. involve the techniques described by Urnow et al. (2005) Nature 435:646. Techniques for gene editing have been described in Andersen et al. (2002) J. Mol. Med. 80:770, Liu et al (2002) Gene Ther. 9:118 and Sørensen et al. (2005) J. Mol. Med. 83:39.

In a preferred embodiment the donor cell is genetically modified by random integration of the genes disclosed herein into the genome of the donor cell.

In another preferred embodiment of the present invention the donor cell is genetically modified (as described in a copending application). The donor cell or nucleus carries a SB tagged genome containing a Flp recombination target site for site specific gene insertion or integration. The SB tagged genome result from the integration of a recombinant target vector comprising a DNA transposon construct and a bicistronic gene cassette comprising (i) a FRT recombination site and (ii) an IRES-driven selection gene. The DNA transposon construct may be any construct in which any DNA transposon is present. In the present invention the DNA transposon construct is the Sleeping Beauty (SB) DNA transposon vector. The FRT recombination site may be embedded in the coding sequence of a selection gene which allows for detecting whether a transposition has occurred. The selection gene of the present invention is not limited to any particular selection gene. In preferred embodiments the selection gene are genes conferring resistance to antibiotics or drugs, such as puromycin, tetracycline, streptomycin or hygromycin resistance genes, or the enhanced green fluorescent protein (eGFP) gene, red fluorescent protein genes or the like. The FRT recombination site may thus be embedded in a SV40 promoter driven fusion variant of the selection gene. However, any promoter suitable for conferring expression of a selection gene may be used according to the present invention. Non-limiting examples of such promoters are CMV (cytomegalovirus) or PGK promoter.

The IRES-driven selection gene is similarly not limited to any particular selection gene. In preferred embodiments the selection gene are genes conferring resistance to antibiotics or drugs, such as puromycin, tetracycline, streptomycin or hygromycin resistance genes, or the enhanced green fluorescent protein (eGFP) gene, red fluorescent protein genes or the like.

The recombinant vector construct may also comprise at least one site for Cre recombinase. The at least one site for Cre recombinase may be located as disclosed in the examples herein.

The donor cell or nucleus may also originate from a genetically modified pig comprising at least one site for integration of at least one transgene. A preferred embodiment is a donor cell or nucleus in the form of a fibroblast, such as a primary fibroblast.

The present invention also relates to a method for producing a porcine cell comprising a SB tagged genome containing a Flp recombination target site for site-specific gene insertion. The method comprises the steps of

a) providing a mammalian cell, b) transfecting the cell of a) with a plasmid expressing a transposase and a recombinant target vector comprising a DNA transposon construct and a bicistronic gene cassette comprising (i) a FRT recombination site and ii) an IRES-driven selection gene, c) selecting SB tagged cells.

As described elsewhere herein the mammalian cell may be any cell. In one embodiment in which the porcine cell is subsequently to be used for producing a genetically modified pig by nuclear transfer according to the hand-made protocol as described herein, the porcine cell is in a preferred embodiment a fibroblast and most preferred a porcine primary fibroblast.

It is appreciated that a desired transgene may be integrated directly into the at least one site for integration present in the genome of the cell. However, the cell in which the genome carries the at least one site for integration is in another embodiment used as a donor cell for the production of a genetically modified pig by for example microinjection of the donor cell or nucleus thereof into a oocyte or by for example somatic nuclear transfer. In a preferred embodiment the donor cell or the nucleus thereof is used for the production of a genetically modified pig by somatic nuclear transfer using the procedure as described elsewhere herein.

The transgene or gene of interest to be integrated in the targeted cells of the present invention is not limited to any particular gene. In one embodiment the gene to be integrated is a disease-causing gene which results in the formation of a genetically modified pig displaying a phenotype of interest. According to the present invention the gene of interest to be integrated into the porcine cell is PPAR-δ and IκB-α.

The integration of the transgene into the at least one site for integration present in the genome of the cell is employed by transfection into the cell of plasmid DNA containing the gene of interest and also FRT sites, and a plasmid expressing the Flp-recombinase used to support integration at the FRT sites.

### Enucleation

The method of enucleation of an oocyte may be selected from the group of methods consisting of aspiration, physical removal, use of DNA-specific fluorochromes, exposure to ultraviolet light and/or chemically assisted enucleation. In one embodiment the present invention relates to the use of DNA-specific fluorochromes.

Enucleation may, however, be performed by exposure with ultraviolet light. In a particular embodiment enucleation is chemically assisted prior to physical removal of the nucleus. Chemically assisted enucleation using for example antineoplastic agents, such as demecolcine (N-deacetyl-N-methyl 1 colchicine), and/or for example etoposide or related agents may be performed prior to enzymatic modification of zona pellucida.

Chemically assisted enucleation comprises culturing matured COCs in maturation medium as described elsewhere herein supplemented with demecolcine for a particular period of time. In the range of 0.1 μg/ml to 10 μg/ml demecolcine, such as 0.2 μg/ml to 10 μg/ml, for example 0.3 μg/ml to 10 μg/ml, such as 0.25 μg/ml to 5 μg/ml, for example 0.3 μg/ml to 1 μg/ml, such as 0.25 μg/ml to 0.5 μg/ml, for example 0.4 μg/ml demecolcin may be supplemented to the maturation medium. Similarly, maturation medium may be supplemented with etoposide for example in the range of 0.1 μg/ml to 10 μg/ml etoposide, such as 0.2 μg/ml to 10 μg/ml, for example 0.3 μg/ml to 10 μg/ml, such as 0.25 μg/ml to 5 μg/ml, for example 0.3 μg/ml to 1 μg/ml, such as 0.25 μg/ml to 0.5 μg/ml, for example 0.4 μg/ml etoposide may be supplemented to the maturation medium. The time for culturing the COCs in the presence of antineoplastic agents ranges from 10 min to 5 hrs, such as 30 minutes to 5 hrs, for example 10 minutes to 2 hrs, such as 30 min to 2 hrs, for example 10 min to 1.5 hrs, such as 20 min to 3 hrs, for example 10 min to 3 hrs, such as 30 min to 1.5 hrs, for example 45 min. In a particular embodiment chemically assisted enucleation is performed using 0.45 μg/ml demecolcine and/or etoposide added to the maturation medium for 45 min.

In a particular embodiment it is preferred that the enucleation is by physical removal of the nucleus. The physical removal may be by separation for example by bisection of the oocyte into two halves (two parts), one which contains the nucleus and the enucleated oocyte half, known as the cytoplast, removing the nucleated half of the oocyte and selecting the resulting cytoplast for further procedures of the invention. Alternatively the separation is by trisection, resulting in three parts of which two parts are cytoplasts. In another embodiment the oocyte may be separated into four parts, resulting in the production of three cytoplasts. The oocyte may even be separated into five parts by physical removal, resulting in four cytoplasts. Similarly, the oocyte may be separated into six parts, for example seven parts, such as eight parts, for example nine parts, such as ten or more parts.

The physical separation of the oocyte and subsequent removal of the nucleus-bearing part of the oocyte may be achieved by the use of a microsurgical blade. The oocytes may be screened to identify which oocytes have been successfully enucleated. Oocyte parts that harbour nuclear DNA may be identified by staining with Hoechst fluorochrome, the staining procedure of which is known to a person skilled in the art. Oocyte parts harbouring nuclear DNA are discarded and the enucleated oocytes (cytoplasts) are selected for further procedures.

### Zona Pellucida

Zona pellucida is a thick, transparent, noncellular layer or envelope of uniform thickness surrounding an oocyte

Generally, an intact zona pellucida is considered to be important in cell nuclear transfer due to a number of parameters. One parameter is to keep the polar body close to the metaphase plate of the oocyte in order to indicate the appropriate site for enucleation. Another parameter relates to the keeping of the donor cell close to the oocyte cytoplast before and during fusion. The zona is also believed to confer protection for the donor cell and cytoplast during fusion. Finally, embryo development after reconstitution and activation is believed to be supported by the zona pellucida.

Modification of at least a part of the zona pellucida can be performed by a number of methods. For example physical manipulation can be used to modify the zona. But also chemical treatment with agents such as acidic solutions (acidic Tyrode) can be employed. One example of chemical agents that can be employed in the present invention is acidic solutions, for example Tyrode. In a particular embodiment of the invention the zona pellucida is modified by enzymatic digestion. Such enzymatic digestion may be performed by enzymes comprising for example trypsin. Alternatively a specific protease may be used, such as pronase.

In a preferred embodiment the enzymatic digestion results in at least a partial digestion of a part of zona pellucida which in a preferred embodiment of the present invention means that at least a part of the zona pellucida is being removed, or that the zona pellucida is partly removed. In the present context the zona pellucida is not completely removed.

According to an especially preferred embodiment of the present invention the partially digested part of zona pellucida is characterized by the zona pellucida still being visible and by the fact that the oocyte has not become misshaped.

The partial digestion may be achieved by exposure to a protease. In another embodiment of the present invention the partial digestion may be accomplished by the use of a pronase. In yet another embodiment the partial digestion may be achieved by a combination of a protease and pronase.

In a preferred embodiment the concentration of pronase is in the range of 0.1 mg/ml to 10 mg/ml, such as 0.5 mg/ml to 10 mg/ml, for example 1 mg/ml to 10 mg/ml, such as 1.5 mg/ml to 10 mg/ml, for example 2 mg/ml to 10 mg/ml, such as 2.5 mg/ml to 10 mg/ml, for example 2.75 mg/ml to 10 mg/ml, such as 3 mg/ml to 10 mg/ml, for example 3.25 mg/ml to 10 mg/ml, such as 3.3 mg/ml to 10 mg/ml, for example 3.5 mg/ml to 10 mg/ml.

A preferred embodiment is a pronase concentration in the range of 2 mg/ml to 5 mg/ml, such as 2.25 mg/ml to 5 mg/ml, for example 2.5 mg/ml to 5 mg/ml, such as 2.75 mg/ml to 5 mg/ml, for example 2.8 mg/ml to 5 mg/ml, such as 2.9 mg/ml to 5 mg/ml, for example 3 mg/ml to 5 mg/ml, such as 3.1 mg/ml to 5 mg/ml, for example 3.2 mg/ml to 5 mg/ml, such as 3.3 mg/ml to 5 mg/ml.

A particular embodiment of the present invention is a pronase concentration in the range of 1 mg/ml to 4 mg/ml, for example 1 mg/ml to 3.9 mg/ml, such as 1 mg/ml to 3.8 mg/ml, for example 1 mg/ml to 3.7 mg/ml, such as 1 mg/ml to 3.6 mg/ml, for example 1 mg/ml to 3.5 mg/ml such as 1 mg/ml to 3.4 mg/ml, for example 1 mg/ml to 3.3 mg/ml.

In a preferred embodiment the pronase concentration is in the range of 2.5 mg/ml to 3.5 mg/ml, such as 2.75 mg/ml to 3.5 mg/ml, for example 3 mg/ml to 3.5 mg/ml. In a special embodiment the pronase concentration is 3.3 mg/ml.

It is clear to the skilled person that the pronase should be dissolved in an appropriate medium, one preferred medium according to the present invention is T33 (Hepes buffered TCM 199 medium containing 33% cattle serum (as described earlier—Vajta, et al., 2003).

The time of incubation of the oocyte in the pronase solution is in the range of 1 second to 30 seconds, such as 2 seconds to 30 seconds, for example 3 seconds to 30 seconds, such as 4 seconds to 30 seconds, such as 5 seconds to 30 seconds.

In another embodiment of the present invention the incubation time is in the range of 2 seconds to 15 seconds, such as 2 seconds to 14 seconds, for example 2 seconds to 13 seconds, such as 2 seconds to 12 seconds, for example 2 seconds to 11 seconds, such as 2 seconds to 10 seconds, for example 2 seconds to 9 seconds, such as 2 seconds to 8 seconds, for example 2 seconds to 7 seconds, such as 2 seconds to 6 seconds, for example 2 seconds to 5 seconds.

In a particular embodiment of the present invention the incubation time is in the range of 3 seconds to 10 seconds, such as 3 seconds to 9 seconds, for example 4 seconds to 10 seconds, such as 3 seconds to 8 seconds, for example 4 seconds to 9 seconds, such as 3 seconds to 7 seconds, for example 4 seconds to 8 seconds, such as 3 seconds to 6 seconds, for example 4 seconds to 7 seconds, such as 3 seconds to 5 seconds, for example 4 seconds to 6 seconds, such as 4 seconds to 5 seconds. An especially preferred incubation time is 5 seconds.

In a preferred embodiment of the present invention the oocyte is treated for 5 seconds in a 3.3 mg/ml pronase solution at 39° C.

### Reconstructed Embryo

By the term ‘reconstructed embryo’ is meant the cell which is formed by insertion of the donor cell or nucleus of the donor cell into the enucleated oocyte which corresponds to a zygote (during normal fertilisation). However, the term ‘reconstructed embryo’ is also referred to as the ‘reconstituted cell’. In the present invention the donor cell is a somatic cell. However, the donor cell may also be derived from a germ line cell.

### Fusion

The transfer of a donor cell or a membrane surrounded nucleus from a donor cell to at least cytoplast is according to the present invention performed by fusion. In the scenarios described below the term ‘donor cell’ also refers to a membrane surrounded nucleus from a donor cell. Fusion may be achieved by a number of methods.

Fusion may be between a donor cell and at least one cytoplast, such as between a donor cell and at least two cytoplasts, for example between a donor cell and at least two cytoplasts, such as between a donor cell and at least three cytoplasts, such as between a donor cell and at least four cytoplasts, for example between a donor cell and at least five cytoplasts, such as between a donor cell and at least six cytoplasts, for example between a donor cell and at least seven cytoplasts, such as between a donor cell and at least eight cytoplasts.

Fusion may be performed according to the listed combinations above simultaneously or sequentially. In one embodiment of the present invention the fusion is performed simultaneously. In another embodiment fusion of the at least one cytoplast and a donor cell is performed sequentially.

For example fusion may be achieved by chemical fusion, wherein a donor cell and the at least one cytoplast are exposed to fusion promoting agents such as for example proteins, glycoproteins, or carbohydrates, or a combination thereof. A variety of fusion-promoting agents are known for example, polyethylene glycol (PEG), trypsin, dimethylsulfoxide (DMSO), lectins, agglutinin, viruses, and Sendai virus. Preferably phytohemaglutinin (PHA) is used. However mannitol and, or polyvinylalcohol may be used.

Alternatively, fusion may be accomplished by induction with a direct current (DC) across the fusion plane. Often an alternating current (AC) is employed to align the donor and recipient cell. Electrofusion produces a sufficiently high pulse of electricity which is transiently able to break down the membranes of the cytoplast and the donor cell and to reform the membranes subsequently. As a result small channels will open between the donor cell and the recipient cell. In cases where the membranes of the donor cell and the recipient cell connect the small channels will gradually increase and eventually the two cells will fuse to one cell.

Alignment of the at least one cytoplast and the donor cell may be performed using alternating current in the range of 0.06 to 0.5 KV/cm, such as 0.1 to 0.4 KV/cm, for example 0.15 to 0.3 KV/cm. In a preferred embodiment alignment of the at least one cytoplast and the donor cell may be performed using alternating current at 0.2 KV/cm.

Fusion may be induced by the application of direct current across the fusion plane of the at least one cytoplast and the donor cell. Direct current in the range of 0.5 to 5 KV/cm, such as 0.75 to 5 KV/cm, for example 1 to 5 KV/cm, such as 1.5 to 5 KV/cm, for example 2 to 5 KV/cm. Another preferred embodiment of the present invention is the application of direct current in the range of 0.5 to 2 KV/cm. In a further preferred embodiment the direct current may be 2 KV/cm.

The direct current may preferably be applied for in the range of 1-15 micro seconds, such as 5 to 15 micro seconds, for example 5 to 10 micro seconds. A particular embodiment may be 9 micro seconds.

In an especially preferred embodiment fusion with direct current may be using a direct current of 2 KV/cm for 9 micro seconds.

Electrofusion and chemical fusion may however be also be combined.

Typically electrofusion is performed in fusion chambers as known to the skilled person.

Fusion may be performed in at least one step, such as in two steps, for example three steps, such as in four steps, for example in five steps, such as six steps, for example seven steps, such as in eight steps.

Fusion may be performed in for example a first step wherein the at least one cytoplast is fused to the donor cell. A second step of fusion may comprise fusion of the fused pair (cytoplast-donor cell, reconstructed embryo) with at least one cytoplast, such as at least two cytoplasts, for example three cytoplasts, such as four cytoplasts, for example five cytoplasts, such as six cytoplasts, for example seven cytoplasts, such as eight cytoplasts. The second step of fusion with fusion of at least one cytoplast and the fused pair may be performed sequentially or simultaneously. In one embodiment the at least two cytoplasts are fused to the fused pair simultaneously. In another embodiment the at least two cytoplasts are fused to the fused pair sequentially.

In one embodiment of the invention the second step of fusion may also be an activation step wherein the reconstructed embryo is activated to enter mitosis. As described elsewhere herein.

### Activation

In a preferred embodiment the reconstructed embryo may be allowed to rest prior to activation for a period of time in order to allow for the nucleus of the donor cell to reset its genome and gain toti potency in the novel surroundings of the enucleated cytoplast. The reconstructed embryo may for example rest for one hour prior to activation.

Preferably, the reconstructed embryo may be activated in order to induce mitosis. Methods for activation may preferably be selected from the group of consisting of electric pulse, chemically induced shock, increasing intracellular levels of divalent cations or reducing phosphorylation. A combination of methods may be preferred for activation.

In one particular embodiment of the invention the activation and the second step of fusion may be performed simultaneously. However, the activation of the reconstituted embryo and the at least one additional step of fusion between the reconstructed embryo and the at least one cytoplast may be performed sequentially.

Reducing the phosphorylation of cellular proteins in the reconstructed embryo by known methods such as for example by the addition of kinase inhibitors may activate the reconstituted embryo. A preferred embodiment may involve the use of agents that inhibit protein synthesis, for example cycloheximide. A further preferred embodiment may be using agents that inhibit spindle body formation, for example cytochalasin B.

In one embodiment of the invention the intracellular levels of divalent cations may be increased. Divalent cations such as for example calcium may be in comprised in the activation medium. Preferably, the cations may enter the reconstructed embryo, particularly upon subjecting the reconstructed embryo to an electric pulse. In a preferred embodiment the electric pulse may cause entering of calcium into the reconstructed embryo.

The application of an electrical pulse using direct current may be an activation step. However, in a preferred embodiment the electrical pulse applied for activation may also serve as an additional fusion step.

Prior to applying an electrical pulse using direct current the at least one cytoplast and the at least one reconstructed embryo may be aligned by the application of alternating current. The alternating current may be in the range of the range of 0.06 to 0.5 KV/cm, such as 0.1 to 0.4 KV/cm, for example 0.15 to 0.3 KV/cm. In a preferred embodiment alignment of the at least one cytoplast and the donor cell may be performed using alternating current at 0.2 KV/cm.

Activation may be induced by the application of direct current across the fusion plane of the at least one cytoplast and the donor cell. Direct current in the range of 0.2 to 5 KV/cm, such as 0.4 to 5 KV/cm, for example 0.5 to 5 KV/cm. Another preferred embodiment of the present invention is the application of direct current in the range of 0.5 to 2 KV/cm. In a further preferred embodiment the direct current may be 0.7 KV/cm.

The direct current may preferably be applied for in the range of 10 to 200 micro seconds, such as 25 to 150 micro seconds, for example 50 to 100 micro seconds. A particular embodiment may be 80 micro seconds.

In an especially preferred embodiment fusion with direct current may be using a direct current of 0.7 KV/cm for 80 micro seconds.

An especially preferred embodiment of activation according to the present invention may be use of an electrical pulse in combination with subjecting the reconstructed embryo to agents that inhibit protein synthesis, spindle body formation, and divalent cations.

Activation may be performed by any combination of the methods described above.

### In Vitro Culture of Embryos

One aspect of the invention relates to a method of in vitro culturing embryos, whereby the blastocyst rate increased to 25.3%. Thus, a method of culturing a reconstructed embryo is within the scope of the present invention, comprising the steps of a) establishing at least one oocyte having at least a part of zona pellucida, b) separating the oocyte into at least two parts obtaining an oocyte having a nucleus and at least one cytoplast, c) establishing a donor cell or cell nucleus having desired genetic properties, d) fusing at least one cytoplast with the donor cell or membrane surrounded cell nucleus, e) obtaining the reconstructed embryo, f) activating the reconstructed embryo to form an embryo, and e) culturing said embryo.

Another aspect of the invention relates to a method of cell nuclear transfer in which a step of culturing the embryo is included.

In a preferred embodiment in relation to the methods described herein embryos are cultured in vitro in a sequential set of media. Preferably the blastocysts are grown in traditional medium such as for example NCSU37 or equivalent medium as known to a person skilled in the art, wherein glucose is removed and substituted by other agents. One agent may be pyruvate. Another agent may be lactate. The agents may also be combined and replace glucose in the traditional medium.

The embryos may be cultured in the substituted media as described above from Day 0 to Day 3, such as from Day 0 to Day 2.

The pyruvate concentration may range from 0.05 to 1 mM, such as 0.1 to 1 mM, for example 0.125 to 1 mM, such as 0.15 to 1 mM. However the concentration of sodium pyruvate may also range from 0.05 mM to 0.9 mM, such as 0.05 to 0.8 mM, for example 0.05 to 0.7 mM, such as 0.05 to 0.6 mM, for example 0.05 to 0.5 mM, such as 0.05 to 0.4 mM, for example 0.05 to 0.3 mM, such as 0.05 to 0.2 mM. Preferably the concentration ranges between 0.05 to 0.17 mM. A preferred concentration of sodium pyruvate is 0.17 mM.

The lactate concentration may range from 0.5 to 10 mM, such as 0.75 to 10 mM, for example 1 to 10 mM, such as 1.5 to 10 mM, such as 1.75 to 10 mM, for example 2 to 10 mM, such as 2.5 to 10 mM. However the concentration of sodium lactate may also range from 0.5 mM to 9 mM, such as 0.5 to 8 mM, for example 0.5 to 7 mM, such as 0.5 to 6 mM, for example 0.5 to 5 mM, such as 0.5 to 4 mM, for example 0.5 to 03 mM. Preferably the concentration ranges between 1 to 5 mM, such as 2 to 4 mM, for example 2 to 3 mM. A preferred concentration of sodium lactate is 2.73 mM.

After the initial glucose-free incubation medium glucose is again replacing the pyruvate and lactate. The embryos may be cultured in the glucose containing medium from Day 4 to Day 3, preferably from Day 3 to Day 7. The glucose concentration may range from 1 to 10 mM, such as 2 to 10 mM, for example 3 to 10 mM, such as 4 to 10 mM, for example 5 to 10 mM. However, the glucose concentration may also range from 1 to 9 mM, such as 2 to 8 mM, for example 3 to 7 mM, such as 4-6 mM. A preferred concentration of glucose according to the present invention is 5.5 mM of glucose.

### Organ or Tissue Donation

In one embodiment, the animals of the invention may be used as a source for organ or tissue donation for humans or other animals, either animals of the same species or animal of other species. Transfer between species is usually termed xenotransplantation. Entire organs that may be transplanted include the heart, kidney, liver, pancreas or lung. Alternatively, parts of organs, such as specific organ tissues may be transplanted or transferred to humans or other animals. In a yet further embodiment, an individual cell or a population of individual cells from an animal of the invention may be transferred to a human being or another animal for therapeutic purposes.

### Cryopreservation

The term ‘cryopreserving’ as used herein can refer to vitrification of an oocyte, cytoplast, a cell, embryo, or pig of the invention. The temperatures employed for cryopreservation is preferably lower than −80 degree C., and more preferably at temperatures lower than −196 degree C. Oocytes, cells and embryos of the invention can be cryopreserved for an indefinite amount of time. It is known that biological materials can be cryopreserved for more than fifty years.

It is within the scope of the present invention that embryos may be cryopreserved prior to transfer to a host pig when employing methods for producing a genetically engineered or transgenic non-human mammal. Such cryopreservation prior to transfer may be at the blastocyst stage the of embryo development. Vitrification is a form of cryopreservation where living cells are rapidly cooled so that the fluid of the cell does not form into ice. Thus, vitrification relates to the process of cooling where cells or whole tissues are preserved by cooling to low sub-zero temperatures, such as (typically) −80 C or −196 C

In particular the invention relates to the vitrification of an oocyte, however, the invention also relates to the vitrification of embryos, preferably embryos at the blastocyst stage.I one embodiment, the embryo is cultured to blastocyst stage prior to vitrification. Especially pig embryos are covered by the present invention. Also vitrified cytoplasts are covered by the present invention, as are cells.

Yet another aspect of the invention relates to the cryopreservation of a pig embryo derived by a method for cell nuclear transfer as described herein comprising a step of vitrifying a pig embryo. A further aspect of the invention relates to pig embryos obtained, or obtainable by the methods provided herein.

### Mitochondria

Cells of the tissue of the genetically modified non-human mammals and/or non-human embryos obtainable by the present invention may harbour mitochondria of different maternal sources. In a preferred embodiment the non-human mammals and/or non-human embryos may harbour mitochondria from only one maternal source, However, in another preferred embodiment the non-human mammals and/or non-human embryos may harbour mitochondria from at least two maternal sources, such as three maternal sources, for example four maternal sources, such as five maternal sources, for example six maternal sources, such as seven maternal sources, for example eight maternal sources, such as nine maternal sources, for example ten maternal sources. The probability of having a specific number of maternal sources can be calculated based on the observed types of mitochondria.

### Evaluation of Treatment

The treatment offered to a patient suffering from psoriasis varies due to the fact that the effectiveness of a certain type of treatment varies from one patient to another. The treatment offered depends on the type of psoriasis, the location, extent and severity. If a patient is receiving treatment for diseases other than psoriasis in addition to treatment of psoriasis the influence of the treatment for diseases other than psoriasis is considered when deciding on the treatment for psoriasis.

In general, the first step for treating psoriasis is topical treatment, mediated ointments or creams applied to the skin. Such topical treatment includes as an active ingredient coal tar, dithranol (anthralin), corticosteroids, vitamin D₃ analogues such as calcipotriol, and retinoids. A typical next step if the first step is unsuccessful is the exposure of the skin to ultraviolet radiation also known as phototherapy. Phototherapy is in some cases combined with topical (coal tar, calcipotriol) or systemic treatment (retinoids) as a synergy in their combination has been observed. A third step is systemic treatment involving orally administered or injected medication. The traditional choice of medicaments in systemic treatments are immunosupressant drugs methotrexate and ciclosporin, and retinoids, which are synthetic forms of vitamin A. Other additional drugs, not specifically licensed for psoriasis, have been found to be effective. These include the antimetabolite tioguanine, the cytotoxic agent hydroxyurea, sulfasalazine, the immunosupressants mycophenolate mofetil, azathioprine and oral tacrolimus. The type of treatment of a given patient may be varied over time in order to avoid resistance to the treatment and also to reduce the risk of adverse reactions.

The present invention offers a method for screening the efficacy of a pharmaceutical composition, wherein the method comprises the steps of i) providing the pig model of the present invention, ii) expressing in said pig model the genetic determinant and exerting said phenotype for said disease, iii) administering to the pig model a pharmaceutical composition the efficacy of which is to be evaluated, and iv) evaluating the effect, if any, of the pharmaceutical composition on the phenotype exerted by the genetic determinant when expressed in the pig model.

Furthermore, within the scope of the present invention is a method for evaluating the response of a therapeutical treatment of psoriasis, wherein the method comprises the steps of i) providing the pig model of the present invention, ii) treating said pig model with a pharmaceutical composition exerting an effect on said phenotype, and iii) evaluating the effect observed. Based on the evaluation one could further advise on the treatment based on the observed effects.

In addition, the present invention relates to a method for treatment of a human being suffering from psoriasis, wherein the method comprises the initial steps of i) providing the pig model of the present invention, ii) expressing in said pig model said genetic determinant and exerting said phenotype for said disease, iii) administering to said pig model a pharmaceutical composition the efficacy of which is to be evaluated, and v) evaluating the effect observed, and v) treating said human being suffering from psoriasis based on the effects observed in the pig model.

It is therefore appreciated that the pig model according to the present invention may also receive medicaments for diseases other than psoriasis in order to test the combined effect of a drug for psoriasis and other drugs administered to the pig.

### EXAMPLES

### Genes and Promoter-Transgene Constructs for Integration into the Transgenic Porcine Fibroblast Cell

### Abnormal Epidermal Proliferation and Differentiation Characterize the Inflammatory Skin Disease Psoriasis.

Psoriatic human epidermis is unbalanced with respect to the gene regulators PPAR-δ and NFκB. Down-regulating NFκB by expression of a dominant negative variant of IκB-α and up-regulating PPAR-δ is obtained by integrating said genes into a tagged fibroblast cell comprising integration sites as described elsewhere herein. Thus, pig epidermal tissue with psoriatic-like dysregulation can be studied in the pig model of the present invention.

Similarly, the promoter-transgene constructs K5-STAT3c (Sano et al Nat Immunol 2005), Involucrine—Integrin beta 1 (Carol) et al Cell 1995), Involucrine—Integrin alpha 2(Carrol et al Cell 1995), Involucrine—MEK1(Hobbs et al J Invest derm 2004), K14—Amphiregulin (Cook et al J Clin Invest 1997), K10—BMP-6 (Blessing et al J Cell biol 1996; Kaiser et al J Invest Dermatol 1998), K14—VEGF (Kunstfeldt et al Blood 2004, Xia et al Blood 2003), K5—JunBΔec-JunΔep (Zenz et al 2005), K14—IL-1a (Groves et al J Clin Invest 1996; Groves et al PNAS1995), K5—TGF.beta 1 (Li et al Derm Symp Proc 2005; Li et al EMBO 2004), CD18 hypo (Bullard et al PNAS 1996; Barlow et al Am J pathol 2003), K14—Cre-IIKK2 fl7 μl (Pasparakis et al Nature 2002), K1—Dsg1 or K1—Dsg3 (Merrit et al Mol Cell Biol 2002), SCCE (Ny et al Act Derm Venerol 2004), K14—TGF-a (Vassar et al Genes devel 1991), K14—TNF-a (Genes Dev. 1992 August; 6(8):1444-56), K14—IL-20 (Blumberg et al Cell 2001), Involucrine—IFN-gamma (Carroll et al J Invest dermatol 1997), LIG1 KO (Suzuki et al FEBS 2002), K14—KGF (Guo et al EMBO 1993), K14—IL-6 (Turksen et al PNAS1992), PAFR (sato et al Arch Dermatol Res 1999), K14—Cre/Ikk2FL/FL, K14—p40 (Kopp et al, J Invest Dermatol. 2001 September; 117(3):618-26), K14—Tie2 (Voskas et al, Am J. Pathol. 2005 March; 166(3):843-55), K14—IL-1 Ra (Shepherd et al, J Invest Dermatol. 2004 March; 122(3):665-9), K14—IKK2 (M. Pasparakis et al., Nature 417(6891), 2002, pp. 861-866), or K14—LIG-1 (Y. Suzuki et al., FEBS Lett. 521(1-3), 2002, pp. 67-71) are integrated into the fibroblast cell line carrying in its genome integration sites as described herein.

### Establishing a Transgenic Porcine Fibroblast Cell

Based on the well-described mechanisms of SB transposition (4-8) and Flp recombination (9, 10), the present invention discloses a new target vector for site-specific integration into the genome. This vector carries within the context of a SB transposon vector a bicistronic gene cassette containing (i) the FRT recombination site embedded in the coding sequence of eGFP and (ii) an IRES-driven puromycin resistance gene. We demonstrate efficient selective plasmid insertion into SB-tagged genomic loci. In an attempt to further improve the performance of these vectors, we have analyzed the effect of insulator elements, believed to protect inserted foreign genes against transcriptional silencing, within the context of SB vectors. Our investigations indicate that insulators flanking the FRT gene expression cassette may serve to maintain and stabilize gene expression of Flp-inserted transgenes.

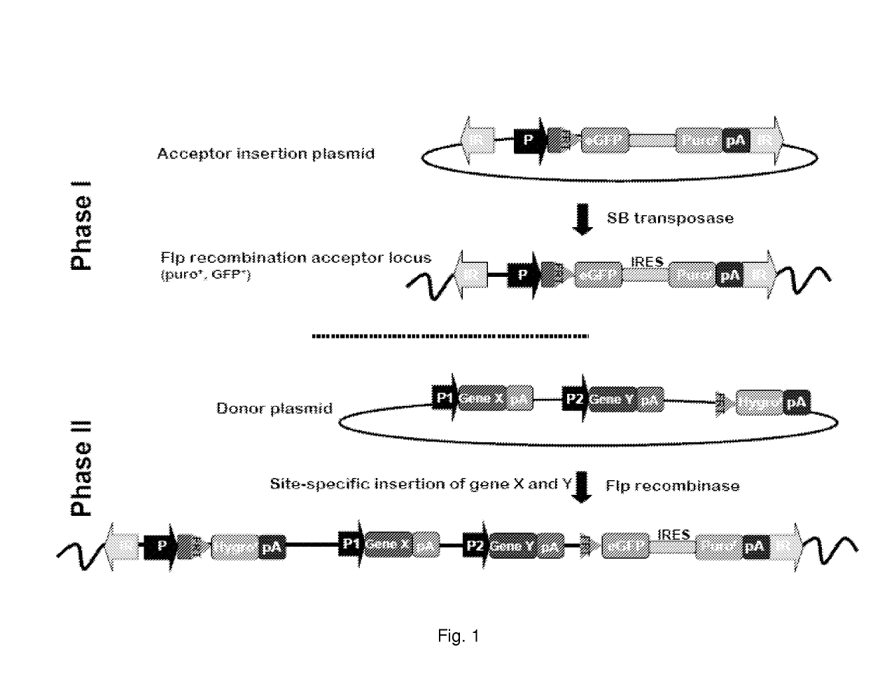

Two nonviral integration technologies are employed in the present invention, the SB transposon system and the Flp recombinase, in a combined effort to achieve active locus detection, mediated by SB, and site-directed insertion at an attractive site, mediated by Flp. A bi-phased technology is disclosed in which an integrating SB vector, carrying a reporter gene and a selective marker gene, may first serve as a reporter for continuous gene expression and hence as a target for gene insertion (FIG. 1). By using an actively integrated vector as opposed to plasmid DNA that is randomly recombined into the genome we certify (i) that only a single copy, and not concatemers, of the vector are inserted and, moreover, (ii) that the reporter cassette is not flanked by sequences derived from the bacterial plasmid backbone which may have a detrimental effect on the locus activity over time. In a second modification step this vector may serve as a target for insertion of one or more gene expression cassettes in a well-characterized locus.

### Vector Construction

The SB transposon-based vector used in this study was derived from the pSBT/SV40-GFIP.loxP vector. This vector contains, within the context of a SB transposon, a bicistronic FRTeGFP-IRES-puro (GFIP) cassette flanked upstream by an ATG start codon and downstream by a poly A sequence. Moreover, the vector contains a recognition site for the Cre recombinase (loxP) located between the upper inverted repeat of the vector and the SV40 promoter driving expression of the FRTeGFP-IRES-puro cassette.

Construction of pSBT/SV40-GFIP.loxP Vector

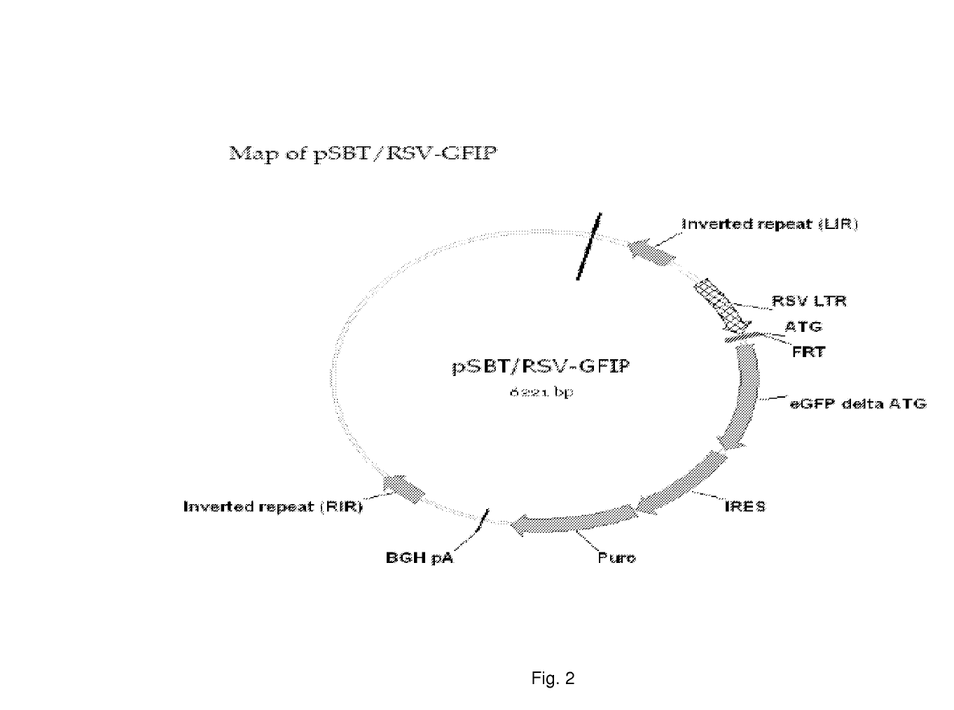

The pSBT/RSV-GFIP vector contains the terminal inverted of the SB DNA transposon flanking a FRT-GFP.IRES.puro bicistronic gene cassette driven by a promotor derived from Rous sarcoma virus (RSV). The eGFP sequence was amplified from peGFP.N1 (Clontech) using a forward primer containing the 48-bp FRT sequence. To analyze FRT-GFP functionality, the FRT-eGFP fusion was inserted into an expression vector containing the SV40 promoter. The PCR-fragment containing FRT-tagged eGFP fusion gene was digested with Mlul and Xmal and inserted into Mlul/Xmal-digested pSBT/RSV-hAAT (pT/hAAT in ref. (8), obtained from Mark Kay, Stanford University, USA), generating a transposon vector with RSV-driven eGFP expression (pSBT/RSV-eGFP). An IRES-puro cassette was PCR-amplified from pecoenv-IRES-puro (provided by Finn Skou Pedersen, University of Aarhus, Denmark), digested with Xmal, and inserted into Xmal-digested pSBT/RSV-eGFP, generating pSBT/RSV-GFIP (see FIG. 2). Alternative versions of this vector containing the SV40 promoter (pSBT/SV40-GFIP) and the promoter derived from the human ubiquitin gene (pSBT/Ubi-GFIP), were generated. In addition, by inserting a Cre recombination target site (loxP) into the MluI site located between the left inverted repeat of the transposon and the SV40 promoter of pSBT/SV40-GFIP, the vector pSBT/SV40-GFIP.loxP was created. The donor plasmid pcDNA5/FRT, containing a FRT-hygro fusion gene without a start codon, was obtained from Invitrogen. The Flp-encoding plasmid, pCMV-Flp was obtained from A. Francis Stewart, University of California San Francisco, USA). This plasmid encodes the enhanced Flp variant designated Flpx9 (11). A SB-vector containing two copies of the 1.2-kb chicken DNase hypersensitive site 4 (cHS4)-derived insulator element (12, 13) was generated by inserting PCR-amplified cHS4 sequences and an intervening linker into NotI/SpeI-digested pSBT/PGK-puro (obtained from Mark Kay, Stanford University, USA). The PGK-puro cassette was cloned back into construct by using restiction sites located in the linker, generating pSBT/cHS4.PGK-puro.cHS4

For further use in pigs an alternative Cre recognition site (loxP-257) was inserted into a unique AscI site that was created by mutagenesis at a position located between the poly A sequence and the lower inverted repeat of the vector. This vector was designated pSBT/loxP.SV40-GFIP.loxP257. The presence of two Cre recombination sites allows Cre recombinase-mediated cassette exchange after Flp-based plasmid insertion, thereby facilitating, if needed, removal of plasmid sequences and selection genes.

### SB Transposition in Primary Pig Fibroblasts

The SB transposon vectors, either SBT/PGK-puro or the target transposon SBT/loxP.RSV-GFIP.loxP257, were inserted into the genome of pig fibroblast by co-transfecting (using Fugene-6 from Roche) 1.5 μg pSBT/lox.RSV-GFIP.loxP257 (or pSBT/PGK-puro) with 1.5 μg pCMV-SB (or 1.5 μg pCMV-mSB as a negative control). pCMV-SB (rights held by Perry Hackett, University of Minnesota, Minnesota, USA) encodes the Sleeping Beauty transposase reconstructed from fossil DNA transposable elements of salmoid fish. pCMV-SB, pCMV-mSB, and the hyperactive variant pCMV-HSB3 were obtained from Mark Kay, Stanford University, USA. SB-tagged cell clones appeared as a result of selecting transfected cells with puromycin (0.5 μg/ml). Colonies were fixed and stained in methylene blue in methanol and subsequently counted.

### Solid SB Transposition in Primary Pig Fibroblasts

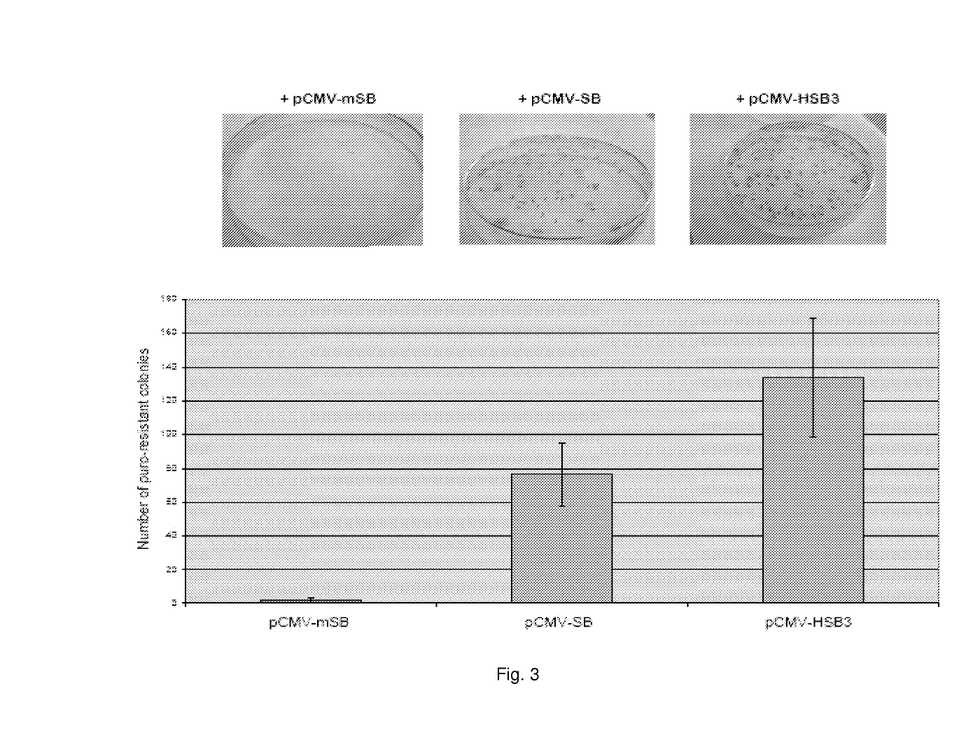

SB transposes efficiently in most mammal cells but with higher efficacy in human cells than in murine cells. Transposition of SB vectors has never been analyzed in porcine cells, and we therefore initially tested activity in primary pig fibroblasts. We utilized a standard transposon encoding a puromycin resistance gene (SBT/PGK-puro) and found decent levels of transposition, resulting in about 75 drug-resistant colonies in cultures of fibroblasts co-transfected with pSBT/PGK-puro and pCMV-SB (FIG. 3). Less than 3 colonies appeared after transfection with pSBT/PGK-puro and pCMV-mSB, the latter which encodes an inactive version of the transposase. Interestingly, a mean of almost 140 colonies was obtained using the hyperactive transposase variant HSB3, indicating that HSB3 also in porcine cells mediates higher levels of transposition compared to the original SB transposase.

### Efficient Insertion of a FRT-Tagged SB Vector in Pig Fibroblasts

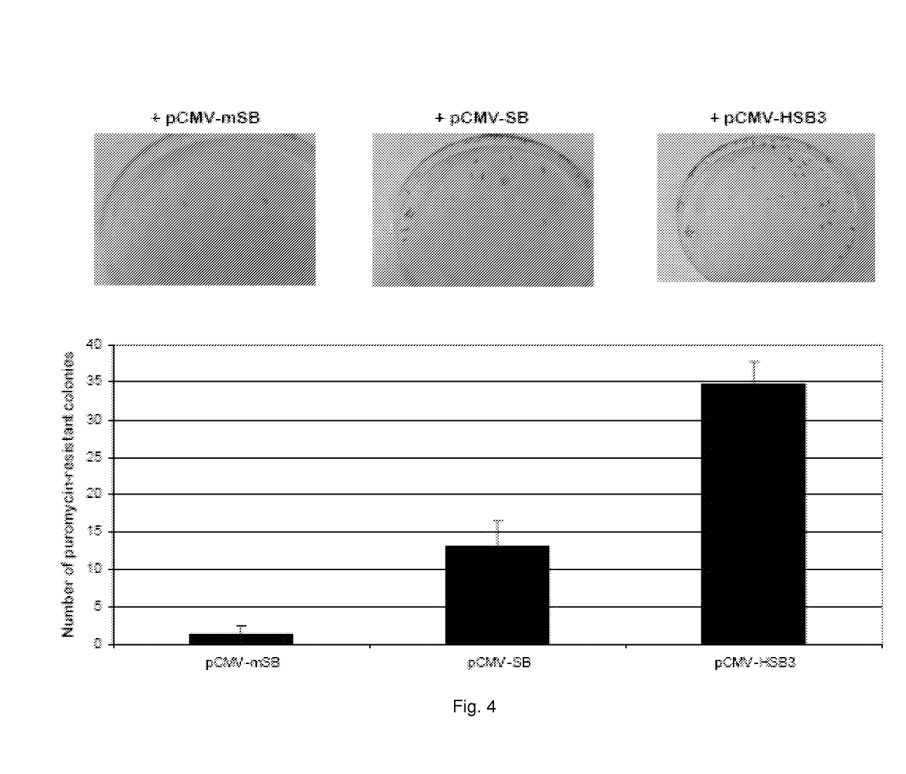

To generate SB-tagged cell clones containing a Flp recombination target site for site-specific gene insertion, we co-transfected the pSBT/loxP.SV40-lopP257 plasmid with pCMV-mSB, pCMV-SB, and pCMV-HSB3, respectively. HSB3 again showed the highest activity, resulting in about 30 drug-resistant colonies after transfection of 3H 10⁴ fibroblasts (FIG. 4).

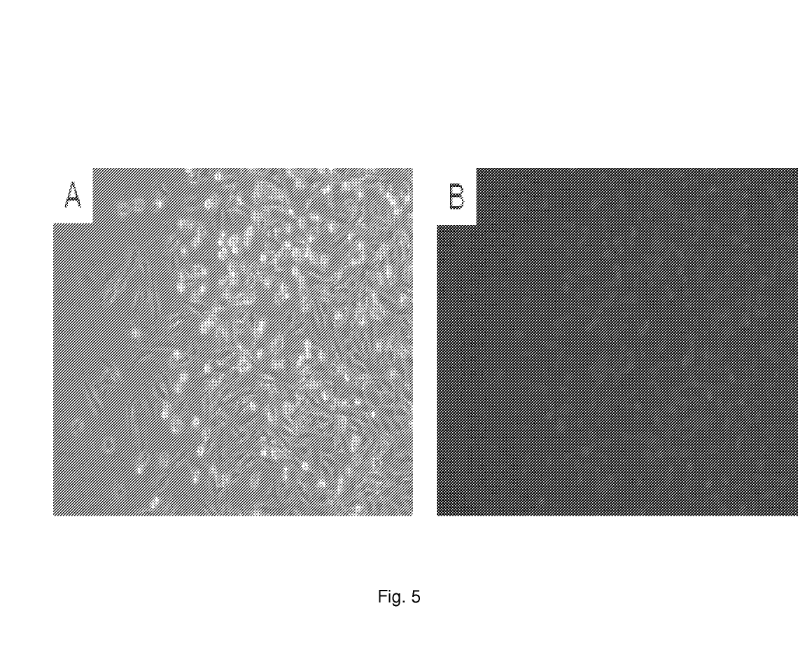

Puromycin-resistant colonies were isolated and expanded. Clone analysis by fluorescence microscopy demonstrated efficient FRTeGFP expression (FIG. 5), demonstrating vector functionality and easy FRTeGFP detection in pig fibroblasts. These fluorescent cell clones carrying the Flp FRT recombination sequence are currently being used for creation of cloned transgenic animals by hand-made cloning.

Verification of SBT/loxP.SV40-GFIP.loxP257 as Target for Flp Recombination

Due to limitations of long-term growth of primary pig fibroblasts in tissue culture we were not able to demonstrate Flp-based gene insertion into FRT-tagged SB vectors in pig fibroblasts. We therefore chose to test functionality of the FRT-containing vector by a standard set of recombination experiments carried out in HEK-293 cells. We generated clones of HEK-293 cells containing the transposed SBT/loxP.SV40-GFIP.loxP257 vector. By co-transfection of such clones with (i) a pcDNA5/FRT-derived substrate plasmid containing a FRT-hygro fusion gene and a red fluorescent protein (RFP) expression cassette and (ii) a plasmid encoding the Flp recombinase (pCMV-Flpx9), we subsequently identified hygromycin B resistant colonies. By fluorescence microscopy we observed that site-specifically engineered clones, as expected, turned-off eGFP expression and turned-on RFP expression (data not shown). This ‘green-to-red’ phenotypic change indicates that the integrated SB-derived target vector serves as acceptor site for Flp-based recombination.

In conclusion, the Sleeping Beauty DNA transposon-based vector of the present invention serves in its integrated form as a target for recombinase-based gene insertion. The SB vector is efficiently transferred by cut-and-paste transposition into the genome of primary porcine fibroblasts and therefore is not flanked by plasmid-derived bacterial sequences. Use of these genetically engineered primary cells in for example microinjection and hand-made cloning allows subsequent detailed analyses of SB vector-derived eGFP expression in cloned pigs and identification of animals with attractive expression profiles (e.g. ubiquitous, tissue-specific). Primary fibroblasts from such ‘master pigs’ is further modified by Flp-based recombination, allowing site-directed gene insertion in a SB vector-tagged locus which is not silenced in the tissue of interest. Cloned pigs harboring a site-specifically inserted disease gene of interest or a shRNA expression cassette for downregulation of endogenous genes can be generated by a second round of animal cloning.

### Further Examples

Except where otherwise indicated all chemicals were obtained from Sigma Chemical Co. (St Louis, Mo., USA).

### Oocyte Collection and In Vitro Maturation (IVM)

Cumulus-oocyte complexes (COCs) were aspirated from 2-6 mm follicles from slaughterhouse-derived sow or gilt ovaries. COCs were matured in groups of 50 in 400 μl bicarbonate-buffered TCM-199 (GIBCO BRL) supplemented with 10% (v/v) cattle serum (CS), 10% (v/v) pig follicular fluid, 10 IU/ml eCG, 5 IU/ml hCG (Suigonan Vet; Skovlunde, Denmark) at 38.5° C. in the “Submarine Incubation System” (SIS; Vajta, et al. 1997) in 5% CO₂ in humidified air for 41-44 hours.

### In Vitro Fertilization (IVF)

IVF experiments were performed with in vitro matured oocytes in 3 identical replicates. After maturation, COCs were washed twice with mTBM containing 2 mM caffeine (mTBM_{fert}) and transferred in groups of 50 to 400 μl mTBM_{fert}. Freshly ejaculated semen was treated as described previously (Booth, et al., in press). After 2 h capacitation at 38.5° C. and in 5% CO₂ in humidified air, sperm was added to the oocytes with the adjusted final concentration of 1×10⁵ sperm/ml. Fertilization was performed at 38.5° C. and in 5% CO₂ in humidified air in the SIS for 3 h. After the insemination, the presumptive zygotes were vortexed in mTBM_{fert} to remove cumulus cells before washing in IVC medium and placing in culture dishes (see Embryo culture and evaluation).

### Handmade Cloning (HMC)

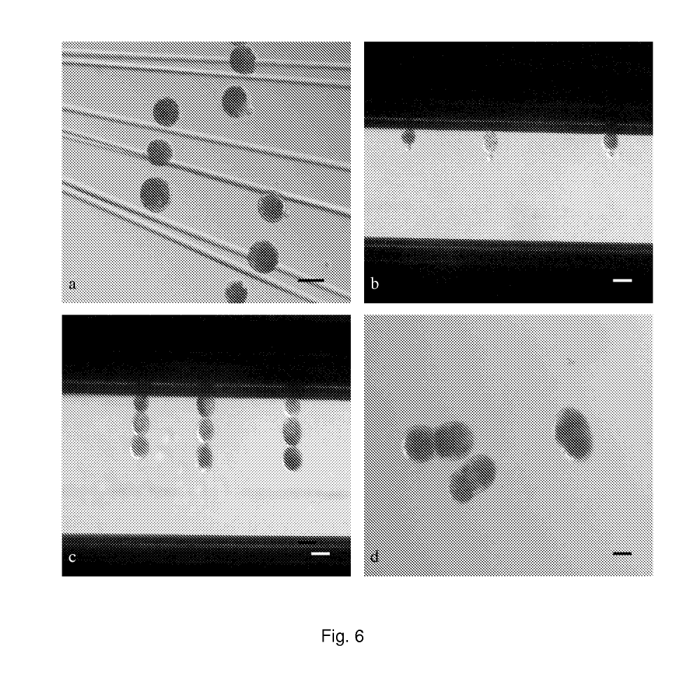

The applied HMC method was based on our previous work in cattle and pig (Kragh, et al., 2004; Peura and Vajta, 2003; Vajta, et al., 2003), but with significant modifications. Briefly, at 41 h after the start of maturation, the cumulus investment of the COCs was removed by repeated pipetting in 1 mg/ml hyaluronidase in Hepes-buffered TCM199. From this point (except where otherwise indicated), all manipulations were performed on a heated stage adjusted to 39° C., and all drops used for handling oocytes were of 20 μl volume covered with mineral oil. Oocytes were briefly incubated in 3.3 mg/ml pronase dissolved in T33 (T for Hepes-buffered TCM 199 medium; the number means percentage (v/v) of CS supplement, here 33%) for 5 s. Before the oocytes started to become misshaped in pronase solution, they were picked out and washed quickly in T2 and T20 drops. Oocytes with partially digested but still visible zona were lined up in drops of T2 supplemented with 3 mg/ml polyvinyl alcohol (TPVA) and 2.5 μg/ml cytochalasin B. Trisection instead of bisection was performed manually under stereomicroscopic control with Ultra Sharp Splitting Blades (AB Technology, Pullman, Wash., USA; FIG. 6a). Fragments of trisected oocytes were collected and stained with 5 μg/ml Hoechst 33342 fluorochrome in TPVA drops for 5 min, then placed into 1 μl drops of the TPVA medium on the bottom of a 60 mm Falcon Petri dish covered with oil (3-4 fragments per drop). Using an inverted microscope and UV light, positions of fragments without chromatin staining (cytoplasts) were registered and later collected under a stereomicroscope in T10 drops until the start of the fusion.

Fetal fibroblast cells were prepared as described previously (Kragh, et al., in press). Fusion was performed in two steps where the second one included the initiation of activation, as well. For the first step, one third of the selected cytoplasts (preferably the smaller parts) were used. With a finely drawn and fire-polished glass pipette, 10 cytoplasts were transferred as a group to 1 mg/ml of phytohaemagglutinin (PHA; ICN Pharmaceuticals, Australia) for 3 s, then quickly dropped onto one of the few fibroblast cells individually that were sedimented in a T2 drop. After attachment, 10 cytoplast-fibroblast cell pairs were equilibrated in fusion medium (0.3 M mannitol and 0.01% PVA) for 10 s. Using an alternative current (AC) of 0.6 KV/cm and 700 KHz, cell pairs were aligned to the wire of a fusion chamber (BTX microslide 0.5 mm fusion chamber, model 450; BTX, SanDiego, Calif., USA) with the donor cells farthest from the wire (FIG. 6b), then fused with a direct current (DC) of 2.0 KV/cm for 9 μs. After the electrical pulse, cell pairs were removed carefully from the wire, transferred to T10 drops and incubated to observe whether fusion had occurred.

Approximately 1 hour after the first fusion, fused pairs together with the remaining two thirds of cytoplasts were equilibrated in activation medium drops separately (0.3 M mannitol, 0.1 mM MgSO₄, 0.1 mM CaCl₂ and 0.01% polyvinylalcohol (PVA)). Under a 0.6 KV/cm AC, cytoplast—fused pair—cytoplast triplets were aligned sequentially to the wire in groups of 10, with fused pairs located in the middle (FIG. 6c). A single DC pulse of 0.7 KV/cm for 80 μs was used for the second fusion and initiation of activation. The triplets were then removed from the wire and transferred carefully to T10 drops to check the fusion (FIG. 6d). Reconstructed embryos were incubated in culture medium (see Embryo culture and evaluation) supplemented with 5 μg/ml cytochalasin B and 10 μg/ml cycloheximide for 4 h at 38.5° C. in 5% O₂, 5% O₂ and 90% N₂ with maximum humidity, then washed thoroughly for 3 times in IVC medium before culture.

### Parthenogenetic Activation (PA)

Parthenogenetically activated oocytes were produced either separately or in parallel with HMC. Oocytes were denuded in the same way as above except that a longer incubation in pronase was used to get the zona pellucida completely removed. Zona free (ZF) oocytes were then equilibrated for 10 s in activation medium (0.3 M mannitol, 0.1 mM MgSO₄, 0.1 mM CaCl₂ and 0.01% PVA) and transferred to the fusion chamber (BTX microslide 0.5 mm fusion chamber, model 450; BTX, SanDiego, Calif., USA). A single DC pulse of 0.85 KV/cm for 80 μs was generated with a BLS CF-150/B cell fusion machine (BLS, Budapest, Hungary) and applied to ZF oocytes. For zona intact (ZI) oocytes, a single DC pulse of 1.25 KV/cm for 80 μs was used (according to our unpublished preliminary experiments, these parameters resulted in the highest activation and subsequent in vitro development for ZI and ZF oocytes, respectively). The procedure after the electrical pulse was the same as for HMC reconstructed embryos.

### Embryo Culture and Evaluation

All porcine embryos produced by the above treatments were cultured in a modified NCSU37 medium (Kikuchi, et al., 2002) containing 4 mg/ml BSA at 38.5° C. in 5% O₂, 5% CO₂ and 90% N₂ with maximum humidity. The culture medium was supplied with 0.17 mm sodium pyruvate and 2.73 mm sodium lactate from Day 0 (the day for fertilization and activation) to Day 2, then sodium lactate and sodium pyruvate was replaced with 5.5 mm glucose from Day 2 to Day 7. All ZF embryos were cultured in the WOW system (Vajta, et al., 2000) in the same culture medium and gas mixture as used above, with careful medium change on Day 2 without removing the embryos from the WOWs. The blastocyst rate was registered on Day 7. To determine total cell numbers, blastocysts were fixed and mounted to a glass microscopic slide in glycerol containing 20 μg/μl Hoechst 33342 fluorochrome. After staining for 24 h, embryos were observed under a Diaphot 200 inverted microscope with epifluorescent attachment and UV-2A filter (Nikon, Tokyo, Japan).

### Example 1

Differences in developmental competence between sow (2.5 years, 170 Kg in weight) derived oocytes and gilt (5.5˜6 months, 75 Kg in weight) derived oocytes were investigated through ZF and ZI PA after 44 h in vitro maturation. Four combined groups were investigated in 3 identical replicates: (1) ZF oocytes from sows (2) ZI oocytes from sows (3) ZF oocytes from gilts (4) ZI oocytes from gilts. For ZF activation, a single DC pulse of 0.85 KV/cm for 80 μs was applied, while a single 1.25 KV/cm pulse was used to activate ZI oocytes. Following 7 days culture as described above, the percentage of blastocysts per activated embryo was determined.

The in vitro developmental competence of parthenogenetically activated oocytes derived from either sows or gilts was investigated. As shown in Table 1, the blastocyst rates of parthenogenetically activated oocytes from sows were significantly higher than those from gilts, either after ZF or ZI PA.

TABLE 1
Blastocyst development of Day 7 parthenogenetically
activated sow and gilt oocytes

| | |
|---|---|
| Zona Free | Zona Intact |

| | No. of | No. of | No. of | No. of |
|---|---|---|---|---|
| | activated | blastocysts | activated | blastocysts |
| | oocytes | (%)* | oocytes | (%)* |
| sow | 103 | 43(42 ± 4)^{a} | 110 | 61(55 ± 6)^{c} |
| gilt | 85 | 17(20 ± 2)^{b} | 137 | 36(26 ± 5)^{d} |

^{a,b}Different superscripts mean significant differences (p < 0.05).
^{c.d}Different superscripts mean significant differences (p < 0.05).
*Percentage (Mean ± S.E.M) of embryos developed to blastocysts.

The difference in oocytes developmental competence between sows and gilts has been examined in in vitro production (IVP) and somatic cell nuclear transfer (SCNT) embryos separately, resulting in a similar conclusion as in the earlier publication of other research groups (Sherrer, et al., 2004; Hyun, et al., 2003), i.e. that embryos from sow-derived oocytes are superior to those from gilt-derived oocytes in supporting blastocyst development. Although gilts used in our study were at the borderline of maturity, the difference between Day 7 blastocyst rates after PA was significant, proving the superior developmental competence of sow oocytes.

### Example 2

The feasibility of modified porcine HMC was investigated in 6 identical replicates, with IVF and in parallel ZF PA as controls. The more competent sow oocytes (according to Example 1) were used in Example 2. Seven days after reconstruction and/or activation, the number of blastocysts per reconstructed embryo and total cell numbers of randomly selected blastocysts were determined.

More than 90% of oocyte fragments derived from morphologically intact oocytes could be recovered for HMC after the trisection. In average, 37 embryos could be reconstructed out of 100 matured oocytes. The developmental competence of all sources of porcine embryos is shown in Table 2. On Day 7, the development of reconstructed embryos to the blastocyst stage was 17±4% with mean cell number of 46±5, while the blastocyst rates for IVF, and ZF PA were 30±6% and 47±4% (n=243, 170, 97) respectively.

TABLE 2
In vitro development of embryos
produced by HMC, IVF and ZF PA

| | No. of | | blastocyst | Mean cell |
|---|---|---|---|---|
| Embryo | embryos/oocytes | No. of | rates (Mean ± | number of |
| origins | in culture | blastocysts | S.E.M). | blastocysts |
| HMC | 243 | 41 | 17 ± 4^{a} | 46 ± 5^{d} |
| IVF | 170 | 52 | 30 ± 6^{b} | 74 ± 6^{e} |
| ZF PA | 97 | 46 | 47 ± 4^{c} | 53 ± 7^{d} |

^{a,b,c}Different superscripts mean significant differences (p < 0.05).
^{d,e}Different superscripts mean significant differences (p < 0.05).

Although the theoretical maximum efficiency was still not approached, the integration of zona partial digestion and oocyte trisection almost doubled the number of reconstructed embryos compared to our earlier system (Kragh, et al., 2004 Reprod. Fertil. Dev 16, 315-318). This increase in reconstruction efficiency may have special benefits in porcine cloning since oocyte recovery after aspiration is more demanding and time-consuming than in cattle. An even more important point is the high embryo number required for establishment of pregnancies following porcine nuclear transfer. IVC in pigs is also regarded as a demanding and inefficient procedure (Reed, et al., 1992 Theriogeneology 37, 95-109). A disadvantage of ZF systems is that the embryos have to reach at least the compacted morula or early blastocyst stage in vitro to avoid disintegration in the oviduct without the protective layer of the zona pellucida. On the other hand, once in the blastocyst stage, zona free embryos can be transferred successfully as proved by calves born after either embryonic or somatic cell nuclear transfer (Peura et al., 1998; Tecirlioglu et al., 2004; Oback et al., 2003; Vajta, et al., 2004) and also by the piglets born after zona-free IVP of oocytes (Wu, et al., 2004). NCSU37 medium has been the most widely and successfully used medium for the culture of pig embryos. However, despite the improved embryo development compared with other media, the viability of IVP porcine embryos is still compromised after IVC.

Some reports suggested that glucose is not metabolized readily by early porcine embryos before the eight-cell stage but used in higher amounts in embryos between the compacted morula and blastocysts stages (Flood, et al., 1988). The replacement of glucose with pyruvate and lactate in NCSU37 for the first 2 days culture resulted in a blastocyst rate of 25.3% for IVP porcine embryos in Kikuchi's study (Kukuchi, et al., 2002), which was further corroborated by our present studies with an IVP blastocysts rate of 30% in average. Moreover, the first evaluation of this sequential culture system on porcine HMC and ZF PA embryos has resulted in blastocyst rates of 17% and 47% respectively. Sometimes, the blastocyst rate of ZI PA could even reach levels as high as 90% (Du, unpublished)

### Statistical Analysis

ANOVA analysis was performed using SPSS 11.0. A probability of P<0.05 was considered to be statistically significant.

### Example 3

### Vitrification of Hand-Made Cloned Porcine Blastocysts Produced from Delipated In Vitro Matured Oocytes

Recently a noninvasive procedure was published for delipation of porcine embryos with centrifugation but without subsequent micromanipulation (Esaki et al. 2004 Biol Reprod. 71, 432-6).

Cryopreservation of embryos/blastocysts was carried out by vitrification using Cryotop (Kitazato Supply Co, Fujinomiya Japan) as described previously (Kuwayama et al. 2005a; 2005b). At the time of vitrification, embryos/blastocysts were transferred into equilibration solution (ES) consisting of 7.5% (V/V) ethylene glycol (EG) and 7.5% dimethylsulfoxide (DMSO) in TCM199 supplemented with 20% synthetic serum substitute (SSS) at 39° C. for 5 to 15 min. After an initial shrinkage, embryos regained their original volume. 4-6 embryos/blastocysts were transferred into 20 ul drop of vitrification solution (VS) consisting of 15% (V/V) EG and 15% (DMSO) and 0.5M sucrose dissolved in TCM199 supplemented with 20% SSS. After incubation for 20 s, embryos were loaded on Cryotop and plunged into liquid nitrogen. The process from exposure in VS to plunging was completed with 1 min.

Embryos/blastocysts were thawed by immersing Cryotop directly into thawing solution (TS) consisting of 1.0M sucrose in TCM199 plus 20% SSS for 1 min, then transferred to dilution solution (DS) consisting of 0.5 M sucrose in TCM199 plus 20% SSS for 3 min. To remove cryoprotectant, embryos/blastocysts were kept twice in a washing solution (WS; TCM199 plus 20% SSS), 5 min for each time. Survival of vitrified blastocysts was determined according to reexpansion rates after 24 h recovery in culture medium supplemented with 10% calf serum (CS).

The non-invasive delipation method was applied to in vitro matured porcine oocytes and further development of delipated oocytes after parthenogenetic activation was investigated in 4 identical replicates. Oocytes were randomly separated into delipation and control groups.

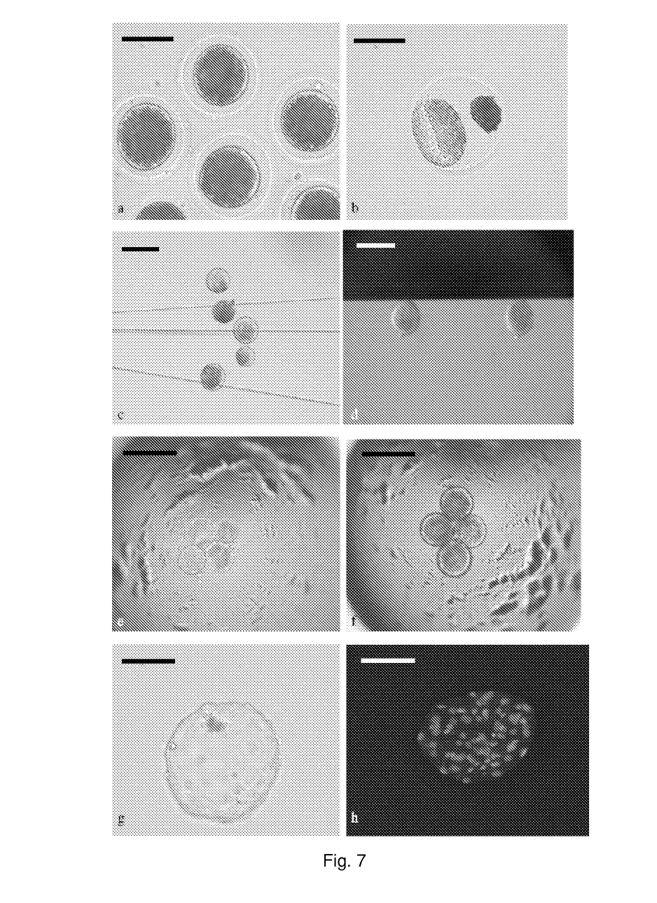

For delipation, oocytes were digested with 1 mg/ml pronase in the presence of 50% cattle serum (CS) for 3 min, and washed in Hepes-buffered TCM-199 medium supplemented with 20% CS which results in partial zona pellucida digestion (FIG. 7a). Subsequently 40-50 oocytes were centrifuged (12000×g, 20 min) at room temperature in Hepes-buffered TCM-199 medium supplemented with 2% CS, 3 mg/ml PVA and 7.5 μg/ml cytochalasin B (CB) (FIG. 7b). Zonae pellucidea of both centrifuged and intact oocytes were removed completely with further digestion in 2 mg/ml pronase solution. For activation, a single direct current of 85 Kv/cm for 80 us was applied to both groups, followed by 4 h treatment with 5 μg/ml CB and 10 μg/ml cycloheximide (CHX). All embryos were then cultured in the modified NCSU37 medium. Day 7 blastocysts were vitrified and warmed by using the Cryotop technique (Kuwayama et al., RBM Online, in press) at 38.5° C. Survival of vitrified blastocysts was determined according to reexpansion rates after 24 h recovery in culture medium supplemented with 10% CS. Cell numbers of reexpanded blastocysts from both groups were determined after Hoechst staining. Results were compared by ANOVA analysis. Partial zona digestion and centrifugation resulted in successful delipation in 173/192 (90%) of oocytes. The development to blastocysts was not different between delipated and intact oocytes (28±7% vs. 28±5% respectively; P>0.05). However, survival rates of blastocysts derived from delipated oocytes were significantly higher than those developed from intact oocytes (85±6% vs. 32±7% respectively; P<0.01). There is no difference in average cell number of reexpanded blastocysts derived from either delipated or intact oocytes (36±7 vs. 38±9, respectively; P>0.05). The results demonstrate that the simple delipation technique does not hamper the in vitro development competence of activated porcine oocytes, and improves the cryosurvival of the derived blastocysts without significant loss in cell number.

After delipation, zona pellucida of oocytes from both groups was removed completely. The same parameters as described above for electrical activation were applied to both groups. Seven days after activation, blastocyst rates and blastocyst cell numbers were determined.

The feasibility of applying a non-invasive delipation technique to in vitro matured porcine oocytes was investigated. 90% (173/192) oocytes can be delipated successfully. As shown in table 3, the development to blastocysts was not different between delipated and intact oocytes (28±7% vs. 28±5% respectively; P>0.05). However, survival rates of blastocysts derived from delipated oocytes were significantly higher than those developed from intact oocytes (85±6% vs. 32±7% respectively; P<0.01). There is no difference in average cell number of reexpanded blastocysts derived from either delipated or intact oocytes (36±7 vs. 38±9, respectively; P>0.05).

TABLE 3
Developmental competence and cryosurvival of vitrified-thawed
embryos from delipated and intact activated oocytes.

| | | | Reexpanded | Mean cell number |
|---|---|---|---|---|
| Oocyte | Activated | Blastocyst | blastocyst after | of reexpanded |
| treatment | oocyte | rate (%) | warming (%) | blastocysts |
| Delipated | 173 | 28 ± 7 | 85 ± 6 | 36 ± 7 |
| Intact | 156 | 28 ± 5 | 32 ± 7 | 39 ± 9 |

### Handmade Cloning of Delipated Oocytes

Delipated oocytes were used for HMC in 5 replicates. Four identical replicates of non-delipated oocytes for HMC were used as a control system. Seven days after reconstruction, blastocysts produced from both groups were vitrified with Cryotop. Survival rates and cell numbers of re-expanded blastocysts were determined as described for the blastocysts produced by PA.

Except where otherwise indicated, all manipulations were performed on a heated stage adjusted to 39° C., and all drops used for handling oocytes were of 20 μl volume covered with mineral oil. For somatic cell nuclear transfer, the handmade cloning (HMC) described in our previous work (Du, et al., 2005) was applied with a single modification: for enucleation of both delipated and control oocytes, bisection instead of trisection was applied.

Briefly, after the removal of cumulus investment, control oocytes were incubated in 3.3 mg/ml pronase dissolved in T33 for 10 s. Before the oocytes started to become misshaped in pronase solution, they were picked out and washed quickly in T2 and T20 drops. Delipated oocytes after centrifugation were digested in the 3.3 mg/ml pronase solution for an additional 5 s.

Both control and delipated oocytes with partially digested, distended and softened zonae pellucidae were lined up in T2 drops supplemented with 2.5 μg/ml cytochalasin B. Bisection was performed manually under stereomicroscopic control (FIG. 7c) with Ultra Sharp Splitting Blades (AB Technology, Pullman, Wash., USA). Halves were collected and stained with 5 μg/ml Hoechst 33342 fluorochrome in T2 drops for 5 min, and then placed into 1 μl drops of T2 medium on the bottom of a 60 mm Falcon Petri dish covered with oil (3-4 halves per drop). Using an inverted microscope and UV light, positions of halves without chromatin staining (cytoplasts) were registered. Cytoplasts were later collected under a stereomicroscope and stored in T10 drops.

Porcine foetal fibroblast cells were prepared with trypsin digestion from monolayers as described previously (Kragh, et al., 2005). Fusion was performed in two steps where the second one included the initiation of activation, as well. For the first step, 50% of the available cytoplasts were transferred into 1 mg/ml of phytohaemagglutinin (PHA; ICN Pharmaceuticals, Australia) dissolved in TO for 3 s, then quickly dropped over single fibroblast cells. After attachment, cytoplast-fibroblast cell pairs were equilibrated in fusion medium (0.3 M mannitol and 0.01% PVA) for 10 s and transferred to the fusion chamber. Using an alternating current (AC) of 0.6 KV/cm and 700 KHz, pairs were aligned to the wire of a fusion chamber with the somatic cells farthest from the wire (FIG. 7d), then fused with a direct current of 2.0 KV/cm for 9 μs. After the electrical pulse, cell pairs were removed carefully from the wire, transferred to T10 drops and incubated to observe whether fusion had occurred.

Approximately 1 hour after the first fusion, each pair was fused with another cytoplast in activation medium. AC current and a single DC pulse of 0.7 KV/cm for 80 μs were applied as described above. Fusion was detected in T10 drops, then reconstructed embryos were transferred into IVC0-2 medium (see Embryo culture and evaluation) supplemented with 5 μg/ml cytochalasin B and 10 μg/ml cycloheximide. After a 4 h incubation at 38.5° C. in 5% O₂, 5% O₂ and 90% N₂ with maximum humidity, embryos were washed 3 times in IVC0-2 medium before culture.

TABLE 4
Developmental competence and cryosurvival of
vitrified-thawed embryos of SCNT porcine embryos
derived from delipated and intact oocytes.

| | | | | Mean cell |
|---|---|---|---|---|
| | No. of | | Reexpanded | number of |
| HMC | reconstructed | Blastocyst | blastocyst after | reexpanded |
| group | embryos | rate (%)* | warming (%)* | blastocysts* |
| Delipated | 240 | 21 ± 6^{a} | 79 ± 6^{b} | 41 ± 7^{d} |
| Intact | 150 | 23 ± 6^{a} | 32 ± 8^{c} | 39 ± 5^{d} |

Different superscripts mean significant differences (p < 0.05).
*mean ± S.E.M.

In vitro developmental competence was observed in HMC with delipated oocytes when Day 7 blastocyst rates were compared with control HMC group (21±6% vs. 23±6% respectively; P>0.05; Table 4). Cryosurvival rate after vitrification of cloned blastocysts derived from delipated oocytes was significantly higher than those developed from intact oocytes (79±6% vs. 32±8, respectively; P<0.01).

### Example 4

### Chemically Assisted Handmade Enucleation (CAHE) and Comparison to Existing Methods

After 41-42 h maturation in vitro, COCs were further cultured for 45 min in the same solution supplemented by 0.4 μg/ml demecolcine. Cumulus cells were then removed by pipetting in 1 mg/ml hyaluronidase dissolved in Hepes-buffered TCM-199. From this point (except where otherwise indicated), all manipulations were performed on a heated stage adjusted to 39° C. All drops used for handling oocytes were of 20 μl in volume, and were covered with mineral oil.

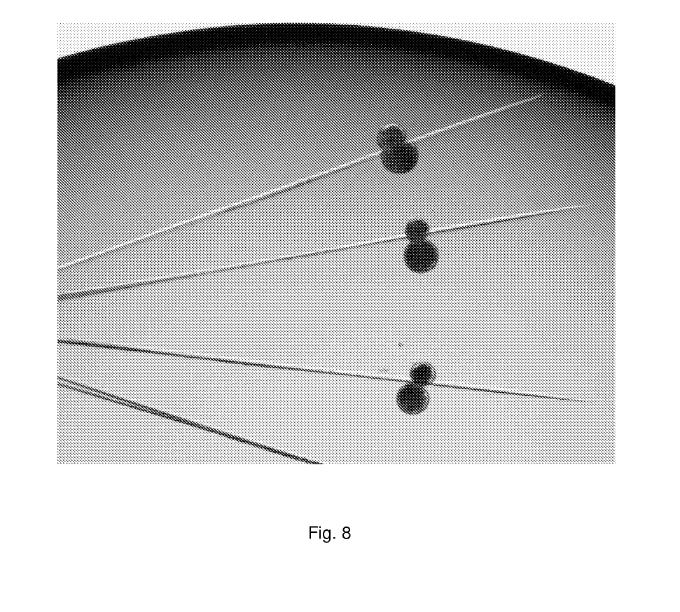

Basic steps of the HMC procedure have been described elsewhere herein. Briefly, oocytes without cumulus cells were incubated in 3.3 mg/ml pronase dissolved in T33 (T for Hepes-buffered TCM 199 medium; the number means percentage [v/v] of CS supplement, here 33%) for 20 s. When partial lyses of zonae pellucidae and slight deformation of oocytes occurred, they were picked up and washed quickly in T2 and T20 drops. Nine oocytes were lined up in one T2 drop supplemented with 2.5 μg/ml cytochalasin B (CB). By using a finely drawn and fire-polished glass pipette, oocytes were rotated to find a light extrusion cone and/or strongly attached polar body on the surface, and oriented bisection was performed manually under stereomicroscopic control with a microblade (AB Technology, Pullman, Wash., USA). Less than half of the cytoplasm (close to the extrusion or PB) was separated from the remaining part (FIG. 8). After bisection of all 9 oocytes in the drop, larger parts and smaller parts (with the extrusion or attached PB) were collected and placed into separate drops of T2, respectively.

Oriented Handmade Enucleation without Demecolcine Treatment (OHE)

All steps were similar to the previously described procedure, but demecolcine preincubation was not applied.

### Random Handmade Bisection for Enucleation (RHE)

Demecolcine preincubation was omitted from the pretreatment of this group, as well. After removal of cumulus cells, zonae pellucidae were partially digested by pronase as described above. Random handmade equal bisection was applied in drops of T2 supplemented with 2.5 μg/ml CB. All demi-oocytes were selected and stained with 10 μg/ml Hoechst 33342 in T2 drops for 10 min, then placed into 1 μl drops of T2 medium covered with mineral oil (three demi-oocytes into each drop). Using an inverted microscope and UV light, the positions of chromatin free demi-oocytes, i.e. cytoplasts were registered. These cytoplasts were later collected under a stereomicroscope and stored in T2 drops before further manipulations.

### Fusion and Initiation of Activation

Porcine fetal fibroblast cells were prepared as described previously (Kragh, et al., 2005, Du, et al., 2005). Fusion was performed in two steps, where the second one included the initiation of activation as well. For the first step, with a finely drawn and fire-polished glass pipette, approximately 100 somatic cells were placed into a T2 drop, and 20-30 cytoplasts were placed into a T10 drop. After a short equilibration, groups of 3 cytoplasts were transferred to 1 mg/ml of phytohaemagglutinin (PHA) for 2-3 sec, then each was quickly dropped over a single somatic cell. Following attachment, cytoplast-somatic cell pairs were picked up again and transferred to a fusion medium (0.3 M mannitol supplemented with 0.01% [w/v] PVA). By using an alternative current (AC) of 0.6 KV/cm and 700 KHz, equilibrated pairs were aligned to one wire of a fusion chamber (BTX microslide 0.5 mm fusion chamber, model 450; BTX, San Diego, Calif.) with the somatic cells farthest from the wire, then fused with a single direct current (DC) impulse of 2.0 KV/cm for 9 μsec. Pairs were then removed carefully from the wire to a T10 drop, and incubated further to observe whether fusion had occurred.

Approximately 1 h after the fusion, fused pairs and the remaining cytoplasts were separately equilibrated in activation medium (0.3 M mannitol, 0.1 mM MgSO₄, 0.1 mM CaCl₂, supplemented with 0.01% [w/v] PVA). By using a 0.6 KV/cm AC, one pair and one cytoplast was aligned to one wire of the fusion chamber, with fused pairs contacting the wire. A single DC pulse of 0.86 KV/cm for 80 μsec was used for the second fusion and initiation of activation. Fusion was checked in after incubation in T10 drops.

### Traditional Cloning (TC)

Micromanipulation was conducted with a Diaphot 200 inverted microscope (Nikon, Tokyo, Japan), as described before (Chen et al., 1999; Zhang et al., 2005). Briefly, after 42-44 h in vitro maturation, the cumulus cells were removed as described above. All manipulations were performed on a heated stage adjusted to 39° C. A single 50 μL micromanipulation solution drop was made in the central area on a lid of 60 mm culture dish and covered with mineral oil. Groups of 20-30 oocytes and fetal fibroblast cells were placed in the same drop. After incubation for 15-30 min, the oocyte was secured with a holding pipette (inner diameter=25-35 μm and outer diameter=80-100 μm). After being placed at the position of 5-6 o′ clock, the first polar body and the adjacent cytoplasm (approx. 10% of the total volume of the oocyte) presumptively containing metaphase plate were aspirated and removed with a beveled injection pipette (inner diameter=20 μm). A fetal fibroblast cell was then injected into the space through the same slit. After nuclear transfer (NT), reconstructed couplets were transferred into drops of media covered with mineral oil for recovery for 1-1.5 h until fusion and activation was conducted. The recovery medium was NCSU-23 supplemented with 4 mg/mL BSA and 7.5 μg/mL CB. Reconstructed couplets were incubated in fusion medium for 4 min. Couplets were aligned manually using a finely pulled and polished glass capillary to make the contact plane parallel to electrodes. A single, 30 μsec, direct current pulse of 2.0 kV/cm was then applied. After culture in drops of IVC0-2 (specified in “Embryo culture and evaluation”) supplemented with 7.5 μg/mL CB for 30-60 min, fusion results were examined under a stereomicroscope. Fused couplets were subjected to a second pulse in activation solution. After 30 min incubation in T10 they were transferred to IVC0-2 to evaluate in vitro development.

### Further Steps of Activation

After the activation impulse, all reconstructed embryos were incubated in IVC0-2 supplemented with 5 μg/ml CB and 10 μg/ml cycloheximide at 38.5° C. in 5% CO₂, 5% O₂, and 90% N₂, with maximum humidity.

### Embryo Culture and Evaluation

4 h later, all reconstructed and activated embryos were washed and cultured in Nunc four-well dishes in 400 μl IVC0-2 covered by mineral oil at 38.5° C. in 5% O₂, 5% O₂, and 90% N₂, with maximum humidity. IVC0-2 was a modified NCSU37 medium (Kikuchi, et al., 1999), containing 4 mg/ml BSA, 0.17 mM sodium pyruvate, and 2.73 mM sodium lactate from Day 0 (the day for activation) to Day 2. Sodium pyruvate and sodium lactate were replaced with 5.5 mM glucose from Day 2 to Day 7 (IVC2-7). All zonae free embryos were cultured in the Well of the Well (WOW) system (Vajta et al., 2000) in the same culture medium and gas mixture as used above, with careful medium change on Day 2 without removing the embryos from the WOWs. TC embryos were cultured in groups of 15 to 30 in wells of four-well dishes by using the same medium amount and composition. Cleavage and blastocyst rates were registered on Day 2 and Day 7, respectively. To determine total cell numbers, blastocysts were fixed and mounted to a glass microscope slide in a small amount (<2 μl) of glycerol containing 10 μg/ml Hoechst 33342. After staining for several hours at room temperature, embryos were observed under a Diaphot 200 inverted microscope with epifluorescent attachment and UV-2A filter (Nikon, Tokyo, Japan).

Comparison of Efficiency of CAHE vs. OHE

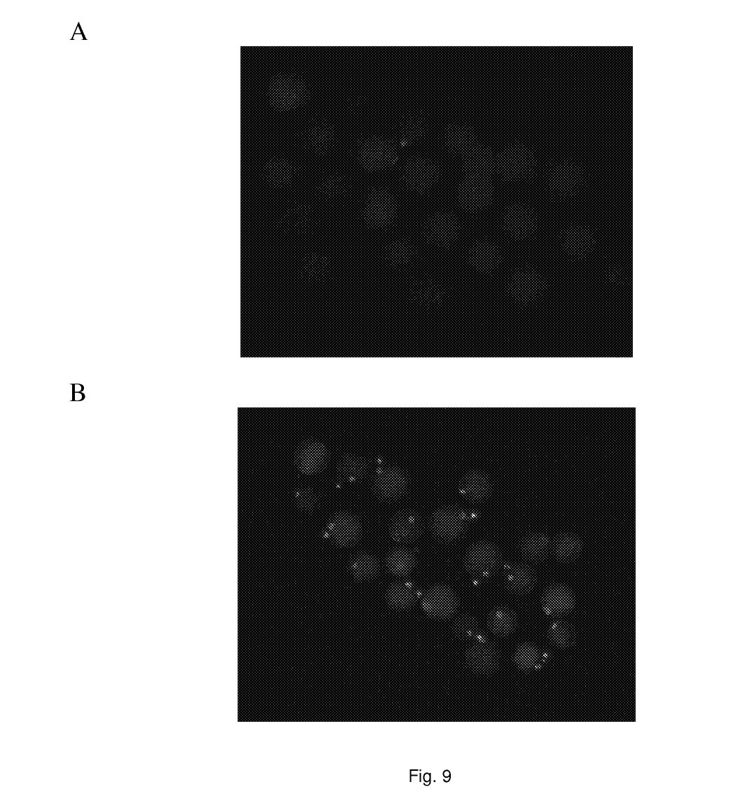

The efficiency and reliability of CAHE was tested in 12 identical replicates by using a total of 620 oocytes. After 41-42 h maturation, oocytes were subjected to demecolcine incubation. Oriented bisection was performed in oocytes where an extrusion cone and/or a strongly attached PB was detected after partial pronase digestion. Percentages of bisected vs. total oocytes and surviving vs. bisected oocytes were registered. Subsequently both putative cytoplasts and karyoplasts were collected separately and stained with Hoechst 33342 (10 μg/ml in T2 for 10 min). The presence or absence of chromatin was detected under an inverted fluorescent microscope (FIG. 9).

The efficiency and reliability of OHE was investigated in 9 identical replicates using a total of 414 oocytes. After 42-43 h in vitro maturation, oriented bisection was performed in matured oocytes where an extrusion cone and/or a PB was detected after partial pronase digestion. Results were evaluated as described in the previous paragraph.

The results are shown in Table 5.

TABLE 5
The efficiency of chemically assisted handmade enucleation
(CAHE) and oriented handmade enucleation (OHE)

| | No. of | Bisected/ | | Cytoplast/ |
|---|---|---|---|---|
| | treated | total | Cytoplast/ | total |
| Groups | oocytes | oocytes (%)* | bisection (%)* | oocyte (%)* |
| CAHE | 620 | 96 ± 1^{a} | 94 ± 2^{b} | 90 ± 3^{c} |
| OHE | 414 | 92 ± 2^{a} | 88 ± 3^{b} | 81 ± 4^{d} |

*mean ± A.D. (absolute deviations)
Different superscripts mean difference (P < 0.05)

No differences between groups regarding extrusion cones and/or attached polar bodies allowing oriented bisection or in the lysis rates were detected, and the successful enucleation per bisected oocyte ratio was also similar. However the overall efficiency of the procedure measured by the cytoplast per total oocyte number was higher in the CAHE than in the OHE group.

Comparison of in vitro development of embryos produced with CAHE, RHE and TC

In 8 replicates, a total of 468 in vitro matured oocytes were randomly distributed and subjected to three of the enucleation procedures described above. Fusion rates between cytoplast and donor fibroblasts were registered. Reconstructed embryos were activated and cultured as described earlier. Cleavage and blastocyst rates were determined on Day 2 and Day 7, respectively. Stereomicroscopic characteristics of the developed blastocysts were compared between groups.

TABLE 6
Developmental competence of embryos derived from chemically assisted
handmade enucleation (CAHE), random handmade enucleation (RHE)
and traditional, micromanipulator based cloning (TC).

| | No. of | | | | Cell no. of |
|---|---|---|---|---|---|
| | reconstructed | Fusion | Cleavage | Blastocyst | blastocysts |
| Groups | embryos | rate (%)* | rate (%)* | rate (%)* | (Day 7) |
| CAHE | 150 | 87 ± 7^{a} | 97 ± 6^{b} | 28 ± 9^{d} | 57 ± 6^{e} |
| RHE | 86 | 81 ± 4^{a} | 87 ± 8^{b} | 21 ± 9^{d} | 49 ± 7^{e} |
| TC | 178 | 81 ± 10^{a} | 69 ± 9^{c} | 21 ± 6^{d} | 53 ± 6^{e} |

*mean ± A.D. (absolute deviations)
Different superscripts mean difference (P < 0.05).

Fusion rates after enucleation were similar between CAHE, RHE and TC, respectively. The second fusion and activation resulted in negligible (<1%) losses in the first two groups. Although TC resulted in lower cleavage per reconstructed embryo rates than the other two groups, this difference was not present in the blastocyst per reconstructed embryo rates.

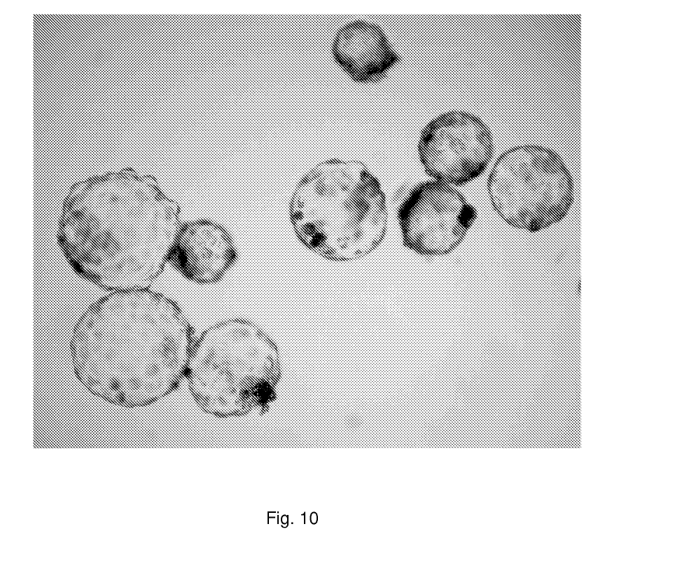

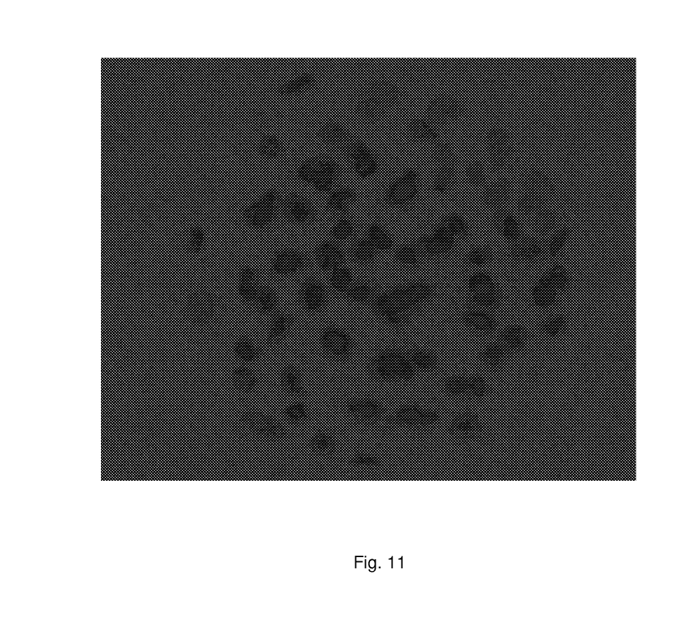

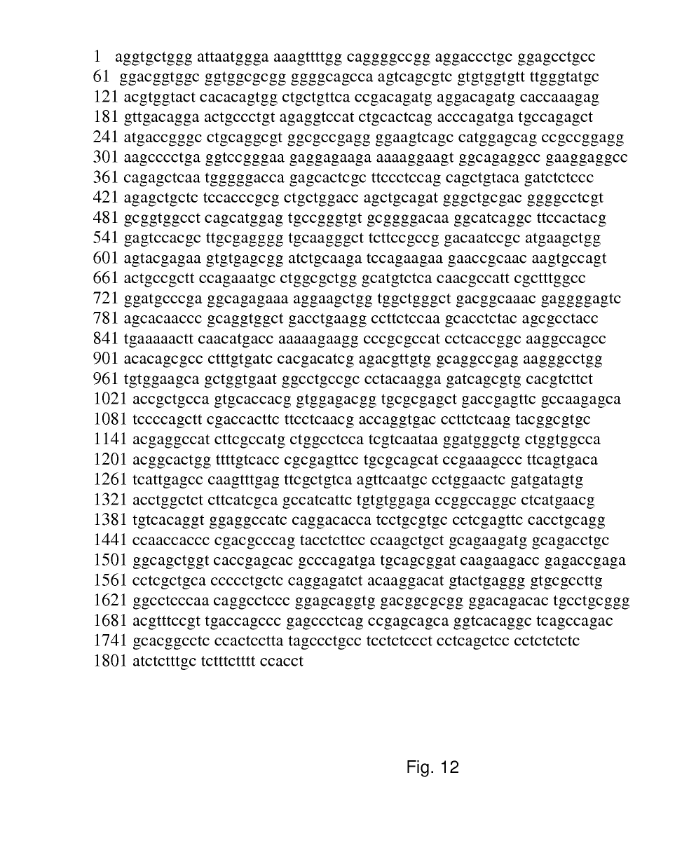

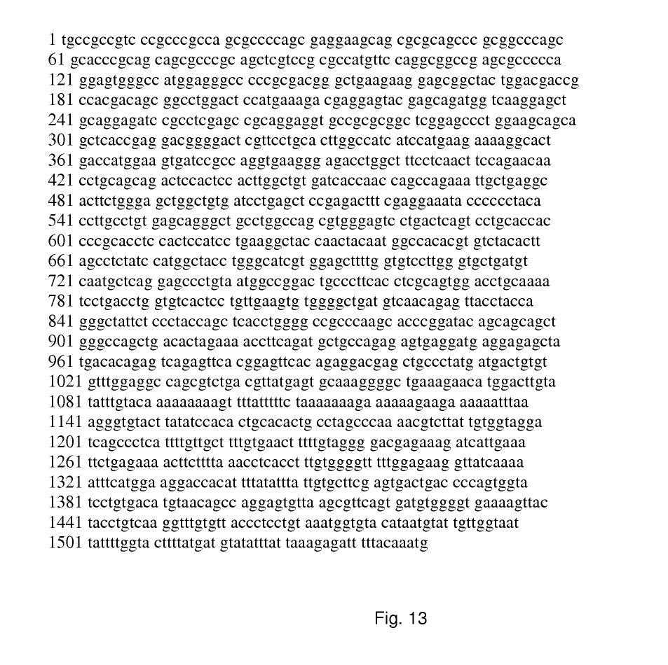

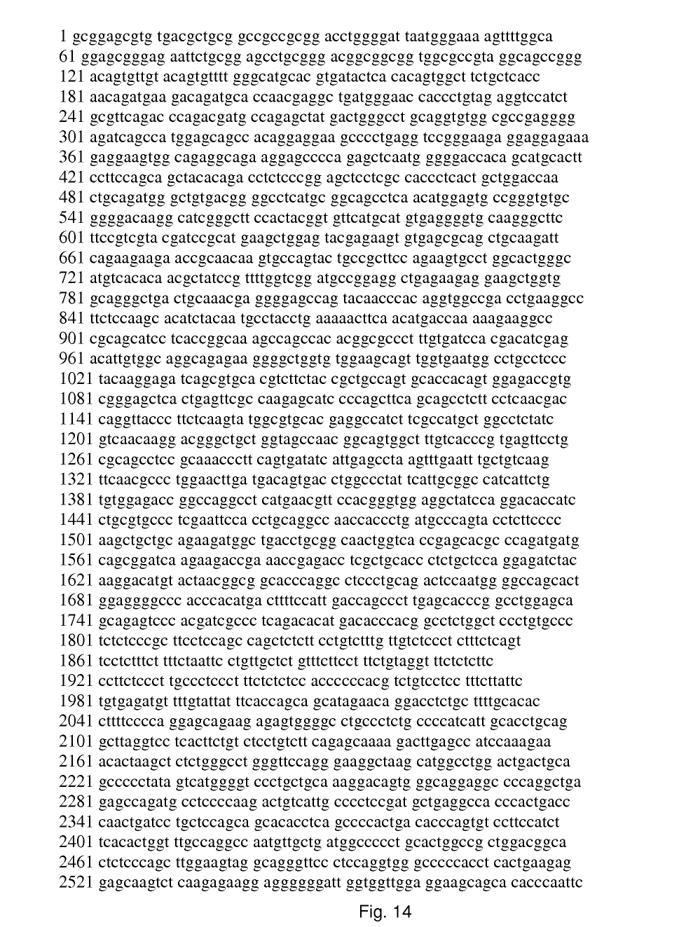

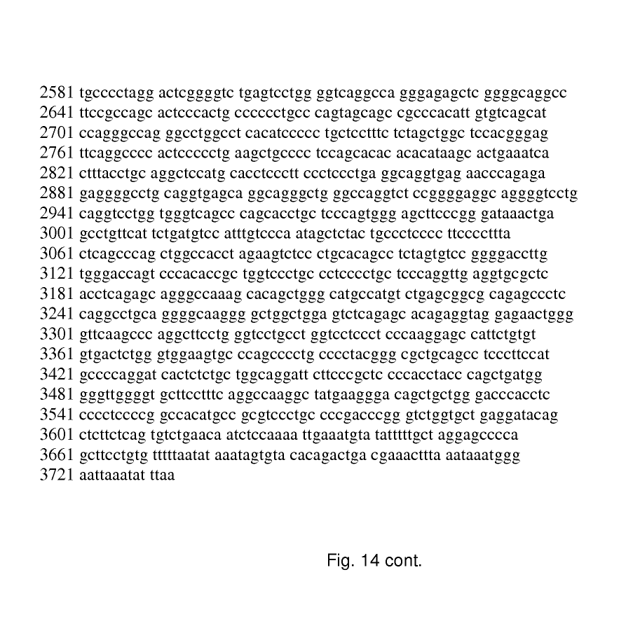

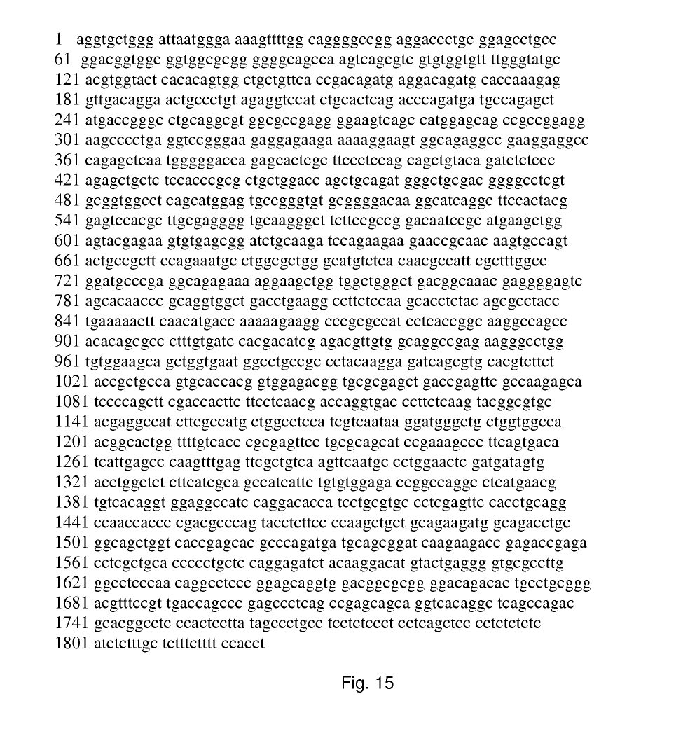

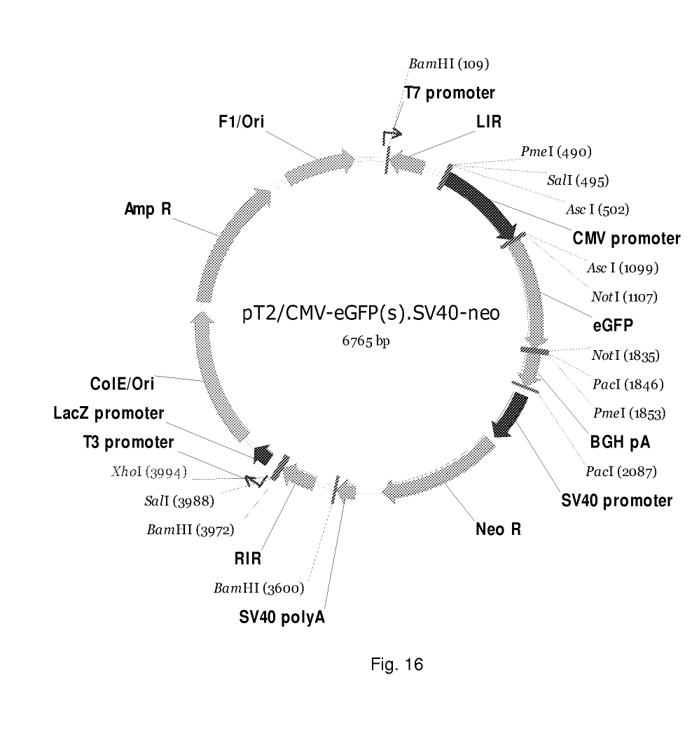

Stereomicroscopic characteristics (size; estimated proportion and outlines of the inner cell mass) did not differ between groups. Cell numbers (57±6 vs. 49±7 vs. 53±6) of the produced blastocysts from CAHE, RHE and TC are shown in Table 6, FIG. 10 and FIG. 11.

### Statistical Analysis

AVEDEV was performed by Microsoft XP Excel software and ANOVA was performed by SAS system. A probability of P<0.05 was considered to be statistically significant.

### Example 5

Handmade cloning (HMC) and establishment of pregnancies for examples 1, 2, 3, 4 and 5.

For the cloning and delivery of transgenic fibroblasts are used in HMC. Recipient sows receive a total of in the range of 60-70 of a mixture of day 5 and/or 6 blastocysts.

Except where otherwise indicated all chemicals were obtained from Sigma Chemical Co. (St Louis, Mo., USA).

### Oocyte Collection and In Vitro Maturation (IVM)

Cumulus-oocyte complexes (COCs) are aspirated from 2 to 6 mm follicles from slaughterhouse-derived sow ovaries and matured in groups of 50 in 400 μl IVM medium consisting of bicarbonate-buffered TCM-199 (GIBCO BRL) supplemented with 10% (v/v) cattle serum (CS), 10% (v/v) pig follicular fluid, 10 IU/ml eCG, 5 IU/ml hCG (Suigonan Vet; Skovlunde, Denmark) at 38.5° C. in 5% CO₂ in humidified air in the Submarine Incubation System (SIS; Vajta et al., 1997) for 41-44 h.

HMC is performed by a procedure based on partial digestion of the zona pellucida, as described earlier (Du et al., 2005 and 2007). Matured COCs are freed from cumulum cells in 1 mg/ml hyaluronidase in Hepes-buffered TCM-199. From this point (except where otherwise indicated) all manipulations are performed on a heated stage adjusted to 39° C., and all drops used for handling oocytes are of 20 μl covered with mineral oil. Zonae pellucidae of are partially digested with 3.3 mg/ml pronase solution dissolved in T33 (T for Hepes-buffered TCM 199 medium; the number means percentage (v:v) of CS supplement, here 33%) for 20 s, then oocytes are washed quickly in T2 and T20 drops. Oocytes with distended and softened zonae pellucidae are lined up in T20 drops supplemented with 2.5 μg/ml cytochalasin B. With a finely drawn glass pipette, oocytes are rotated to locate the polar body on the surface. By oriented bisection with an Ultra Sharp Splitting Blade (AB Technology, Pullman, Wash., USA) less than half of the cytoplasm close to the polar body is removed manually from the remaining putative cytoplast.

Transgenic donor fibroblasts grown to a confluent monolayer in DMEM supplemented with 10% FCS were trypsinized and kept in T20 (Kragh et al., 2004). Fusion is performed in two steps. For the first step, 50% of the available cytoplasts are transferred into 1 mg/ml of phytohemagglutinin (PHA; ICN Pharmaceuticals, Australia) dissolved in TO for 3 s, then each one was quickly dropped over a single transgenic fibroblast. After attachment, cytoplast-fibroblast cell pairs are equilibrated in fusion medium (0.3 M mannitol and 0.01% PVA) for 10 s and transferred to the fusion chamber (BTX microslide 0.5 mm fusion chamber, model 450; BTX, SanDiego, Calif., USA). Using an alternating current (AC) of 0.6 kV/cm and 700 kHz, pairs are aligned to the wire of a fusion chamber with the somatic cells farthest from the wire, then fused with a direct current of 2.0 kV/cm for 9 μs. After the electrical pulse, cell pairs are incubated in T10 drops to observe whether fusion has occurred. Approximately 1 h after the first fusion, each pair is fused with another cytoplast and activated simultaneously in activation medium (0.3 M mannitol, 0.1 mM MgSO₄, 0.1 mM CaCl₂ and 0.01% PVA). By using an AC of 0.6 kV/cm and 700 kHz, one fused pair and one cytoplast was aligned to one wire of the fusion chamber, with fused pairs contacting the wire, followed by a single DC pulse of 0.85 kV/cm for 80 μs. When fusion has been observed in T10 drops, reconstructed embryos are transferred into porcine zygote medium 3 (PZM-3; Yoshioka et al., 2002) supplemented with 5 μg/ml cytochalasin B and 10 μg/ml cycloheximide. After a 4 h incubation at 38.5° C. in 5% CO₂, 5% O₂ and 90% N₂ with maximum humidity, embryos are washed three times in PZM-3 medium before culture

### Embryo Culture and Transfer

Embryos are cultured at 38.5° C. in 5% CO₂, 5% O₂ and 90% N₂ with maximum humidity in PZM-3 medium in the well of well system (WOWs; Vajta et al., 2000). Day 5 and 6 blastocysts with clearly visible inner cell mass are surgically transferred to Danish landrace sows on day 4 or 5 after weaning. Pregnancies are diagnosed by ultrasonography on day 21 and confirmed every second week. Piglets are delivered by Caesarean section on day 114, 24 h after treatment with prostaglandin F2.

### Example 6

### Production of Piglets

### Handmade Cloning (HMC)

Forty one hrs after the start of in vitro maturation, the cumulus investment of the COCs was removed by repeated pipetting in 1 mg/ml hyaluronidase in Hepes-buffered TCM199. From this point (except where otherwise indicated) all manipulations were performed on a heated stage adjusted to 39° C., and all drops used for handling oocytes were of 20 μl volume covered with mineral oil. Oocytes were briefly incubated in 3.3 mg/ml pronase dissolved in T33 (T for Hepes-buffered TCM 199 medium; the number means percentage (v/v) of calf serum (CS) supplement, here 33%) for 20 sec and then quickly washed in T2 and T20 drops. Oocytes with partially digested but still visible zona were lined up in drops of T2 supplemented with 2.5 μg/ml cytochalasin B (CB). With a finely drawn and fire-polished glass pipette, oocytes were rotated to find the polar body (PB) on the surface, and oriented bisection was performed manually under stereomicroscopic control with a microblade (AB Technology, Pullman, Wash., USA). Thus, less than half of the oocyte cytoplasm (close to the extrusion or PB) was removed from the remaining putative cytoplast. Cytoplasts were washed twice in T2 drops and collected in a T10 drop.

Fetal fibroblast cells were prepared as described previously (Kragh, P. M. et al. Theriogenology 64, 1536-1545 (2005).

Fusion was performed in two steps where the second one included the initiation of activation, as well. For the first step, halves of putative cytoplasts were used. With a finely drawn and fire-polished glass pipette, 10 cytoplasts were transferred as a group to 1 mg/ml of phytohaemagglutinin (PHA; ICN Pharmaceuticals, Australia) for 3 sec, then quickly dropped individually onto one of the few fibroblast cells that were sedimented in a T2 drop. After attachment, 10 cytoplast-fibroblast cell pairs were equilibrated in fusion medium (0.3 M mannitol and 0.01% PVA) for 10 sec. Using an alternative current (AC) of 0.6 KV/cm and 700 KHz, cell pairs were aligned to the wire of a fusion chamber (BTX microslide 0.5 mm fusion chamber, model 450; BTX, SanDiego, Calif., USA) with the somatic cells farthest from the wire, then fused with a direct current (DC) of 2.0 KV/cm for 9 μsec. After the electrical pulse, cell pairs were removed carefully from the wire, transferred to T10 drops and incubated to observe whether fusion had occurred.

Approximately 1 hr after the first fusion, fused pairs together with the remaining cytoplasts were equilibrated in activation medium drops separately (0.3 M mannitol, 0.1 mM MgSO₄, 0.1 mM CaCl₂ and 0.01% PVA). Under a 0.6 KV/cm AC, cytoplast—fused pair were aligned sequentially to the wire in groups of 10, with fused pairs far from the wire. A single DC pulse of 0.7 KV/cm for 80 μsec was used for the second fusion and initiation of activation. The pairs were then removed from the wire and transferred carefully to T10 drops to check the fusion. Reconstructed embryos were incubated in PZM-3 medium supplemented with 5 μg/ml CB and 10 μg/ml cycloheximide for 4 hr at 38.5° C. in 5% O₂, 5% O₂ and 90% N₂ with maximum humidity, then washed thoroughly before culture.

### Traditional Cloning (TC)

Micromanipulation was conducted with a Diaphot 200 inverted microscope (Nikon, Tokyo, Japan). Cumulus cells were removed as described above after 42 to 44 hr maturation. All manipulations were performed on a heated stage adjusted to 39□. A single 50 μL drop of micromanipulation solution (NCSU-23 supplemented with 4 mg/mL BSA and 7.5 μg/mL CB) was made in the central area on a lid of 60 mm culture dish and covered with mineral oil. Groups of 20 to 30 oocytes and fetal fibroblast cells were placed in the same drop. After incubation for 15 to 30 min, one oocyte was secured with a holding pipette (inner diameter=25-35 μm and outer diameter=80-100 μm). After being placed at the position of 5-6 o′ clock, the first polar body and the adjacent cytoplasm (approx. 10% of the total volume of the oocyte) presumptively containing metaphase plate were aspirated and removed with a beveled injection pipette (inner diameter=20 μm). A fetal fibroblast cell was then injected into the space through the same slot. After nuclear transfer (NT), reconstructed couplets were transferred into drops of media covered with mineral oil for recovery for 1 to 1.5 hrs until fusion and activation was conducted. Reconstructed couplets were incubated in fusion medium for 4 min. Couplets were aligned manually using a finely pulled and polished glass capillary to make the contact plane parallel to electrodes. A single, 30 μsec, direct current pulse of 2.0 kV/cm was then applied. After culture in drops of PZM-3 medium supplemented with 7.5 μg/mL CB for 30-60 min, fusion results were examined under a stereomicroscope. Fused couplets were subjected to a second pulse in activation solution. After 30 min incubation in T10 they were transferred to PZM-3 medium to evaluate in vitro development.

### Embryo Culture and Transfer

Reconstructed embryos were cultured in PZM-3 medium (Dobrinsky, J. T. et al. Biol Reprod 55, 1069-1074 (1996) supplemented with 4 mg/ml BSA. Zona-free embryos produced from HMC were cultured in the modified WOWs system (Feltrin, C. Et al. Reprod Fertil Dev 18, 126 (2006). Two different cell lines (LW1-2 for HMC, LW2 for TC) were used as nuclear donor cells for HMC and TC to allow the identification of the offspring from the two procedures. LW1-2 and LW2 originate from fetuses from a cross (with Duroc) and pure Danish landrace, respectively.

The average blastocyst per reconstructed embryo rate after in vitro culture for 7 days was 50.1±2.8% (mean±S.E.M), which is significantly higher (p<0.01) for HMC than that of TC performed in parallel in our laboratory (Table 7) and also the highest one that has ever been reported in pig cloning.

TABLE 7
In vitro development of embryos produced from
handmade cloning and traditional cloning

| | | No. of | | |
|---|---|---|---|---|
| | Somatic cell | reconstructed | Cleavage | Blastocyst |
| Group | donor | embryos | rate (%) | rate (%) |
| HMC | LW1-2 | 643 | 83.7 ± 4.90^{a} | 50.06 ± 2.80^{a} |
| TC | LW2 | 831 | 74.86 ± 13.16^{b} | 28.98 ± 2.84^{b} |

^{a,b}Values of different superscripts within columns are significantly different (p < 0.05).
*mean ± S.E.M.

Mixed blastocysts produced from both HMC and TC were surgically transferred to 11 naturally synchronized sows on Day 4 or 5 of estrous cycle. Six (55%) recipients were diagnosed pregnant by ultrasonography, 2 aborted and by the time of writing 2 have delivered 3 and 10 piglets, respectively. A litter size of 10 cloned piglets is, according to our knowledge, the largest litter size so far achieved in pig cloning. All of them are healthy and behave normally except one showed rigid flexure of distal joint of one foreleg. %).

Preliminary results suggest that when embryos of similar stages were transferred, recipients on Day 4 of the estrous cycle supported pregnancy establishment better than those of Day 5 (Table 8).

TABLE 8
In vivo development of cloned porcine embryos

| | Embryos | | | | | No. of piglets born | | |
|---|---|---|---|---|---|---|---|---|
| | transferred | | Embryo | Recipient | | piglets | No. | Gestation |
| Recipient | HMC | TC | stage | cycle | Pregnancy | from | piglets | length |
| number | embryo | embryo | (Day) | (Day) | status | HMC | from TC | (Day) |
| 1327 | 22 | 10 | D 5, 6, 7 | 4 | Y | 2 | 1 | 116 |
| 1539 | 36 | 10 | D 7 | 4 | Y | 8 | 2 | 115 |
| 1309 | 30 | 28 | D 5, 6 | 4 | Y | | | |
| 1553 | 45 | 44 | D 5, 6 | 4 | Y | | | |
| 1668 | 48 | 18 | D 5, 6 | 5 | Y, aborted | | | |
| 1428 | 78 | 22 | D 5, 6 | 5 | Y, aborted | | | |
| 1725 | 44 | 4 | D 5, 6, 7 | 5 | N | — | — | — |
| 1643 | 22 | 11 | D 5, 6, 7 | 4 | N | — | — | — |
| 1520 | 30 | 26 | D 5, 6 | 4 | N | — | — | — |
| 1363 | 37 | 7 | D 6, 7 | 5 | N | — | — | — |
| 1560 | 99 | 42 | D 5, 6, 7 | 5 | N | — | — | — |

### Microsatellite Analysis

Parental analysis using 10 different porcine microsatellite markers confirmed the identical genotype of cloned piglets and donor cells used for nuclear transfer. Identification was done by microsatellite analysis of genomic DNA from each of the newborn piglets, the surrogate sow, and the donor skin fibroblasts LW1-2 and LW2 originating from two fetuses that represent Danish landrace and Duroc, respectively. Ten polymorphic microsatellite loci (SW886, SW58, SW2116, SW1989, SW152, SW378, KS139, S0167, SW1987, SW957) located on different porcine chromosomes were amplified by 3-color multiplex PCR and the products analyzed on the Genetic Analyzer 3130×1 (Applied Biosystems) using the program Gene Mapper 3.7.

For the second recipient, the offspring per embryo rate (22%) was the highest one ever reported so far in pig cloning (Walker, S. C. et al. Cloning Stem Cells 7, 105-112 (2005); Hoshino, Y. et al. Cloning Stem Cells 7, 17-26 (2005)). Comparable live birth/transferred embryo efficiencies were obtained in HMC (17%) and TC (15%).

### Statistical Analysis

Differences between the experimental groups were evaluated using independent-samples t-test by SPSS 11.5. P<0.05 was considered significant.

1. A genetically modified pig as a model for studying psoriasis, wherein the modified pig expresses at least one phenotype associated with psoriasis; and/or
a modified pig comprising at least one mutation in an endogenous ILK-1Ra, JunB/cJun, CD18, IKK2, and/or LIG1 gene or part thereof, transcriptional and/or translational product or part thereof,
and/or
a modified pig comprising at least one human, porcineor murine gene selected from PPARs, PPAR-6, IκB-α, STAT3c, Integrin beta 1, Integrin alpha 2, MEK1, Amphiregulin, BMP-6, VEGF, JunBΔec-JunΔep, IL-1a, TGF.beta 1, CD18 hypo, Cre-lIKK2 fl7fl, Dsg1, SCCE, TGF-a, TNF-a, IL-20, IFN-g, LIG1 KO, KGF, IL-6, PAFR, Cre/Ikk2FL/FL, IL1R, Dsg3, IFN-gamma, p40, IL1Ra, IKK2, JunB/c-Jun, and LIG1 gene or part thereof, transcriptional and/or translational product or part thereof.

2. The genetically modified pig according to claim 1, wherein the pig is a mini-pig.

3. (canceled)

4. (canceled)

5. The genetically modified pig according to claim 1, wherein the pig belongs to the species of S. domesticus.

6. (canceled)

7. (canceled)

8. The genetically modified pig according to claim 1, wherein said human, murine or porcine gene, transcriptional or translational product, or part thereof is expressed from a heterologous promoter.

9. (canceled)

10. The genetically modified pig according to claim 1, wherein said pig is transgenic due to insertion of at least a porcine PPAR δ gene or part thereof, transcriptional and/or translational product or part thereof.

11. The genetically modified pig according to claim 1, wherein said pig is transgenic due to insertion of at least a human IκB-α gene or part thereof, transcriptional and/or translational product or part thereof.

12. The genetically modified pig according to claim 1, wherein said pig is transgenic due to insertion of at least a porcine PPAR δ gene or part thereof, transcriptional or translational product or part thereof, and a human IKB-α gene or part thereof, transcriptional and/or translational product or part thereof.

13. (canceled)

14. (canceled)

15. (canceled)

16. The genetically modified pig according to claim 1, wherein said pig is transgenic due to insertion of at least a porcine PPAR δ cDNA or part thereof, transcriptional or translational product or part thereof, and a human IKB-α cDNA or part thereof, transcriptional and/or translational product or part thereof.

17. (canceled)

18. (canceled)

19. The genetically modified pig according to claim 1, wherein said at least one phenotype is selected from the group consisting of plaque psoriasis, guttate psoriasis, flexural psoriasis, erythrodermic psoriasis, pustular psoriasis and psoriatic arthritis.

20. The genetically modified pig according to claim 1, wherein said at least one phenotype is selected from the group consisting of white scales, skin inflammation, raised skin, red skin, skin shedding, nail changing, yellowish discoloration of nails, and hair loss.

21. (canceled)

22. (canceled)

23. (canceled)

24. (canceled)

25. (canceled)

26. (canceled)

27. (canceled)

28. A genetically modified porcine blastocyst derived from the genetically modified pig as defined in claim 1.

29. A genetically modified porcine embryo derived from the genetically modified pig as defined in claim 1.

30. A genetically modified porcine fetus derived from the genetically modified pig as defined in claim 1.

31. A genetically modified porcine donor cell or cell nucleus derived from the genetically modified pig as defined in claim 1.

32. The genetically modified pig according to claim 1, or a porcine blastocyst, embryo, fetus, or donor cell used to make said modified pig, obtainable by nuclear transfer comprising the steps of
i) establishing at least one oocyte having at least a part of a modified zona pellucida,
ii) separating the oocyte into at least two parts whereby an oocyte having a nucleus and at least one cytoplast are obtained,
iii) establishing a donor cell or membrane surrounded cell nucleus with genetic properties that produce a phenotypic or genetic modification according to claim 1,
iv) fusing said at least one cytoplast with the donor cell or membrane surrounded cell nucleus,
v) obtaining a reconstructed embryo,
vi) activating the reconstructed embryo to form an embryo and culturing said embryo, and
vii) transferring said cultured embryo to a host mammal such that the embryo develops into a genetically modified fetus,
wherein said genetically modified embryo is obtainable by nuclear transfer comprising steps i) to v) and optionally vi),
wherein said genetically modified blastocyst is obtainable by nuclear transfer comprising steps i) to vi) optionally vii), and
wherein said genetically modified fetus is obtainable by nuclear transfer comprising steps i) to vii).

33. A method for producing a transgenic pig, porcine blastocyst, embryo, fetus or donor cell as a model for psoriasis comprising:
i) establishing at least one oocyte
ii) separating the oocyte into at least three parts whereby at least one cytoplast is obtained,
iii) establishing a donor cell or membrane surrounded cell nucleus having genetic properties that produce a phenotypic or genetic modification according to claim 1,
iv) fusing said at least one cytoplast with the donor cell or membrane surrounded cell nucleus,
v) obtaining a reconstructed embryo,
vi) activating the reconstructed embryo to form an embryo and culturing said embryo, and
vii) transferring said cultured embryo to a host mammal such that the embryo develops into a genetically modified fetus,
wherein said transgenic embryo is produced by a process comprising steps i) to v) and optionally vi), wherein said transgenic blastocyst is produced by a process comprising steps i) to vi) and optionally vii), and
wherein said transgenic fetus is produced by a process comprising steps i) to vii).

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. A method for evaluating the effect of a therapeutic treatment of psoriasis, said method comprising the steps of
i) providing the modified pig according to claim 1,
ii) treating said pig with a pharmaceutical composition exerting an effect on said phenotype, and
iii) evaluating the effect observed.

47. (canceled)

48. A method for screening the efficacy of a pharmaceutical composition, said method comprising the steps of
i) providing the modified pig according to claim 1,
ii) expressing in said pig said genetic determinant and exerting said phenotype,
iii) administering to said pig a pharmaceutical composition the efficacy of which is to be evaluated, and
iv) evaluating the effect, if any, of the pharmaceutical composition on the phenotype exerted by the genetic determinant when expressed in the pig.

49. A method for treatment of a human being suffering from psoriasis, said method comprising the initial steps of
i) providing the modified pig according to claim 1,
ii) expressing in said pig said genetic determinant and exerting said phenotype,
iii) administering to said pig a pharmaceutical composition the efficacy of which is to be evaluated, and
iv) evaluating the effect observed, and
v) treating said human being suffering from psoriasis based on the effects observed in the pig.

50. (canceled)

51. (canceled)

52. The genetically modified porcine blastocyst according to claim 28 obtainable by nuclear transfer comprising the steps of
i) establishing at least one oocyte having at least a part of a modified zona pellucida,
ii) separating the oocyte into at least two parts and obtaining an oocyte having a nucleus and at least one cytoplast,
iii) establishing a donor cell or membrane surrounded cell nucleus with genetic properties of the blastocyst of claim 28,
iv) fusing said at least one cytoplast with the donor cell or membrane surrounded cell nucleus
v) obtaining a reconstructed embryo, and
vi) activating the reconstructed embryo to form an embryo and culturing said embryo.

53. The genetically modified porcine embryo according to claim 29 obtainable by nuclear transfer comprising the steps of
i) establishing at least one oocyte having at least a part of a modified zona pellucida,
ii) separating the oocyte into at least two parts and obtaining an oocyte having a nucleus and at least one cytoplast,
iii) establishing a donor cell or membrane surrounded cell nucleus with genetic properties of the embryo of claim 29,
iv) fusing said at least one cytoplast with the donor cell or membrane surrounded cell nucleus,
v) obtaining a reconstructed embryo, and
vi) activating the reconstructed embryo to form an embryo and culturing said embryo.

54. The genetically modified pig fetus according to claim 30 obtainable by nuclear transfer comprising the steps of
i) establishing at least one oocyte having at least a part of a modified zona pellucida,
ii) separating the oocyte into at least two parts and obtaining an oocyte having a nucleus and at least one cytoplast,
iii) establishing a donor cell or membrane surrounded cell nucleus with genetic properties of the fetus according to claim 30,
iv) fusing said at least one cytoplast with the donor cell or membrane surrounded cell nucleus,
v) obtaining a reconstructed embryo,
vi) activating the reconstructed embryo to form an embryo and culturing said embryo, and
vii) transferring said cultured embryo to a host mammal such that the embryo develops into a genetically modified fetus.

55. The genetically modified porcine donor cell according to claim 31 obtainable by nuclear transfer comprising the steps of
i) establishing at least one oocyte having at least a part of a modified zona pellucida,
ii) separating the oocyte into at least two parts and obtaining an oocyte having a nucleus and at least one cytoplast,
iii) establishing a donor cell or membrane surrounded cell nucleus with genetic properties of the donor cell according to claim 31,
iv) fusing said at least one cytoplast with the donor cell or membrane surrounded cell nucleus
v) obtaining a reconstructed embryo, and
vi) activating the reconstructed embryo to form an embryo and culturing said embryo.

56. A genetically modified pig as a model for studying psoriasis, the modified pig expressing at least one phenotype associated with psoriasis;
wherein said modified pig comprises at least one mutation in an endogenous ILK-1Ra, JunB/cJun, CD18, IKK2, or LIG1 gene or part thereof, or a transcriptional or translational product or part thereof;
or wherein said modified pig comprises at least one human, porcine, or murine gene selected from PPARs, PPAR-6, IκB-α, STAT3c, Integrin beta 1, Integrin alpha 2, MEK1, Amphiregulin, BMP-6, VEGF, JunBΔec-JunΔep, IL-1a, TGF.beta 1, CD18 hypo, Cre-lIKK2 fl7fl, Dsg1, SCCE, TGF-a, TNF-a, IL-20, IFN-g, LIG1 KO, KGF, IL-6, PAFR, Cre/Ikk2FL/FL, IL1R, Dsg3, IFN-gamma, p40, IL1Ra, IKK2, JunB/c-Jun, and LIG1, or part thereof, or a transcriptional or translational product or part thereof.
